(19) United States
(12) Patent Application Publication
(10) Pub. No.: US 2026/0015325 A1
(43) Pub. Date: Jan. 15, 2026

(54) ALKOXY HETEROARYL- CARBOXAMIDE OR THIOAMIDE COMPOUNDS

(71) Applicant: SYNGENTA CROP PROTECTION AG, Basel (CH)

(72) Inventors: Ottmar Franz HUETER, Stein (CH); André JEANGUENAT, Stein (CH); Mattia Riccardo MONACO, Stein (CH)

(73) Assignee: SYNGENTA CROP PROTECTION AG, Basel (CH)

(21) Appl. No.: 18/865,029

(22) PCT Filed: May 11, 2023
(86) PCT No.: PCT/EP2023/062656

§ 371 Date: Nov. 12, 2024

(30) Foreign Application Priority Data
May 12, 2022 (EP) 22172906.4

(51) Int. Cl. C07D 213/82; A01N 25/10; A01N 25/12; A01N 43/40; A01N 43/58; A01P 7/04; C07D 213/81; C07D 213/85; C07D 237/24; C07D 239/34; C07D 241/24; C07D 401/12; C07D 401/14; C07D 405/12; C07D 409/12; C07D 413/12; C07D 417/12

(52) U.S. Cl. CPC C07D 213/82; A01N 25/10; A01N 25/12; A01N 43/40; A01N 43/58; A01P 7/04; C07D 213/81; C07D 213/85; C07D 237/24; C07D 239/34; C07D 241/24; C07D 401/12; C07D 401/14; C07D 405/12; C07D 409/12; C07D 413/12; C07D 417/12

(57) ABSTRACT

Compounds of formula (I) wherein the substituents are as defined in claim 1, and the agrochemically acceptable salts, stereoisomers, enantiomers, tautomers and N-oxides of those compounds, can be used as insecticides.

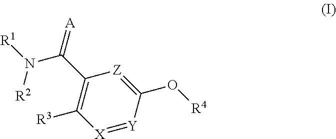

The present invention relates to pesticidally active, in particular insecticidally active alkoxy heteroaryl-carboxamide or thioamide compounds, preferably substituted pyridinyl, pyrazinyl, or pyridazinyl compounds thereof, to processes for their preparation, to compositions comprising the compounds, and to their use for controlling animal pests, including arthropods and in particular insects.

WO2021153720 describes certain alkoxy benzoic acid amide derivatives.

There have now been found certain novel pesticidally active alkoxy and cycloalkoxy heteroaryl-carboxamide or thioamide compounds.

The present invention accordingly relates, in a first aspect, to a compound of the formula (I)

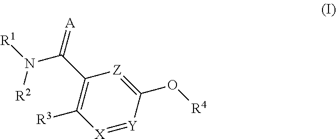

wherein:
A is O, or S;
X is N, or CR⁸;
Y is N or CR⁸;
Z is N or CR⁸;
with the provision that at least one of X, Y, Z is N;
R¹ is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkoxy-C₁-C₆-alkyl, C₃-C₆-cycloalkyl-C₁-C₆-alkyl, C₃-C₆-halocycloalkyl-C₁-C₆-alkyl, cyano-C₁-C₆-alkyl, cyano-C₃-C₆-cycloalkyl, cyano-C₃-C₆-cycloalkyl-C₁-C₆-alkyl, cyano-heterocycloalkyl, hydroxy-C₃-C₆-cycloalkyl, C₁-C₆-alkyl-carbonyloxy-C₃-C₆-cycloalkyl, C₁-C₆-alkylsulfonyloxy-C₃-C₆-cycloalkyl, C₁-C₆-alkoxy-C₃-C₆-cycloalkyl, C₁-C₆-haloalkoxy-C₃-C₆-cycloalkyl, C₁-C₆-alkylthio-C₃-C₆-cycloalkyl, C₁-C₆-haloalkylthio-C₃-C₆-cycloalkyl, C₁-C₆-alkylsulfinyl-C₃-C₆-cycloalkyl, C₁-C₆-haloalkylsulfinyl-C₃-C₆-cycloalkyl, C₁-C₆-alkylsulfonyl-C₃-C₆-cycloalkyl, C₁-C₆-haloalkylsulfonyl-C₃-C₆-cycloalkyl, C₂-C₆-alkynyl-C₃-C₆-cycloalkyl, C₁-C₆-alkoxycarbonyl-C₃-C₆-cycloalkyl, carboxy-C₃-C₆-cycloalkyl, carbamoyl-C₃-C₆-cycloalkyl, C₁-C₆-alkylaminocarbonyl-C₃-C₆-cycloalkyl (which may be mono- or poly-substituted by R⁸), C₄-C₈-bicycloalkyl, C₁-C₆-alkoxyimino, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono- or poly-substituted by R¹⁰), heteroaryl-C₃-C₆-cycloalkyl (the heteroaryl may be mono- or poly-substituted by R¹⁰), heterocycloalkyl (which may be substituted by R⁸), heterocycloalkyl-C₁-C₆-alkyl (which may be substituted by R⁸), or heterocycloalkyl-C₃-C₆-cycloalkyl (the heterocycloalkyl may be substituted by R⁸);

R² is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-alkenyl, C₂-C₆-haloalkenyl, C₂-C₆-alkynyl, C₂-C₆-haloalkynyl, C₁-C₆-alkoxy, C₁-C₆-alkoxy-C₁-C₆-alkyl, C₁-C₆-haloalkoxy, C₃-C₆-cycloalkyl-C₁-C₆-alkyl, C₃-C₆-halocycloalkyl-C₁-C₆-alkyl, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono-substituted or polysubstituted by R¹⁰), cyano, cyano-C₁-C₆-alkyl, cyano-C₃-C₆-cycloalkyl, hydroxy, formyl, C₁-C₆-alkylcarbonyl, C₁-C₆-alkoxycarbonyl, C₃-C₆-cycloalkylcarbonyl, benzoyl that is mono or polysubstituted by R¹⁰, C₁-C₆-haloalkoxycarbonyl, C₂-C₆-alkenyloxycarbonyl, carbamoyl, C₁-C₆-alkylaminocarbonyl (the amino group may be substituted by R⁸), C₁-C₆-alkoxycarbonyl C₁-C₆-alkylamino (the amino group may be substituted by R⁸), C₁-C₆-alkoxycarbonyl amino (the amino group may be substituted by R⁸), (C₁-C₆-alkylthio)carbonyl, (C₁-C₆-alkyl)thiocarbonyl, (C₁-C₆-alkoxy)thiocarbonyl, thiocarbamoyl, C₁-C₆-alkylamino-thiocarbonyl (the amino group may be substituted by R⁸), C₁-C₆-alkylsulfonyl, C₁-C₆-haloalkylsulphonyl, sulfamoyl, C₁-C₆-alkylaminosulfonyl (the amino group may be substituted by R⁸), C₁-C₆-alkoxycarbonyl-C₁-C₆-alkyl, aminocarbonyl-C₁-C₆-alkyl, C₁-C₆-alkylaminocarbonyl-C₁-C₆-alkyl (the amino group may be substituted by R⁸), C₃-C₆-cycloalkylaminocarbonyl-C₁-C₆-alkyl (the amino group may be substituted by R⁸), C₁-C₆-haloalkylaminocarbonyl-C₁-C₆-alkyl (the amino group may be substituted by R⁸), C₁-C₆-alkylcarbonyl-C₁-C₆-alkyl, C₁-C₆-haloalkylcarbonyl-C₁-C₆-alkyl, C₁-C₆-alkylcarbonyloxy-C₁-C₆-alkyl, C₁-C₆-alkoxycarbonyloxy-C₁-C₆-alkyl, C₁-C₆-alkoxycarbonylamino-C₁-C₆-alkyl (the amino group may be substituted by R⁸), hydroxyimino, hydroxyamino-C₁-C₆-alkyl, C₁-C₆-alkoxyimino, C₁-C₆-alkoxyimino-C₁-C₆-alkyl, or C₁-C₆-haloalkoxyimino-C₁-C₆-alkyl;
R³ is halogen, cyano, C₁-C₆-alkyl, C₁-C₆-alkoxy, C₃-C₆-cycloalkyl, C₁-C₆-haloalkyl, or C₁-C₆-haloalkoxy;
R⁴ is

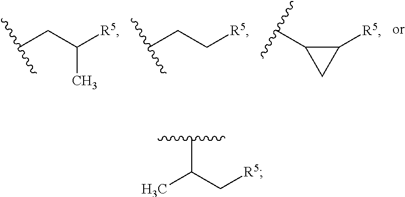

R⁵ is C₁-C₃-alkylsulfonylamino, C₁-C₃-haloalkylsulfonylamino, or C₃-C₆-cycloalkylsulfonylamino, wherein any of said amino-groups is unsubstituted or substituted by R⁶; and wherein any of said cycloalkyl-groups may be mono- or polysubstituted by R⁹;
R⁶ is C₁-C₆-alkyl, C₁-C₆-alkoxy, C₁-C₆-alkoxy-C₁-C₆-alkyl, cyano-C₁-C₆-alkyl, C₁-C₆-haloalkyl, C₃-C₆-cycloalkyl, C₃-C₆-cycloalkyl-C₁-C₆-alkyl, C₁-C₆-alkylcarbonyl, or C₁-C₆-alkoxycarbonyl;
R³ is hydrogen, halogen, C₁-C₃-alkyl, C₁-C₃-alkoxy, C₁-C₃-haloalkyl, or cyano;
R⁹ is halogen, C₁-C₃-alkyl, C₁-C₃-alkoxy, C₁-C₃-haloalkyl, or cyano; and
R¹⁰ is halogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulphonyl, C₁-C₆-alkylthio-C₁-C₆-alkyl, C₁-C₆-haloalkylthio-C₁-C₆-alkyl, C₁-C₆-alkylsulfonyloxy, C₁-C₆-haloalkylsulfonyloxy, phenyl (which may be mono- or poly-substituted independently selected from halogen, C₁-C₆-alkyl and C₁-C₆-haloalkyl), phenyl-C₁-C₆-alkyl, phenyl-C₁-C₆-alkoxy, cyano, or nitro; or
an agrochemically acceptable salt, stereoisomer, enantiomer, tautomer and N-oxide of the compound of formula (I).

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di-ortrihydroxy-lower-alkylamine, for example mono-, di- or triethanolamine.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula (I) according to the invention also include hydrates which may be formed during the salt formation.

The term “C₁-Cₙ-alkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₂-Cₙ-alkenyl” as used herein refers to a straight or branched alkenyl chain moiety having from two to n carbon atoms and one or two double bonds, for example, ethenyl, prop-I-enyl, but-2-enyl.

The term “C₂-Cₙ-alkynyl” as used herein refers to a straight or branched alkynyl chain moiety having from two to n carbon atoms and one triple bond, for example, ethynyl, prop-2-ynyl, but-3-ynyl,

The term “C₃-Cₙ-cycloalkyl” as used herein refers to 3-n membered cycloalkyl radical such as cyclopropyl, cyclobutyl, cyclopentyl and cyclohexyl.

The term “C₁-Cₙ-alkoxy” as used herein refers to a straight-chain or branched saturated alkyl radical having 1 to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy and 1,1-dimethylethoxy. The term “C₂-Cₙ-alkenyloxy” as used herein refers to a straight-chain or branched alkenyl chain having 1 to n carbon atoms (as mentioned above) which is attached via an oxygen atom.

The term “C₁-Cₙ-alkoxy-C₁-Cₙ-alkyl” as used herein refers to an alkyl radical (as mentioned above) substituted with a C₁-Cₙ-alkoxy group. Examples are methoxymethyl, methoxyethyl, ethoxymethyl and propoxymethyl.

The term “C₃-Cₙ-cycloalkyl-C₁-Cₙ-alkyl” as used herein refers to an alkyl radical (as mentioned above) substituted with a C₃-Cₙ-cycloalkyl group. Examples are cyclopropylmethyl, cyclopropylethyl. Similarly, the term “C₃-Cₙ-halocycloalkyl-C₁-Cₙ-alkyl” refers to an alkyl radical substituted with cycloalkyl group, wherein the cycloalkyl group is substituted by one or more of the same or different halogen atoms. Examples are 3,3-difluorobutylmethyl, and 1-chlorocyclopropylmethyl.

The term “heterocycloalkyl” or “heterocyclyl” as used herein refers to a stable 3-, 4-, 5- or 6-membered non-aromatic monocyclic ring radical which comprises 1, 2, or 3 heteroatoms/groups individually selected from nitrogen, oxygen, sulfur, S═O and SO₂. The heterocyclyl radical may be bonded to the rest of the molecule via a carbon atom or heteroatom. Examples of heterocyclyl include, but are not limited to, epoxide, aziridinyl, pyrrolinyl, pyrrolidyl, tetrahydrofuranyl, tetrahydrothienyl, tetrahydrothiopyranyl, piperidyl, piperazinyl, tetrahydropyranyl, dioxolanyl, morpholinyl, oxazinanyl, oxetanyl, 1,1-dioxothietan-3-yl, or δ-lactamyl. The heterocycloalkyl radical may be substituted on the heteroatom and/or carbon atom. The term “cyanoheterocycloalkyl” refers to carbon atom on the heterocycloalkyl radical being substituted by a cyano group.

The term “heterocycloalkyl-C₃-Cₙ-cycloalkyl” as used herein refers to a cycloalkyl radical (as mentioned above) substituted with a heterocycloalkyl group. The heterocycloalkyl-C₃-Cₙ-cycloalkyl group may be substituted on the heterocycloalkyl group and/or cycloalkyl group. Examples are 1-(tetrahydro-3-furanyl)cyclopropyl, 1-(tetrahydro-2-furanyl)cyclopropyl, 1-(tetrahydro-3-thienyl)cyclopropyl, 1-(3-pyrrolidinyl)cyclopropyl, 1-(tetrahydro-2H-pyran-4-yl)cyclopropyl, 1-(tetrahydro-3-furanyl)cyclobutanyl, 1- and (Tetrahydro-3-furanyl)cyclopentanyl.

The term “heterocycloalkyl-C₁-Cₙ-alkyl” as used herein refers to an alkyl radical (as mentioned above) substituted with a heterocycloalkyl group. The heterocycloalkyl-C₁-Cₙ-alkyl radical may be substituted on the heterocycloalkyl group and/or alkyl group. Examples are (tetrahydrofuran-3-yl)methanyl (or (tetra hydrofuran-3-yl)methyl), 1,3-dioxolane-2-methanyl (or 1,3-dioxolane-2-methyl), 2-pyrrolidinemethanyl (or 2-pyrrolidinemethyl), and α-methyl-2-pyrrolidinemethanyl (or α-methyl-2-pyrrolidinemethyl).

The term “cyano-C₁-Cₙ-alkyl” as used herein refers to C₁-Cₙ-alkyl radical having 1 to n carbon atoms (as mentioned above), where one of the hydrogen atoms in the radical is be replaced by a cyano group: for example, cyano-methyl, 2-cyano-ethyl, 2-cyano-propyl, 3-cyano-propyl, 1-(cyano-methyl)-2-ethyl, 1-(methyl)-2-cyano-ethyl, 4-cyanobutyl, and the like. Similarly, the term “cyano-C₃-Cₙ-cycloalkyl” refers to a C₃-Cₙ-cycloalkyl radical substituted with one of the hydrogen atoms by a cyano group; and the term “cyano-C₃-Cₙ-cycloalkyl-C₁-Cₙ-alkyl” refers to an C₁-Cₙ-alkyl radical having a cyano-C₃-Cₙ-cycloalkyl group. Halogen is generally fluorine, chlorine, bromine or iodine. This also applies, correspondingly, to halogen in combination with other meanings, such as haloalkyl, haloalkenyl, haloalkynyl, haloalkoxy, and halocycloalkyl.

The term “C₁-Cₙ-haloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl. Similarly, the term “C₂-Cₙ-haloalkenyl” or “C₂-Cₙ-haloalkynyl” as used herein refers to a C₂-Cₙ-alkenyl or C₂-Cₙ-alkynyl radical respectively substituted with one or more halo atoms which may be the same or different. Similarly, the term “C₃-Cₙ-halocycloalkyl” or “C₁-Cₙ-haloalkoxy” as used herein refers to a C₃-Cₙ-cycloalkyl radical or C₁-Cₙ-alkoxyl radical respectively substituted with one or more halo atoms which may be the same or different.

The term “hydroxy-C₃-Cₙ-cycloalkyl” or “carbamoyl-C₃-Cₙ-cycloalkyl” as used herein refers to a 3-n membered cycloalkyl radical substituted by a hydroxy (OH) group or a carbamoyl (NHC(═O)) group.

The term “C₁-Cₙ-alkyl-carbonyloxy-C₃-Cₙ-cycloalkyl” as used herein refers to a 3-n membered cycloalkyl radical substituted by RₐC(═O)O— moiety wherein Rₐ is a C₁-Cₙ-alkyl group.

The term “carboxy-C₃-Cₙ-cycloalkyl” as used herein refers to a 3-n membered cycloalkyl radical substituted by C(═O)(O)— moiety.

The term “C₁-Cₙ-alkoxycarbonyl-C₃-Cₙ-cycloalkyl” as used herein refers to a 3-n membered cycloalkyl radical substituted by R_{c}C(═O)— moiety wherein R_{c} is the C₁-Cₙ-alkoxy group as defined above. The term “C₁-Cₙ-alkylsulfonyloxy-C₃-Cₙ-cycloalkyl” as used herein refers to a 3-n membered cycloalkyl radical substituted by RₐS(O)₂O— moiety wherein Rₐ is a C₁-Cₙ-alkyl group.

The term “C₁-Cₙ-alkoxy-C₃-Cₙ-cycloalkyl” as used herein refers to a 3-n membered cycloalkyl radical substituted by a C₁-Cₙ-alkoxy group as defined above, Similarly, the term “C₁-Cₙ-haloalkoxy-C₃-Cₙ-cycloalkyl” or “C₂-Cₙ-alkynyl-C₃-Cₙ-cycloalkyl” as used herein refers to a 3-n membered cycloalkyl radical substituted by a C₁-Cₙ-haloalkoxy group or a C₂-Cₙ-alkynyl group respectively as defined above.

The term “C₁-Cₙ-alkylthio” or “C₁-Cₙ-alkylsulfanyl” as used herein refers to a C₁-Cₙ-alkyl group linked through a sulfur atom.

The term “C₁-Cₙ-haloalkylthio” or “C₁-Cₙ-haloalkylsulfanyl” as used herein refers to a C₁-Cₙhaloalkyl group linked through a sulfur atom.

The term “C₁-Cₙ-alkylsulfinyl” as used herein refers to a C₁-Cₙalkyl group linked through the sulfur atom of a sulfinyl (or S(═O)—) group.

The term “C₁-Cₙ-alkylsulfonyl” as used herein refers to a C₁-Cₙalkyl group linked through the sulfur atom of a sulfonyl (or S(═O)₂—) group.

The term “C₁-Cₙ-alkylsulfonyl-C₁-Cₙ-alkyl” as used herein refers to an a C₁-Cₙalkyl radical substituted with a C₁-Cₙalkylsulfonyl group.

The term “C₁-Cₙ-alkylthio-C₃-Cₙ-cycloalkyl” as used herein refers to a 3-n membered cycloalkyl radical substituted by a C₁-Cₙ-akylthio (or RₐS—) moiety wherein Rₐ is a C₁-Cₙ-alkyl group. Similarly, the term “C₁-Cₙ-alkylsulfinyl-C₃-Cₙ-cycloalkyl” or “C₁-Cₙ-alkylsulfonyl-C₃-Cₙ-cycloalkyl” as used herein refers to a 3-n membered cycloalkyl radical substituted by a C₁-Cₙ-akylsulfinyl (or RₐS(O)—) or C₁-Cₙ-akylsulfonyl (or RₐS(O)₂—) moiety respectively wherein Rₐ is a C₁-Cₙ-alkyl group.

The term “C₁-Cₙ-haloalkylthio-C₃-Cₙ-cycloalkyl” as used herein refers to a 3-n membered cycloalkyl radical substituted by a C₁-Cₙ-haloakylthio (or R_{b}S—) moiety wherein R_{b} is a C₁-Cₙ-haloalkyl group. Similarly, the term “C₁-Cₙ-haloalkylsulfinyl-C₃-Cₙ-cycloalkyl” or “C₁-Cₙ-haloalkylsulfonyl-C₃-Cₙ-cycloalkyl” as used herein refers to a 3-n membered cycloalkyl radical substituted by a C₁-Cₙ-haloakylsulfinyl (or R_{b}S(O)—) or C₁-Cₙ-haloakylsulfonyl (or R_{b}S(O)₂—) moiety respectively, wherein R_{b} is C₁-Cₙ-haloalkyl group.

The term “C₁-Cₙ-haloalkylsulfonylamino” as used herein refers to a C₁-Cₙ-haloakylsulfonyl (or R_{b}S(O)₂—) group (wherein R_{b} is the C₁-Cₙ-haloalkyl group as defined above) linked through the nitrogen atom of an amino (or N—H) group.

The term “C₁-Cₙ-alkylcarbonyl” as used herein refers to a C₁-Cₙ-alkyl group linked through the carbon atom of a carbonyl (C═O) group.

The term “C₁-Cₙ-alkoxycarbonyl” as used herein refers to a C₁-Cₙ-alkoxy moiety linked through a carbon atom of a carbonyl (or C═O) group.

The term “C₁-Cₙ-alkoxycarbonyl-C₁-C₆-alkyl” as used herein refers to a C₁-Cₙ-alkyl radical substituted by a “C₁-Cₙ-alkoxycarbonyl group.

The term “benzoyl” as used herein refers to a phenyl group linked through the carbon atom of a carbonyl (C═O) group.

The term “C₁-Cₙ-haloalkoxycarbonyl” as used herein refers to a C₁-Cₙ-haloalkoxy group linked through the carbon atom of a carbonyl (C═O) group.

The term “C₂-Cₙ-alkenyloxycarbonyl” as used herein refers to a C₂-Cₙ-alkenyloxycarbonyl group linked through the carbon atom of a carbonyl (C═O) group.

The term “C₁-Cₙ-alkylaminocarbonyl” as used herein refers to a C₁-Cₙ-alkylamino group (or RₐNHC(═O)—, wherein Rₐ is a C₁-Cₙ-alkyl group) linked through the carbon atom of a carbonyl (C═O) group.

The term “aminocarbonyl-C₁-Cₙ-alkyl” as used herein refers to a C₁-Cₙ-alkyl radical substituted by a aminocarbonyl (or NH₂C(═O)—) group.

The term “C₁-Cₙ-alkylaminocarbonyl-C₁-Cₙ-alkyl” as used herein refers to a C₁-Cₙ-alkyl radical substituted by a C₁-Cₙ-alkylaminocarbonyl (or RₐNHC(═O)—) group, wherein Rₐ is a C₁-Cₙ-alkyl group. The C₁-Cₙ-alkyl group linked to the nitrogen may be substituted.

The term “C₃-Cₙ-cycloalkylaminocarbonyl-C₁-Cₙ-alkyl” as used herein refers to a C₁-Cₙ-alkyl radical substituted by a C₃-Cₙ-cycloalkylaminocarbonyl (or R_{d}NHC(═O)—) group, wherein R_{d} is a C₃-Cₙ-cycloalkyl group. The C₃-Cₙ-cycloalkyl group linked to the nitrogen may be substituted.

The term “C₁-Cₙ-haloalkylaminocarbonyl-C₁-Cₙ-alkyl” as used herein refers to a C₁-Cₙ-alkyl radical substituted by a C₁-Cₙ-haloalkylaminocarbonyl (or R_{b}NHC(═O)—) group, wherein R_{b} is a C₁-Cₙ-haloalkyl group. The C₁-Cₙ-haloalkyl group linked to the nitrogen may be substituted.

The term “C₁-Cₙ-haloalkylcarbonyl-C₁-Cₙ-alkyl” as used herein refers to a C₁-Cₙ-alkyl radical substituted by a C₁-Cₙ-haloalkylcarbonyl (or R_{b}C(═O)—) group, wherein R_{b} is a C₁-Cₙ-haloalkyl group.

The term “C₁-Cₙ-alkylcarbonyloxy-C₁-Cₙ-alkyl” as used herein refers to a C₁-Cₙ-alkyl radical substituted by a C₁-Cₙ-alkylcarbonyloxy (or RₐC(═O)O—) group, wherein Rₐ is a C₁-Cₙ-haloalkyl group.

The term “C₁-Cₙ-alkoxycarbonyloxy-C₁-Cₙ-alkyl” as used herein refers to a C₁-Cₙ-alkyl radical substituted by a C₁-Cₙ-alkoxycarbonyloxy (or R_{c}C(═O)O—) group, wherein R_{c} is a C₁-Cₙ-alkoxy group. The C₁-Cₙ-alkoxy group linked to the nitrogen may be substituted.

The term “C₁-Cₙ-alkylaminocarbonyl-C₃-Cₙ-cycloalkyl” as used herein refers to a C₃-Cₙ-cycloalkyl radical substituted by a C₁-Cₙ-alkylaminocarbonyl (or RₐNHC(═O)— group, wherein Rₐ is a C₁-Cₙ-alkyl group). The C₁-Cₙ-alkylaminocarbonyl-C₃-Cₙ-cycloalkyl radical may be substituted on alkyl group and/or cycloalkyl group.

The term “C₄-Cₙ-bicycloalkyl” as used herein refers to is an annulated non-aromatic bicyclic ring system comprising two rings fused together (i.e., sharing two carbon atoms), and consisting solely of carbon and hydrogen atoms. Examples are bicyclo[3.1.0]hexan-6-yl, bicyclo[4.1.0]heptan-7-yl, bicyclo[3.2.0]heptan-6-yl, bicyclo[3.2.0]heptan-3-yl, octahydro-2-pentalenyl, octahydro-1-pentalenyl.

The term “C₁-Cₙ-alkoxyimino” as used herein refers to an a C₁-Cₙ-alkoxy group linked through the nitrogen atom of an imino (N═) moiety.

The term “hydroxyimino” as used herein refers to an a hydroxy group linked through the nitrogen atom of an imino (N═) moiety.

The term “hydroxyamino-C₁-Cₙ-alkyl” as used herein refers to a C₁-Cₙ-alkyl radical substituted by a hydroxyamino (or (OH)NH—) moiety.

The term “C₁-Cₙ-alkoxyimino-C₁-Cₙ-alkyl” as used herein refers to a C₁-Cₙ-alkyl radical substituted by C₁-Cₙ-alkoxyimino moiety.

The term “C₁-Cₙ-haloalkoxyimino-C₁-Cₙ-alkyl” as used herein refers to a C₁-Cₙ-alkyl radical substituted by C₁-Cₙ-alkoxyimino moiety, wherein the C₁-Cₙ-alkoxy group is substituted by one or more same of different halogen atoms.

The term “phenyl-C₁-Cₙ-alkyl” refers to a C₁-Cₙ-alkyl radical substituted by a phenyl ring. Examples include benzyl. The phenyl-C₁-Cₙ-alkyl radical may be substituted on alkyl group and/or phenyl group.

The term “heteroaryl” refers to a 5- or 6-membered aromatic monocyclic ring radical which comprises 1, 2, 3 or 4 heteroatoms individually selected from N, O and S. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl. The term “heteroaryl-C₁-Cₙ-alkyl” or “heteroaryl-C₃-Cₙ-cycloalkyl” refers to an C₁-Cₙ-alkyl or C₃-Cₙ-cycloalkyl radical respectively substituted by a heteroaryl group. The heteroaryl-C₁-Cₙ-alkyl or heteroaryl-C₃-Cₙ-cycloalkyl radical may be substituted on heteroaryl, alkyl and/or cycloalkyl group as appropriate.

The term “sulfamoyl” as used herein refers to an amino (NH₂) moiety linked through the sulfur atom of an sulfonyl (SO₂) moiety.

The term “C₁-Cₙ-alkylaminosulfonyl” as used herein refers to a C₁-Cₙ-alkylamino (or RₐNHC(═O)—) moiety, wherein Rₐ is a C₁-Cₙ-alkyl group) linked through the sulfur atom of an sulfonyl (SO₂) moiety.

As used herein, the term “may be substituted” means that substituents may or may not carry one or more identical or different substituents, e.g., one, two or three Rx substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not be limited to, CH₂ClO—, CHCl₂O—, CCl₃O—, CH₂FO—, CHF₂O—, CF₃O—, CF₃CH₂O— or CH₃CF₂O— groups. The term “may be substituted” can be used interchangeably ith the term “unsubstituted or substituted”.

As used herein, the term “controlling” refers to reducing the number of pests, eliminating pests and/or preventing further pest damage such that damage to a plant or to a plant derived product is reduced.

As used herein, the term “pest” refers to insects, and molluscs that are found in agriculture, horticulture, forestry, the storage of products of vegetable origin (such as fruit, grain and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

As used herein, the term “room temperature” or “RT” or “rt” refer to a temperature of about 15° C. to about 35° C. For example, rt can refer to a temperature of about 20° C. to about 30° C., rt can refer to a temperature of about 25° C.

An effective amount is readily determined by the skilled person in the art, by the use of known techniques and by observing results obtained under analogous circumstances. In determining the effective amount a number of factors are considered including, but not limited to: the type of plant or derived product to be applied; the pest to be controlled & its lifecycle; the particular compound applied; the type of application; and other relevant circumstances.

In one embodiment of the invention, compound of formula (I)

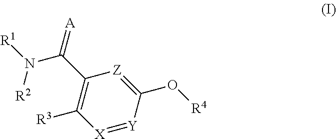

wherein:
A is O, or S;
X is N, or CR⁸;
Y is N or CR⁸;
Z is N or CR⁸;
provided at least one of X, Y, Z is N;
R¹ is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkoxy-C₁-C₆-alkyl, C₃-C₆-cycloalkyl-C₁-C₆-alkyl, C₃-C₆-halocycloalkyl-C₁-C₆-alkyl, cyano-C₁-C₆-alkyl, cyano-C₃-C₆-cycloalkyl, cyano-C₃-C₆-cycloalkyl-C₁-C₆-alkyl, cyano-heterocycloalkyl, hydroxy-C₃-C₆-cycloalkyl, C₁-C₆-alkyl-carbonyloxy-C₃-C₆-cycloalkyl, C₁-C₆-alkylsulfonyloxy-C₃-C₆-cycloalkyl, C₁-C₆-alkoxy-C₃-C₆-cycloalkyl, C₁-C₆-haloalkoxy-C₃-C₆-cycloalkyl, C₁-C₆-alkylthio-C₃-C₆-cycloalkyl, C₁-C₆-haloalkylthio-C₃-C₆-cycloalkyl, C₁-C₆-alkylsulfinyl-C₃-C₆-cycloalkyl, C₁-C₆-haloalkylsulfinyl-C₃-C₆-cycloalkyl, C₁-C₆-alkylsulfonyl-C₃-C₆-cycloalkyl, C₁-C₆-haloalkylsulfonyl-C₃-C₆-cycloalkyl, C₂-C₆-alkynyl-C₃-C₆-cycloalkyl, C₁-C₆-alkoxycarbonyl-C₃-C₆-cycloalkyl, carboxy-C₃-C₆-cycloalkyl, carbamoyl-C₃-C₆-cycloalkyl, C₁-C₆-alkylaminocarbonyl-C₃-C₆-cycloalkyl (which may be mono- or poly-substituted by R⁸), C₄-C₈-bicycloalkyl, C₁-C₆-alkoxyimino, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono- or poly-substituted by R¹⁰), heteroaryl-C₃-C₆-cycloalkyl (the heteroaryl may be mono- or poly-substituted by R¹⁰), heterocycloalkyl (which may be substituted by R⁸), heterocycloalkyl-C₁-C₆-alkyl (which may be substituted by R⁸), or heterocycloalkyl-C₃-C₆-cycloalkyl (the heterocycloalkyl may be substituted by R⁸);
R² is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-alkenyl, C₂-C₆-haloalkenyl, C₂-C₆-alkynyl, C₂-C₆-haloalkynyl, C₁-C₆-alkoxy, C₁-C₆-alkoxy-C₁-C₆-alkyl, C₁-C₆-haloalkoxy, C₃-C₆-cycloalkyl-C₁-C₆-alkyl, C₃-C₆-halocycloalkyl-C₁-C₆-alkyl, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono-substituted or polysubstituted by R¹⁰), cyano, cyano-C₁-C₆-alkyl, cyano-C₃-C₆-cycloalkyl, hydroxy, formyl, C₁-C₆-alkylcarbonyl, C₁-C₆-alkoxycarbonyl, C₃-C₆-cycloalkylcarbonyl, benzoyl that is mono or polysubstituted by R¹⁰, C₁-C₆-haloalkoxycarbonyl, C₂-C₆-alkenyloxycarbonyl, carbamoyl, C₁-C₆-alkylaminocarbonyl (the amino group may be substituted by R⁸), C₁-C₆-alkoxycarbonyl C₁-C₆-alkylamino (the amino group may be substituted by R⁸), C₁-C₆-alkoxycarbonyl amino (the amino group may be substituted by R⁸), (C₁-C₆-alkylthio)carbonyl, (C₁-C₆-alkyl)thiocarbonyl, (C₁-C₆-alkoxy)thiocarbonyl, thiocarbamoyl, C₁-C₆-alkylamino-thiocarbonyl (the amino group may be substituted by R⁸), C₁-C₆-alkylsulfonyl, C₁-C₆-haloalkylsulphonyl, sulfamoyl, C₁-C₆-alkylaminosulfonyl (the amino group may be substituted by R⁸), C₁-C₆-alkoxycarbonyl-C₁-C₆-alkyl, aminocarbonyl-C₁-C₆-alkyl, C₁-C₆-alkylaminocarbonyl-C₁-C₆-alkyl (the amino group may be substituted by R⁸), C₃-C₆-cycloalkylaminocarbonyl-C₁-C₆-alkyl (the amino group may be substituted by R⁸), C₁-C₆-haloalkylaminocarbonyl-C₁-C₆-alkyl (the amino group may be substituted by R⁸), C₁-C₆-alkylcarbonyl-C₁-C₆-alkyl, C₁-C₆-haloalkylcarbonyl-C₁-C₆-alkyl, C₁-C₆-alkylcarbonyloxy-C₁-C₆-alkyl, C₁-C₆-alkoxycarbonyloxy-C₁-C₆-alkyl, C₁-C₆-alkoxycarbonylamino-C₁-C₆-alkyl (the amino group may be substituted by R⁸), hydroxyimino, hydroxyamino-C₁-C₆-alkyl, C₁-C₆-alkoxyimino, C₁-C₆-alkoxyimino-C₁-C₆-alkyl, or C₁-C₆-haloalkoxyimino-C₁-C₆-alkyl;
R³ is halogen, cyano, C₁-C₆-alkyl, C₁-C₆-alkoxy, C₃-C₆-cycloalkyl, C₁-C₆-haloalkyl, or C₁-C₆-haloalkoxy;
R⁴ is C₁-C₆-alkyl that is mono- or polysubstituted by R⁵, or C₃-C₆-cycloalkyl that is mono- or polysubstituted by R⁵;
R⁵ is independently selected from halogen, C₁-C₆-alkyl, C₃-C₆-cycloalkyl, C₁-C₆-alkoxy, C₃-C₆-cycloalkoxy, C₁-C₆-haloalkyl, C₃-C₆-halocycloalkyl, C₁-C₆-haloalkoxy, C₃-C₆-halocycloalkoxy, C₁-C₆-alkylthio, C₁-C₆-haloalkylthio, C₃-C₆-cycloalkylthio, C₁-C₆-alkylsulfonylamino (the amino group may be substituted by R⁶), C₁-C₆-haloalkylsulfonylamino (the amino group may be substituted by R⁶), and C₃-C₆-cycloalkylsulfonylamino that may be mono or polysubstituted by R⁸ (the amino group may be substituted by R⁶);
R⁶ is independently selected from C₁-C₆-alkyl, C₁-C₆-alkoxy, C₁-C₆-alkoxy-C₁-C₆-alkyl, cyano-C₁-C₆-alkyl, C₁-C₆-haloalkyl, C₃-C₆-cycloalkyl, C₃-C₆-cycloalkyl-C₁-C₆-alkyl, C₁-C₆-alkylcarbonyl, and C₁-C₆-alkoxycarbonyl;
R⁸ is independently selected from hydrogen, halogen, C₁-C₃-alkyl, C₁-C₃-alkoxy, C₁-C₃-haloalkyl, and cyano;
R⁹ is independently selected from halogen, C₁-C₃-alkyl, C₁-C₃-alkoxy, C₁-C₃-haloalkyl, and cyano; and
R¹⁰ is independently selected from halogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulphonyl, C₁-C₆-alkylthio-C₁-C₆-alkyl, C₁-C₆-haloalkylthio-C₁-C₆-alkyl, C₁-C₆-alkylsulfonyloxy, C₁-C₆-haloalkylsulfonyloxy, phenyl (which may be mono- or poly-substituted independently selected from halogen, C₁-C₆-alkyl and C₁-C₆-haloalkyl), phenyl-C₁-C₆-alkyl, phenyl-C₁-C₆-alkoxy, cyano, and nitro; or an agrochemically acceptable salt, stereoisomer, enantiomer, tautomer and N-oxide of the compound of formula (I).

In an embodiment, a compound of formula (I) is represented by the formula (I-1), (I-2), (I-3), or (I-4), where A, R¹, R², R³ and R⁴ are as defined in the first aspect.

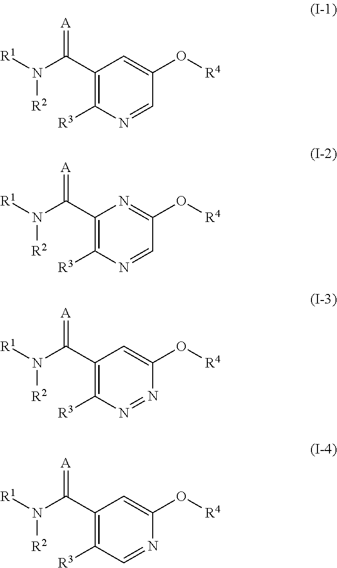

In an embodiment, a compound of formula (I) is represented by the formula (I-1a), (I-2a), (I-3a), or (I-4a), where R¹, R², R³ and R⁶ are as defined in the first aspect; and R⁷ is, independent of (I-1a), (I-2a), (I-3a), and (I-4a), selected from C₁-C₆-alkyl, C₁-C₆-haloalkyl, and C₃-C₆-cycloalkyl that may be mono or polysubstituted by R⁹, where the methyl group connected to the carbon designated by a star (*) can be can be in the form of (S) or the (R) enantiomer; or a mixture thereof.

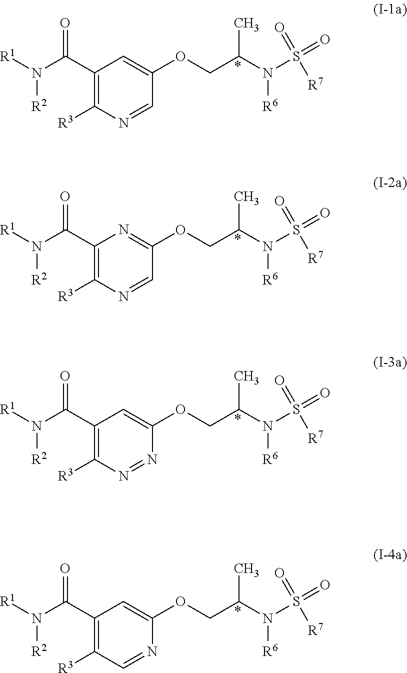

In an embodiment, the compound as represented by the formulae (I-1a), (I-2a), (I-3a), and (I-4a), is an (S) enantiomer.

Embodiments according to the invention are provided as set out below.

In an embodiment of each aspect of the invention, A is
A. oxygen; or
B. sulfur.

In an embodiment of each aspect of the invention,
A. one or two of X, Y and Z is N, and the rest CR⁸; or
B. X is N, and Z and Y are each CR⁸; or
C. X and Z are N, and Y is CR⁸; or
D. X and Y are N, and Z is CR⁸; or
E. Y is N, and X and Z are each CR⁸
F. X is N, and Z and Y are each CH; or
G. X and Z are N, and Y is CH; or
H. X and Y are N, and Z is CH or
I. Y is N, and X and Z are each CH.

In an embodiment of each aspect of the invention, R¹ is
A. hydrogen, C₁-C₆-alkyl, C₁-C₆-haloalkyl, C₂-C₆-alkynyl, C₃-C₆-cycloalkyl, cyano-C₃-C₆-cycloalkyl, C₃-C₆-halocycloalkyl, C₃-C₆-cycloalkyl-C₁-C₆-alkyl, heterocyclyl, or C₁-C₆-alkoxy-C₃-C₆-cycloalkyl; or
B. hydrogen, C₁-C₃-alkyl, C₁-C₃-haloalkyl, C₂-C₄-alkynyl, C₃-C₄-cycloalkyl, cyano-C₃-C₄-cycloalkyl, C₃-C₄-halocycloalkyl, C₃-C₄-cycloalkyl-C₁-C₃-alkyl, 4- or 5-membered heterocyclyl, or C₁-C₃-alkoxy-C₃-C₄-cycloalkyl; or
C. C₁-C₆-alkyl, C₁-C₆-haloalkyl, C₂-C₆-alkynyl, C₃-C₆-cycloalkyl, C₃-C₆-halocycloalkyl, C₃-C₆-cycloalkyl-C₁-C₆-alkyl, 4-membered heterocyclyl, or cyano-C₃-C₆-cycloalkyl; or
D. C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl having a SO₂, or cyano-C₃-C₄-cycloalkyl; or
E. ethyl, isopropyl, propargyl, 2,2,2-trifluoroethyl, 2,2-difluoroethyl, cyclopropyl, 1-cyanocyclopropyl, 1-cyanocyclobutyl, 3,3-difluorocyclobutyl, 1,1-dioxothietan-3-yl, or cyclopropylmethyl.

In an embodiment of each aspect of the invention, R² is
A. hydrogen, C₁-C₆-alkyl, C₁-C₆-haloalkyl, C₃-C₆-cycloalkyl, C₃-C₆-cycloalkyl-C₁-C₆-alkyl, C₁-C₆-alkoxy-C₃-C₆-cycloalkyl, C₁-C₆-alkoxy-C₁-C₆-alkyl, or C₁-C₆-alkylcarbonyl; or
B. hydrogen, C₁-C₃-alkyl, C₁-C₃-haloalkyl, C₃-C₄-cycloalkyl, C₃-C₄-cycloalkyl-C₁-C₃-alkyl, C₁-C₃-alkoxy-C₃-C₄-cycloalkyl, C₁-C₃-alkoxy-C₁-C₃-alkyl, or C₁-C₃-alkylcarbonyl; or
C. hydrogen, C₁-C₆-alkyl, C₁-C₆-alkylcarbonyl, or C₁-C₆-alkoxycarbonyl; or
D. hydrogen, C₁-C₃-alkyl, C₁-C₃-alkylcarbonyl, or C₁-C₃-alkoxycarbonyl; or
E. hydrogen, methyl, acetyl or 2-methyl propanoyl.

In an embodiment of each aspect of the invention, R³ is
A. halogen, C₁-C₆-alkyl, or cyano; or
B. halogen, C₁-C₃-alkyl, or cyano; or
C. chlorine, methyl, or cyano; or
D. chlorine or bromine; or
E. chlorine.

In an embodiment of each aspect of the invention, R⁴ is

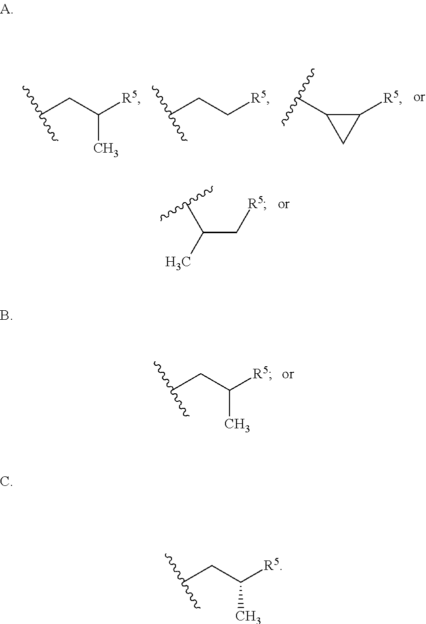

In an embodiment of each aspect of the invention, R⁵ is
A. C₁-C₆-alkylsulfonylamino (the amino group may be substituted by R⁶), C₁-C₆-haloalkylsulfonylamino (the amino group may be substituted by R⁶), and C₃-C₆-cycloalkylsulfonylamino that may be mono or polysubstituted by R⁹ (the amino group may be substituted by R⁶); or
B. C₁-C₃-alkylsulfonylamino (the amino group may be substituted by R⁶), C₁-C₃-haloalkylsulfonylamino (the amino group may be substituted by R⁶), or C₃-C₆-cycloalkylsulfonylamino that may be mono or polysubstituted by R⁹ (the amino group may be substituted by R⁶); or
C. isopropylsulfonylamino, difluoromethylsulfonylamino, trifluoromethylsulfonylamino, difluoromethylsulfonylamino, dichloromethylsulfonylamino, or cyclopropylsulfonylamino; or
D. isopropylsulfonylamino, difluoromethylsulfonylamino, trifluoromethylsulfonylamino, difluoromethylsulfonylamino, or cyclopropylsulfonylamino; or
E. difluromethylsulfonylamino, or trifluoromethylsulfonylamino.

In an embodiment of each aspect of the invention, R⁶ is
A. hydrogen, C₁-C₆-alkyl, C₁-C₆-alkoxy-C₁-C₆-alkyl, C₃-C₆-cycloalkyl-C₁-C₆-alkyl, or C₁-C₆-alkylcarbonyl; or
B. hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl; or
C. hydrogen, methyl, methoxymethyl, cyclopropylmethyl, or acetyl.

In an embodiment of each aspect of the invention, R⁷ is
A. C₁-C₃-alkyl, C₁-C₃-haloalkyl, or C₃-C₄-cycloalkyl that may be mono or polysubstituted by R⁹, or
B. methyl, ethyl, iso-propyl, trifluoromethyl, difluoromethyl, or cyclopropyl; or
C. methyl, ethyl, iso-propyl, trifluoromethyl, difluoromethyl, dichloromethyl, or cyclopropyl; or
D. iso-propyl, trifluoromethyl, difluoromethyl, or cyclopropyl.

In an embodiment of each aspect of the invention, R³ is
A. hydrogen, halogen, C₁-C₃-alkyl, C₁-C₃-alkoxy, C₁-C₃-haloalkyl, or cyano; or
B. hydrogen, chloro, bromo, fluoro, methyl, ethyl, iso-propyl, methoxy, trifluoromethyl, diifluoromethyl, 2,2,2-trifluoroethyl, or cyano; or
C. hydrogen.

In an embodiment of each aspect of the invention, R⁹ is
A. halogen, C₁-C₃-alkyl, C₁-C₃-alkoxy, C₁-C₃-haloalkyl, or cyano; or
B. chloro, bromo, fluoro, methyl, ethyl, iso-propyl, methoxy, trifluoromethyl, diifluoromethyl, 2,2,2-trifluoroethyl, or cyano.

In an embodiment of each aspect of the invention, R¹⁰ is
A. halogen, C₁-C₃-alkyl, C₁-C₃-haloalkyl, C₃-C₄-cycloalkyl, C₃-C₄-halocycloalkyl, C₃-C₄-cycloalkyl-C₁-C₃-alkyl, C₃-C₄-halocycloalkyl-C₁-C₃-alkyl, C₁-C₃-alkoxy, C₁-C₃-haloalkoxy, C₁-C₃-alkylthio, C₁-C₃-haloalkylthio, C₁-C₃-alkylsulfinyl, C₁-C₃-haloalkylsulfinyl, C₁-C₃-alkylsulfonyl, C₁-C₃-haloalkylsulphonyl, C₁-C₃-alkylthio-C₁-C₃-alkyl, C₁-C₃-haloalkylthio-C₁-C₃-alkyl, C₁-C₃-alkylsulfonyloxy, C₁-C₃-haloalkylsulfonyloxy, phenyl (which may be mono- or poly-substituted independently selected from halogen, C₁-C₆-alkyl and C₁-C₆-haloalkyl), phenyl-C₁-C₃-alkyl, phenyl-C₁-C₃-alkoxy, cyano, or nitro; or
B. chloro, bromo, fluoro, methyl, ethyl, iso-propyl, methoxy, trifluoromethyl, diifluoromethyl, 2,2,2-trifluoroethyl, or cyano.

The present invention, accordingly, makes available a compound of formula (I) having A, X, Y, Z, R¹, R², R³, R⁴, R⁵, R⁶, R⁸, R⁹, and R¹⁰ as defined above in all combinations/each permutation.

Accordingly, made available, for example, is a compound of formula (I) with A being embodiment A (i.e A is oxygen); X, Y and Z being embodiment A (i.e one or two of X, Y and Z is N, and the rest CR⁸); R¹ being embodiment B (i.e. R¹ is hydrogen, C₁-C₃-alkyl, C₁-C₃-haloalkyl, C₂-C₄-alkynyl, C₃-C₄-cycloalkyl, cyano-C₃-C₄-cycloalkyl, C₃-C₄-halocycloalkyl, C₃-C₄-cycloalkyl-C₁-C₃-alkyl, 4- or 5-membered heterocyclyl, or C₁-C₃-alkoxy-C₃-C₄-cycloalkyl); R² being embodiment C (i.e. R² is hydrogen, C₁-C₆-alkyl, C₁-C₆-alkylcarbonyl, or C₁-C₆-alkoxycarbonyl); R³ being embodiment B (i.e. R³ is halogen, C₁-C₃-alkyl, or cyano); R⁴ being of the first aspect (i.e. R⁴ is C₁-C₆-alkyl that is mono- or polysubstituted by R⁵, or C₃-C₆-cycloalkyl that is mono- or polysubstituted by R⁵); R⁵ being embodiment B (i.e. R⁵ is C₁-C₃-alkylsulfonylamino (the amino group may be substituted by R⁶), C₁-C₃-haloalkylsulfonylamino (the amino group may be substituted by R⁶), and C₃-C₆-cycloalkylsulfonylamino that may be mono or polysubstituted by R⁹ (the amino group may be substituted by R⁶); R⁶ being embodiment B (i.e. R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl); and R⁹ being of the first aspect (i.e. R⁹ is halogen, C₁-C₃-alkyl, C₁-C₃-alkoxy, C₁-C₃-haloalkyl, or cyano).

Preferably a compound of formula (I) wherein
A being embodiment A (i.e A is oxygen);
X, Y and Z being embodiment A (i.e one or two of X, Y and Z is N, and the rest CR⁸);
R¹ being embodiment B (i.e. R¹ is hydrogen, C₁-C₃-alkyl, C₁-C₃-haloalkyl, C₂-C₄-alkynyl, C₃-C₄-cycloalkyl, cyano-C₃-C₄-cycloalkyl, C₃-C₄-halocycloalkyl, C₃-C₄-cycloalkyl-C₁-C₃-alkyl, 4- or 5-membered heterocyclyl, or C₁-C₃-alkoxy-C₃-C₄-cycloalkyl);
R² being embodiment C (i.e. R² is hydrogen, C₁-C₆-alkyl, C₁-C₆-alkylcarbonyl, or C₁-C₆-alkoxycarbonyl);
R³ being embodiment B (i.e. R³ is halogen, C₁-C₃-alkyl, or cyano);

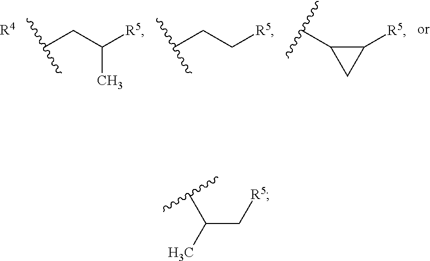

R⁵ being embodiment C (i.e. R⁵ is C₁-C₃-alkylsulfonylamino (the amino group may be substituted by R⁶), C₁-C₃-haloalkylsulfonylamino (the amino group may be substituted by R⁶), and C₃-C₆-cycloalkylsulfonylamino that may be mono or polysubstituted by R⁹ (the amino group may be substituted by R⁶);
R⁶ being embodiment B (i.e. R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl); and
R⁹ being of the first aspect (i.e. R⁹ is (i.e. R⁹ is halogen, C₁-C₃-alkyl, C₁-C₃-alkoxy, C₁-C₃-haloalkyl, or cyano).

In an embodiment of each aspect of the invention, the compound of the formula (I-1), (I-2), (I-3), or (I-4) wherein
R¹ is hydrogen, C₁-C₆-alkyl, C₁-C₆-haloalkyl, C₂-C₆-alkynyl, C₃-C₆-cycloalkyl, cyano-C₃-C₆-cycloalkyl, C₃-C₆-halocycloalkyl, C₃-C₆-cycloalkyl-C₁-C₆-alkyl, heterocyclyl, or C₁-C₆-alkoxy-C₃-C₆-cycloalkyl;
R² is hydrogen, C₁-C₆-alkyl, C₁-C₆-haloalkyl, C₃-C₆-cycloalkyl, C₃-C₆-cycloalkyl-C₁-C₆-alkyl, C₁-C₆-alkoxy-C₃-C₆-cycloalkyl, C₁-C₆-alkoxy-C₁-C₆-alkyl, or C₁-C₆-alkylcarbon;
R³ is halogen, C₁-C₆-alkyl, or cyano;
R⁴ is

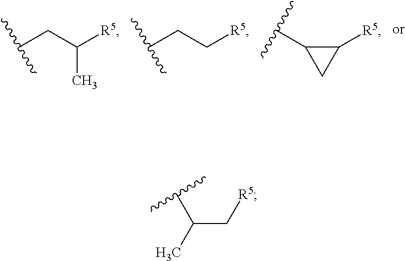

R⁵ is C₁-C₃-alkylsulfonylamino (the amino group may be substituted by R⁶), C₁-C₃-alkylsulfonylamino (the amino group may be substituted by R⁶), and C₃-C₆-cycloalkylsulfonylamino that may be mono or polysubstituted by R⁹ (the amino group may be substituted by R⁶).

R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl; and
R⁹ is halogen, C₁-C₃-alkyl, C₁-C₃-alkoxy, C₁-C₃-haloalkyl, or cyano.

In an embodiment of each aspect of the invention, the compound of the formula (I-1), (I-2), (I-3), or (I-4) wherein
R¹ is C₁-C₆-alkyl, C₁-C₆-haloalkyl, C₂-C₆-alkynyl, C₃-C₆-cycloalkyl, C₃-C₆-halocycloalkyl, C₃-C₆-cycloalkyl-C₁-C₆-alkyl, 4-membered heterocyclyl, or cyano-C₃-C₆-cycloalkyl;
R² is hydrogen, C₁-C₆-alkyl, C₁-C₆-alkylcarbonyl, or C₁-C₆-alkoxycarbonyl;
R³ is halogen, C₁-C₃-alkyl, or cyano;
R⁴ is

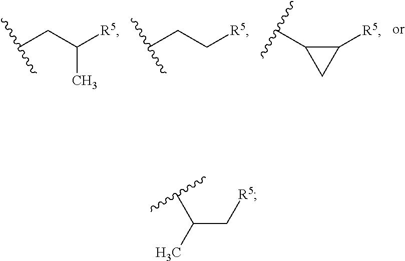

R⁵ is C₁-C₃-alkylsulfonylamino (the amino group may be substituted by R⁶), C₁-C₃-haloalkylsulfonylamino (the amino group may be substituted by R⁶), and C₃-C₆-cycloalkylsulfonylamino that may be mono or polysubstituted by R⁹ (the amino group may be substituted by R⁶).

R⁶ is hydrogen, C₁-C₃-alkyl, C₁-C₆-alkoxy-C₁-C₃-alkyl, C₃-C₄-cycloalkyl-C₁-C₄-alkyl, or C₁-C₃-alkylcarbonyl; and
R⁹ is halogen, C₁-C₃-alkyl, C₁-C₃-alkoxy, C₁-C₃-haloalkyl, or cyano.

In an embodiment of each aspect of the invention, the compound of the formula (I-1), (I-2), (I-3), or (I-4) wherein
R¹ is ethyl, isopropyl, propargyl, 2,2,2-trifluoroethyl, 2,2-difluoroethyl, cyclopropyl, 1-cyanocyclopropyl, 1-cyanocyclobutyl, 3,3-difluorocyclobutyl, 1,1-dioxothietan-3-yl, or cyclopropylmethyl;
R² is hydrogen, methyl, acetyl or 2-methyl propanoyl;
R³ is chlorine, methyl, or cyano;
R⁴ is

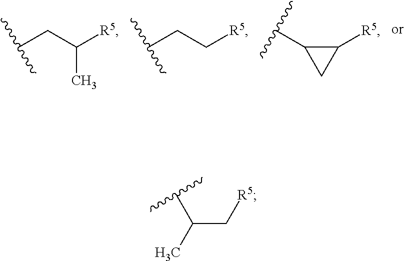

R⁵ is C₁-C₃-alkylsulfonylamino (the amino group may be substituted by R⁶), C₁-C₃-haloalkylsulfonylamino (the amino group may be substituted by R⁶), and C₃-C₆-cycloalkylsulfonylamino that may be mono or polysubstituted by R⁹ (the amino group may be substituted by R⁶);
R⁶ is hydrogen, methyl, methoxymethyl, cyclopropylmethyl, or acetyl; and
R⁹ is halogen, C₁-C₃-alkyl, C₁-C₃-alkoxy, C₁-C₃-haloalkyl, or cyano.

In an embodiment of each aspect of the invention, the compound of the formula (I-1), (I-2), (I-3) or (I-4) wherein
R¹ is ethyl, isopropyl, propargyl, 2,2,2-trifluoroethyl, 2,2-difluoroethyl, cyclopropyl, 1-cyanocyclopropyl, 1-cyanocyclobutyl, 3,3-difluorocyclobutyl, 1,1-dioxothietan-3-yl, or cyclopropylmethyl;
R² is hydrogen, methyl, acetyl or 2-methyl propanoyl;
R³ is chlorine, bromine or methyl;
R⁴ is

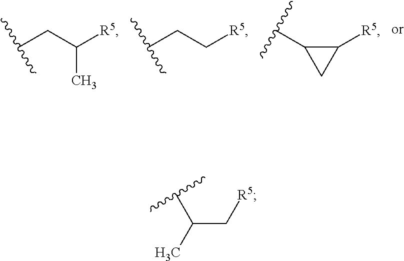

R⁵ is C₁-C₃-alkylsulfonylamino (the amino group may be substituted by R⁶), C₁-C₃-haloalkylsulfonylamino (the amino group may be substituted by R⁶), and C₃-C₆-cycloalkylsulfonylamino that may be mono or polysubstituted by R⁹ (the amino group may be substituted by R⁶);
R⁶ is hydrogen, methyl, methoxymethyl, cyclopropylmethyl, or acetyl; and
R⁹ is halogen, C₁-C₃-alkyl, C₁-C₃-alkoxy, C₁-C₃-haloalkyl, or cyano.

In an embodiment of each aspect of the invention, the compound of the formula (I-1a), (I-2a), (I-3a) or (I-4a) has as R¹ hydrogen, C₁-C₆-alkyl, C₁-C₆-haloalkyl, C₂-C₆-alkynyl, C₃-C₆-cycloalkyl, cyano-C₃-C₆-cycloalkyl, C₃-C₆-halocycloalkyl, C₃-C₆-cycloalkyl-C₁-C₆-alkyl, C₁-C₆-alkyl, heterocyclyl, or C₁-C₆-alkoxy-C₃-C₆-cycloalkyl; as R² hydrogen, C₁-C₆-alkyl, C₁-C₆-haloalkyl, C₃-C₆-cycloalkyl, C₃-C₆-cycloalkyl-C₁-C₆-alkyl, C₁-C₆-alkoxy-C₃-C₆-cycloalkyl, C₁-C₆-alkoxy-C₁-C₆-alkyl, or C₁-C₆-alkylcarbon; as R³ halogen, C₁-C₆-alkyl, or cyano; as R⁶ hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl; and as R⁷ C₁-C₃-alkyl, C₁-C₃-haloalkyl, or C₃-C₄-cycloalkyl that may be mono or polysubstituted by R⁹ (wherein R⁹ is halogen, C₁-C₃-alkyl, C₁-C₃-alkoxy, C₁-C₃-haloalkyl, or cyano).

In an embodiment of each aspect of the invention, the compound of the formula (I-1a), (I-2a), (I-3a) or (I-4a) has as R¹ ethyl, isopropyl, propargyl, 2,2,2-trifluoroethyl, 2,2-difluoroethyl, cyclopropyl, 1-cyanocyclopropyl, 1-cyanocyclobutyl, 3,3-difluorocyclobutyl, 1,1-dioxothietan-3-yl, or cyclopropylmethyl; as R² hydrogen, methyl, acetyl or 2-methyl propanoyl; as R³ chlorine, bromine or methyl; as R⁶ hydrogen, methyl, methoxymethyl, cyclopropylmethyl, or acetyl; and as R⁷ C₁-C₃-alkyl, C₁-C₃-haloalkyl, or C₃-C₄-cycloalkyl that may be mono or polysubstituted by R⁹ (wherein R⁹ is halogen, C₁-C₃-alkyl, C₁-C₃-alkoxy, C₁-C₃-haloalkyl, or cyano).

In an embodiment of each aspect of the invention, the compound of the formula (I-1a), (I-2a), (I-3a) or (I-4a) has as R¹ ethyl, isopropyl, propargyl, 2,2,2-trifluoroethyl, 2,2-difluoroethyl, cyclopropyl, 1-cyanocyclopropyl, 1-cyanocyclobutyl, 3,3-difluorocyclobutyl, 1,1-dioxothietan-3-yl, or cyclopropylmethyl; as R² hydrogen, methyl, acetyl or 2-methyl propanoyl; as R³ chlorine, bromine or methyl; as R⁶ hydrogen, methyl, methoxymethyl, cyclopropylmethyl, or acetyl; and as R⁷ C₁-C₃-alkyl, C₁-C₃-haloalkyl, or C₃-C₄-cycloalkyl that may be mono or polysubstituted by R⁹ (wherein R⁹ is halogen, C₁-C₃-alkyl, C₁-C₃-alkoxy, C₁-C₃-haloalkyl, or cyano).

In a further embodiment of the invention, the compound of formula (I) is a compound of formula (I-1),

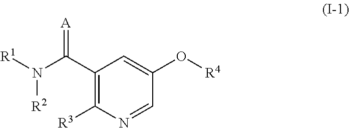

wherein
A is O;
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R² is hydrogen, C₁-C₃-alkyl, C₁-C₃-alkylcarbonyl, or C₁-C₃-alkoxycarbonyl;
R³ is halogen;
R⁴ is

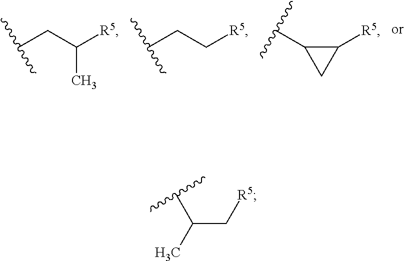

R⁵ is C₁-C₃-alkylsulfonylamino, C₁-C₃-haloalkylsulfonylamino, or C₃-C₆-cycloalkylsulfonylamino, wherein any of said amino-groups is unsubstituted or substituted by R⁶; and wherein any of said cycloalkyl-groups may be mono- or polysubstituted by R⁹;
R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl; and R⁹ is halogen, C₁-C₃-alkyl, C₁-C₃-alkoxy, C₁-C₃-haloalkyl, or cyano.

In a further embodiment of the invention, the compound of formula (I) is a compound of formula (I-1a),

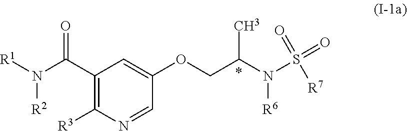

wherein
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R² is hydrogen, C₁-C₃-alkyl, C₁-C₃-alkylcarbonyl, or C₁-C₃-alkoxycarbonyl;
R³ is halogen;
R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl;
R⁷ is C₁-C₃-alkyl, C₁-C₃-haloalkyl, or C₃-C₄-cycloalkyl that may be mono or polysubstituted by R⁹; and
R⁹ is halogen, C₁-C₃-alkyl, C₁-C₃-alkoxy, C₁-C₃-haloalkyl, or cyano.

In a further embodiment of the invention, the compound of formula (I) is a compound of formula (I-1a),

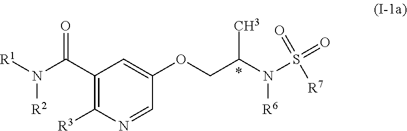

wherein
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R² is hydrogen, C₁-C₃-alkyl, C₁-C₃-alkylcarbonyl, or C₁-C₃-alkoxycarbonyl;
R³ is halogen;
R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl;
R⁷ is C₁-C₃-alkyl, C₁-C₃-haloalkyl, or C₃-C₄-cycloalkyl that may be mono or polysubstituted by R⁹; and
R⁹ is halogen, C₁-C₃-alkyl, C₁-C₃-alkoxy, C₁-C₃-haloalkyl, or cyano, and
   wherein said compound is an (S) enantiomer.

In a further embodiment of the invention, the compound of formula (I) is a compound of formula (I-1a),

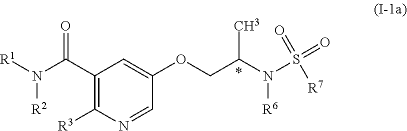

wherein
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R² is hydrogen, C₁-C₃-alkyl, C₁-C₃-alkylcarbonyl, or C₁-C₃-alkoxycarbonyl;
R³ is chlorine or bromine;
R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl;
R⁷ is C₁-C₃-alkyl, C₁-C₃-haloalkyl, or C₃-C₄-cycloalkyl that may be mono or polysubstituted by R⁹; and
R⁹ is halogen, C₁-C₃-alkyl, C₁-C₃-alkoxy, C₁-C₃-haloalkyl, or cyano, and
   wherein said compound is an (S) enantiomer.

In an embodiment, a compound of formula (I) is represented by the formula (I-5a), wherein X, Y, Z, R¹, R², R³ and R⁶ are as defined as for the compound of formula (I); and R⁷ is selected from C₁-C₆-alkyl, C₁-C₆-haloalkyl, or C₃-C₆-cycloalkyl that may be mono or polysubstituted by R⁹.

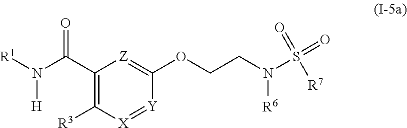

Preferably, in a compound of formula (I-5a),
X is N;
Y, Z are CH;
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R³ is halogen;
R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl;
R⁷ is C₁-C₃-alkyl, C₁-C₃-haloalkyl, or C₃-C₄-cycloalkyl that may be mono or polysubstituted by R⁹; and
R⁹ is halogen, C₁-C₃-alkyl, C₁-C₃-alkoxy, C₁-C₃-haloalkyl, or cyano.

Preferably, in a compound of formula (I-5a),
X is N;
Y, Z are CH;
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R³ is chlorine, bromine, or methyl;
R⁶ is hydrogen;
R⁷ is C₁-C₃-alkyl, C₁-C₃-haloalkyl, or C₃-C₄-cycloalkyl that may be mono or polysubstituted by R⁹; and
R⁹ is halogen, C₁-C₃-alkyl, C₁-C₃-alkoxy, C₁-C₃-haloalkyl, or cyano.

Preferably, in a compound of formula (I-5a),
X is N;
Y, Z are CH;
R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl, or cyano-C₃-C₄-cycloalkyl;
R³ is chlorine, bromine, or methyl;
R⁶ is hydrogen;
R⁷ is C₁-C₃-alkyl, C₁-C₃-haloalkyl, or C₃-C₄-cycloalkyl.

In one embodiment of the invention, the compound of formula (I) according to the invention is selected from compounds listed in any one of Tables 1 to 5.

In another embodiment of the invention the compound of formula (I) according to the invention is selected from compounds listed in Table P (below).

Preferably the compound of formula (I) according to the invention is selected from 2-chloro-N-cyclopropyl-5-[(2S)-2-(trifluoromethylsulfonylamino) propoxy]pyridine-3-carboxamide, 2-chloro-N-(1-cyanocyclopropyl)-5-[(2S)-2-(trifluoromethylsulfonylamino) propoxy]pyridine-3-carboxamide, 2-chloro-N-(2,2,2-trifluoroethyl)-5-[(2S)-2-(trifluoromethylsulfonylamino) propoxy]pyridine-3-carboxamide, 2-chloro-N-cyclopropyl-5-[(2S)-2-(dichloromethylsulfonylamino) propoxy]pyridine-3-carboxamide, N-(1-bicyclo[1.1.1]pentanyl)-2-chloro-5-[(2S)-2-(trifluoromethylsulfonylamino) propoxy]pyridine-3-carboxamide, tert-butyl N-[(1S)-2-[[6-chloro-5-[(1-cyanocyclopropyl)carbamoyl]-3-pyridyl]oxy]-1-methyl-ethyl]-N-(trifluoromethylsulfonyl)carbamate, 2-chloro-N-(1-cyanocyclopropyl)-5-[(2S)-2-[methyl(trifluoromethylsulfonyl) amino]propoxy]pyridine-3-carboxamide, 2-chloro-N-(1-cyanocyclopropyl)-5-[(2S)-2-[cyanomethyl(trifluoromethylsulfonyl) amino]propoxy]pyridine-3-carboxamide, [[(1S)-2-[[6-chloro-5-[(1-cyanocyclopropyl)carbamoyl]-3-pyridyl]oxy]-1-methyl-ethyl]-(trifluoromethylsulfonyl)amino]methyl 2-methylpropanoate, 2-chloro-N-cyclopropyl-5-[(2S)-2-(difluoromethylsulfonylamino) propoxy]pyridine-3-carboxamide, N-tert-butyl-2-chloro-5-[(2S)-2-(trifluoromethylsulfonylamino) propoxy]pyridine-3-carboxamide, N-acetyl-2-chloro-N-cyclopropyl-5-[(2S)-2-[methoxymethyl(trifluoromethylsulfonyl)amino]propoxy]pyridine-3-carboxamide, [[(1S)-2-[[6-chloro-5-(cyclopropylcarbamoyl)-3-pyridyl]oxy]-1-methyl-ethyl]-(trifluoromethylsulfonyl) amino]methyl 2-methylpropanoate, methyl N-[(1S)-2-[[6-chloro-5-(cyclopropylcarbamoyl)-3-pyridyl]oxy]-1-methyl-ethyl]-N-(trifluoromethylsulfonyl)carbamate, 2-chloro-5-[(2S)-2-[cyanomethyl (trifluoromethylsulfonyl)amino]propoxy]-N-cyclopropyl-pyridine-3-carboxamide, 2-chloro-N-cyclopropyl-5-[(2S)-2-[methyl(trifluoromethylsulfonyl)amino]propoxy]pyridine-3-carboxamide, 2-chloro-N-cyclopropyl-5-[2-(trifluoromethylsulfonylamino) ethoxy]pyridine-3-carboxamide, 2-chloro-N-(5-cyano-3-fluoro-2-pyridyl)-5-[(2S)-2-(trifluoromethylsulfonylamino) propoxy]pyridine-3-carboxamide, 2-chloro-N-(5-chloro-3-fluoro-2-pyridyl)-5-[(2S)-2-(trifluoromethylsulfonylamino) propoxy]pyridine-3-carboxamide, 2-chloro-N-(5-chloro-3-methyl-2-pyridyl)-5-[(2S)-2-(trifluoromethylsulfonylamino) propoxy]pyridine-3-carboxamide, 2-chloro-5-[(2S)-2-(trifluoromethylsulfonylamino) propoxy]-N-(2,4,6-trifluorophenyl) pyridine-3-carboxamide, 2-chloro-N-(4-cyano-2-fluoro-phenyl)-5-[(2S)-2-(trifluoromethylsulfonylamino) propoxy]pyridine-3-carboxamide, 2-chloro-N-(2,4-difluorophenyl)-5-[(2S)-2-(trifluoromethylsulfonylamino) propoxy]pyridine-3-carboxamide, 2-chloro-N-(2-fluoro-4-methylsulfonyl-phenyl)-5-[(2S)-2-(trifluoromethylsulfonylamino) propoxy]pyridine-3-carboxamide, 2-chloro-N-(4-chloro-2-fluoro-phenyl)-5-[(2S)-2-(trifluoromethylsulfonylamino) propoxy]pyridine-3-carboxamide, N-(1-bicyclo[1.1.1]pentanyl)-2-methyl-5-[(2S)-2-(trifluoromethylsulfonylamino) propoxy]pyridine-3-carboxamide, N-[1-[2-(5-cyano-2-pyridyl)-1,2,4-triazol-3-yl]ethyl]-2-methyl-5-[(2S)-2-(trifluoromethylsulfonylamino) propoxy]pyridine-3-carboxamide, N-[(5-chloro-2-pyridyl)methyl]-2-methyl-5-[(2S)-2-(trifluoromethylsulfonylamino) propoxy]pyridine-3-carboxamide, N-[(2E)-2-methoxyimino-1,1-dimethyl-ethyl]-2-methyl-5-[(2S)-2-(trifluoromethylsulfonylamino) propoxy]pyridine-3-carboxamide, 2-methyl-N-[3-oxo-2-(2,2,2-trifluoroethyl) isoxazolidin-4-yl]-5-[(2S)-2-(trifluoromethylsulfonylamino) propoxy]pyridine-3-carboxamide, N-(1-cyclopropylethyl)-2-methyl-5-[(2S)-2-(trifluoromethylsulfonylamino) propoxy]pyridine-3-carboxamide, N-[(3-chloro-2-pyridyl)methyl]-2-methyl-5-[(2S)-2-(trifluoromethylsulfonylamino) propoxy]pyridine-3-carboxamide, N-(2,4-difluorophenyl)-2-methyl-5-[(2S)-2-(trifluoromethylsulfonylamino) propoxy]pyridine-3-carboxamide, N-isopropyl-2-methyl-5-[(2S)-2-(trifluoromethylsulfonylamino) propoxy]pyridine-3-carboxamide, N-[1-(4-fluorophenyl)-2-oxo-pyrrolidin-3-yl]-2-methyl-5-[(2S)-2-(trifluoromethylsulfonylamino) propoxy]pyridine-3-carboxamide, N-ethyl-2-methyl-5-[(2S)-2-(trifluoromethylsulfonylamino) propoxy]pyridine-3-carboxamide, N-(3,3-difluorocyclobutyl)-2-methyl-5-[(2S)-2-(trifluoromethylsulfonylamino) propoxy]pyridine-3-carboxamide, 2-methyl-N-[1-(5-methyl-1,2,4-oxadiazol-3-yl)ethyl]-5-[(2S)-2-(trifluoromethylsulfonylamino) propoxy]pyridine-3-carboxamide, N-(2,2-difluoroethyl)-2-methyl-5-[(2S)-2-(trifluoromethylsulfonylamino) propoxy]pyridine-3-carboxamide, N-cyclopropyl-N,2-dimethyl-5-[(2S)-2-(trifluoromethylsulfonylamino) propoxy]pyridine-3-carboxamide, N-(cyclopropylmethoxy)-2-methyl-5-[(2S)-2-(trifluoromethylsulfonylamino) propoxy]pyridine-3-carboxamide, tert-butyl N-[(1S)-2-[[6-chloro-5-(cyclopropylcarbamoyl)-3-pyridyl]oxy]-1-methyl-ethyl]-N-(trifluoromethylsulfonyl)carbamate, 5-[(2S)-2-[acetyl(trifluoromethylsulfonyl)amino]propoxy]-2-chloro-N-cyclopropyl-pyridine-3-carboxamide, N-benzyl-2-chloro-5-[(2S)-2-(trifluoromethylsulfonylamino) propoxy]pyridine-3-carboxamide, 2-chloro-N-(1-cyclopropylethyl)-5-[(2S)-2-(trifluoromethylsulfonylamino) propoxy]pyridine-3-carboxamide, 2-chloro-N-[(1-cyanocyclopropyl) methyl]-5-[(2S)-2-(trifluoromethylsulfonylamino)propoxy]pyridine-3-carboxamide, 2-chloro-N-(1-cyanocyclopropyl)-N-methyl-5-[(2S)-2-(trifluoromethylsulfonylamino)propoxy]pyridine-3-carboxamide, 2-chloro-N-(1-cyanocyclobutyl)-5-[(2S)-2-(trifluoromethylsulfonylamino)propoxy]pyridine-3-carboxamide, 2-chloro-N-[(4S)-2-ethyl-3-oxo-isoxazolidin-4-yl]-5-[(2S)-2-(trifluoromethylsulfonylamino)propoxy]pyridine-3-carboxamide, 2-chloro-N-ethyl-5-[(2S)-2-(trifluoromethylsulfonylamino)propoxy]pyridine-3-carboxamide, 2-chloro-N-(3-methyltetrahydrofuran-3-yl)-5-[(2S)-2-(trifluoromethylsulfonylamino)propoxy]pyridine-3-carboxamide, 2-chloro-N-prop-2-ynyl-5-[(2S)-2-(trifluoromethylsulfonylamino)propoxy]pyridine-3-carboxamide, 2-chloro-N-[2-(cyclopropylmethylamino)-2-oxo-ethyl]-5-[(2S)-2-(trifluoromethylsulfonylamino)propoxy]pyridine-3-carboxamide, 2-chloro-N-[3-oxo-2-(2,2,2-trifluoroethyl) isoxazolidin-4-yl]-5-[(2S)-2-(trifluoromethylsulfonylamino) propoxy]pyridine-3-carboxamide, 2-chloro-N-(2,2-difluoroethyl)-5-[(2S)-2-(trifluoromethylsulfonylamino)propoxy]pyridine-3-carboxamide, 2-chloro-N-phenyl-5-[(2S)-2-(trifluoromethylsulfonylamino)propoxy]pyridine-3-carboxamide, 2-chloro-N-(1-cyano-1-methyl-ethyl)-5-[(2S)-2-(trifluoromethylsulfonylamino)propoxy]pyridine-3-carboxamide, 2-chloro-N-cyclopropyl-N-methyl-5-[(2S)-2-(trifluoromethylsulfonylamino)propoxy]pyridine-3-carboxamide, 2-chloro-N-(2,2-difluoroethyl)-N-methyl-5-[(2S)-2-(trifluoromethylsulfonylamino)propoxy]pyridine-3-carboxamide, 2-chloro-N-(4-fluoro-2-methyl-phenyl)-5-[(2S)-2-(trifluoromethylsulfonylamino)propoxy]pyridine-3-carboxamide, N-cyclopropyl-5-[(2S)-2-[methoxymethyl (trifluoromethylsulfonyl) amino]propoxy]-2-methyl-pyridine-3-carboxamide, N-cyclopropyl-2-methyl-5-[(2S)-2-(trifluoromethylsulfonylamino)propoxy]pyridine-3-carboxamide, 2-chloro-5-[(2S)-2-[methoxymethyl(trifluoromethylsulfonyl)amino]propoxy]-N-(2,2,2-trifluoroethyl)pyridine-3-carboxamide, 2-chloro-N-cyclopropyl-5-[(2S)-2-[methoxymethyl(trifluoromethyl sulfonyl)amino]propoxy]pyridine-3-carboxamide, 2-chloro-N-[(4R)-2-ethyl-3-oxo-isoxazolidin-4-yl]-5-[(2S)-2-(trifluoromethylsulfonylamino)propoxy]pyridine-3-carboxamide, N-(1-bicyclo[1.1.1]pentanyl)-5-methyl-2-[(2S)-2-(trifluoromethylsulfonylamino)propoxy]pyridine-4-carboxamide, N-(1-cyanocyclopropyl)-N,5-dimethyl-2-[(2S)-2-(trifluoromethylsulfonylamino)propoxy]pyridine-4-carboxamide, N-(4-ethylthiazol-5-yl)-5-methyl-2-[(2S)-2-(trifluoromethylsulfonylamino)propoxy]pyridine-4-carboxamide, N-(1,3-dihydroisobenzofuran-5-yl methyl)-5-methyl-2-[(2S)-2-(trifluoromethylsulfonylamino)propoxy]pyridine-4-carboxamide, 5-methyl-N-[2-oxo-1-(2,2,2-trifluoroethyl) pyrrolidin-3-yl]-2-[(2S)-2-(trifluoromethylsulfonylamino)propoxy]pyridine-4-carboxamide, N-[2-(2-fluorophenoxy)ethyl]-5-methyl-2-[(2S)-2-(trifluoromethylsulfonylamino)propoxy]pyridine-4-carboxamide, N-(5-methoxy-1,1-dimethyl-pent-2-ynyl)-5-methyl-2-[(2S)-2-(trifluoromethylsulfonylamino)propoxy]pyridine-4-carboxamide, N-(1-cyclopropylethyl)-5-methyl-2-[(2S)-2-(trifluoromethylsulfonylamino)propoxy]pyridine-4-carboxamide, N-[1-(2-ethylthiazol-4-yl)ethyl]-5-methyl-2-[(2S)-2-(trifluoromethylsulfonylamino)propoxy]pyridine-4-carboxamide, N-(3-cyano-2-fluoro-phenyl)-5-methyl-2-[(2S)-2-(trifluoromethylsulfonylamino)propoxy]pyridine-4-carboxamide, 5-methyl-N-[1-(3-pyridyl)ethyl]-2-[(2S)-2-(trifluoromethylsulfonylamino)propoxy]pyridine-4-carboxamide, N-benzyl-5-methyl-2-[(2S)-2-(trifluoromethylsulfonylamino)propoxy]pyridine-4-carboxamide, N-[(4R)-2-ethyl-3-oxo-isoxazolidin-4-yl]-2-[(2S)-2-[methoxymethyl(trifluoromethylsulfonyl) amino]propoxy]-5-methyl-pyridine-4-carboxamide, N-(1-cyanocyclopropyl)-2-[(2S)-2-[methoxymethyl(trifluoromethylsulfonyl) amino]propoxy]-5-methyl-pyridine-4-carboxamide, 5-methyl-N-(2,2,2-trifluoroethyl)-2-[(2S)-2-(trifluoromethylsulfonylamino)propoxy]pyridine-4-carboxamide, 5-bromo-N-cyclopropyl-2-[(2S)-2-[methoxymethyl(trifluoromethylsulfonyl)amino]propoxy]pyridine-4-carboxamide, 5-bromo-N-(1-cyanocyclopropyl)-2-[(2S)-2-[methoxymethyl(trifluoromethylsulfonyl)amino]propoxy]pyridine-4-carboxamide, 5-bromo-N-cyclopropyl-2-[(2S)-2-(trifluoromethylsulfonylamino)propoxy]pyridine-4-carboxamide, 5-bromo-N-[(4R)-2-ethyl-3-oxo-isoxazolidin-4-yl]-2-[(2S)-2-(trifluoromethylsulfonylamino) propoxy]pyridine-4-carboxamide, 5-bromo-N-(2,2,2-trifluoroethyl)-2-[(2S)-2-(trifluoromethylsulfonylamino) propoxy]pyridine-4-carboxamide, 5-bromo-N-(1-cyanocyclopropyl)-2-[(2S)-2-(trifluoromethylsulfonyl amino) propoxy]pyridine-4-carboxamide, 5-chloro-N-cyclopropyl-2-[(2S)-2-(trifluoromethylsulfonylamino)propoxy]pyridine-4-carboxamide, 3-chloro-N-cyclopropyl-6-[(2S)-2-(trifluoromethylsulfonylamino)propoxy]pyridazine-4-carboxamide, 3-chloro-N-(1-cyanocyclopropyl)-6-[(2S)-2-(trifluoromethylsulfonylamino)propoxy]pyridazine-4-carboxamide, or N-(1-bicyclo[1.1.1]pentanyl)-3-chloro-6-[(2S)-2-(trifluoromethylsulfonylamino)propoxy]pyridazine-4-carboxamide.

More preferably the compound of formula (I) according to the present invention is selected from 2-chloro-N-cyclopropyl-5-[(2S)-2-(trifluoromethylsulfonylamino)propoxy]pyridine-3-carboxamide, 2-chloro-N-(1-cyanocyclopropyl)-5-[(2S)-2-(trifluoromethylsulfonylamino)propoxy]pyridine-3-carboxamide, or 2-chloro-N-(2,2,2-trifluoroethyl)-5-[(2S)-2-(trifluoromethylsulfonylamino)propoxy]pyridine-3-carboxamide.

In a second aspect, the present invention makes available a composition comprising a compound of formula (I) as defined in the first aspect, one or more auxiliaries and diluent, and optionally one more other active ingredient.

In a third aspect, the present invention makes available a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in the first aspect or a composition as defined in the second aspect.

In a fourth aspect, the present invention makes available a method for the protection of plant propagation material from the attack by insects, acarines, nematodes or molluscs, which comprises treating the propagation material or the site, where the propagation material is planted, with an effective amount of a compound of formula (I) as defined in the first aspect or a composition as defined in the second aspect.

In a fifth aspect, the present invention makes available a plant propagation material, such as a seed, comprising, or treated with or adhered thereto, a compound of formula (I) as defined in the first aspect or a composition as defined in the second aspect.

The present invention in a further aspect provides a method of controlling parasites in or on an animal in need thereof comprising administering an effective amount of a compound of the first aspect. The present invention further provides a method of controlling ectoparasites on an animal in need thereof comprising administering an effective amount of a compound of formula (I) as defined om the first aspect. The present invention further provides a method for preventing and/or treating diseases transmitted by ectoparasites comprising administering an effective amount of a compound of formula (I) as defined in the first aspect, to an animal in need thereof.

Compounds of formula (I) can be prepared by those skilled in the art following known methods. More specifically compounds of formula I, and intermediates therefor can be prepared as described below in the schemes and examples. Certain stereogenic centers have been left unspecified for the clarity and are not intended to limit the teaching of the schemes in any way.

The compounds of formula (I) according to the invention, wherein R¹, R², R³, R⁴, X, Y, Z are as defined for formula (I) and A is oxygen, can be obtained by transformation of a compound of formula (II), wherein R³, R⁴, X, Y, Z are as defined for formula (I) with a compound of formula (III), wherein R¹ and R² are as defined for formula (I), via an intermediate acid chloride or activated acylating agent as described below. This is shown in Scheme 1 below:

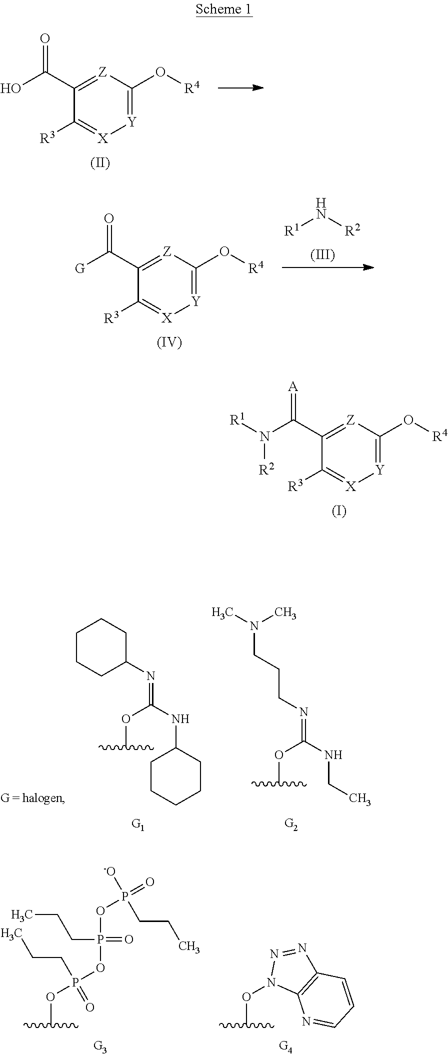

As shown in Scheme 1, compound II, are activated to compounds of formula IV by methods known to those skilled in the art and described for example in Tetrahedron 2005, 61 (46), 10827-10852. For example, compounds of formula IV where G is halogen are formed by treatment of compounds of formula II with for example, oxalyl chloride or thionyl chloride in the presence of catalytic quantities of DMF in inert solvents such as methylene dichloride or THF at temperatures between 25 to 170° C. preferably 25 to 80° C. Treatment of IV with compounds of formula III, wherein R¹ and R² are as defined in formula I, optionally in the presence of a base, e.g. triethylamine or pyridine leads to compounds of formula I. Alternatively, compounds of formula I can be prepared by treatment of compounds of formula II with dicyclohexyl carbodiimide (DCC) or 1-ethyl-3-(3-dimethylaminopropyl)carbodiimide (EDC) to give the activated species IV, wherein G is G₁ or G₂, in an inert solvent, e.g. pyridine, or THF optionally in the presence of a base, e.g. triethylamine, at temperatures between rt and 180° C. In addition, an acid of the formula II can also be activated by reaction with a coupling reagent such as propanephosphonic acid anhydride (T3P®) or O-(7-Aza-1-benzotriazolyl)-N,N,N′,N′-tetramethyluronium-hexafluorophosphat (HATU) to provide compounds of formula IV wherein G is G₃ and G₄ as described for example in Synthesis 2013, 45, 1569 and J. Prakt. Chemie 1998, 340, 581. Subsequent reaction with an amine of the formula III provides compounds of formula I.

The compounds of formula (II), wherein R³, R⁴, X, Y, Z are as defined for formula (I), can be obtained by transformation of a compound of formula (V), wherein R³, R⁴, X, Y, Z are as defined for formula (I) and R²⁰ is C₁-C₆alkyl, in the presence of a base or an acid. This is shown in Scheme 2 below.

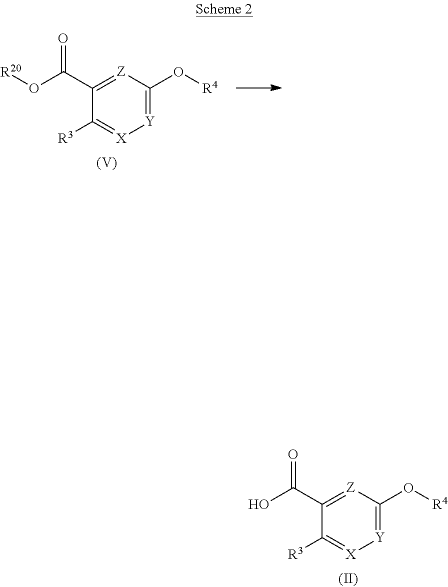

As shown in Scheme 2, compounds (V) can undergo a hydrolysis reaction under basic conditions to compounds (II) by methods known to those skilled in the art and described for example in ACS Chemical Neuroscience 2017, 2374. For example, the reaction can be performed in water or in organic solvent, such as methanol, THF or methylene dichloride, or in mixtures; in the presence of a base, such as lithium hydroxide, sodium hydroxide or potassium hydroxide. Alternatively, the conversion of compounds (V) into compounds (II) can be performed under acidic conditions by methods known to those skilled in the art and described for example in J. Med. Chem. 1986, 2363. For example, the reaction can be performed in water or in mixtures of water and an organic solvent, such as methanol, THF or dioxane; in the presence of an acid, such as trifluoroacetic acid, hydrochloric acid or sulfuric acid.

The compounds of formula (Va), wherein R³, X, Y, Z are as defined for formula (I), and R²⁰ is C₁-C₆ alkyl, and R²³ is hydrogen or methyl, can be obtained by transformation of a compound of formula (VI), wherein R³, X, Y, Z are as defined for formula (I), and R²⁰ is C₁-C₆ alkyl, and R²³ is hydrogen or methyl, in the presence of a sulfonylating agent of formula (VII), wherein R⁷ is C₁-C₆ alkyl, C₁-C₆ haloalkyl, C₁-C₆ cycloalkyl or C₁-C₆ halocycloalkyl and G is an halogen or an activating group of formula G5. This is shown in Scheme 3 below.

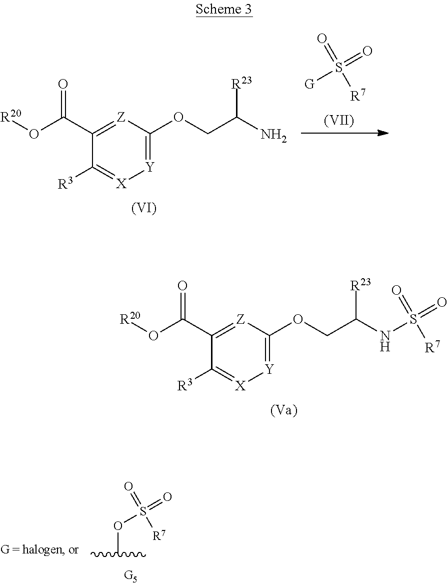

As shown in Scheme 3, compounds (VI) can be converted to compounds (Va) by methods known to those skilled in the art and described for example in Angew. Chem. Int. Ed. 2016, 5299; J. Am. Chem. Soc. 2018, 5322; WO2021153720, WO2010150192. For example, the reaction can be performed in inert organic solvent, such as methylene dichloride, dioxane or THF, in the presence of a base, such as triethylamine, pyridine or 1,8-Diazabicyclo[5.4.0]undec-7-ene. Alternatively, as known to those skilled in the art, compound (VI) in a salt form (e.g. hydrochloric salt, hydrobromic salt, trifluoroacetic salt, paratoluensulfonic salt) can be similarly used in a conversion to compound (Va).

The compounds of formula (VI), wherein R³, X, Y, Z are as defined for formula (I), and R²⁰ is C₁-C₆ alkyl, and R²³ is hydrogen or methyl, can be obtained by transformation of a compound of formula (VIII), wherein R³, X, Y, Z are as defined for formula (I), R²⁰ and R²¹ are C₁-C₆ alkyl, and R²³ is hydrogen or methyl. This is shown in Scheme 4 below.

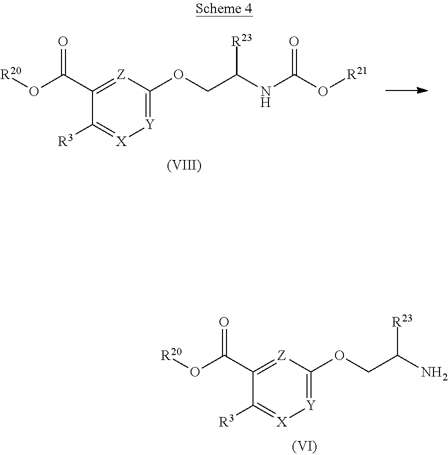

As shown in Scheme 4, compounds (VIII) can be converted to compounds (VI) by methods known to those skilled in the art and described for example in WO2002059117, J. Med. Chem. 2003, 5238, Org. Lett. 2022, 2064. For example, the reaction can be performed in organic solvent, such as methylene dichloride, dioxane or THF, in the presence of an organic or inorganic acid, such as hydrochloric acid, trifluoroacetic acid or paratoluensulfonic acid. Compounds (VI) can also be obtained and used in following transformations in their salt form (e.g. hydrochloric salt, hydrobromic salt, trifluoroacetic salt, paratoluensulfonic salt).

The compounds of formula (VIII), wherein R³, X, Y, Z are as defined for formula (I), R²⁰ and R²¹ are C₁-C₆ alkyl, and R²³ is hydrogen or methyl, can be obtained by transformation of a compound of formula (IX), wherein R³, X, Y, Z are as defined for formula (I) and R²⁰ is C₁-C₆ alkyl and R²³ is hydrogen or methyl, with an alkylating agent of formula (Xa), wherein R²¹ is C₁-C₆alkyl and Q is halogen, hydroxyl of sulfonate group. This is shown in Scheme 5 below.

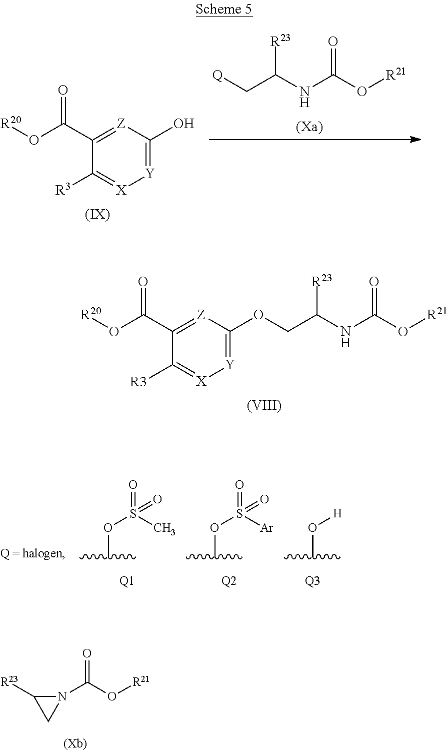

As shown in Scheme 5, compounds (IX) can be converted by an alkylative reaction with Xa (when Q is i.e. halogen or sulfonate Q1 or Q2) to compounds (VIII) by methods known to those skilled in the art and described for example in Angew. Chem. Int. Ed. 2017, 12518; WO2012101244, WO2004050619, WO2007072041. For example, the reaction can be performed in organic solvent, such as dimethylformamide, acetone or acetonitrile in the presence of a base, such as cesium carbonate, sodium hydride or sodium carbonate. Alternatively, compounds (IX) can be converted by a Mitsunobu reaction with Xa (when Q is equal to OH) by methods known to those skilled in the art and described for example in WO2012074126; Bioorg. Med. Chem. Lett. 1992, 481, Beilstein J. Org. Chem.2006, 2, 21. For example the reaction can be performed in organic solvent, such as toluene or THF in the presence of an azadicarboxylate reagent, such as diethyl azadicarboxylate or diisopropyl azadicarboxylate, and a phospine reagent, such as triphenyl phosphine. Alterantively, compounds (IX) can be converted by an alkylative reaction with aziridine Xb to compounds (VIII) by methods known to those skilled in the art and described for example in WO2007070201. For example the reaction can be performed in organic solvent, such as acetonitrile in the presence of a base, such as potassium carbonate, sodium hydride or sodium carbonate.

Compounds of formula (IX), wherein R³, X, Y, Z are as defined for formula (I) and R²⁰ is C₁-C₆ alkyl, can be obtained in two steps by transformation of a compound of formula (XII), wherein R³, X, Y, Z are as defined for formula (I) and R²⁰ is C₁-C₆ alkyl and R²² is halogen via and intermediate compound of formula (XI), wherein R³, X, Y, Z are as defined for formula (I) and R²⁰ is C₁-C₆ alkyl. This is shown in Scheme 6 below.

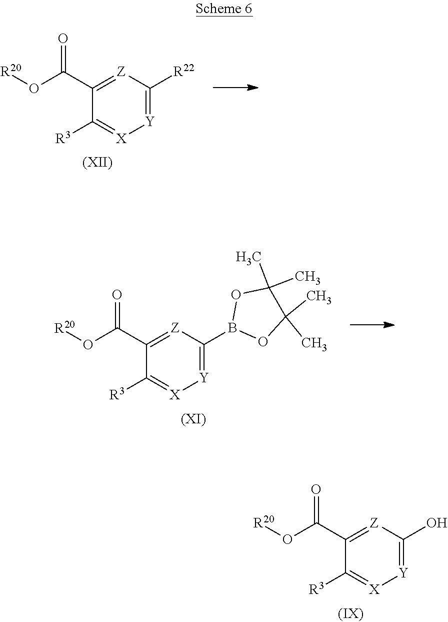

As shown in Scheme 6, compounds (XII) can be converted to compounds (XII) by methods known to those skilled in the art with the name of Miyaura borylation reaction and described for example in J. Org. Chem., 1995, 7508; J. Org. Chem., 2021, 103. For example, the reaction can be performed in organic solvent, such as THF or dioxane, in the presence of a base, such as potassium carbonate or potassium acetate and a palladium catalyst in combination with an appropriate ligand, such as 1,1′-bis(diphenylphosphino)ferrocene dichloropalladium. Intermediate (XI) can be converted into compounds of formula (IX) by methods known to those skilled in the art and described for example in Tetrahedron Letters 2014, 2082; Tetrahedron Lett. 2017, 3323; Tetrahedron 2005, 1417. For example the reaction can be performed in mixtures of water and an organic solvent, such as ethanol, acetonitrile or methylene dichloride, in the presence of hydrogen peroxide.

Alternatively, compounds of formula (VIII), wherein R³, X, Y, Z are as defined for formula (I), R²⁰ and R²¹ are C₁-C₆ alkyl, and R²³ is hydrogen or methyl, can be obtained by transformation of a compound of formula (XII), wherein R³, X, Y, Z are as defined for formula (I), R²⁰ is C₁-C₆ alkyl, R²² is a halogen and R²³ is hydrogen or methyl, with a compound of formula (Xa), wherein R²¹ is C₁-C₆ alkyl and Q is hydroxy. This is shown in Scheme 7 below.

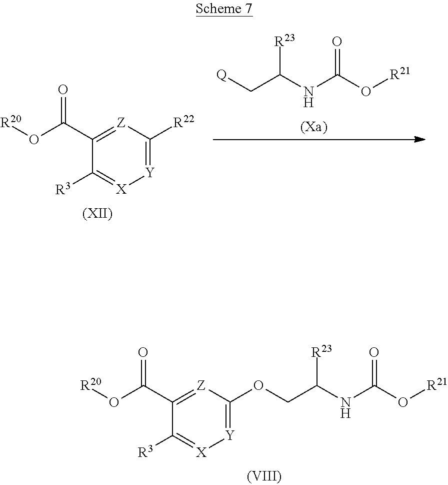

As shown in Scheme 7, compounds (XII) can be converted to compounds (VIII) by methods known to those skilled in the art and described for example in Tetrahedron 1988, 91; J. Am. Chem. Soc. 2010, 11592, WO2020211836. For example, the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction as shown in WO2017147700 and in J. Med. Chem. 2021, 11570.

Compounds of formula (Ia), wherein R¹, R², R³, R⁶, R⁷, X, Y and Z are as defined for formula (I), and R²³ is hydrogen or methyl, can be obtained by transformation of a compound of formula (Ib), R¹, R², R³, R⁷, X, Y and Z are as defined for formula (I) and R²³ is hydrogen or methyl, with a compound of formula (XIIa), wherein R⁶ is as defined for formula (I) and Q is halogen or sulfonate. This is shown in Scheme 8 below.

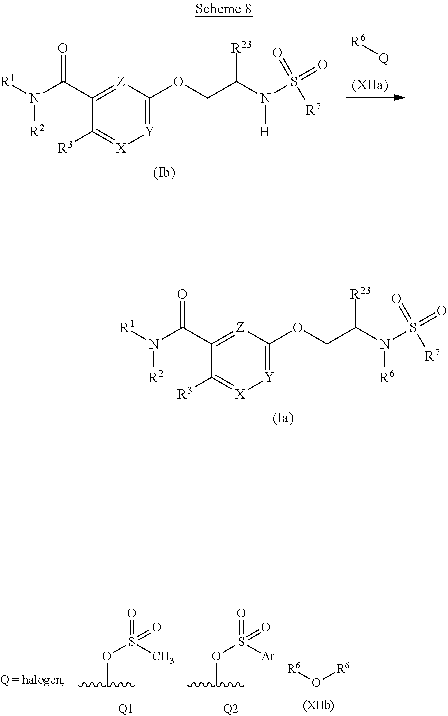

As shown in Scheme 8, compounds (Ib) can be converted to compounds (Ia) by methods known to those skilled in the art and described for example in ACS Combinatorial Science 2016, 569; WO202115372. For example, the reaction can be performed in organic solvent, such as methylene dichloride, dimethylformamide or acetonitrile, in the presence of a base, such as potassium carbonate ortriethylamine.

Compounds of formula (Id), wherein R¹, R², R³, R⁴, X, Y and Z are as defined for formula (I), can be obtained by transformation of a compound of formula (Ic), R¹, R², R³, R⁴, X, Y and Z are as defined for formula (I). This is shown in Scheme 9 below.

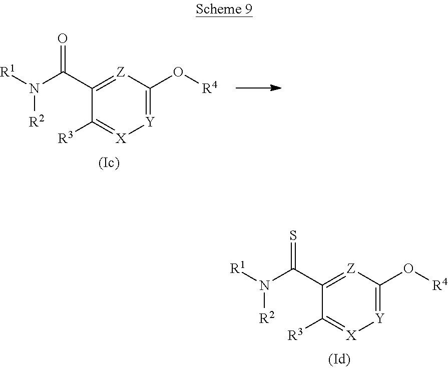

As shown in Scheme 9, compounds (Ic) can be converted to compounds (Id) by methods known to those skilled in the art and described for example in Org. Lett. 2019, 5848; Tetrahedron Lett. 2008, 4579; WO2021153720. For example, the reaction can be performed in organic solvent, such as toluene or chlorobenzene, in the presence of a thiation reagent, such as 2,4-Bis(4-methoxyphenyl)-1,3,2,4-dithiadiphosphetane-2,4-dithione (Lawesson's reagent).

Alternatively, compounds of formula (Ic) according to the invention, wherein R¹, R², R³, R⁷, X, Y, Z are as defined for formula (I), and R²³ is hydrogen or methyl, can be obtained by transformation of a compound of formula (XIII), wherein R¹, R², R³, X, Y and Z are as defined for formula (I), and R²³ is hydrogen or methyl, in the presence of a sulfonylating agent of formula (VII), wherein R⁷ is C₁-C₆ alkyl, C₁-C₆ haloalkyl, C₁-C₆ cycloalkyl or C₁-C₆ halocycloalkyl and G is a halogen or an activating group of formula G5. This is shown in Scheme 9 below. This is shown in Scheme 10 below:

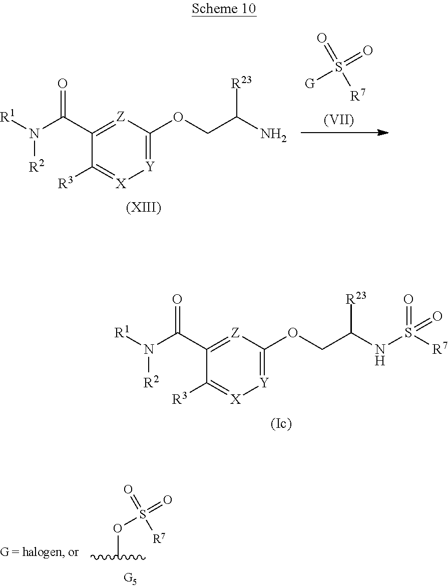

As shown in Scheme 10, compounds (XIII) can be converted to compounds (Ic) by methods known to those skilled in the art and described for example in Angew. Chem. Int. Ed. 2016, 5299; J. Am. Chem. Soc. 2018, 5322; WO2021153720, WO2010150192. For example, the reaction can be performed in inert organic solvent, such as methylene dichloride, dioxane or THF, in the presence of a base, such as triethylamine, pyridine or 1,8-Diazabicyclo[5.4.0]undec-7-ene. Alternatively, as known to those skilled in the art, compound (XIII) in a salt form (e.g. hydrochloric salt, hydrobromic salt, trifluoroacetic salt, paratoluensulfonic salt) can be similarly used in a conversion to compound (Ic).

The compounds of formula (XIII), wherein R¹, R², R³, X, Y and Z are as defined for formula (I), and R²³ is hydrogen or methyl, can be obtained by transformation of a compound of formula (XIV), wherein R¹, R², R³, X, Y, Z are as defined for formula (I), R²¹ is C₁-C₆ alkyl and R²³ is hydrogen or methyl. This is shown in Scheme 11 below.

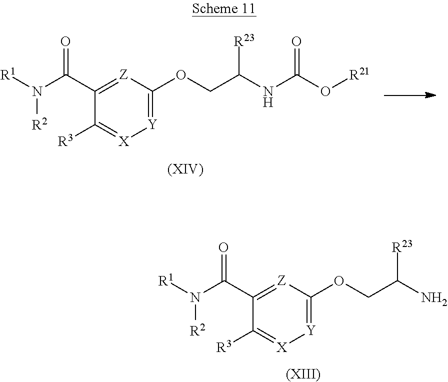

As shown in Scheme 11, compounds (XIV) can be converted to compounds (XII) by methods known to those skilled in the art and described for example in WO2002059117, J. Med. Chem. 2003, 5238, Org. Lett. 2022, 2064. For example, the reaction can be performed in organic solvent, such as methylene dichloride, dioxane or THF, in the presence of an organic or inorganic acid, such as hydrochloric acid, trifluoroacetic acid or paratoluensulfonic acid. Compounds (XIII) can also be obtained and used in following transformations in their salt form (e.g. hydrochloric salt, hydrobromic salt, trifluoroacetic salt, paratoluensulfonic salt).

The compounds of formula (XIV), wherein R¹, R², R³, X, Y, Z are as defined for formula (I) R²³ is hydrogen or methyl, and R²¹ is C₁-C₆ alkyl, can be obtained by transformation of a compound of formula (XV), wherein R¹, R², R³, X, Y, Z are as defined for formula (I) with an alkylating agent of formula (Xa), wherein R²¹ is C₁-C₆ alkyl, R²³ is hydrogen or methyl, and Q is halogen, hydroxyl of sulfonate group. This is shown in Scheme 12 below.

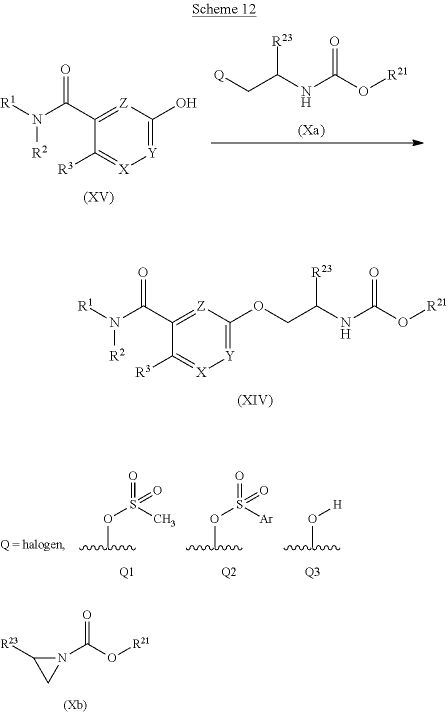

As shown in Scheme 12, compounds (XV) can be converted by an alkylative reaction with Xa (when Q is i.e. halogen or sulfonate Q1 or Q2) to compounds (XIV) by methods known to those skilled in the art and described for example in Angew. Chem. Int. Ed. 2017, 12518; WO2012101244, WO2004050619, WO2007072041. For example, the reaction can be performed in organic solvent, such as dimethylformamide, acetone or acetonitrile in the presence of a base, such as cesium carbonate, sodium hydride or sodium carbonate. Alternatively, compounds (XV) can be converted by a Mitsunobu reaction with Xa (when Q is equal to OH) by methods known to those skilled in the art and described for example in WO2012074126; Bioorg. Med. Chem. Lett. 1992, 481, Beilstein J. Org. Chem. 2006, 2, 21. For example the reaction can be performed in organic solvent, such as toluene or THF in the presence of an azadicarboxylate reagent, such as diethyl azadicarboxylate or diisopropyl azadicarboxylate, and a phospine reagent, such as triphenyl phosphine. Alterantively, compounds (XV) can be converted by an alkylative reaction with aziridine Xb to compounds (XIV) by methods known to those skilled in the art and described for example in WO2007070201. For example the reaction can be performed in organic solvent, such as acetonitrile in the presence of a base, such as potassium carbonate, sodium hydride or sodium carbonate.

The compounds of formula (XV) according to the invention, wherein R¹, R², R³, X, Y and Z are as defined for formula (I), can be obtained by transformation of a compound of formula (XVI), wherein R³, X, Y, Z are as defined for formula (I) with a compound of formula (III), wherein R¹ and R² are as defined for formula (I), via an intermediate acid chloride or activated acylating agent as described below. This is shown in Scheme 13 below:

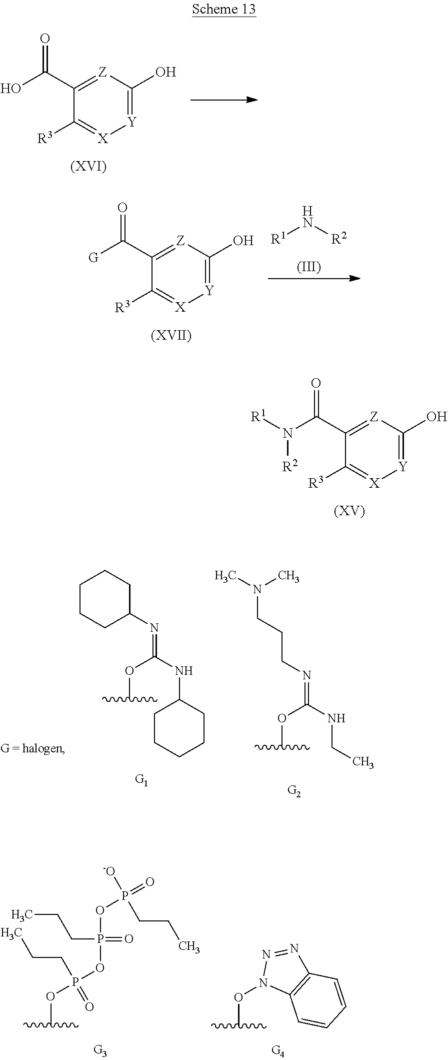

As shown in Scheme 13, compound XVI, is activated to compounds of formula XVII by methods known to those skilled in the art and described for example in Tetrahedron 2005, 61 (46), 10827-10852. For example, compounds of formula XVII where G is halogen are formed by treatment of compounds of formula XVI with for example, oxalyl chloride or thionyl chloride in the presence of catalytic quantities of DMF in inert solvents such as methylene dichloride or THF at temperatures between 25 to 170° C. preferably 25 to 80° C. Treatment of XVII with compounds of formula III, wherein R¹ and R² are as defined in formula I, optionally in the presence of a base, e.g. triethylamine or pyridine leads to compounds of formula I. Alternatively, compounds of formula XV can be prepared by treatment of compounds of formula XVII with dicyclohexyl carbodiimide (DCC) or 1-ethyl-3-(3-dimethylaminopropyl)carbodiimide (EDC) to give the activated species XVII, wherein G is G₁ or G₂, in an inert solvent, e.g. pyridine, or THF optionally in the presence of a base, e.g. triethylamine, at temperatures between rt and 180° C. In addition, an acid of the formula XVI can also be activated by reaction with a coupling reagent such as propanephosphonic acid anhydride (T3P®) or O-(7-Aza-1-benzotriazolyl)-N,N,N′,N′-tetramethyluronium-hexafluorophosphat (HATU) to provide compounds of formula XVII wherein G is G₃ and G₄ as described for example in Synthesis 2013, 45, 1569 and J. Prakt. Chemie 1998, 340, 581. Subsequent reaction with an amine of the formula III provides compounds of formula XV.

The compounds of formula (XVI), wherein R³, X, Y and Z are as defined for formula (I), can be obtained by transformation of a compound of formula (IX), wherein R³, X, Y, Z are as defined for formula (I) and R²⁰ is C₁-C₆alkyl, in the presence of a base or an acid. This is shown in Scheme 14 below.

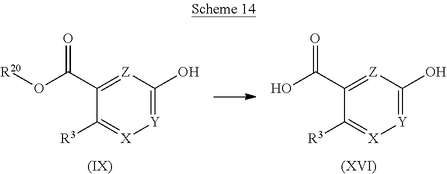

As shown in Scheme 14, compounds (IX) can undergo a hydrolysis reaction under basic conditions to compounds (XVI) by methods known to those skilled in the art and described for example in ACS Chemical Neuroscience 2017, 2374. For example, the reaction can be performed in water or in organic solvent, such as methanol, THF or methylene dichloride, or in mixtures; in the presence of a base, such as lithium hydroxide, sodium hydroxide or potassium hydroxide. Alternatively, the conversion of compounds (IX) into compounds (XVI) can be performed under acidic conditions by methods known to those skilled in the art and described for example in J. Med. Chem.1986, 2363. For example, the reaction can be performed in water or in mixtures of water and an organic solvent, such as methanol, THF or dioxane; in the presence of an acid, such as trifluoroacetic acid, hydrochloric acid or sulfuric acid.

Alternatively, compounds of formula (XIV), wherein R¹, R², R³, X, Y, Z are as defined for formula (I), R²¹ is C₁-C₆ alkyl, and R²³ is hydrogen or methyl, can be obtained by transformation of a compound of formula (XVIII), wherein R¹, R², R³, X, Y, Z are as defined for formula (I), and R²² is a halogen with a compound of formula (Xa), wherein R²¹ is C₁-C₆ alkyl, R²³ is hydrogen or methyl, and Q is hydroxy. This is shown in Scheme 15 below.

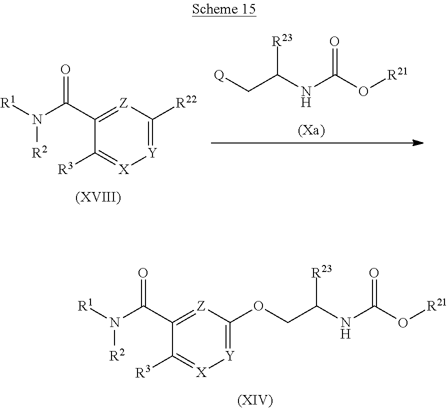

As shown in Scheme 15, compounds (XVIII) can be converted to compounds (XIV) by methods known to those skilled in the art and described for example in Tetrahedron 1988, 91; J. Am. Chem. Soc. 2010, 11592, WO2020211836. For example, the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction as shown in WO2017147700 and in J. Med. Chem. 2021, 11570.

The compounds of formula (XVIII) according to the invention, wherein R¹, R², R³, X, Y, Z are as defined for formula (I) and R²² is halogen, can be obtained by transformation of a compound of formula (XIX), wherein R³, X, Y, Z are as defined for formula (I) and R²² is halogen with a compound of formula (III), wherein R¹ and R² are as defined for formula (I), via an intermediate acid chloride or activated acylating agent as described below. This is shown in Scheme 16 below:

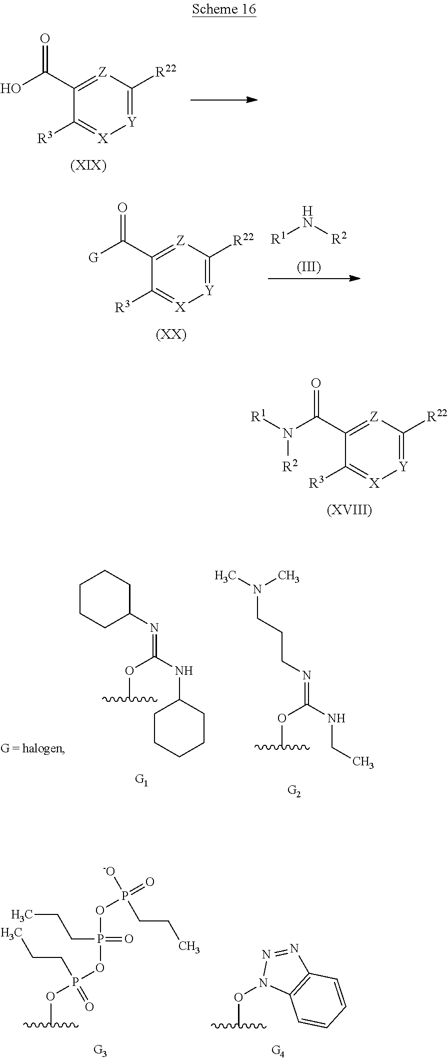

As shown in Scheme 16, compound XIX, is activated to compounds of formula XX by methods known to those skilled in the art and described for example in Tetrahedron 2005, 61 (46), 10827-10852. For example, compounds of formula XX where G is halogen are formed by treatment of compounds of formula II with for example, oxalyl chloride or thionyl chloride in the presence of catalytic quantities of DMF in inert solvents such as methylene dichloride or THF at temperatures between 25 to 170° C. preferably 25 to 80° C. Treatment of XX with compounds of formula III, wherein R¹ and R² are as defined in formula I, optionally in the presence of a base, e.g. triethylamine or pyridine leads to compounds of formula I. Alternatively, compounds of formula XVIII can be prepared by treatment of compounds of formula XIX with dicyclohexyl carbodiimide (DCC) or 1-ethyl-3-(3-dimethylaminopropyl)carbodiimide (EDC) to give the activated species IV, wherein G is G1 or G₂, in an inert solvent, e.g. pyridine, or THF optionally in the presence of a base, e.g. triethylamine, at temperatures between rt and 180° C. In addition, an acid of the formula XIX can also be activated by reaction with a coupling reagent such as propanephosphonic acid anhydride (T3P®) or O-(7-Aza-1-benzotriazolyl)-N,N,N′,N′-tetramethyluronium-hexafluorophosphat (HATU) to provide compounds of formula XX wherein G is G₃ and G₄ as described for example in Synthesis 2013, 45, 1569 and J. I Prakt. Chemie 1998, 340, 581. Subsequent reaction with an amine of the formula III provides compounds of formula XVIII.

The compounds of formula (XIX), wherein R³, X, Y and Z are as defined for formula (I), and R²² is halogen can be obtained by transformation of a compound of formula (XII), wherein R³, X, Y, Z are as defined for formula (I) and R²² is halogen, in the presence of a base or an acid. This is shown in Scheme 17 below.

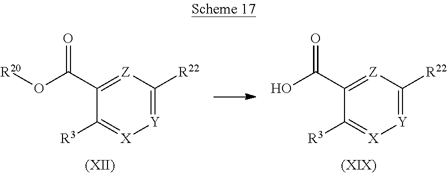

As shown in Scheme 17, compounds (XII) can undergo a hydrolysis reaction under basic conditions to compounds (XIX) by methods known to those skilled in the art and described for example in ACS Chem. Neurosci. 2017, 2374. For example, the reaction can be performed in water or in organic solvent, such as methanol, THF or methylene dichloride, or in mixtures; in the presence of a base, such as lithium hydroxide, sodium hydroxide or potassium hydroxide. Alternatively, the conversion of compounds (XII) into compounds (XIX) can be performed under acidic conditions by methods known to those skilled in the art and described for example in J. Med. Chem. 1986, 2363. For example, the reaction can be performed in water or in mixtures of water and an organic solvent, such as methanol, THF or dioxane; in the presence of an acid, such as trifluoroacetic acid, hydrochloric acid or sulfuric acid.

Compounds of formula (XV), wherein R¹, R², R³, X, Y, Z are as defined for formula (I), can be obtained in two steps by transformation of a compound of formula (XVIII), wherein R¹, R², R³, X, Y, Z are as defined for formula (I) and R²² is halogen via and intermediate compound of formula (XXI), wherein R¹, R², R³, X, Y, Z are as defined for formula (I) and R²⁰ is C₁-C₆ alkyl. This is shown in Scheme 18 below.

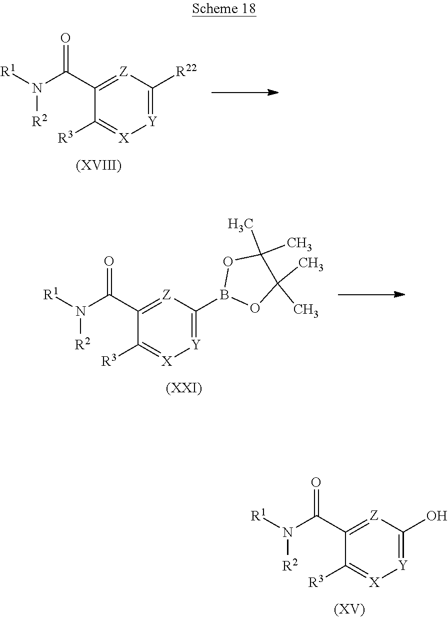

As shown in Scheme 18, compounds (XVIII) can be converted to compounds (XXI) by methods known to those skilled in the art with the name of Miyaura borylation reaction and described for example in WO2021076728; J. Med. Chem. 2022, 65, 15433. For example, the reaction can be performed in organic solvent, such as THF or dioxane, in the presence of a base, such as potassium carbonate or potassium acetate and a palladium catalyst in combination with an appropriate ligand, such as 1,1′-bis(diphenylphosphino)ferrocene dichloropalladium. Intermediate (XXI) can be converted into compounds of formula (XV) by methods known to those skilled in the art and described for example in WO2021127071, Tetrahedron Lett. 2014, 2082; Tetrahedron Lett. 2017, 3323; Tetrahedron 2005, 1417. For example the reaction can be performed in mixtures of water and an organic solvent, such as ethanol, acetonitrile or methylene dichloride, in the presence of hydrogen peroxide.

The compounds of formula (Ie) wherein R¹, R², R⁴, X, Y, Z are as defined for formula (I), A is oxygen and R³ is cyano, C₁-C₆-alkyl, C₁-C₆-alkoxy, C₃-C₆-cycloalkyl, C₁-C₆-haloalkoxy can be obtained by transformation of a compound of formula (If), wherein R¹, R², R⁴, X, Y, Z are as defined for formula (I) A is oxygen and R³ is halogen. This is shown in Scheme 19 below:

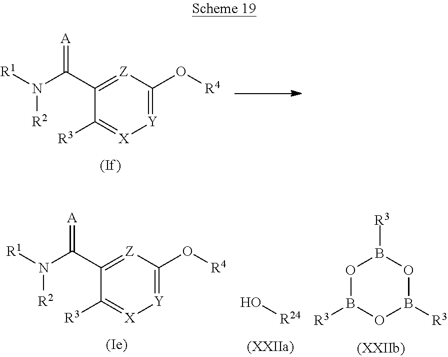

As shown in Scheme 19, compounds (If) can reacted with compounds of formula (XXIIa) wherein R²⁴ is C₁-C₆-alkyl, C₃-C₆-cycloalkyl and C₁-C₆-haloalkyl to compounds (Ie) wherein R³ is C₁-C₆-alkoxy and C₁-C₆-haloalkoxy by methods known to those skilled in the art and described for example in J. Med. Chem. 2021, J. Am. Chem. Soc. 2010, 11592. For example the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction. Alternatively, compounds (If) can reacted with compounds of formula (XXIIb) wherein R³ is C₁-C₆-alkyl, C₃-C₆-cycloalkyl and C₁-C₆-haloalkyl to compounds (Ie) by methods known to those skilled in the art and described for example in WO2021156787, WO2020210828, Tetrahedron Lett. 2000, 41, 6237. For example the reaction can be performed in organic solvent, such as dioxane, THF, dimethylformamide or toluene in the presence of a base, such as potassium carbonate or cesium carbonate and in the presence of a palladium catalyst with an appropriate ligand such as [1,1′-Bis(diphenylphosphino)ferrocene]dichloropalladium. Alternatively, compounds (If) can be reacted with cyanide salts such as potassium ferricyanide, sodium cyanide, potassium cyanide or zinc cyanide to compounds (Ie) wherein R³ is cyano by methods known to those skilled in the art and described for example in Adv. Synth. Cat. 2017, 359, 2345, US20090062541, Synthesis 2008, 20, 3351. For example the reaction can be performed in organic solvent, such as dioxane, THF, methyl-THF, dimethylformamide, dimethyl sulfoxide or toluene in the presence of an organometallic catalyst such as cuprous iodide, [1,1′-Bis(diphenylphosphino)ferrocene]dichloropalladium, cupric acetate, XantPhos Pd G3, in the optional presence of a ligand such as butylimidazole, N,N′-dimethylethylenediamine, 1-methylimidazole, 1,10-Phenanthroline; in the optional presence of a base, such as potassium carbonate, sodium carbonate or cesium carbonate, in the optional presence of additives such as polyethylene glycol dodecyl ether and polymethylhydrosiloxane.

The compounds of formula (Va) wherein R⁴, X, Y, Z are as defined for formula (I), R²⁰ is C₁-C₆alkyl and R³ is cyano, C₁-C₆-alkyl, C₁-C₆-alkoxy, C₃-C₆-cycloalkyl, C₁-C₆-haloalkoxy can be obtained by transformation of a compound of formula (Vb), wherein R⁴, X, Y, Z are as defined for formula (I) R²⁰ is C₁-C₆alkyl and R³ is halogen. This is shown in Scheme 20 below:

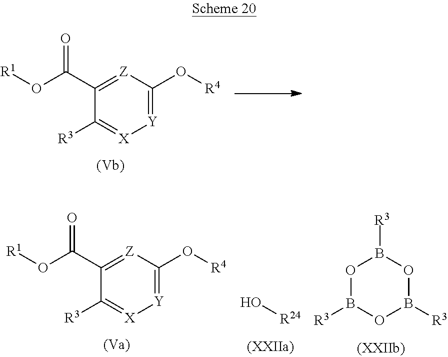

As shown in Scheme 20, compounds (Vb) can reacted with compounds of formula (XXIIa) wherein R²⁴ is C₁-C₆-alkyl, C₃-C₆-cycloalkyl and C₁-C₆-haloalkyl to compounds (Va) wherein R³ is C₁-C₆-alkoxy and C₁-C₆-haloalkoxy by methods known to those skilled in the art and described for example in J. Med. Chem. 2021, J. Am. Chem. Soc. 2010, 11592. For example the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction. Alternatively, compounds (Vb) can reacted with compounds of formula (XXIIb) wherein R³ is C₁-C₆-alkyl, C₃-C₆-cycloalkyl and C₁-C₆-haloalkyl to compounds (Va) by methods known to those skilled in the art and described for example in WO2021156787, WO2020210828, Tetrahedron Letters 2000, 41, 6237. For example the reaction can be performed in organic solvent, such as dioxane, THF, dimethylformamide or toluene in the presence of a base, such as potassium carbonate or cesium carbonate and in the presence of a palladium catalyst with an appropriate ligand such as [1,1′-Bis(diphenylphosphino)ferrocene]dichloropalladium. Alternatively, compounds (Vb) can be reacted with cyanide salts such as potassium ferricyanide, sodium cyanide, potassium cyanide or zinc cyanide to compounds (Va) wherein R³ is cyano by methods known to those skilled in the art and described for example in Adv. Synth. Cat. 2017, 359, 2345, US20090062541, Synthesis (2008), (20), 3351. For example the reaction can be performed in organic solvent, such as dioxane, THF, methyl-THF, dimethylformamide, dimethyl sulfoxide or toluene in the presence of an organometallic catalyst such as cuprous iodide, [1,1′-Bis(diphenylphosphino)ferrocene]dichloropalladium, cupric acetate, XantPhos Pd G3, in the optional presence of a ligand such as butylimidazole, N,N′-dimethylethylenediamine, 1-methylimidazole, 1,10-Phenanthroline; in the optional presence of a base, such as potassium carbonate, sodium carbonate or cesium carbonate, in the optional presence of additives such as polyethylene glycol dodecyl ether and polymethylhydrosiloxane.

The compounds of formula (VIIIa) wherein X, Y, Z are as defined for formula (I), R²⁰ is C₁-C₆alkyl, R²¹ is C₁-C₆alkyl, R²³ is hydrogen or methyl and R³ is cyano, C₁-C₆-alkyl, C₁-C₆-alkoxy, C₃-C₆-cycloalkyl, C₁-C₆-haloalkoxy can be obtained by transformation of a compound of formula (VIIIb), wherein X, Y, Z are as defined for formula (I) R²⁰ is C₁-C₆alkyl, R²¹ is C₁-C₆ alkyl, R²³ is hydrogen or methyl and R³ is halogen. This is shown in Scheme 21 below:

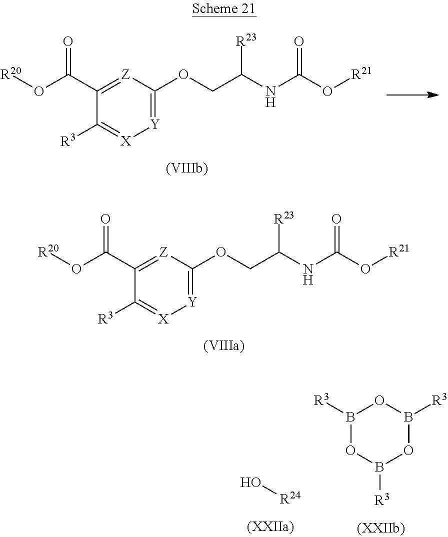

As shown in Scheme 21, compounds (VIIIb) can reacted with compounds of formula (XXIIa) wherein R²⁴ is C₁-C₆-alkyl, C₃-C₆-cycloalkyl and C₁-C₆-haloalkyl to compounds (VIIIa) wherein R³ is C₁-C₆-alkoxy and C₁-C₆-haloalkoxy by methods known to those skilled in the art and described for example in J. Med. Chem. 2021, J. Am. Chem. Soc. 2010, 11592. For example the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction. Alternatively, compounds (VIIIb) can reacted with compounds of formula (XXIIb) wherein R³ is C₁-C₆-alkyl, C₃-C₆-cycloalkyl and C₁-C₆-haloalkyl to compounds (VIIIa) by methods known to those skilled in the art and described for example in WO2021156787, WO2020210828, Tetrahedron Lett. 2000, 41, 6237. For example the reaction can be performed in organic solvent, such as dioxane, THF, dimethylformamide or toluene in the presence of a base, such as potassium carbonate or cesium carbonate and in the presence of a palladium catalyst with an appropriate ligand such as [1,1′-Bis(diphenylphosphino)ferrocene]dichloropalladium. Alternatively, compounds (VIIIb) can be reacted with cyanide salts such as potassium ferricyanide, sodium cyanide, potassium cyanide or zinc cyanide to compounds (VIIIa) wherein R³ is cyano by methods known to those skilled in the art and described for example in Adv. Synth. Cat. 2017, 359, 2345, US20090062541, Synthesis 2008, (20), 3351. For example the reaction can be performed in organic solvent, such as dioxane, THF, methyl-THF, dimethylformamide, dimethyl sulfoxide or toluene in the presence of an organometallic catalyst such as cuprous iodide, [1,1′-Bis(diphenylphosphino)ferrocene]dichloropalladium, cupric acetate, XantPhos Pd G3, in the optional presence of a ligand such as butylimidazole, N,N′-dimethylethylenediamine, 1-methylimidazole, 1,10-Phenanthroline; in the optional presence of a base, such as potassium carbonate, sodium carbonate or cesium carbonate, in the optional presence of additives such as polyethylene glycol dodecyl ether and polymethylhydrosiloxane.

The compounds of formula (XIVa) wherein R¹, R², X, Y, Z are as defined for formula (I), R²¹ is C₁-C₆ alkyl, R²³ is hydrogen or methyl and R³ is cyano, C₁-C₆-alkyl, C₁-C₆-alkoxy, C₃-C₆-cycloalkyl, C₁-C₆-haloalkoxy can be obtained by transformation of a compound of formula (XIVb), wherein R¹, R², X, Y, Z are as defined for formula (I), R²¹ is C₁-C₆ alkyl, R²³ is hydrogen or methyl and R³ is halogen. This is shown in Scheme 22 below:

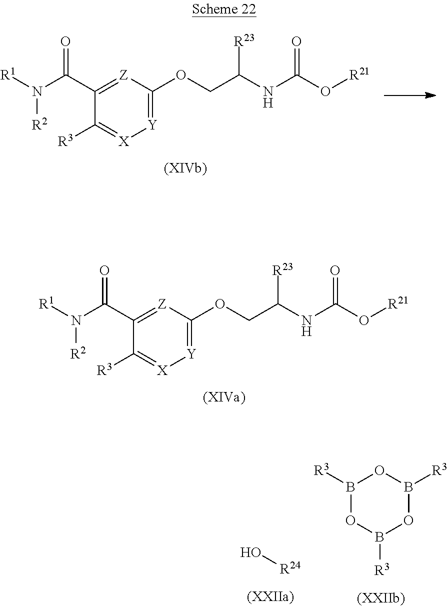

As shown in Scheme 21, compounds (XIVb) can reacted with compounds of formula (XXIIa) wherein R²⁴ is C₁-C₆-alkyl, C₃-C₆-cycloalkyl and C₁-C₆-haloalkyl to compounds (XIVa) wherein R³ is C₁-C₆-alkoxy and C₁-C₆-haloalkoxy by methods known to those skilled in the art and described for example in J. Med. Chem. 2021, J. Am. Chem. Soc. 2010, 11592. For example the reaction can be performed in organic solvent, such as dioxane, dimethylformamide or toluene in the presence of a base, such as sodium hydride, potassium carbonate or cesium carbonate. A palladium catalyst and an appropriate ligand can be used to facilitate the reaction. Alternatively, compounds (XIVb) can be reacted with compounds of formula (XXIIb) wherein R³ is C₁-C₆-alkyl, C₃-C₆-cycloalkyl and C₁-C₆-haloalkyl to compounds (XIVa) by methods known to those skilled in the art and described for example in WO2021156787, WO2020210828, Tetrahedron Lett. 2000, 41, 6237. For example the reaction can be performed in organic solvent, such as dioxane, THF, dimethylformamide or toluene in the presence of a base, such as potassium carbonate or cesium carbonate and in the presence of a palladium catalyst with an appropriate ligand such as [1,1′-Bis(diphenylphosphino)ferrocene]dichloropalladium. Alternatively, compounds (XIVb) can reacted with cyanide salts such as potassium ferricyanide, sodium cyanide, potassium cyanide or zinc cyanide to compounds (XIVa) wherein R³ is cyano by methods known to those skilled in the art and described for example in Adv. Synth. Cat. 2017, 359, 2345, US20090062541, Synthesis 2008, (20), 3351. For example the reaction can be performed in organic solvent, such as dioxane, THF, methyl-THF, dimethylformamide, dimethyl sulfoxide or toluene in the presence of an organometallic catalyst such as cuprous iodide, [1,1′-Bis(diphenylphosphino)ferrocene]dichloropalladium, cupric acetate, XantPhos Pd G3, in the optional presence of a ligand such as butylimidazole, N,N′-dimethylethylenediamine, 1-methylimidazole, 1,10-Phenanthroline; in the optional presence of a base, such as potassium carbonate, sodium carbonate or cesium carbonate, in the optional presence of additives such as polyethylene glycol dodecyl ether and polymethylhydrosiloxane.

Compounds of formula (XXIII), wherein R³, X, Y, Z are as defined for formula (I), R²¹ is C₁-C₆ alkyl and R²³ is hydrogen or methyl, can be obtained by transformation of a compound of formula (XIX), wherein R³, X, Y, Z are as defined for formula (I) and R²² is a halogen with a compound of formula (Xa), wherein R²¹ is C₁-C₆ alkyl, R²³ is hydrogen or methyl and Q is hydroxy. This is shown in Scheme 23 below.

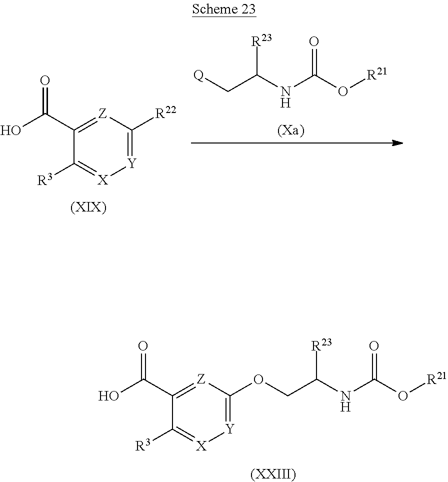

As shown in Scheme 23, compounds (XIX) can be converted to compounds (XXIII) by methods known to those skilled in the art and described for example in Tetrahedron 1988, 91; J. Am. Chem. Soc. 2010, 11592, WO2020211836. For example, the reaction can be performed in organic solvent, such as dioxane, dimethylformamide, dimethyl sulfoxide or toluene in the presence of a base, such as sodium hydride, potassium carbonate, cesium carbonate, potassium tertbutoxide, sodium hydroxide, benzyltrimethylammonium hydroxide. A palladium catalyst and an appropriate ligand can be used to facilitate the reaction as shown in WO2017147700 and in J. Med. Chem. 2021, 11570.

Alternatively, the compounds of formula (XIV) wherein R¹, R², R³, X, Y, Z are as defined for formula (I), R²¹ is C₁-C₆ alkyl and R²³ is hydrogen or methyl, can be obtained by transformation of a compound of formula (XXIII), wherein R³, X, Y, Z are as defined for formula (I)), R²¹ is C₁-C₆ alkyl and R²³ is hydrogen or methyl with a compound of formula (III), wherein R¹ and R² are as defined for formula (I), via an intermediate acid chloride or activated acylating agent as described below. This is shown in Scheme 24 below:

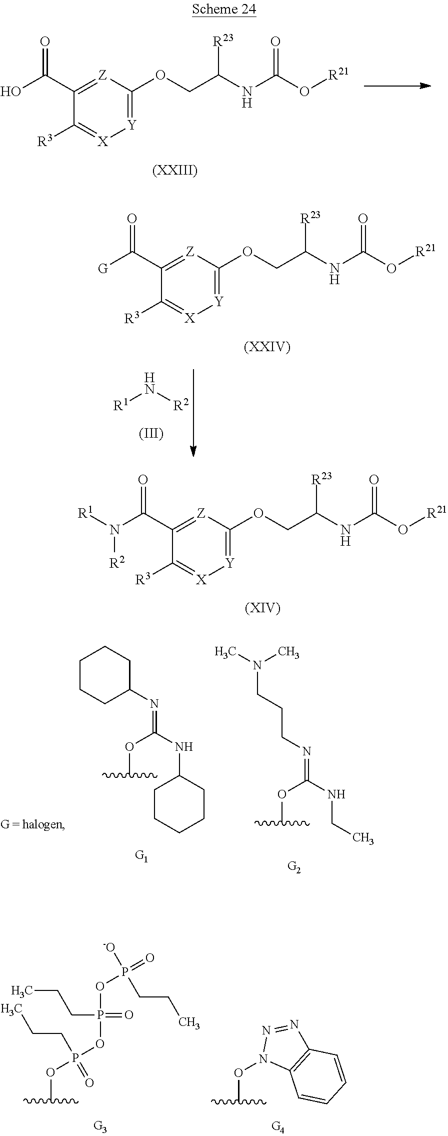

As shown in Scheme 24, compound (XXIII), is activated to compounds of formula (XXIV) by methods known to those skilled in the art and described for example in Tetrahedron 2005, 61 (46), 10827-10852. For example, compounds of formula (XXIV) where G is halogen are formed by treatment of compounds of formula (XXIII) with for example, oxalyl chloride or thionyl chloride in the presence of catalytic quantities of DMF in inert solvents such as methylene dichloride or THF at temperatures between 25 to 170° C. preferably 25 to 80° C. Treatment of (XXIV) with compounds of formula III, wherein R¹ and R² are as defined in formula I, optionally in the presence of a base, e.g. triethylamine or pyridine leads to compounds of formula (XIV). Alternatively, compounds of formula XIV can be prepared by treatment of compounds of formula XXIII with dicyclohexyl carbodiimide (DCC) or 1-ethyl-3-(3-dimethylaminopropyl)carbodiimide (EDC) to give the activated species XXIV, wherein G is G₁ or G2, in an inert solvent, e.g. pyridine, or THF optionally in the presence of a base, e.g. triethylamine, at temperatures between rt and 180° C. In addition, an acid of the formula XXIII can also be activated by reaction with a coupling reagent such as propanephosphonic acid anhydride (T3P®) or O-(7-Aza-1-benzotriazolyl)-N,N,N′,N′-tetramethyluronium-hexafluorophosphat (HATU) to provide compounds of formula XXIV wherein G is G₃ and G₄ as described for example in Synthesis 2013, 45, 1569 and J. Prakt. Chem. 1998, 340, 581. Subsequent reaction with an amine of the formula III provides compounds of formula XIV.

Depending on the procedure or the reaction conditions, the reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula (I) according to the following Tables 1 to 5 can be prepared according to the methods described above. The examples which follow are intended to illustrate the invention and show preferred compounds of formula (I), in the form of a compound of formulae (I-1a), (I-2a), (I-3a), (I-4a) and (I-5a).

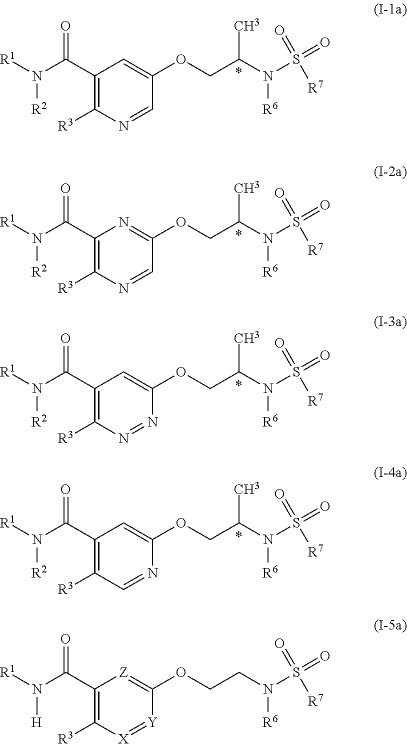

TABLE 1
Makes available 1447 compounds of the formula (I-1a), where the substituents
R¹, R², R³, R⁶ and R⁷ are designated in each row below.

| Index | R² | R³ | R⁶ | R⁷ | R¹ |
|---|---|---|---|---|---|
| 1 | H | Cl | H | CF₃ | ethyl |
| 2 | H | Cl | H | CHF₂ | ethyl |
| 3 | H | Cl | H | iPr | ethyl |
| 4 | H | Cl | H | cyclopropyl | ethyl |
| 5 | H | Cl | methyl | CF₃ | ethyl |
| 6 | H | Cl | methyl | CHF₂ | ethyl |
| 7 | H | Cl | methyl | iPr | ethyl |
| 8 | H | Cl | methyl | cyclopropyl | ethyl |
| 9 | H | Cl | methoxymethyl | CF₃ | ethyl |
| 10 | H | Cl | methoxymethyl | CHF₂ | ethyl |
| 11 | H | Cl | methoxymethyl | iPr | ethyl |
| 12 | H | Cl | methoxymethyl | cyclopropyl | ethyl |
| 13 | H | Cl | acetyl | CF₃ | ethyl |
| 14 | H | Cl | acetyl | CHF₂ | ethyl |
| 15 | H | Cl | acetyl | iPr | ethyl |
| 16 | H | Cl | acetyl | cyclopropyl | ethyl |
| 17 | H | Me | H | CF₃ | ethyl |
| 18 | H | Me | H | CHF₂ | ethyl |
| 19 | H | Me | H | iPr | ethyl |
| 20 | H | Me | H | cyclopropyl | ethyl |
| 21 | H | Me | methyl | CF₃ | ethyl |
| 22 | H | Me | methyl | CHF₂ | ethyl |
| 23 | H | Me | methyl | iPr | ethyl |
| 24 | H | Me | methyl | cyclopropyl | ethyl |
| 25 | H | Me | methoxymethyl | CF₃ | ethyl |
| 26 | H | Me | methoxymethyl | CHF₂ | ethyl |
| 27 | H | Me | methoxymethyl | iPr | ethyl |
| 28 | H | Me | methoxymethyl | cyclopropyl | ethyl |
| 29 | H | Me | acetyl | CF₃ | ethyl |
| 30 | H | Me | acetyl | CHF₂ | ethyl |
| 31 | H | Me | acetyl | iPr | ethyl |
| 32 | H | Me | acetyl | cyclopropyl | ethyl |
| 33 | methyl | Cl | H | CF₃ | ethyl |
| 34 | methyl | Cl | H | CHF₂ | ethyl |
| 35 | methyl | Cl | H | iPr | ethyl |
| 36 | methyl | Cl | H | cyclopropyl | ethyl |
| 37 | methyl | Cl | methyl | CF₃ | ethyl |
| 38 | methyl | Cl | methyl | CHF₂ | ethyl |
| 39 | methyl | Cl | methyl | iPr | ethyl |
| 40 | methyl | Cl | methyl | cyclopropyl | ethyl |
| 41 | methyl | Cl | methoxymethyl | CF₃ | ethyl |
| 42 | methyl | Cl | methoxymethyl | CHF₂ | ethyl |
| 43 | methyl | Cl | methoxymethyl | iPr | ethyl |
| 44 | methyl | Cl | methoxymethyl | cyclopropyl | ethyl |
| 45 | methyl | Cl | acetyl | CF₃ | ethyl |
| 46 | methyl | Cl | acetyl | CHF₂ | ethyl |
| 47 | methyl | Cl | acetyl | iPr | ethyl |
| 48 | methyl | Cl | acetyl | cyclopropyl | ethyl |
| 49 | methyl | Me | H | CF₃ | ethyl |
| 50 | methyl | Me | H | CHF₂ | ethyl |
| 51 | methyl | Me | H | iPr | ethyl |
| 52 | methyl | Me | H | cyclopropyl | ethyl |
| 53 | methyl | Me | methyl | CF₃ | ethyl |
| 54 | methyl | Me | methyl | CHF₂ | ethyl |
| 55 | methyl | Me | methyl | iPr | ethyl |
| 56 | methyl | Me | methyl | cyclopropyl | ethyl |
| 57 | methyl | Me | methoxymethyl | CF₃ | ethyl |
| 58 | methyl | Me | methoxymethyl | CHF₂ | ethyl |
| 59 | methyl | Me | methoxymethyl | iPr | ethyl |
| 60 | methyl | Me | methoxymethyl | cyclopropyl | ethyl |
| 61 | methyl | Me | acetyl | CF₃ | ethyl |
| 62 | methyl | Me | acetyl | CHF₂ | ethyl |
| 63 | methyl | Me | acetyl | iPr | ethyl |
| 64 | methyl | Me | acetyl | cyclopropyl | ethyl |
| 65 | acetyl | Cl | H | CF₃ | ethyl |
| 66 | acetyl | Cl | H | CHF₂ | ethyl |
| 67 | acetyl | Cl | H | iPr | ethyl |
| 68 | acetyl | Cl | H | cyclopropyl | ethyl |
| 69 | acetyl | Cl | methyl | CF₃ | ethyl |
| 70 | acetyl | Cl | methyl | CHF₂ | ethyl |
| 71 | acetyl | Cl | methyl | iPr | ethyl |
| 72 | acetyl | Cl | methyl | cyclopropyl | ethyl |
| 73 | acetyl | Cl | methoxymethyl | CF₃ | ethyl |
| 74 | acetyl | Cl | methoxymethyl | CHF₂ | ethyl |
| 75 | acetyl | Cl | methoxymethyl | iPr | ethyl |
| 76 | acetyl | Cl | methoxymethyl | cyclopropyl | ethyl |
| 77 | acetyl | Cl | acetyl | CF₃ | ethyl |
| 78 | acetyl | Cl | acetyl | CHF₂ | ethyl |
| 79 | acetyl | Cl | acetyl | iPr | ethyl |
| 80 | acetyl | Cl | acetyl | cyclopropyl | ethyl |
| 81 | acetyl | Me | H | CF₃ | ethyl |
| 82 | acetyl | Me | H | CHF₂ | ethyl |
| 83 | acetyl | Me | H | iPr | ethyl |
| 84 | acetyl | Me | H | cyclopropyl | ethyl |
| 85 | acetyl | Me | methyl | CF₃ | ethyl |
| 86 | acetyl | Me | methyl | CHF₂ | ethyl |
| 87 | acetyl | Me | methyl | iPr | ethyl |
| 88 | acetyl | Me | methyl | cyclopropyl | ethyl |
| 89 | acetyl | Me | methoxymethyl | CF₃ | ethyl |
| 90 | acetyl | Me | methoxymethyl | CHF₂ | ethyl |
| 91 | acetyl | Me | methoxymethyl | iPr | ethyl |
| 92 | acetyl | Me | methoxymethyl | cyclopropyl | ethyl |
| 93 | acetyl | Me | acetyl | CF₃ | ethyl |
| 94 | acetyl | Me | acetyl | CHF₂ | ethyl |
| 95 | acetyl | Me | acetyl | iPr | ethyl |
| 96 | acetyl | Me | acetyl | cyclopropyl | ethyl |
| 97 | 2-methyl propanoyl | Cl | H | CF₃ | ethyl |
| 98 | 2-methyl propanoyl | Cl | H | CHF₂ | ethyl |
| 99 | 2-methyl propanoyl | Cl | H | iPr | ethyl |
| 100 | 2-methyl propanoyl | Cl | H | cyclopropyl | ethyl |
| 101 | 2-methyl propanoyl | Cl | methyl | CF₃ | ethyl |
| 102 | 2-methyl propanoyl | Cl | methyl | CHF₂ | ethyl |
| 103 | 2-methyl propanoyl | Cl | methyl | iPr | ethyl |
| 104 | 2-methyl propanoyl | Cl | methyl | cyclopropyl | ethyl |
| 105 | 2-methyl propanoyl | Cl | methoxymethyl | CF₃ | ethyl |
| 106 | 2-methyl propanoyl | Cl | methoxymethyl | CHF₂ | ethyl |
| 107 | 2-methyl propanoyl | Cl | methoxymethyl | iPr | ethyl |
| 108 | 2-methyl propanoyl | Cl | methoxymethyl | cyclopropyl | ethyl |
| 109 | 2-methyl propanoyl | Cl | acetyl | CF₃ | ethyl |
| 110 | 2-methyl propanoyl | Cl | acetyl | CHF₂ | ethyl |
| 111 | 2-methyl propanoyl | Cl | acetyl | iPr | ethyl |
| 112 | 2-methyl propanoyl | Cl | acetyl | cyclopropyl | ethyl |
| 113 | 2-methyl propanoyl | Me | H | CF₃ | ethyl |
| 114 | 2-methyl propanoyl | Me | H | CHF₂ | ethyl |
| 115 | 2-methyl propanoyl | Me | H | iPr | ethyl |
| 116 | 2-methyl propanoyl | Me | H | cyclopropyl | ethyl |
| 117 | 2-methyl propanoyl | Me | methyl | CF₃ | ethyl |
| 118 | 2-methyl propanoyl | Me | methyl | CHF₂ | ethyl |
| 119 | 2-methyl propanoyl | Me | methyl | iPr | ethyl |
| 120 | 2-methyl propanoyl | Me | methyl | cyclopropyl | ethyl |
| 121 | 2-methyl propanoyl | Me | methoxymethyl | CF₃ | ethyl |
| 122 | 2-methyl propanoyl | Me | methoxymethyl | CHF₂ | ethyl |
| 123 | 2-methyl propanoyl | Me | methoxymethyl | iPr | ethyl |
| 124 | 2-methyl propanoyl | Me | methoxymethyl | cyclopropyl | ethyl |
| 125 | 2-methyl propanoyl | Me | acetyl | CF₃ | ethyl |
| 126 | 2-methyl propanoyl | Me | acetyl | CHF₂ | ethyl |
| 127 | 2-methyl propanoyl | Me | acetyl | iPr | ethyl |
| 128 | 2-methyl propanoyl | Me | acetyl | cyclopropyl | ethyl |
| 129 | H | Cl | H | CF₃ | isopropyl |
| 130 | H | Cl | H | CHF₂ | isopropyl |
| 131 | H | Cl | H | iPr | isopropyl |
| 132 | H | Cl | H | cyclopropyl | isopropyl |
| 133 | H | Cl | methyl | CF₃ | isopropyl |
| 134 | H | Cl | methyl | CHF₂ | isopropyl |
| 135 | H | Cl | methyl | iPr | isopropyl |
| 136 | H | Cl | methyl | cyclopropyl | isopropyl |
| 137 | H | Cl | methoxymethyl | CF₃ | isopropyl |
| 138 | H | Cl | methoxymethyl | CHF₂ | isopropyl |
| 139 | H | Cl | methoxymethyl | iPr | isopropyl |
| 140 | H | Cl | methoxymethyl | cyclopropyl | isopropyl |
| 141 | H | Cl | acetyl | CF₃ | isopropyl |
| 142 | H | Cl | acetyl | CHF₂ | isopropyl |
| 143 | H | Cl | acetyl | iPr | isopropyl |
| 144 | H | Cl | acetyl | cyclopropyl | isopropyl |
| 145 | H | Me | H | CF₃ | isopropyl |
| 146 | H | Me | H | CHF₂ | isopropyl |
| 147 | H | Me | H | iPr | isopropyl |
| 148 | H | Me | H | cyclopropyl | isopropyl |
| 149 | H | Me | methyl | CF₃ | isopropyl |
| 150 | H | Me | methyl | CHF₂ | isopropyl |
| 151 | H | Me | methyl | iPr | isopropyl |
| 152 | H | Me | methyl | cyclopropyl | isopropyl |
| 153 | H | Me | methoxymethyl | CF₃ | isopropyl |
| 154 | H | Me | methoxymethyl | CHF₂ | isopropyl |
| 155 | H | Me | methoxymethyl | iPr | isopropyl |
| 156 | H | Me | methoxymethyl | cyclopropyl | isopropyl |
| 157 | H | Me | acetyl | CF₃ | isopropyl |
| 158 | H | Me | acetyl | CHF₂ | isopropyl |
| 159 | H | Me | acetyl | iPr | isopropyl |
| 160 | H | Me | acetyl | cyclopropyl | isopropyl |
| 161 | methyl | Cl | H | CF₃ | isopropyl |
| 162 | methyl | Cl | H | CHF₂ | isopropyl |
| 163 | methyl | Cl | H | iPr | isopropyl |
| 164 | methyl | Cl | H | cyclopropyl | isopropyl |
| 165 | methyl | Cl | methyl | CF₃ | isopropyl |
| 166 | methyl | Cl | methyl | CHF₂ | isopropyl |
| 167 | methyl | Cl | methyl | iPr | isopropyl |
| 168 | methyl | Cl | methyl | cyclopropyl | isopropyl |
| 169 | methyl | Cl | methoxymethyl | CF₃ | isopropyl |
| 170 | methyl | Cl | methoxymethyl | CHF₂ | isopropyl |
| 171 | methyl | Cl | methoxymethyl | iPr | isopropyl |
| 172 | methyl | Cl | methoxymethyl | cyclopropyl | isopropyl |
| 173 | methyl | Cl | acetyl | CF₃ | isopropyl |
| 174 | methyl | Cl | acetyl | CHF₂ | isopropyl |
| 175 | methyl | Cl | acetyl | iPr | isopropyl |
| 176 | methyl | Cl | acetyl | cyclopropyl | isopropyl |
| 177 | methyl | Me | H | CF₃ | isopropyl |
| 178 | methyl | Me | H | CHF₂ | isopropyl |
| 179 | methyl | Me | H | iPr | isopropyl |
| 180 | methyl | Me | H | cyclopropyl | isopropyl |
| 181 | methyl | Me | methyl | CF₃ | isopropyl |
| 182 | methyl | Me | methyl | CHF₂ | isopropyl |
| 183 | methyl | Me | methyl | iPr | isopropyl |
| 184 | methyl | Me | methyl | cyclopropyl | isopropyl |
| 185 | methyl | Me | methoxymethyl | CF₃ | isopropyl |
| 186 | methyl | Me | methoxymethyl | CHF₂ | isopropyl |
| 187 | methyl | Me | methoxymethyl | iPr | isopropyl |
| 188 | methyl | Me | methoxymethyl | cyclopropyl | isopropyl |
| 189 | methyl | Me | acetyl | CF₃ | isopropyl |
| 190 | methyl | Me | acetyl | CHF₂ | isopropyl |
| 191 | methyl | Me | acetyl | iPr | isopropyl |
| 192 | methyl | Me | acetyl | cyclopropyl | isopropyl |
| 193 | acetyl | Cl | H | CF₃ | isopropyl |
| 194 | acetyl | Cl | H | CHF₂ | isopropyl |
| 195 | acetyl | Cl | H | iPr | isopropyl |
| 196 | acetyl | Cl | H | cyclopropyl | isopropyl |
| 197 | acetyl | Cl | methyl | CF₃ | isopropyl |
| 198 | acetyl | Cl | methyl | CHF₂ | isopropyl |
| 199 | acetyl | Cl | methyl | iPr | isopropyl |
| 200 | acetyl | Cl | methyl | cyclopropyl | isopropyl |
| 201 | acetyl | Cl | methoxymethyl | CF₃ | isopropyl |
| 202 | acetyl | Cl | methoxymethyl | CHF₂ | isopropyl |
| 203 | acetyl | Cl | methoxymethyl | iPr | isopropyl |
| 204 | acetyl | Cl | methoxymethyl | cyclopropyl | isopropyl |
| 205 | acetyl | Cl | acetyl | CF₃ | isopropyl |
| 206 | acetyl | Cl | acetyl | CHF₂ | isopropyl |
| 207 | acetyl | Cl | acetyl | iPr | isopropyl |
| 208 | acetyl | Cl | acetyl | cyclopropyl | isopropyl |
| 209 | acetyl | Me | H | CF₃ | isopropyl |
| 210 | acetyl | Me | H | CHF₂ | isopropyl |
| 211 | acetyl | Me | H | iPr | isopropyl |
| 212 | acetyl | Me | H | cyclopropyl | isopropyl |
| 213 | acetyl | Me | methyl | CF₃ | isopropyl |
| 214 | acetyl | Me | methyl | CHF₂ | isopropyl |
| 215 | acetyl | Me | methyl | iPr | isopropyl |
| 216 | acetyl | Me | methyl | cyclopropyl | isopropyl |
| 217 | acetyl | Me | methoxymethyl | CF₃ | isopropyl |
| 218 | acetyl | Me | methoxymethyl | CHF₂ | isopropyl |
| 219 | acetyl | Me | methoxymethyl | iPr | isopropyl |
| 220 | acetyl | Me | methoxymethyl | cyclopropyl | isopropyl |
| 221 | acetyl | Me | acetyl | CF₃ | isopropyl |
| 222 | acetyl | Me | acetyl | CHF₂ | isopropyl |
| 223 | acetyl | Me | acetyl | iPr | isopropyl |
| 224 | acetyl | Me | acetyl | cyclopropyl | isopropyl |
| 225 | 2-methyl propanoyl | Cl | H | CF₃ | isopropyl |
| 226 | 2-methyl propanoyl | Cl | H | CHF₂ | isopropyl |
| 227 | 2-methyl propanoyl | Cl | H | iPr | isopropyl |
| 228 | 2-methyl propanoyl | Cl | H | cyclopropyl | isopropyl |
| 229 | 2-methyl propanoyl | Cl | methyl | CF₃ | isopropyl |
| 230 | 2-methyl propanoyl | Cl | methyl | CHF₂ | isopropyl |
| 231 | 2-methyl propanoyl | Cl | methyl | iPr | isopropyl |
| 232 | 2-methyl propanoyl | Cl | methyl | cyclopropyl | isopropyl |
| 233 | 2-methyl propanoyl | Cl | methoxymethyl | CF₃ | isopropyl |
| 234 | 2-methyl propanoyl | Cl | methoxymethyl | CHF₂ | isopropyl |
| 235 | 2-methyl propanoyl | Cl | methoxymethyl | iPr | isopropyl |
| 236 | 2-methyl propanoyl | Cl | methoxymethyl | cyclopropyl | isopropyl |
| 237 | 2-methyl propanoyl | Cl | acetyl | CF₃ | isopropyl |
| 238 | 2-methyl propanoyl | Cl | acetyl | CHF₂ | isopropyl |
| 239 | 2-methyl propanoyl | Cl | acetyl | iPr | isopropyl |
| 240 | 2-methyl propanoyl | Cl | acetyl | cyclopropyl | isopropyl |
| 241 | 2-methyl propanoyl | Me | H | CF₃ | isopropyl |
| 242 | 2-methyl propanoyl | Me | H | CHF₂ | isopropyl |
| 243 | 2-methyl propanoyl | Me | H | iPr | isopropyl |
| 244 | 2-methyl propanoyl | Me | H | cyclopropyl | isopropyl |
| 245 | 2-methyl propanoyl | Me | methyl | CF₃ | isopropyl |
| 246 | 2-methyl propanoyl | Me | methyl | CHF₂ | isopropyl |
| 247 | 2-methyl propanoyl | Me | methyl | iPr | isopropyl |
| 248 | 2-methyl propanoyl | Me | methyl | cyclopropyl | isopropyl |
| 249 | 2-methyl propanoyl | Me | methoxymethyl | CF₃ | isopropyl |
| 250 | 2-methyl propanoyl | Me | methoxymethyl | CHF₂ | isopropyl |
| 251 | 2-methyl propanoyl | Me | methoxymethyl | iPr | isopropyl |
| 252 | 2-methyl propanoyl | Me | methoxymethyl | cyclopropyl | isopropyl |
| 253 | 2-methyl propanoyl | Me | acetyl | CF₃ | isopropyl |
| 254 | 2-methyl propanoyl | Me | acetyl | CHF₂ | isopropyl |
| 255 | 2-methyl propanoyl | Me | acetyl | iPr | isopropyl |
| 256 | 2-methyl propanoyl | Me | acetyl | cyclopropyl | isopropyl |
| 257 | H | Cl | H | CF₃ | propargyl |
| 258 | H | Cl | H | CHF₂ | propargyl |
| 259 | H | Cl | H | iPr | propargyl |
| 260 | H | Cl | H | cyclopropyl | propargyl |
| 261 | H | Cl | methyl | CF₃ | propargyl |
| 262 | H | Cl | methyl | CHF₂ | propargyl |
| 263 | H | Cl | methyl | iPr | propargyl |
| 264 | H | Cl | methyl | cyclopropyl | propargyl |
| 265 | H | Cl | methoxymethyl | CF₃ | propargyl |
| 266 | H | Cl | methoxymethyl | CHF₂ | propargyl |
| 267 | H | Cl | methoxymethyl | iPr | propargyl |
| 268 | H | Cl | methoxymethyl | cyclopropyl | propargyl |
| 269 | H | Cl | acetyl | CF₃ | propargyl |
| 270 | H | Cl | acetyl | CHF₂ | propargyl |
| 271 | H | Cl | acetyl | iPr | propargyl |
| 272 | H | Cl | acetyl | cyclopropyl | propargyl |
| 273 | H | Me | H | CF₃ | propargyl |
| 274 | H | Me | H | CHF₂ | propargyl |
| 275 | H | Me | H | iPr | propargyl |
| 276 | H | Me | H | cyclopropyl | propargyl |
| 277 | H | Me | methyl | CF₃ | propargyl |
| 278 | H | Me | methyl | CHF₂ | propargyl |
| 279 | H | Me | methyl | iPr | propargyl |
| 280 | H | Me | methyl | cyclopropyl | propargyl |
| 281 | H | Me | methoxymethyl | CF₃ | propargyl |
| 282 | H | Me | methoxymethyl | CHF₂ | propargyl |
| 283 | H | Me | methoxymethyl | iPr | propargyl |
| 284 | H | Me | methoxymethyl | cyclopropyl | propargyl |
| 285 | H | Me | acetyl | CF₃ | propargyl |
| 286 | H | Me | acetyl | CHF₂ | propargyl |
| 287 | H | Me | acetyl | iPr | propargyl |
| 288 | H | Me | acetyl | cyclopropyl | propargyl |
| 289 | methyl | Cl | H | CF₃ | propargyl |
| 290 | methyl | Cl | H | CHF₂ | propargyl |
| 291 | methyl | Cl | H | iPr | propargyl |
| 292 | methyl | Cl | H | cyclopropyl | propargyl |
| 293 | methyl | Cl | methyl | CF₃ | propargyl |
| 294 | methyl | Cl | methyl | CHF₂ | propargyl |
| 295 | methyl | Cl | methyl | iPr | propargyl |
| 296 | methyl | Cl | methyl | cyclopropyl | propargyl |
| 297 | methyl | Cl | methoxymethyl | CF₃ | propargyl |
| 298 | methyl | Cl | methoxymethyl | CHF₂ | propargyl |
| 299 | methyl | Cl | methoxymethyl | iPr | propargyl |
| 300 | methyl | Cl | methoxymethyl | cyclopropyl | propargyl |
| 301 | methyl | Cl | acetyl | CF₃ | propargyl |
| 302 | methyl | Cl | acetyl | CHF₂ | propargyl |
| 303 | methyl | Cl | acetyl | iPr | propargyl |
| 304 | methyl | Cl | acetyl | cyclopropyl | propargyl |
| 305 | methyl | Me | H | CF₃ | propargyl |
| 306 | methyl | Me | H | CHF₂ | propargyl |
| 307 | methyl | Me | H | iPr | propargyl |
| 308 | methyl | Me | H | cyclopropyl | propargyl |
| 309 | methyl | Me | methyl | CF₃ | propargyl |
| 310 | methyl | Me | methyl | CHF₂ | propargyl |
| 311 | methyl | Me | methyl | iPr | propargyl |
| 312 | methyl | Me | methyl | cyclopropyl | propargyl |
| 313 | methyl | Me | methoxymethyl | CF₃ | propargyl |
| 314 | methyl | Me | methoxymethyl | CHF₂ | propargyl |
| 315 | methyl | Me | methoxymethyl | iPr | propargyl |
| 316 | methyl | Me | methoxymethyl | cyclopropyl | propargyl |
| 317 | methyl | Me | acetyl | CF₃ | propargyl |
| 318 | methyl | Me | acetyl | CHF₂ | propargyl |
| 319 | methyl | Me | acetyl | iPr | propargyl |
| 320 | methyl | Me | acetyl | cyclopropyl | propargyl |
| 321 | acetyl | Cl | H | CF₃ | propargyl |
| 322 | acetyl | Cl | H | CHF₂ | propargyl |
| 323 | acetyl | Cl | H | iPr | propargyl |
| 324 | acetyl | Cl | H | cyclopropyl | propargyl |
| 325 | acetyl | Cl | methyl | CF₃ | propargyl |
| 326 | acetyl | Cl | methyl | CHF₂ | propargyl |
| 327 | acetyl | Cl | methyl | iPr | propargyl |
| 328 | acetyl | Cl | methyl | cyclopropyl | propargyl |
| 329 | acetyl | Cl | methoxymethyl | CF₃ | propargyl |
| 330 | acetyl | Cl | methoxymethyl | CHF₂ | propargyl |
| 331 | acetyl | Cl | methoxymethyl | iPr | propargyl |
| 332 | acetyl | Cl | methoxymethyl | cyclopropyl | propargyl |
| 333 | acetyl | Cl | acetyl | CF₃ | propargyl |
| 334 | acetyl | Cl | acetyl | CHF₂ | propargyl |
| 335 | acetyl | Cl | acetyl | iPr | propargyl |
| 336 | acetyl | Cl | acetyl | cyclopropyl | propargyl |
| 337 | acetyl | Me | H | CF₃ | propargyl |
| 338 | acetyl | Me | H | CHF₂ | propargyl |
| 339 | acetyl | Me | H | iPr | propargyl |
| 340 | acetyl | Me | H | cyclopropyl | propargyl |
| 341 | acetyl | Me | methyl | CF₃ | propargyl |
| 342 | acetyl | Me | methyl | CHF₂ | propargyl |
| 343 | acetyl | Me | methyl | iPr | propargyl |
| 344 | acetyl | Me | methyl | cyclopropyl | propargyl |
| 345 | acetyl | Me | methoxymethyl | CF₃ | propargyl |
| 346 | acetyl | Me | methoxymethyl | CHF₂ | propargyl |
| 347 | acetyl | Me | methoxymethyl | iPr | propargyl |
| 348 | acetyl | Me | methoxymethyl | cyclopropyl | propargyl |
| 349 | acetyl | Me | acetyl | CF₃ | propargyl |
| 350 | acetyl | Me | acetyl | CHF₂ | propargyl |
| 351 | acetyl | Me | acetyl | iPr | propargyl |
| 352 | acetyl | Me | acetyl | cyclopropyl | propargyl |
| 353 | 2-methyl propanoyl | Cl | H | CF₃ | propargyl |
| 354 | 2-methyl propanoyl | Cl | H | CHF₂ | propargyl |
| 355 | 2-methyl propanoyl | Cl | H | iPr | propargyl |
| 356 | 2-methyl propanoyl | Cl | H | cyclopropyl | propargyl |
| 357 | 2-methyl propanoyl | Cl | methyl | CF₃ | propargyl |
| 358 | 2-methyl propanoyl | Cl | methyl | CHF₂ | propargyl |
| 359 | 2-methyl propanoyl | Cl | methyl | iPr | propargyl |
| 360 | 2-methyl propanoyl | Cl | methyl | cyclopropyl | propargyl |
| 361 | 2-methyl propanoyl | Cl | methoxymethyl | CF₃ | propargyl |
| 362 | 2-methyl propanoyl | Cl | methoxymethyl | CHF₂ | propargyl |
| 363 | 2-methyl propanoyl | Cl | methoxymethyl | iPr | propargyl |
| 364 | 2-methyl propanoyl | Cl | methoxymethyl | cyclopropyl | propargyl |
| 365 | 2-methyl propanoyl | Cl | acetyl | CF₃ | propargyl |
| 366 | 2-methyl propanoyl | Cl | acetyl | CHF₂ | propargyl |
| 367 | 2-methyl propanoyl | Cl | acetyl | iPr | propargyl |
| 368 | 2-methyl propanoyl | Cl | acetyl | cyclopropyl | propargyl |
| 369 | 2-methyl propanoyl | Me | H | CF₃ | propargyl |
| 370 | 2-methyl propanoyl | Me | H | CHF₂ | propargyl |
| 371 | 2-methyl propanoyl | Me | H | iPr | propargyl |
| 372 | 2-methyl propanoyl | Me | H | cyclopropyl | propargyl |
| 373 | 2-methyl propanoyl | Me | methyl | CF₃ | propargyl |
| 374 | 2-methyl propanoyl | Me | methyl | CHF₂ | propargyl |
| 375 | 2-methyl propanoyl | Me | methyl | iPr | propargyl |
| 376 | 2-methyl propanoyl | Me | methyl | cyclopropyl | propargyl |
| 377 | 2-methyl propanoyl | Me | methoxymethyl | CF₃ | propargyl |
| 378 | 2-methyl propanoyl | Me | methoxymethyl | CHF₂ | propargyl |
| 379 | 2-methyl propanoyl | Me | methoxymethyl | iPr | propargyl |
| 380 | 2-methyl propanoyl | Me | methoxymethyl | cyclopropyl | propargyl |
| 381 | 2-methyl propanoyl | Me | acetyl | CF₃ | propargyl |
| 382 | 2-methyl propanoyl | Me | acetyl | CHF₂ | propargyl |
| 383 | 2-methyl propanoyl | Me | acetyl | iPr | propargyl |
| 384 | 2-methyl propanoyl | Me | acetyl | cyclopropyl | propargyl |
| 385 | H | Cl | H | CF₃ | 2,2,2-trifluoroethyl |
| 386 | H | Cl | H | CHF₂ | 2,2,2-trifluoroethyl |
| 387 | H | Cl | H | iPr | 2,2,2-trifluoroethyl |
| 388 | H | Cl | H | cyclopropyl | 2,2,2-trifluoroethyl |
| 389 | H | Cl | methyl | CF₃ | 2,2,2-trifluoroethyl |
| 390 | H | Cl | methyl | CHF₂ | 2,2,2-trifluoroethyl |
| 391 | H | Cl | methyl | iPr | 2,2,2-trifluoroethyl |
| 392 | H | Cl | methyl | cyclopropyl | 2,2,2-trifluoroethyl |
| 393 | H | Cl | methoxymethyl | CF₃ | 2,2,2-trifluoroethyl |
| 394 | H | Cl | methoxymethyl | CHF₂ | 2,2,2-trifluoroethyl |
| 395 | H | Cl | methoxymethyl | iPr | 2,2,2-trifluoroethyl |
| 396 | H | Cl | methoxymethyl | cyclopropyl | 2,2,2-trifluoroethyl |
| 397 | H | Cl | acetyl | CF₃ | 2,2,2-trifluoroethyl |
| 398 | H | Cl | acetyl | CHF₂ | 2,2,2-trifluoroethyl |
| 399 | H | Cl | acetyl | iPr | 2,2,2-trifluoroethyl |
| 400 | H | Cl | acetyl | cyclopropyl | 2,2,2-trifluoroethyl |
| 401 | H | Me | H | CF₃ | 2,2,2-trifluoroethyl |
| 402 | H | Me | H | CHF₂ | 2,2,2-trifluoroethyl |
| 403 | H | Me | H | iPr | 2,2,2-trifluoroethyl |
| 404 | H | Me | H | cyclopropyl | 2,2,2-trifluoroethyl |
| 405 | H | Me | methyl | CF₃ | 2,2,2-trifluoroethyl |
| 406 | H | Me | methyl | CHF₂ | 2,2,2-trifluoroethyl |
| 407 | H | Me | methyl | iPr | 2,2,2-trifluoroethyl |
| 408 | H | Me | methyl | cyclopropyl | 2,2,2-trifluoroethyl |
| 409 | H | Me | methoxymethyl | CF₃ | 2,2,2-trifluoroethyl |
| 410 | H | Me | methoxymethyl | CHF₂ | 2,2,2-trifluoroethyl |
| 411 | H | Me | methoxymethyl | iPr | 2,2,2-trifluoroethyl |
| 412 | H | Me | methoxymethyl | cyclopropyl | 2,2,2-trifluoroethyl |
| 413 | H | Me | acetyl | CF₃ | 2,2,2-trifluoroethyl |
| 414 | H | Me | acetyl | CHF₂ | 2,2,2-trifluoroethyl |
| 415 | H | Me | acetyl | iPr | 2,2,2-trifluoroethyl |
| 416 | H | Me | acetyl | cyclopropyl | 2,2,2-trifluoroethyl |
| 417 | methyl | Cl | H | CF₃ | 2,2,2-trifluoroethyl |
| 418 | methyl | Cl | H | CHF₂ | 2,2,2-trifluoroethyl |
| 419 | methyl | Cl | H | iPr | 2,2,2-trifluoroethyl |
| 420 | methyl | Cl | H | cyclopropyl | 2,2,2-trifluoroethyl |
| 421 | methyl | Cl | methyl | CF₃ | 2,2,2-trifluoroethyl |
| 422 | methyl | Cl | methyl | CHF₂ | 2,2,2-trifluoroethyl |
| 423 | methyl | Cl | methyl | iPr | 2,2,2-trifluoroethyl |
| 424 | methyl | Cl | methyl | cyclopropyl | 2,2,2-trifluoroethyl |
| 425 | methyl | Cl | methoxymethyl | CF₃ | 2,2,2-trifluoroethyl |
| 426 | methyl | Cl | methoxymethyl | CHF₂ | 2,2,2-trifluoroethyl |
| 427 | methyl | Cl | methoxymethyl | iPr | 2,2,2-trifluoroethyl |
| 428 | methyl | Cl | methoxymethyl | cyclopropyl | 2,2,2-trifluoroethyl |
| 429 | methyl | Cl | acetyl | CF₃ | 2,2,2-trifluoroethyl |
| 430 | methyl | Cl | acetyl | CHF₂ | 2,2,2-trifluoroethyl |
| 431 | methyl | Cl | acetyl | iPr | 2,2,2-trifluoroethyl |
| 432 | methyl | Cl | acetyl | cyclopropyl | 2,2,2-trifluoroethyl |
| 433 | methyl | Me | H | CF₃ | 2,2,2-trifluoroethyl |
| 434 | methyl | Me | H | CHF₂ | 2,2,2-trifluoroethyl |
| 435 | methyl | Me | H | iPr | 2,2,2-trifluoroethyl |
| 436 | methyl | Me | H | cyclopropyl | 2,2,2-trifluoroethyl |
| 437 | methyl | Me | methyl | CF₃ | 2,2,2-trifluoroethyl |
| 438 | methyl | Me | methyl | CHF₂ | 2,2,2-trifluoroethyl |
| 439 | methyl | Me | methyl | iPr | 2,2,2-trifluoroethyl |
| 440 | methyl | Me | methyl | cyclopropyl | 2,2,2-trifluoroethyl |
| 441 | methyl | Me | methoxymethyl | CF₃ | 2,2,2-trifluoroethyl |
| 442 | methyl | Me | methoxymethyl | CHF₂ | 2,2,2-trifluoroethyl |
| 443 | methyl | Me | methoxymethyl | iPr | 2,2,2-trifluoroethyl |
| 444 | methyl | Me | methoxymethyl | cyclopropyl | 2,2,2-trifluoroethyl |
| 445 | methyl | Me | acetyl | CF₃ | 2,2,2-trifluoroethyl |
| 446 | methyl | Me | acetyl | CHF₂ | 2,2,2-trifluoroethyl |
| 447 | methyl | Me | acetyl | iPr | 2,2,2-trifluoroethyl |
| 448 | methyl | Me | acetyl | cyclopropyl | 2,2,2-trifluoroethyl |
| 449 | acetyl | Cl | H | CF₃ | 2,2,2-trifluoroethyl |
| 450 | acetyl | Cl | H | CHF₂ | 2,2,2-trifluoroethyl |
| 451 | acetyl | Cl | H | iPr | 2,2,2-trifluoroethyl |
| 452 | acetyl | Cl | H | cyclopropyl | 2,2,2-trifluoroethyl |
| 453 | acetyl | Cl | methyl | CF₃ | 2,2,2-trifluoroethyl |
| 454 | acetyl | Cl | methyl | CHF₂ | 2,2,2-trifluoroethyl |
| 455 | acetyl | Cl | methyl | iPr | 2,2,2-trifluoroethyl |
| 456 | acetyl | Cl | methyl | cyclopropyl | 2,2,2-trifluoroethyl |
| 457 | acetyl | Cl | methoxymethyl | CF₃ | 2,2,2-trifluoroethyl |
| 458 | acetyl | Cl | methoxymethyl | CHF₂ | 2,2,2-trifluoroethyl |
| 459 | acetyl | Cl | methoxymethyl | iPr | 2,2,2-trifluoroethyl |
| 460 | acetyl | Cl | methoxymethyl | cyclopropyl | 2,2,2-trifluoroethyl |
| 461 | acetyl | Cl | acetyl | CF₃ | 2,2,2-trifluoroethyl |
| 462 | acetyl | Cl | acetyl | CHF₂ | 2,2,2-trifluoroethyl |
| 463 | acetyl | Cl | acetyl | iPr | 2,2,2-trifluoroethyl |
| 464 | acetyl | Cl | acetyl | cyclopropyl | 2,2,2-trifluoroethyl |
| 465 | acetyl | Me | H | CF₃ | 2,2,2-trifluoroethyl |
| 466 | acetyl | Me | H | CHF₂ | 2,2,2-trifluoroethyl |
| 467 | acetyl | Me | H | iPr | 2,2,2-trifluoroethyl |
| 468 | acetyl | Me | H | cyclopropyl | 2,2,2-trifluoroethyl |
| 469 | acetyl | Me | methyl | CF₃ | 2,2,2-trifluoroethyl |
| 470 | acetyl | Me | methyl | CHF₂ | 2,2,2-trifluoroethyl |
| 471 | acetyl | Me | methyl | iPr | 2,2,2-trifluoroethyl |
| 472 | acetyl | Me | methyl | cyclopropyl | 2,2,2-trifluoroethyl |
| 473 | acetyl | Me | methoxymethyl | CF₃ | 2,2,2-trifluoroethyl |
| 474 | acetyl | Me | methoxymethyl | CHF₂ | 2,2,2-trifluoroethyl |
| 475 | acetyl | Me | methoxymethyl | iPr | 2,2,2-trifluoroethyl |
| 476 | acetyl | Me | methoxymethyl | cyclopropyl | 2,2,2-trifluoroethyl |
| 477 | acetyl | Me | acetyl | CF₃ | 2,2,2-trifluoroethyl |
| 478 | acetyl | Me | acetyl | CHF₂ | 2,2,2-trifluoroethyl |
| 479 | acetyl | Me | acetyl | iPr | 2,2,2-trifluoroethyl |
| 480 | acetyl | Me | acetyl | cyclopropyl | 2,2,2-trifluoroethyl |
| 481 | 2-methyl propanoyl | Cl | H | CF₃ | 2,2,2-trifluoroethyl |
| 482 | 2-methyl propanoyl | Cl | H | CHF₂ | 2,2,2-trifluoroethyl |
| 483 | 2-methyl propanoyl | Cl | H | iPr | 2,2,2-trifluoroethyl |
| 484 | 2-methyl propanoyl | Cl | H | cyclopropyl | 2,2,2-trifluoroethyl |
| 485 | 2-methyl propanoyl | Cl | methyl | CF₃ | 2,2,2-trifluoroethyl |
| 486 | 2-methyl propanoyl | Cl | methyl | CHF₂ | 2,2,2-trifluoroethyl |
| 487 | 2-methyl propanoyl | Cl | methyl | iPr | 2,2,2-trifluoroethyl |
| 488 | 2-methyl propanoyl | Cl | methyl | cyclopropyl | 2,2,2-trifluoroethyl |
| 489 | 2-methyl propanoyl | Cl | methoxymethyl | CF₃ | 2,2,2-trifluoroethyl |
| 490 | 2-methyl propanoyl | Cl | methoxymethyl | CHF₂ | 2,2,2-trifluoroethyl |
| 491 | 2-methyl propanoyl | Cl | methoxymethyl | iPr | 2,2,2-trifluoroethyl |
| 492 | 2-methyl propanoyl | Cl | methoxymethyl | cyclopropyl | 2,2,2-trifluoroethyl |
| 493 | 2-methyl propanoyl | Cl | acetyl | CF₃ | 2,2,2-trifluoroethyl |
| 494 | 2-methyl propanoyl | Cl | acetyl | CHF₂ | 2,2,2-trifluoroethyl |
| 495 | 2-methyl propanoyl | Cl | acetyl | iPr | 2,2,2-trifluoroethyl |
| 496 | 2-methyl propanoyl | Cl | acetyl | cyclopropyl | 2,2,2-trifluoroethyl |
| 497 | 2-methyl propanoyl | Me | H | CF₃ | 2,2,2-trifluoroethyl |
| 498 | 2-methyl propanoyl | Me | H | CHF₂ | 2,2,2-trifluoroethyl |
| 499 | 2-methyl propanoyl | Me | H | iPr | 2,2,2-trifluoroethyl |
| 500 | 2-methyl propanoyl | Me | H | cyclopropyl | 2,2,2-trifluoroethyl |
| 501 | 2-methyl propanoyl | Me | methyl | CF₃ | 2,2,2-trifluoroethyl |
| 502 | 2-methyl propanoyl | Me | methyl | CHF₂ | 2,2,2-trifluoroethyl |
| 503 | 2-methyl propanoyl | Me | methyl | iPr | 2,2,2-trifluoroethyl |
| 504 | 2-methyl propanoyl | Me | methyl | cyclopropyl | 2,2,2-trifluoroethyl |
| 505 | 2-methyl propanoyl | Me | methoxymethyl | CF₃ | 2,2,2-trifluoroethyl |
| 506 | 2-methyl propanoyl | Me | methoxymethyl | CHF₂ | 2,2,2-trifluoroethyl |
| 507 | 2-methyl propanoyl | Me | methoxymethyl | iPr | 2,2,2-trifluoroethyl |
| 508 | 2-methyl propanoyl | Me | methoxymethyl | cyclopropyl | 2,2,2-trifluoroethyl |
| 509 | 2-methyl propanoyl | Me | acetyl | CF₃ | 2,2,2-trifluoroethyl |
| 510 | 2-methyl propanoyl | Me | acetyl | CHF₂ | 2,2,2-trifluoroethyl |
| 511 | 2-methyl propanoyl | Me | acetyl | iPr | 2,2,2-trifluoroethyl |
| 512 | 2-methyl propanoyl | Me | acetyl | cyclopropyl | 2,2,2-trifluoroethyl |
| 513 | H | Cl | H | CF₃ | 2,2-difluoroethyl |
| 514 | H | Cl | H | CHF₂ | 2,2-difluoroethyl |
| 515 | H | Cl | H | iPr | 2,2-difluoroethyl |
| 516 | H | Cl | H | cyclopropyl | 2,2-difluoroethyl |
| 517 | H | Cl | methyl | CF₃ | 2,2-difluoroethyl |
| 518 | H | Cl | methyl | CHF₂ | 2,2-difluoroethyl |
| 519 | H | Cl | methyl | iPr | 2,2-difluoroethyl |
| 520 | H | Cl | methyl | cyclopropyl | 2,2-difluoroethyl |
| 521 | H | Cl | methoxymethyl | CF₃ | 2,2-difluoroethyl |
| 522 | H | Cl | methoxymethyl | CHF₂ | 2,2-difluoroethyl |
| 523 | H | Cl | methoxymethyl | iPr | 2,2-difluoroethyl |
| 524 | H | Cl | methoxymethyl | cyclopropyl | 2,2-difluoroethyl |
| 525 | H | Cl | acetyl | CF₃ | 2,2-difluoroethyl |
| 526 | H | Cl | acetyl | CHF₂ | 2,2-difluoroethyl |
| 527 | H | Cl | acetyl | iPr | 2,2-difluoroethyl |
| 528 | H | Cl | acetyl | cyclopropyl | 2,2-difluoroethyl |
| 529 | H | Me | H | CF₃ | 2,2-difluoroethyl |
| 530 | H | Me | H | CHF₂ | 2,2-difluoroethyl |
| 531 | H | Me | H | iPr | 2,2-difluoroethyl |
| 532 | H | Me | H | cyclopropyl | 2,2-difluoroethyl |
| 533 | H | Me | methyl | CF₃ | 2,2-difluoroethyl |
| 534 | H | Me | methyl | CHF₂ | 2,2-difluoroethyl |
| 535 | H | Me | methyl | iPr | 2,2-difluoroethyl |
| 536 | H | Me | methyl | cyclopropyl | 2,2-difluoroethyl |
| 537 | H | Me | methoxymethyl | CF₃ | 2,2-difluoroethyl |
| 538 | H | Me | methoxymethyl | CHF₂ | 2,2-difluoroethyl |
| 539 | H | Me | methoxymethyl | iPr | 2,2-difluoroethyl |
| 540 | H | Me | methoxymethyl | cyclopropyl | 2,2-difluoroethyl |
| 541 | H | Me | acetyl | CF₃ | 2,2-difluoroethyl |
| 542 | H | Me | acetyl | CHF₂ | 2,2-difluoroethyl |
| 543 | H | Me | acetyl | iPr | 2,2-difluoroethyl |
| 544 | H | Me | acetyl | cyclopropyl | 2,2-difluoroethyl |
| 545 | methyl | Cl | H | CF₃ | 2,2-difluoroethyl |
| 546 | methyl | Cl | H | CHF₂ | 2,2-difluoroethyl |
| 547 | methyl | Cl | H | iPr | 2,2-difluoroethyl |
| 548 | methyl | Cl | H | cyclopropyl | 2,2-difluoroethyl |
| 549 | methyl | Cl | methyl | CF₃ | 2,2-difluoroethyl |
| 550 | methyl | Cl | methyl | CHF₂ | 2,2-difluoroethyl |
| 551 | methyl | Cl | methyl | iPr | 2,2-difluoroethyl |
| 552 | methyl | Cl | methyl | cyclopropyl | 2,2-difluoroethyl |
| 553 | methyl | Cl | methoxymethyl | CF₃ | 2,2-difluoroethyl |
| 554 | methyl | Cl | methoxymethyl | CHF₂ | 2,2-difluoroethyl |
| 555 | methyl | Cl | methoxymethyl | iPr | 2,2-difluoroethyl |
| 556 | methyl | Cl | methoxymethyl | cyclopropyl | 2,2-difluoroethyl |
| 557 | methyl | Cl | acetyl | CF₃ | 2,2-difluoroethyl |
| 558 | methyl | Cl | acetyl | CHF₂ | 2,2-difluoroethyl |
| 559 | methyl | Cl | acetyl | iPr | 2,2-difluoroethyl |
| 560 | methyl | Cl | acetyl | cyclopropyl | 2,2-difluoroethyl |
| 561 | methyl | Me | H | CF₃ | 2,2-difluoroethyl |
| 562 | methyl | Me | H | CHF₂ | 2,2-difluoroethyl |
| 563 | methyl | Me | H | iPr | 2,2-difluoroethyl |
| 564 | methyl | Me | H | cyclopropyl | 2,2-difluoroethyl |
| 565 | methyl | Me | methyl | CF₃ | 2,2-difluoroethyl |
| 566 | methyl | Me | methyl | CHF₂ | 2,2-difluoroethyl |
| 567 | methyl | Me | methyl | iPr | 2,2-difluoroethyl |
| 568 | methyl | Me | methyl | cyclopropyl | 2,2-difluoroethyl |
| 569 | methyl | Me | methoxymethyl | CF₃ | 2,2-difluoroethyl |
| 570 | methyl | Me | methoxymethyl | CHF₂ | 2,2-difluoroethyl |
| 571 | methyl | Me | methoxymethyl | iPr | 2,2-difluoroethyl |
| 572 | methyl | Me | methoxymethyl | cyclopropyl | 2,2-difluoroethyl |
| 573 | methyl | Me | acetyl | CF₃ | 2,2-difluoroethyl |
| 574 | methyl | Me | acetyl | CHF₂ | 2,2-difluoroethyl |
| 575 | methyl | Me | acetyl | iPr | 2,2-difluoroethyl |
| 576 | methyl | Me | acetyl | cyclopropyl | 2,2-difluoroethyl |
| 577 | acetyl | Cl | H | CF₃ | 2,2-difluoroethyl |
| 578 | acetyl | Cl | H | CHF₂ | 2,2-difluoroethyl |
| 579 | acetyl | Cl | H | iPr | 2,2-difluoroethyl |
| 580 | acetyl | Cl | H | cyclopropyl | 2,2-difluoroethyl |
| 581 | acetyl | Cl | methyl | CF₃ | 2,2-difluoroethyl |
| 582 | acetyl | Cl | methyl | CHF₂ | 2,2-difluoroethyl |
| 583 | acetyl | Cl | methyl | iPr | 2,2-difluoroethyl |
| 584 | acetyl | Cl | methyl | cyclopropyl | 2,2-difluoroethyl |
| 585 | acetyl | Cl | methoxymethyl | CF₃ | 2,2-difluoroethyl |
| 586 | acetyl | Cl | methoxymethyl | CHF₂ | 2,2-difluoroethyl |
| 587 | acetyl | Cl | methoxymethyl | iPr | 2,2-difluoroethyl |
| 588 | acetyl | Cl | methoxymethyl | cyclopropyl | 2,2-difluoroethyl |
| 589 | acetyl | Cl | acetyl | CF₃ | 2,2-difluoroethyl |
| 590 | acetyl | Cl | acetyl | CHF₂ | 2,2-difluoroethyl |
| 591 | acetyl | Cl | acetyl | iPr | 2,2-difluoroethyl |
| 592 | acetyl | Cl | acetyl | cyclopropyl | 2,2-difluoroethyl |
| 593 | acetyl | Me | H | CF₃ | 2,2-difluoroethyl |
| 594 | acetyl | Me | H | CHF₂ | 2,2-difluoroethyl |
| 595 | acetyl | Me | H | iPr | 2,2-difluoroethyl |
| 596 | acetyl | Me | H | cyclopropyl | 2,2-difluoroethyl |
| 597 | acetyl | Me | methyl | CF₃ | 2,2-difluoroethyl |
| 598 | acetyl | Me | methyl | CHF₂ | 2,2-difluoroethyl |
| 599 | acetyl | Me | methyl | iPr | 2,2-difluoroethyl |
| 600 | acetyl | Me | methyl | cyclopropyl | 2,2-difluoroethyl |
| 601 | acetyl | Me | methoxymethyl | CF₃ | 2,2-difluoroethyl |
| 602 | acetyl | Me | methoxymethyl | CHF₂ | 2,2-difluoroethyl |
| 603 | acetyl | Me | methoxymethyl | iPr | 2,2-difluoroethyl |
| 604 | acetyl | Me | methoxymethyl | cyclopropyl | 2,2-difluoroethyl |
| 605 | acetyl | Me | acetyl | CF₃ | 2,2-difluoroethyl |
| 606 | acetyl | Me | acetyl | CHF₂ | 2,2-difluoroethyl |
| 607 | acetyl | Me | acetyl | iPr | 2,2-difluoroethyl |
| 608 | acetyl | Me | acetyl | cyclopropyl | 2,2-difluoroethyl |
| 609 | 2-methyl propanoyl | Cl | H | CF₃ | 2,2-difluoroethyl |
| 610 | 2-methyl propanoyl | Cl | H | CHF₂ | 2,2-difluoroethyl |
| 611 | 2-methyl propanoyl | Cl | H | iPr | 2,2-difluoroethyl |
| 612 | 2-methyl propanoyl | Cl | H | cyclopropyl | 2,2-difluoroethyl |
| 613 | 2-methyl propanoyl | Cl | methyl | CF₃ | 2,2-difluoroethyl |
| 614 | 2-methyl propanoyl | Cl | methyl | CHF₂ | 2,2-difluoroethyl |
| 615 | 2-methyl propanoyl | Cl | methyl | iPr | 2,2-difluoroethyl |
| 616 | 2-methyl propanoyl | Cl | methyl | cyclopropyl | 2,2-difluoroethyl |
| 617 | 2-methyl propanoyl | Cl | methoxymethyl | CF₃ | 2,2-difluoroethyl |
| 618 | 2-methyl propanoyl | Cl | methoxymethyl | CHF₂ | 2,2-difluoroethyl |
| 619 | 2-methyl propanoyl | Cl | methoxymethyl | iPr | 2,2-difluoroethyl |
| 620 | 2-methyl propanoyl | Cl | methoxymethyl | cyclopropyl | 2,2-difluoroethyl |
| 621 | 2-methyl propanoyl | Cl | acetyl | CF₃ | 2,2-difluoroethyl |
| 622 | 2-methyl propanoyl | Cl | acetyl | CHF₂ | 2,2-difluoroethyl |
| 623 | 2-methyl propanoyl | Cl | acetyl | iPr | 2,2-difluoroethyl |
| 624 | 2-methyl propanoyl | Cl | acetyl | cyclopropyl | 2,2-difluoroethyl |
| 625 | 2-methyl propanoyl | Me | H | CF₃ | 2,2-difluoroethyl |
| 626 | 2-methyl propanoyl | Me | H | CHF₂ | 2,2-difluoroethyl |
| 627 | 2-methyl propanoyl | Me | H | iPr | 2,2-difluoroethyl |
| 628 | 2-methyl propanoyl | Me | H | cyclopropyl | 2,2-difluoroethyl |
| 629 | 2-methyl propanoyl | Me | methyl | CF₃ | 2,2-difluoroethyl |
| 630 | 2-methyl propanoyl | Me | methyl | CHF₂ | 2,2-difluoroethyl |
| 631 | 2-methyl propanoyl | Me | methyl | iPr | 2,2-difluoroethyl |
| 632 | 2-methyl propanoyl | Me | methyl | cyclopropyl | 2,2-difluoroethyl |
| 633 | 2-methyl propanoyl | Me | methoxymethyl | CF₃ | 2,2-difluoroethyl |
| 634 | 2-methyl propanoyl | Me | methoxymethyl | CHF₂ | 2,2-difluoroethyl |
| 635 | 2-methyl propanoyl | Me | methoxymethyl | iPr | 2,2-difluoroethyl |
| 636 | 2-methyl propanoyl | Me | methoxymethyl | cyclopropyl | 2,2-difluoroethyl |
| 637 | 2-methyl propanoyl | Me | acetyl | CF₃ | 2,2-difluoroethyl |
| 638 | 2-methyl propanoyl | Me | acetyl | CHF₂ | 2,2-difluoroethyl |
| 639 | 2-methyl propanoyl | Me | acetyl | iPr | 2,2-difluoroethyl |
| 640 | 2-methyl propanoyl | Me | acetyl | cyclopropyl | 2,2-difluoroethyl |
| 641 | H | Cl | H | CF₃ | cyclopropyl |
| 642 | H | Cl | H | CHF₂ | cyclopropyl |
| 643 | H | Cl | H | iPr | cyclopropyl |
| 644 | H | Cl | H | cyclopropyl | cyclopropyl |
| 645 | H | Cl | methyl | CF₃ | cyclopropyl |
| 646 | H | Cl | methyl | CHF₂ | cyclopropyl |
| 647 | H | Cl | methyl | iPr | cyclopropyl |
| 648 | H | Cl | methyl | cyclopropyl | cyclopropyl |
| 649 | H | Cl | methoxymethyl | CF₃ | cyclopropyl |
| 650 | H | Cl | methoxymethyl | CHF₂ | cyclopropyl |
| 651 | H | Cl | methoxymethyl | iPr | cyclopropyl |
| 652 | H | Cl | methoxymethyl | cyclopropyl | cyclopropyl |
| 653 | H | Cl | acetyl | CF₃ | cyclopropyl |
| 654 | H | Cl | acetyl | CHF₂ | cyclopropyl |
| 655 | H | Cl | acetyl | iPr | cyclopropyl |
| 656 | H | Cl | acetyl | cyclopropyl | cyclopropyl |
| 657 | H | Me | H | CF₃ | cyclopropyl |
| 658 | H | Me | H | CHF₂ | cyclopropyl |
| 659 | H | Me | H | iPr | cyclopropyl |
| 660 | H | Me | H | cyclopropyl | cyclopropyl |
| 661 | H | Me | methyl | CF₃ | cyclopropyl |
| 662 | H | Me | methyl | CHF₂ | cyclopropyl |
| 663 | H | Me | methyl | iPr | cyclopropyl |
| 664 | H | Me | methyl | cyclopropyl | cyclopropyl |
| 665 | H | Me | methoxymethyl | CF₃ | cyclopropyl |
| 666 | H | Me | methoxymethyl | CHF₂ | cyclopropyl |
| 667 | H | Me | methoxymethyl | iPr | cyclopropyl |
| 668 | H | Me | methoxymethyl | cyclopropyl | cyclopropyl |
| 669 | H | Me | acetyl | CF₃ | cyclopropyl |
| 670 | H | Me | acetyl | CHF₂ | cyclopropyl |
| 671 | H | Me | acetyl | iPr | cyclopropyl |
| 672 | H | Me | acetyl | cyclopropyl | cyclopropyl |
| 673 | methyl | Cl | H | CF₃ | cyclopropyl |
| 674 | methyl | Cl | H | CHF₂ | cyclopropyl |
| 675 | methyl | Cl | H | iPr | cyclopropyl |
| 676 | methyl | Cl | H | cyclopropyl | cyclopropyl |
| 677 | methyl | Cl | methyl | CF₃ | cyclopropyl |
| 678 | methyl | Cl | methyl | CHF₂ | cyclopropyl |
| 679 | methyl | Cl | methyl | iPr | cyclopropyl |
| 680 | methyl | Cl | methyl | cyclopropyl | cyclopropyl |
| 681 | methyl | Cl | methoxymethyl | CF₃ | cyclopropyl |
| 682 | methyl | Cl | methoxymethyl | CHF₂ | cyclopropyl |
| 683 | methyl | Cl | methoxymethyl | iPr | cyclopropyl |
| 684 | methyl | Cl | methoxymethyl | cyclopropyl | cyclopropyl |
| 685 | methyl | Cl | acetyl | CF₃ | cyclopropyl |
| 686 | methyl | Cl | acetyl | CHF₂ | cyclopropyl |
| 687 | methyl | Cl | acetyl | iPr | cyclopropyl |
| 688 | methyl | Cl | acetyl | cyclopropyl | cyclopropyl |
| 689 | methyl | Me | H | CF₃ | cyclopropyl |
| 690 | methyl | Me | H | CHF₂ | cyclopropyl |
| 691 | methyl | Me | H | iPr | cyclopropyl |
| 692 | methyl | Me | H | cyclopropyl | cyclopropyl |
| 693 | methyl | Me | methyl | CF₃ | cyclopropyl |
| 694 | methyl | Me | methyl | CHF₂ | cyclopropyl |
| 695 | methyl | Me | methyl | iPr | cyclopropyl |
| 696 | methyl | Me | methyl | cyclopropyl | cyclopropyl |
| 697 | methyl | Me | methoxymethyl | CF₃ | cyclopropyl |
| 698 | methyl | Me | methoxymethyl | CHF₂ | cyclopropyl |
| 699 | methyl | Me | methoxymethyl | iPr | cyclopropyl |
| 700 | methyl | Me | methoxymethyl | cyclopropyl | cyclopropyl |
| 701 | methyl | Me | acetyl | CF₃ | cyclopropyl |
| 702 | methyl | Me | acetyl | CHF₂ | cyclopropyl |
| 703 | methyl | Me | acetyl | iPr | cyclopropyl |
| 704 | methyl | Me | acetyl | cyclopropyl | cyclopropyl |
| 705 | acetyl | Cl | H | CF₃ | cyclopropyl |
| 706 | acetyl | Cl | H | CHF₂ | cyclopropyl |
| 707 | acetyl | Cl | H | iPr | cyclopropyl |
| 708 | acetyl | Cl | H | cyclopropyl | cyclopropyl |
| 709 | acetyl | Cl | methyl | CF₃ | cyclopropyl |
| 710 | acetyl | Cl | methyl | CHF₂ | cyclopropyl |
| 711 | acetyl | Cl | methyl | iPr | cyclopropyl |
| 712 | acetyl | Cl | methyl | cyclopropyl | cyclopropyl |
| 713 | acetyl | Cl | methoxymethyl | CF₃ | cyclopropyl |
| 714 | acetyl | Cl | methoxymethyl | CHF₂ | cyclopropyl |
| 715 | acetyl | Cl | methoxymethyl | iPr | cyclopropyl |
| 716 | acetyl | Cl | methoxymethyl | cyclopropyl | cyclopropyl |
| 717 | acetyl | Cl | acetyl | CF₃ | cyclopropyl |
| 718 | acetyl | Cl | acetyl | CHF₂ | cyclopropyl |
| 719 | acetyl | Cl | acetyl | iPr | cyclopropyl |
| 720 | acetyl | Cl | acetyl | cyclopropyl | cyclopropyl |
| 721 | acetyl | Me | H | CF₃ | cyclopropyl |
| 722 | acetyl | Me | H | CHF₂ | cyclopropyl |
| 723 | acetyl | Me | H | iPr | cyclopropyl |
| 724 | acetyl | Me | H | cyclopropyl | cyclopropyl |
| 725 | acetyl | Me | methyl | CF₃ | cyclopropyl |
| 726 | acetyl | Me | methyl | CHF₂ | cyclopropyl |
| 727 | acetyl | Me | methyl | iPr | cyclopropyl |
| 728 | acetyl | Me | methyl | cyclopropyl | cyclopropyl |
| 729 | acetyl | Me | methoxymethyl | CF₃ | cyclopropyl |
| 730 | acetyl | Me | methoxymethyl | CHF₂ | cyclopropyl |
| 731 | acetyl | Me | methoxymethyl | iPr | cyclopropyl |
| 732 | acetyl | Me | methoxymethyl | cyclopropyl | cyclopropyl |
| 733 | acetyl | Me | acetyl | CF₃ | cyclopropyl |
| 734 | acetyl | Me | acetyl | CHF₂ | cyclopropyl |
| 735 | acetyl | Me | acetyl | iPr | cyclopropyl |
| 736 | acetyl | Me | acetyl | cyclopropyl | cyclopropyl |
| 737 | 2-methyl propanoyl | Cl | H | CF₃ | cyclopropyl |
| 738 | 2-methyl propanoyl | Cl | H | CHF₂ | cyclopropyl |
| 739 | 2-methyl propanoyl | Cl | H | iPr | cyclopropyl |
| 740 | 2-methyl propanoyl | Cl | H | cyclopropyl | cyclopropyl |
| 741 | 2-methyl propanoyl | Cl | methyl | CF₃ | cyclopropyl |
| 742 | 2-methyl propanoyl | Cl | methyl | CHF₂ | cyclopropyl |
| 743 | 2-methyl propanoyl | Cl | methyl | iPr | cyclopropyl |
| 744 | 2-methyl propanoyl | Cl | methyl | cyclopropyl | cyclopropyl |
| 745 | 2-methyl propanoyl | Cl | methoxymethyl | CF₃ | cyclopropyl |
| 746 | 2-methyl propanoyl | Cl | methoxymethyl | CHF₂ | cyclopropyl |
| 747 | 2-methyl propanoyl | Cl | methoxymethyl | iPr | cyclopropyl |
| 748 | 2-methyl propanoyl | Cl | methoxymethyl | cyclopropyl | cyclopropyl |
| 749 | 2-methyl propanoyl | Cl | acetyl | CF₃ | cyclopropyl |
| 750 | 2-methyl propanoyl | Cl | acetyl | CHF₂ | cyclopropyl |
| 751 | 2-methyl propanoyl | Cl | acetyl | iPr | cyclopropyl |
| 752 | 2-methyl propanoyl | Cl | acetyl | cyclopropyl | cyclopropyl |
| 753 | 2-methyl propanoyl | Me | H | CF₃ | cyclopropyl |
| 754 | 2-methyl propanoyl | Me | H | CHF₂ | cyclopropyl |
| 755 | 2-methyl propanoyl | Me | H | iPr | cyclopropyl |
| 756 | 2-methyl propanoyl | Me | H | cyclopropyl | cyclopropyl |
| 757 | 2-methyl propanoyl | Me | methyl | CF₃ | cyclopropyl |
| 758 | 2-methyl propanoyl | Me | methyl | CHF₂ | cyclopropyl |
| 759 | 2-methyl propanoyl | Me | methyl | iPr | cyclopropyl |
| 760 | 2-methyl propanoyl | Me | methyl | cyclopropyl | cyclopropyl |
| 761 | 2-methyl propanoyl | Me | methoxymethyl | CF₃ | cyclopropyl |
| 762 | 2-methyl propanoyl | Me | methoxymethyl | CHF₂ | cyclopropyl |
| 763 | 2-methyl propanoyl | Me | methoxymethyl | iPr | cyclopropyl |
| 764 | 2-methyl propanoyl | Me | methoxymethyl | cyclopropyl | cyclopropyl |
| 765 | 2-methyl propanoyl | Me | acetyl | CF₃ | cyclopropyl |
| 766 | 2-methyl propanoyl | Me | acetyl | CHF₂ | cyclopropyl |
| 767 | 2-methyl propanoyl | Me | acetyl | iPr | cyclopropyl |
| 768 | 2-methyl propanoyl | Me | acetyl | cyclopropyl | cyclopropyl |
| 769 | H | Cl | H | CF₃ | 1-cyanocyclopropyl |
| 770 | H | Cl | H | CHF₂ | 1-cyanocyclopropyl |
| 771 | H | Cl | H | iPr | 1-cyanocyclopropyl |
| 772 | H | Cl | H | cyclopropyl | 1-cyanocyclopropyl |
| 773 | H | Cl | methyl | CF₃ | 1-cyanocyclopropyl |
| 774 | H | Cl | methyl | CHF₂ | 1-cyanocyclopropyl |
| 775 | H | Cl | methyl | iPr | 1-cyanocyclopropyl |
| 776 | H | Cl | methyl | cyclopropyl | 1-cyanocyclopropyl |
| 777 | H | Cl | methoxymethyl | CF₃ | 1-cyanocyclopropyl |
| 778 | H | Cl | methoxymethyl | CHF₂ | 1-cyanocyclopropyl |
| 779 | H | Cl | methoxymethyl | iPr | 1-cyanocyclopropyl |
| 780 | H | Cl | methoxymethyl | cyclopropyl | 1-cyanocyclopropyl |
| 781 | H | Cl | acetyl | CF₃ | 1-cyanocyclopropyl |
| 782 | H | Cl | acetyl | CHF₂ | 1-cyanocyclopropyl |
| 783 | H | Cl | acetyl | iPr | 1-cyanocyclopropyl |
| 784 | H | Cl | acetyl | cyclopropyl | 1-cyanocyclopropyl |
| 785 | H | Me | H | CF₃ | 1-cyanocyclopropyl |
| 786 | H | Me | H | CHF₂ | 1-cyanocyclopropyl |
| 787 | H | Me | H | iPr | 1-cyanocyclopropyl |
| 788 | H | Me | H | cyclopropyl | 1-cyanocyclopropyl |
| 789 | H | Me | methyl | CF₃ | 1-cyanocyclopropyl |
| 790 | H | Me | methyl | CHF₂ | 1-cyanocyclopropyl |
| 791 | H | Me | methyl | iPr | 1-cyanocyclopropyl |
| 792 | H | Me | methyl | cyclopropyl | 1-cyanocyclopropyl |
| 793 | H | Me | methoxymethyl | CF₃ | 1-cyanocyclopropyl |
| 794 | H | Me | methoxymethyl | CHF₂ | 1-cyanocyclopropyl |
| 795 | H | Me | methoxymethyl | iPr | 1-cyanocyclopropyl |
| 796 | H | Me | methoxymethyl | cyclopropyl | 1-cyanocyclopropyl |
| 797 | H | Me | acetyl | CF₃ | 1-cyanocyclopropyl |
| 798 | H | Me | acetyl | CHF₂ | 1-cyanocyclopropyl |
| 799 | H | Me | acetyl | iPr | 1-cyanocyclopropyl |
| 800 | H | Me | acetyl | cyclopropyl | 1-cyanocyclopropyl |
| 801 | methyl | Cl | H | CF₃ | 1-cyanocyclopropyl |
| 802 | methyl | Cl | H | CHF₂ | 1-cyanocyclopropyl |
| 803 | methyl | Cl | H | iPr | 1-cyanocyclopropyl |
| 804 | methyl | Cl | H | cyclopropyl | 1-cyanocyclopropyl |
| 805 | methyl | Cl | methyl | CF₃ | 1-cyanocyclopropyl |
| 806 | methyl | Cl | methyl | CHF₂ | 1-cyanocyclopropyl |
| 807 | methyl | Cl | methyl | iPr | 1-cyanocyclopropyl |
| 808 | methyl | Cl | methyl | cyclopropyl | 1-cyanocyclopropyl |
| 809 | methyl | Cl | methoxymethyl | CF₃ | 1-cyanocyclopropyl |
| 810 | methyl | Cl | methoxymethyl | CHF₂ | 1-cyanocyclopropyl |
| 811 | methyl | Cl | methoxymethyl | iPr | 1-cyanocyclopropyl |
| 812 | methyl | Cl | methoxymethyl | cyclopropyl | 1-cyanocyclopropyl |
| 813 | methyl | Cl | acetyl | CF₃ | 1-cyanocyclopropyl |
| 814 | methyl | Cl | acetyl | CHF₂ | 1-cyanocyclopropyl |
| 815 | methyl | Cl | acetyl | iPr | 1-cyanocyclopropyl |
| 816 | methyl | Cl | acetyl | cyclopropyl | 1-cyanocyclopropyl |
| 817 | methyl | Me | H | CF₃ | 1-cyanocyclopropyl |
| 818 | methyl | Me | H | CHF₂ | 1-cyanocyclopropyl |
| 819 | methyl | Me | H | iPr | 1-cyanocyclopropyl |
| 820 | methyl | Me | H | cyclopropyl | 1-cyanocyclopropyl |
| 821 | methyl | Me | methyl | CF₃ | 1-cyanocyclopropyl |
| 822 | methyl | Me | methyl | CHF₂ | 1-cyanocyclopropyl |
| 823 | methyl | Me | methyl | iPr | 1-cyanocyclopropyl |
| 824 | methyl | Me | methyl | cyclopropyl | 1-cyanocyclopropyl |
| 825 | methyl | Me | methoxymethyl | CF₃ | 1-cyanocyclopropyl |
| 826 | methyl | Me | methoxymethyl | CHF₂ | 1-cyanocyclopropyl |
| 827 | methyl | Me | methoxymethyl | iPr | 1-cyanocyclopropyl |
| 828 | methyl | Me | methoxymethyl | cyclopropyl | 1-cyanocyclopropyl |
| 829 | methyl | Me | acetyl | CF₃ | 1-cyanocyclopropyl |
| 830 | methyl | Me | acetyl | CHF₂ | 1-cyanocyclopropyl |
| 831 | methyl | Me | acetyl | iPr | 1-cyanocyclopropyl |
| 832 | methyl | Me | acetyl | cyclopropyl | 1-cyanocyclopropyl |
| 833 | acetyl | Cl | H | CF₃ | 1-cyanocyclopropyl |
| 834 | acetyl | Cl | H | CHF₂ | 1-cyanocyclopropyl |
| 835 | acetyl | Cl | H | iPr | 1-cyanocyclopropyl |
| 836 | acetyl | Cl | H | cyclopropyl | 1-cyanocyclopropyl |
| 837 | acetyl | Cl | methyl | CF₃ | 1-cyanocyclopropyl |
| 838 | acetyl | Cl | methyl | CHF₂ | 1-cyanocyclopropyl |
| 839 | acetyl | Cl | methyl | iPr | 1-cyanocyclopropyl |
| 840 | acetyl | Cl | methyl | cyclopropyl | 1-cyanocyclopropyl |
| 841 | acetyl | Cl | methoxymethyl | CF₃ | 1-cyanocyclopropyl |
| 842 | acetyl | Cl | methoxymethyl | CHF₂ | 1-cyanocyclopropyl |
| 843 | acetyl | Cl | methoxymethyl | iPr | 1-cyanocyclopropyl |
| 844 | acetyl | Cl | methoxymethyl | cyclopropyl | 1-cyanocyclopropyl |
| 845 | acetyl | Cl | acetyl | CF₃ | 1-cyanocyclopropyl |
| 846 | acetyl | Cl | acetyl | CHF₂ | 1-cyanocyclopropyl |
| 847 | acetyl | Cl | acetyl | iPr | 1-cyanocyclopropyl |
| 848 | acetyl | Cl | acetyl | cyclopropyl | 1-cyanocyclopropyl |
| 849 | acetyl | Me | H | CF₃ | 1-cyanocyclopropyl |
| 850 | acetyl | Me | H | CHF₂ | 1-cyanocyclopropyl |
| 851 | acetyl | Me | H | iPr | 1-cyanocyclopropyl |
| 852 | acetyl | Me | H | cyclopropyl | 1-cyanocyclopropyl |
| 853 | acetyl | Me | methyl | CF₃ | 1-cyanocyclopropyl |
| 854 | acetyl | Me | methyl | CHF₂ | 1-cyanocyclopropyl |
| 855 | acetyl | Me | methyl | iPr | 1-cyanocyclopropyl |
| 856 | acetyl | Me | methyl | cyclopropyl | 1-cyanocyclopropyl |
| 857 | acetyl | Me | methoxymethyl | CF₃ | 1-cyanocyclopropyl |
| 858 | acetyl | Me | methoxymethyl | CHF₂ | 1-cyanocyclopropyl |
| 859 | acetyl | Me | methoxymethyl | iPr | 1-cyanocyclopropyl |
| 860 | acetyl | Me | methoxymethyl | cyclopropyl | 1-cyanocyclopropyl |
| 861 | acetyl | Me | acetyl | CF₃ | 1-cyanocyclopropyl |
| 862 | acetyl | Me | acetyl | CHF₂ | 1-cyanocyclopropyl |
| 863 | acetyl | Me | acetyl | iPr | 1-cyanocyclopropyl |
| 864 | acetyl | Me | acetyl | cyclopropyl | 1-cyanocyclopropyl |
| 865 | 2-methyl propanoyl | Cl | H | CF₃ | 1-cyanocyclopropyl |
| 866 | 2-methyl propanoyl | Cl | H | CHF₂ | 1-cyanocyclopropyl |
| 867 | 2-methyl propanoyl | Cl | H | iPr | 1-cyanocyclopropyl |
| 868 | 2-methyl propanoyl | Cl | H | cyclopropyl | 1-cyanocyclopropyl |
| 869 | 2-methyl propanoyl | Cl | methyl | CF₃ | 1-cyanocyclopropyl |
| 870 | 2-methyl propanoyl | Cl | methyl | CHF₂ | 1-cyanocyclopropyl |
| 871 | 2-methyl propanoyl | Cl | methyl | iPr | 1-cyanocyclopropyl |
| 872 | 2-methyl propanoyl | Cl | methyl | cyclopropyl | 1-cyanocyclopropyl |
| 873 | 2-methyl propanoyl | Cl | methoxymethyl | CF₃ | 1-cyanocyclopropyl |
| 874 | 2-methyl propanoyl | Cl | methoxymethyl | CHF₂ | 1-cyanocyclopropyl |
| 875 | 2-methyl propanoyl | Cl | methoxymethyl | iPr | 1-cyanocyclopropyl |
| 876 | 2-methyl propanoyl | Cl | methoxymethyl | cyclopropyl | 1-cyanocyclopropyl |
| 877 | 2-methyl propanoyl | Cl | acetyl | CF₃ | 1-cyanocyclopropyl |
| 878 | 2-methyl propanoyl | Cl | acetyl | CHF₂ | 1-cyanocyclopropyl |
| 879 | 2-methyl propanoyl | Cl | acetyl | iPr | 1-cyanocyclopropyl |
| 880 | 2-methyl propanoyl | Cl | acetyl | cyclopropyl | 1-cyanocyclopropyl |
| 881 | 2-methyl propanoyl | Me | H | CF₃ | 1-cyanocyclopropyl |
| 882 | 2-methyl propanoyl | Me | H | CHF₂ | 1-cyanocyclopropyl |
| 883 | 2-methyl propanoyl | Me | H | iPr | 1-cyanocyclopropyl |
| 884 | 2-methyl propanoyl | Me | H | cyclopropyl | 1-cyanocyclopropyl |
| 885 | 2-methyl propanoyl | Me | methyl | CF₃ | 1-cyanocyclopropyl |
| 886 | 2-methyl propanoyl | Me | methyl | CHF₂ | 1-cyanocyclopropyl |
| 887 | 2-methyl propanoyl | Me | methyl | iPr | 1-cyanocyclopropyl |
| 888 | 2-methyl propanoyl | Me | methyl | cyclopropyl | 1-cyanocyclopropyl |
| 889 | 2-methyl propanoyl | Me | methoxymethyl | CF₃ | 1-cyanocyclopropyl |
| 890 | 2-methyl propanoyl | Me | methoxymethyl | CHF₂ | 1-cyanocyclopropyl |
| 891 | 2-methyl propanoyl | Me | methoxymethyl | iPr | 1-cyanocyclopropyl |
| 892 | 2-methyl propanoyl | Me | methoxymethyl | cyclopropyl | 1-cyanocyclopropyl |
| 893 | 2-methyl propanoyl | Me | acetyl | CF₃ | 1-cyanocyclopropyl |
| 894 | 2-methyl propanoyl | Me | acetyl | CHF₂ | 1-cyanocyclopropyl |
| 895 | 2-methyl propanoyl | Me | acetyl | iPr | 1-cyanocyclopropyl |
| 896 | 2-methyl propanoyl | Me | acetyl | cyclopropyl | 1-cyanocyclopropyl |
| 897 | H | Cl | H | CF₃ | 1-cyanocyclobutyl |
| 898 | H | Cl | H | CHF₂ | 1-cyanocyclobutyl |
| 899 | H | Cl | H | iPr | 1-cyanocyclobutyl |
| 900 | H | Cl | H | cyclopropyl | 1-cyanocyclobutyl |
| 901 | H | Cl | methyl | CF₃ | 1-cyanocyclobutyl |
| 902 | H | Cl | methyl | CHF₂ | 1-cyanocyclobutyl |
| 903 | H | Cl | methyl | iPr | 1-cyanocyclobutyl |
| 904 | H | Cl | methyl | cyclopropyl | 1-cyanocyclobutyl |
| 905 | H | Cl | methoxymethyl | CF₃ | 1-cyanocyclobutyl |
| 906 | H | Cl | methoxymethyl | CHF₂ | 1-cyanocyclobutyl |
| 907 | H | Cl | methoxymethyl | iPr | 1-cyanocyclobutyl |
| 908 | H | Cl | methoxymethyl | cyclopropyl | 1-cyanocyclobutyl |
| 909 | H | Cl | acetyl | CF₃ | 1-cyanocyclobutyl |
| 910 | H | Cl | acetyl | CHF₂ | 1-cyanocyclobutyl |
| 911 | H | Cl | acetyl | iPr | 1-cyanocyclobutyl |
| 912 | H | Cl | acetyl | cyclopropyl | 1-cyanocyclobutyl |
| 913 | H | Me | H | CF₃ | 1-cyanocyclobutyl |
| 914 | H | Me | H | CHF₂ | 1-cyanocyclobutyl |
| 915 | H | Me | H | iPr | 1-cyanocyclobutyl |
| 916 | H | Me | H | cyclopropyl | 1-cyanocyclobutyl |
| 917 | H | Me | methyl | CF₃ | 1-cyanocyclobutyl |
| 918 | H | Me | methyl | CHF₂ | 1-cyanocyclobutyl |
| 919 | H | Me | methyl | iPr | 1-cyanocyclobutyl |
| 920 | H | Me | methyl | cyclopropyl | 1-cyanocyclobutyl |
| 921 | H | Me | methoxymethyl | CF₃ | 1-cyanocyclobutyl |
| 922 | H | Me | methoxymethyl | CHF₂ | 1-cyanocyclobutyl |
| 923 | H | Me | methoxymethyl | iPr | 1-cyanocyclobutyl |
| 924 | H | Me | methoxymethyl | cyclopropyl | 1-cyanocyclobutyl |
| 925 | H | Me | acetyl | CF₃ | 1-cyanocyclobutyl |
| 926 | H | Me | acetyl | CHF₂ | 1-cyanocyclobutyl |
| 927 | H | Me | acetyl | iPr | 1-cyanocyclobutyl |
| 928 | H | Me | acetyl | cyclopropyl | 1-cyanocyclobutyl |
| 929 | methyl | Cl | H | CF₃ | 1-cyanocyclobutyl |
| 930 | methyl | Cl | H | CHF₂ | 1-cyanocyclobutyl |
| 931 | methyl | Cl | H | iPr | 1-cyanocyclobutyl |
| 932 | methyl | Cl | H | cyclopropyl | 1-cyanocyclobutyl |
| 933 | methyl | Cl | methyl | CF₃ | 1-cyanocyclobutyl |
| 934 | methyl | Cl | methyl | CHF₂ | 1-cyanocyclobutyl |
| 935 | methyl | Cl | methyl | iPr | 1-cyanocyclobutyl |
| 936 | methyl | Cl | methyl | cyclopropyl | 1-cyanocyclobutyl |
| 937 | methyl | Cl | methoxymethyl | CF₃ | 1-cyanocyclobutyl |
| 938 | methyl | Cl | methoxymethyl | CHF₂ | 1-cyanocyclobutyl |
| 939 | methyl | Cl | methoxymethyl | iPr | 1-cyanocyclobutyl |
| 940 | methyl | Cl | methoxymethyl | cyclopropyl | 1-cyanocyclobutyl |
| 941 | methyl | Cl | acetyl | CF₃ | 1-cyanocyclobutyl |
| 942 | methyl | Cl | acetyl | CHF₂ | 1-cyanocyclobutyl |
| 943 | methyl | Cl | acetyl | iPr | 1-cyanocyclobutyl |
| 944 | methyl | Cl | acetyl | cyclopropyl | 1-cyanocyclobutyl |
| 945 | methyl | Me | H | CF₃ | 1-cyanocyclobutyl |
| 946 | methyl | Me | H | CHF₂ | 1-cyanocyclobutyl |
| 947 | methyl | Me | H | iPr | 1-cyanocyclobutyl |
| 948 | methyl | Me | H | cyclopropyl | 1-cyanocyclobutyl |
| 949 | methyl | Me | methyl | CF₃ | 1-cyanocyclobutyl |
| 950 | methyl | Me | methyl | CHF₂ | 1-cyanocyclobutyl |
| 951 | methyl | Me | methyl | iPr | 1-cyanocyclobutyl |
| 952 | methyl | Me | methyl | cyclopropyl | 1-cyanocyclobutyl |
| 953 | methyl | Me | methoxymethyl | CF₃ | 1-cyanocyclobutyl |
| 954 | methyl | Me | methoxymethyl | CHF₂ | 1-cyanocyclobutyl |
| 955 | methyl | Me | methoxymethyl | iPr | 1-cyanocyclobutyl |
| 956 | methyl | Me | methoxymethyl | cyclopropyl | 1-cyanocyclobutyl |
| 957 | methyl | Me | acetyl | CF₃ | 1-cyanocyclobutyl |
| 958 | methyl | Me | acetyl | CHF₂ | 1-cyanocyclobutyl |
| 959 | methyl | Me | acetyl | iPr | 1-cyanocyclobutyl |
| 960 | methyl | Me | acetyl | cyclopropyl | 1-cyanocyclobutyl |
| 961 | acetyl | Cl | H | CF₃ | 1-cyanocyclobutyl |
| 962 | acetyl | Cl | H | CHF₂ | 1-cyanocyclobutyl |
| 963 | acetyl | Cl | H | iPr | 1-cyanocyclobutyl |
| 964 | acetyl | Cl | H | cyclopropyl | 1-cyanocyclobutyl |
| 965 | acetyl | Cl | methyl | CF₃ | 1-cyanocyclobutyl |
| 966 | acetyl | Cl | methyl | CHF₂ | 1-cyanocyclobutyl |
| 967 | acetyl | Cl | methyl | iPr | 1-cyanocyclobutyl |
| 968 | acetyl | Cl | methyl | cyclopropyl | 1-cyanocyclobutyl |
| 969 | acetyl | Cl | methoxymethyl | CF₃ | 1-cyanocyclobutyl |
| 970 | acetyl | Cl | methoxymethyl | CHF₂ | 1-cyanocyclobutyl |
| 971 | acetyl | Cl | methoxymethyl | iPr | 1-cyanocyclobutyl |
| 972 | acetyl | Cl | methoxymethyl | cyclopropyl | 1-cyanocyclobutyl |
| 973 | acetyl | Cl | acetyl | CF₃ | 1-cyanocyclobutyl |
| 974 | acetyl | Cl | acetyl | CHF₂ | 1-cyanocyclobutyl |
| 975 | acetyl | Cl | acetyl | iPr | 1-cyanocyclobutyl |
| 976 | acetyl | Cl | acetyl | cyclopropyl | 1-cyanocyclobutyl |
| 977 | acetyl | Me | H | CF₃ | 1-cyanocyclobutyl |
| 978 | acetyl | Me | H | CHF₂ | 1-cyanocyclobutyl |
| 979 | acetyl | Me | H | iPr | 1-cyanocyclobutyl |
| 980 | acetyl | Me | H | cyclopropyl | 1-cyanocyclobutyl |
| 981 | acetyl | Me | methyl | CF₃ | 1-cyanocyclobutyl |
| 982 | acetyl | Me | methyl | CHF₂ | 1-cyanocyclobutyl |
| 983 | acetyl | Me | methyl | iPr | 1-cyanocyclobutyl |
| 984 | acetyl | Me | methyl | cyclopropyl | 1-cyanocyclobutyl |
| 985 | acetyl | Me | methoxymethyl | CF₃ | 1-cyanocyclobutyl |
| 986 | acetyl | Me | methoxymethyl | CHF₂ | 1-cyanocyclobutyl |
| 987 | acetyl | Me | methoxymethyl | iPr | 1-cyanocyclobutyl |
| 988 | acetyl | Me | methoxymethyl | cyclopropyl | 1-cyanocyclobutyl |
| 989 | acetyl | Me | acetyl | CF₃ | 1-cyanocyclobutyl |
| 990 | acetyl | Me | acetyl | CHF₂ | 1-cyanocyclobutyl |
| 991 | acetyl | Me | acetyl | iPr | 1-cyanocyclobutyl |
| 992 | acetyl | Me | acetyl | cyclopropyl | 1-cyanocyclobutyl |
| 993 | 2-methyl propanoyl | Cl | H | CF₃ | 1-cyanocyclobutyl |
| 994 | 2-methyl propanoyl | Cl | H | CHF₂ | 1-cyanocyclobutyl |
| 995 | 2-methyl propanoyl | Cl | H | iPr | 1-cyanocyclobutyl |
| 996 | 2-methyl propanoyl | Cl | H | cyclopropyl | 1-cyanocyclobutyl |
| 997 | 2-methyl propanoyl | Cl | methyl | CF₃ | 1-cyanocyclobutyl |
| 998 | 2-methyl propanoyl | Cl | methyl | CHF₂ | 1-cyanocyclobutyl |
| 999 | 2-methyl propanoyl | Cl | methyl | iPr | 1-cyanocyclobutyl |
| 1000 | 2-methyl propanoyl | Cl | methyl | cyclopropyl | 1-cyanocyclobutyl |
| 1001 | 2-methyl propanoyl | Cl | methoxymethyl | CF₃ | 1-cyanocyclobutyl |
| 1002 | 2-methyl propanoyl | Cl | methoxymethyl | CHF₂ | 1-cyanocyclobutyl |
| 1003 | 2-methyl propanoyl | Cl | methoxymethyl | iPr | 1-cyanocyclobutyl |
| 1004 | 2-methyl propanoyl | Cl | methoxymethyl | cyclopropyl | 1-cyanocyclobutyl |
| 1005 | 2-methyl propanoyl | Cl | acetyl | CF₃ | 1-cyanocyclobutyl |
| 1006 | 2-methyl propanoyl | Cl | acetyl | CHF₂ | 1-cyanocyclobutyl |
| 1007 | 2-methyl propanoyl | Cl | acetyl | iPr | 1-cyanocyclobutyl |
| 1008 | 2-methyl propanoyl | Cl | acetyl | cyclopropyl | 1-cyanocyclobutyl |
| 1009 | 2-methyl propanoyl | Me | H | CF₃ | 1-cyanocyclobutyl |
| 1010 | 2-methyl propanoyl | Me | H | CHF₂ | 1-cyanocyclobutyl |
| 1011 | 2-methyl propanoyl | Me | H | iPr | 1-cyanocyclobutyl |
| 1012 | 2-methyl propanoyl | Me | H | cyclopropyl | 1-cyanocyclobutyl |
| 1013 | 2-methyl propanoyl | Me | methyl | CF₃ | 1-cyanocyclobutyl |
| 1014 | 2-methyl propanoyl | Me | methyl | CHF₂ | 1-cyanocyclobutyl |
| 1015 | 2-methyl propanoyl | Me | methyl | iPr | 1-cyanocyclobutyl |
| 1016 | 2-methyl propanoyl | Me | methyl | cyclopropyl | 1-cyanocyclobutyl |
| 1017 | 2-methyl propanoyl | Me | methoxymethyl | CF₃ | 1-cyanocyclobutyl |
| 1018 | 2-methyl propanoyl | Me | methoxymethyl | CHF₂ | 1-cyanocyclobutyl |
| 1019 | 2-methyl propanoyl | Me | methoxymethyl | iPr | 1-cyanocyclobutyl |
| 1020 | 2-methyl propanoyl | Me | methoxymethyl | cyclopropyl | 1-cyanocyclobutyl |
| 1021 | 2-methyl propanoyl | Me | acetyl | CF₃ | 1-cyanocyclobutyl |
| 1022 | 2-methyl propanoyl | Me | acetyl | CHF₂ | 1-cyanocyclobutyl |
| 1023 | 2-methyl propanoyl | Me | acetyl | iPr | 1-cyanocyclobutyl |
| 1024 | 2-methyl propanoyl | Me | acetyl | cyclopropyl | 1-cyanocyclobutyl |
| 1025 | H | Cl | H | CF₃ | 3,3-difluorocyclobutyl |
| 1026 | H | Cl | H | CHF₂ | 3,3-difluorocyclobutyl |
| 1027 | H | Cl | H | iPr | 3,3-difluorocyclobutyl |
| 1028 | H | Cl | H | cyclopropyl | 3,3-difluorocyclobutyl |
| 1029 | H | Cl | methyl | CF₃ | 3,3-difluorocyclobutyl |
| 1030 | H | Cl | methyl | CHF₂ | 3,3-difluorocyclobutyl |
| 1031 | H | Cl | methyl | iPr | 3,3-difluorocyclobutyl |
| 1032 | H | Cl | methyl | cyclopropyl | 3,3-difluorocyclobutyl |
| 1033 | H | Cl | methoxymethyl | CF₃ | 3,3-difluorocyclobutyl |
| 1034 | H | Cl | methoxymethyl | CHF₂ | 3,3-difluorocyclobutyl |
| 1035 | H | Cl | methoxymethyl | iPr | 3,3-difluorocyclobutyl |
| 1036 | H | Cl | methoxymethyl | cyclopropyl | 3,3-difluorocyclobutyl |
| 1037 | H | Cl | acetyl | CF₃ | 3,3-difluorocyclobutyl |
| 1038 | H | Cl | acetyl | CHF₂ | 3,3-difluorocyclobutyl |
| 1039 | H | Cl | acetyl | iPr | 3,3-difluorocyclobutyl |
| 1040 | H | Cl | acetyl | cyclopropyl | 3,3-difluorocyclobutyl |
| 1041 | H | Me | H | CF₃ | 3,3-difluorocyclobutyl |
| 1042 | H | Me | H | CHF₂ | 3,3-difluorocyclobutyl |
| 1043 | H | Me | H | iPr | 3,3-difluorocyclobutyl |
| 1044 | H | Me | H | cyclopropyl | 3,3-difluorocyclobutyl |
| 1045 | H | Me | methyl | CF₃ | 3,3-difluorocyclobutyl |
| 1046 | H | Me | methyl | CHF₂ | 3,3-difluorocyclobutyl |
| 1047 | H | Me | methyl | iPr | 3,3-difluorocyclobutyl |
| 1048 | H | Me | methyl | cyclopropyl | 3,3-difluorocyclobutyl |
| 1049 | H | Me | methoxymethyl | CF₃ | 3,3-difluorocyclobutyl |
| 1050 | H | Me | methoxymethyl | CHF₂ | 3,3-difluorocyclobutyl |
| 1051 | H | Me | methoxymethyl | iPr | 3,3-difluorocyclobutyl |
| 1052 | H | Me | methoxymethyl | cyclopropyl | 3,3-difluorocyclobutyl |
| 1053 | H | Me | acetyl | CF₃ | 3,3-difluorocyclobutyl |
| 1054 | H | Me | acetyl | CHF₂ | 3,3-difluorocyclobutyl |
| 1055 | H | Me | acetyl | iPr | 3,3-difluorocyclobutyl |
| 1056 | H | Me | acetyl | cyclopropyl | 3,3-difluorocyclobutyl |
| 1057 | methyl | Cl | H | CF₃ | 3,3-difluorocyclobutyl |
| 1058 | methyl | Cl | H | CHF₂ | 3,3-difluorocyclobutyl |
| 1059 | methyl | Cl | H | iPr | 3,3-difluorocyclobutyl |
| 1060 | methyl | Cl | H | cyclopropyl | 3,3-difluorocyclobutyl |
| 1061 | methyl | Cl | methyl | CF₃ | 3,3-difluorocyclobutyl |
| 1062 | methyl | Cl | methyl | CHF₂ | 3,3-difluorocyclobutyl |
| 1063 | methyl | Cl | methyl | iPr | 3,3-difluorocyclobutyl |
| 1064 | methyl | Cl | methyl | cyclopropyl | 3,3-difluorocyclobutyl |
| 1065 | methyl | Cl | methoxymethyl | CF₃ | 3,3-difluorocyclobutyl |
| 1066 | methyl | Cl | methoxymethyl | CHF₂ | 3,3-difluorocyclobutyl |
| 1067 | methyl | Cl | methoxymethyl | iPr | 3,3-difluorocyclobutyl |
| 1068 | methyl | Cl | methoxymethyl | cyclopropyl | 3,3-difluorocyclobutyl |
| 1069 | methyl | Cl | acetyl | CF₃ | 3,3-difluorocyclobutyl |
| 1070 | methyl | Cl | acetyl | CHF₂ | 3,3-difluorocyclobutyl |
| 1071 | methyl | Cl | acetyl | iPr | 3,3-difluorocyclobutyl |
| 1072 | methyl | Cl | acetyl | cyclopropyl | 3,3-difluorocyclobutyl |
| 1073 | methyl | Me | H | CF₃ | 3,3-difluorocyclobutyl |
| 1074 | methyl | Me | H | CHF₂ | 3,3-difluorocyclobutyl |
| 1075 | methyl | Me | H | iPr | 3,3-difluorocyclobutyl |
| 1076 | methyl | Me | H | cyclopropyl | 3,3-difluorocyclobutyl |
| 1077 | methyl | Me | methyl | CF₃ | 3,3-difluorocyclobutyl |
| 1078 | methyl | Me | methyl | CHF₂ | 3,3-difluorocyclobutyl |
| 1079 | methyl | Me | methyl | iPr | 3,3-difluorocyclobutyl |
| 1080 | methyl | Me | methyl | cyclopropyl | 3,3-difluorocyclobutyl |
| 1081 | methyl | Me | methoxymethyl | CF₃ | 3,3-difluorocyclobutyl |
| 1082 | methyl | Me | methoxymethyl | CHF₂ | 3,3-difluorocyclobutyl |
| 1083 | methyl | Me | methoxymethyl | iPr | 3,3-difluorocyclobutyl |
| 1084 | methyl | Me | methoxymethyl | cyclopropyl | 3,3-difluorocyclobutyl |
| 1085 | methyl | Me | acetyl | CF₃ | 3,3-difluorocyclobutyl |
| 1086 | methyl | Me | acetyl | CHF₂ | 3,3-difluorocyclobutyl |
| 1087 | methyl | Me | acetyl | iPr | 3,3-difluorocyclobutyl |
| 1088 | methyl | Me | acetyl | cyclopropyl | 3,3-difluorocyclobutyl |
| 1089 | acetyl | Cl | H | CF₃ | 3,3-difluorocyclobutyl |
| 1090 | acetyl | Cl | H | CHF₂ | 3,3-difluorocyclobutyl |
| 1091 | acetyl | Cl | H | iPr | 3,3-difluorocyclobutyl |
| 1092 | acetyl | Cl | H | cyclopropyl | 3,3-difluorocyclobutyl |
| 1093 | acetyl | Cl | methyl | CF₃ | 3,3-difluorocyclobutyl |
| 1094 | acetyl | Cl | methyl | CHF₂ | 3,3-difluorocyclobutyl |
| 1095 | acetyl | Cl | methyl | iPr | 3,3-difluorocyclobutyl |
| 1096 | acetyl | Cl | methyl | cyclopropyl | 3,3-difluorocyclobutyl |
| 1097 | acetyl | Cl | methoxymethyl | CF₃ | 3,3-difluorocyclobutyl |
| 1098 | acetyl | Cl | methoxymethyl | CHF₂ | 3,3-difluorocyclobutyl |
| 1099 | acetyl | Cl | methoxymethyl | iPr | 3,3-difluorocyclobutyl |
| 1100 | acetyl | Cl | methoxymethyl | cyclopropyl | 3,3-difluorocyclobutyl |
| 1101 | acetyl | Cl | acetyl | CF₃ | 3,3-difluorocyclobutyl |
| 1102 | acetyl | Cl | acetyl | CHF₂ | 3,3-difluorocyclobutyl |
| 1103 | acetyl | Cl | acetyl | iPr | 3,3-difluorocyclobutyl |
| 1104 | acetyl | Cl | acetyl | cyclopropyl | 3,3-difluorocyclobutyl |
| 1105 | acetyl | Me | H | CF₃ | 3,3-difluorocyclobutyl |
| 1106 | acetyl | Me | H | CHF₂ | 3,3-difluorocyclobutyl |
| 1107 | acetyl | Me | H | iPr | 3,3-difluorocyclobutyl |
| 1108 | acetyl | Me | H | cyclopropyl | 3,3-difluorocyclobutyl |
| 1109 | acetyl | Me | methyl | CF₃ | 3,3-difluorocyclobutyl |
| 1110 | acetyl | Me | methyl | CHF₂ | 3,3-difluorocyclobutyl |
| 1111 | acetyl | Me | methyl | iPr | 3,3-difluorocyclobutyl |
| 1112 | acetyl | Me | methyl | cyclopropyl | 3,3-difluorocyclobutyl |
| 1113 | acetyl | Me | methoxymethyl | CF₃ | 3,3-difluorocyclobutyl |
| 1114 | acetyl | Me | methoxymethyl | CHF₂ | 3,3-difluorocyclobutyl |
| 1115 | acetyl | Me | methoxymethyl | iPr | 3,3-difluorocyclobutyl |
| 1116 | acetyl | Me | methoxymethyl | cyclopropyl | 3,3-difluorocyclobutyl |
| 1117 | acetyl | Me | acetyl | CF₃ | 3,3-difluorocyclobutyl |
| 1118 | acetyl | Me | acetyl | CHF₂ | 3,3-difluorocyclobutyl |
| 1119 | acetyl | Me | acetyl | iPr | 3,3-difluorocyclobutyl |
| 1120 | acetyl | Me | acetyl | cyclopropyl | 3,3-difluorocyclobutyl |
| 1121 | 2-methyl propanoyl | Cl | H | CF₃ | 3,3-difluorocyclobutyl |
| 1122 | 2-methyl propanoyl | Cl | H | CHF₂ | 3,3-difluorocyclobutyl |
| 1123 | 2-methyl propanoyl | Cl | H | iPr | 3,3-difluorocyclobutyl |
| 1124 | 2-methyl propanoyl | Cl | H | cyclopropyl | 3,3-difluorocyclobutyl |
| 1125 | 2-methyl propanoyl | Cl | methyl | CF₃ | 3,3-difluorocyclobutyl |
| 1126 | 2-methyl propanoyl | Cl | methyl | CHF₂ | 3,3-difluorocyclobutyl |
| 1127 | 2-methyl propanoyl | Cl | methyl | iPr | 3,3-difluorocyclobutyl |
| 1128 | 2-methyl propanoyl | Cl | methyl | cyclopropyl | 3,3-difluorocyclobutyl |
| 1129 | 2-methyl propanoyl | Cl | methoxymethyl | CF₃ | 3,3-difluorocyclobutyl |
| 1130 | 2-methyl propanoyl | Cl | methoxymethyl | CHF₂ | 3,3-difluorocyclobutyl |
| 1131 | 2-methyl propanoyl | Cl | methoxymethyl | iPr | 3,3-difluorocyclobutyl |
| 1132 | 2-methyl propanoyl | Cl | methoxymethyl | cyclopropyl | 3,3-difluorocyclobutyl |
| 1133 | 2-methyl propanoyl | Cl | acetyl | CF₃ | 3,3-difluorocyclobutyl |
| 1134 | 2-methyl propanoyl | Cl | acetyl | CHF₂ | 3,3-difluorocyclobutyl |
| 1135 | 2-methyl propanoyl | Cl | acetyl | iPr | 3,3-difluorocyclobutyl |
| 1136 | 2-methyl propanoyl | Cl | acetyl | cyclopropyl | 3,3-difluorocyclobutyl |
| 1137 | 2-methyl propanoyl | Me | H | CF₃ | 3,3-difluorocyclobutyl |
| 1138 | 2-methyl propanoyl | Me | H | CHF₂ | 3,3-difluorocyclobutyl |
| 1139 | 2-methyl propanoyl | Me | H | iPr | 3,3-difluorocyclobutyl |
| 1140 | 2-methyl propanoyl | Me | H | cyclopropyl | 3,3-difluorocyclobutyl |
| 1141 | 2-methyl propanoyl | Me | methyl | CF₃ | 3,3-difluorocyclobutyl |
| 1142 | 2-methyl propanoyl | Me | methyl | CHF₂ | 3,3-difluorocyclobutyl |
| 1143 | 2-methyl propanoyl | Me | methyl | iPr | 3,3-difluorocyclobutyl |
| 1144 | 2-methyl propanoyl | Me | methyl | cyclopropyl | 3,3-difluorocyclobutyl |
| 1145 | 2-methyl propanoyl | Me | methoxymethyl | CF₃ | 3,3-difluorocyclobutyl |
| 1146 | 2-methyl propanoyl | Me | methoxymethyl | CHF₂ | 3,3-difluorocyclobutyl |
| 1147 | 2-methyl propanoyl | Me | methoxymethyl | iPr | 3,3-difluorocyclobutyl |
| 1148 | 2-methyl propanoyl | Me | methoxymethyl | cyclopropyl | 3,3-difluorocyclobutyl |
| 1149 | 2-methyl propanoyl | Me | acetyl | CF₃ | 3,3-difluorocyclobutyl |
| 1150 | 2-methyl propanoyl | Me | acetyl | CHF₂ | 3,3-difluorocyclobutyl |
| 1151 | 2-methyl propanoyl | Me | acetyl | iPr | 3,3-difluorocyclobutyl |
| 1152 | 2-methyl propanoyl | Me | acetyl | cyclopropyl | 3,3-difluorocyclobutyl |
| 1153 | H | Cl | H | CF₃ | 1,1-dioxothietan-3-yl |
| 1154 | H | Cl | H | CHF₂ | 1,1-dioxothietan-3-yl |
| 1155 | H | Cl | H | iPr | 1,1-dioxothietan-3-yl |
| 1156 | H | Cl | H | cyclopropyl | 1,1-dioxothietan-3-yl |
| 1157 | H | Cl | methyl | CF₃ | 1,1-dioxothietan-3-yl |
| 1158 | H | Cl | methyl | CHF₂ | 1,1-dioxothietan-3-yl |
| 1159 | H | Cl | methyl | iPr | 1,1-dioxothietan-3-yl |
| 1160 | H | Cl | methyl | cyclopropyl | 1,1-dioxothietan-3-yl |
| 1161 | H | Cl | methoxymethyl | CF₃ | 1,1-dioxothietan-3-yl |
| 1162 | H | Cl | methoxymethyl | CHF₂ | 1,1-dioxothietan-3-yl |
| 1163 | H | Cl | methoxymethyl | iPr | 1,1-dioxothietan-3-yl |
| 1164 | H | Cl | methoxymethyl | cyclopropyl | 1,1-dioxothietan-3-yl |
| 1165 | H | Cl | acetyl | CF₃ | 1,1-dioxothietan-3-yl |
| 1166 | H | Cl | acetyl | CHF₂ | 1,1-dioxothietan-3-yl |
| 1167 | H | Cl | acetyl | iPr | 1,1-dioxothietan-3-yl |
| 1168 | H | Cl | acetyl | cyclopropyl | 1,1-dioxothietan-3-yl |
| 1169 | H | Me | H | CF₃ | 1,1-dioxothietan-3-yl |
| 1170 | H | Me | H | CHF₂ | 1,1-dioxothietan-3-yl |
| 1171 | H | Me | H | iPr | 1,1-dioxothietan-3-yl |
| 1172 | H | Me | H | cyclopropyl | 1,1-dioxothietan-3-yl |
| 1173 | H | Me | methyl | CF₃ | 1,1-dioxothietan-3-yl |
| 1174 | H | Me | methyl | CHF₂ | 1,1-dioxothietan-3-yl |
| 1175 | H | Me | methyl | iPr | 1,1-dioxothietan-3-yl |
| 1176 | H | Me | methyl | cyclopropyl | 1,1-dioxothietan-3-yl |
| 1177 | H | Me | methoxymethyl | CF₃ | 1,1-dioxothietan-3-yl |
| 1178 | H | Me | methoxymethyl | CHF₂ | 1,1-dioxothietan-3-yl |
| 1179 | H | Me | methoxymethyl | iPr | 1,1-dioxothietan-3-yl |
| 1180 | H | Me | methoxymethyl | cyclopropyl | 1,1-dioxothietan-3-yl |
| 1181 | H | Me | acetyl | CF₃ | 1,1-dioxothietan-3-yl |
| 1182 | H | Me | acetyl | CHF₂ | 1,1-dioxothietan-3-yl |
| 1183 | H | Me | acetyl | iPr | 1,1-dioxothietan-3-yl |
| 1184 | H | Me | acetyl | cyclopropyl | 1,1-dioxothietan-3-yl |
| 1185 | methyl | Cl | H | CF₃ | 1,1-dioxothietan-3-yl |
| 1186 | methyl | Cl | H | CHF₂ | 1,1-dioxothietan-3-yl |
| 1187 | methyl | Cl | H | iPr | 1,1-dioxothietan-3-yl |
| 1188 | methyl | Cl | H | cyclopropyl | 1,1-dioxothietan-3-yl |
| 1189 | methyl | Cl | methyl | CF₃ | 1,1-dioxothietan-3-yl |
| 1190 | methyl | Cl | methyl | CHF₂ | 1,1-dioxothietan-3-yl |
| 1191 | methyl | Cl | methyl | iPr | 1,1-dioxothietan-3-yl |
| 1192 | methyl | Cl | methyl | cyclopropyl | 1,1-dioxothietan-3-yl |
| 1193 | methyl | Cl | methoxymethyl | CF₃ | 1,1-dioxothietan-3-yl |
| 1194 | methyl | Cl | methoxymethyl | CHF₂ | 1,1-dioxothietan-3-yl |
| 1195 | methyl | Cl | methoxymethyl | iPr | 1,1-dioxothietan-3-yl |
| 1196 | methyl | Cl | methoxymethyl | cyclopropyl | 1,1-dioxothietan-3-yl |
| 1197 | methyl | Cl | acetyl | CF₃ | 1,1-dioxothietan-3-yl |
| 1198 | methyl | Cl | acetyl | CHF₂ | 1,1-dioxothietan-3-yl |
| 1199 | methyl | Cl | acetyl | iPr | 1,1-dioxothietan-3-yl |
| 1200 | methyl | Cl | acetyl | cyclopropyl | 1,1-dioxothietan-3-yl |
| 1201 | methyl | Me | H | CF₃ | 1,1-dioxothietan-3-yl |
| 1202 | methyl | Me | H | CHF₂ | 1,1-dioxothietan-3-yl |
| 1203 | methyl | Me | H | iPr | 1,1-dioxothietan-3-yl |
| 1204 | methyl | Me | H | cyclopropyl | 1,1-dioxothietan-3-yl |
| 1205 | methyl | Me | methyl | CF₃ | 1,1-dioxothietan-3-yl |
| 1206 | methyl | Me | methyl | CHF₂ | 1,1-dioxothietan-3-yl |
| 1207 | methyl | Me | methyl | iPr | 1,1-dioxothietan-3-yl |
| 1208 | methyl | Me | methyl | cyclopropyl | 1,1-dioxothietan-3-yl |
| 1209 | methyl | Me | methoxymethyl | CF₃ | 1,1-dioxothietan-3-yl |
| 1210 | methyl | Me | methoxymethyl | CHF₂ | 1,1-dioxothietan-3-yl |
| 1211 | methyl | Me | methoxymethyl | iPr | 1,1-dioxothietan-3-yl |
| 1212 | methyl | Me | methoxymethyl | cyclopropyl | 1,1-dioxothietan-3-yl |
| 1213 | methyl | Me | acetyl | CF₃ | 1,1-dioxothietan-3-yl |
| 1214 | methyl | Me | acetyl | CHF₂ | 1,1-dioxothietan-3-yl |
| 1215 | methyl | Me | acetyl | iPr | 1,1-dioxothietan-3-yl |
| 1216 | methyl | Me | acetyl | cyclopropyl | 1,1-dioxothietan-3-yl |
| 1217 | acetyl | Cl | H | CF₃ | 1,1-dioxothietan-3-yl |
| 1218 | acetyl | Cl | H | CHF₂ | 1,1-dioxothietan-3-yl |
| 1219 | acetyl | Cl | H | iPr | 1,1-dioxothietan-3-yl |
| 1220 | acetyl | Cl | H | cyclopropyl | 1,1-dioxothietan-3-yl |
| 1221 | acetyl | Cl | methyl | CF₃ | 1,1-dioxothietan-3-yl |
| 1222 | acetyl | Cl | methyl | CHF₂ | 1,1-dioxothietan-3-yl |
| 1223 | acetyl | Cl | methyl | iPr | 1,1-dioxothietan-3-yl |
| 1224 | acetyl | Cl | methyl | cyclopropyl | 1,1-dioxothietan-3-yl |
| 1225 | acetyl | Cl | methoxymethyl | CF₃ | 1,1-dioxothietan-3-yl |
| 1226 | acetyl | Cl | methoxymethyl | CHF₂ | 1,1-dioxothietan-3-yl |
| 1227 | acetyl | Cl | methoxymethyl | iPr | 1,1-dioxothietan-3-yl |
| 1228 | acetyl | Cl | methoxymethyl | cyclopropyl | 1,1-dioxothietan-3-yl |
| 1229 | acetyl | Cl | acetyl | CF₃ | 1,1-dioxothietan-3-yl |
| 1230 | acetyl | Cl | acetyl | CHF₂ | 1,1-dioxothietan-3-yl |
| 1231 | acetyl | Cl | acetyl | iPr | 1,1-dioxothietan-3-yl |
| 1232 | acetyl | Cl | acetyl | cyclopropyl | 1,1-dioxothietan-3-yl |
| 1233 | acetyl | Me | H | CF₃ | 1,1-dioxothietan-3-yl |
| 1234 | acetyl | Me | H | CHF₂ | 1,1-dioxothietan-3-yl |
| 1235 | acetyl | Me | H | iPr | 1,1-dioxothietan-3-yl |
| 1236 | acetyl | Me | H | cyclopropyl | 1,1-dioxothietan-3-yl |
| 1237 | acetyl | Me | methyl | CF₃ | 1,1-dioxothietan-3-yl |
| 1238 | acetyl | Me | methyl | CHF₂ | 1,1-dioxothietan-3-yl |
| 1239 | acetyl | Me | methyl | iPr | 1,1-dioxothietan-3-yl |
| 1240 | acetyl | Me | methyl | cyclopropyl | 1,1-dioxothietan-3-yl |
| 1241 | acetyl | Me | methoxymethyl | CF₃ | 1,1-dioxothietan-3-yl |
| 1242 | acetyl | Me | methoxymethyl | CHF₂ | 1,1-dioxothietan-3-yl |
| 1243 | acetyl | Me | methoxymethyl | iPr | 1,1-dioxothietan-3-yl |
| 1244 | acetyl | Me | methoxymethyl | cyclopropyl | 1,1-dioxothietan-3-yl |
| 1245 | acetyl | Me | acetyl | CF₃ | 1,1-dioxothietan-3-yl |
| 1246 | acetyl | Me | acetyl | CHF₂ | 1,1-dioxothietan-3-yl |
| 1247 | acetyl | Me | acetyl | iPr | 1,1-dioxothietan-3-yl |
| 1248 | acetyl | Me | acetyl | cyclopropyl | 1,1-dioxothietan-3-yl |
| 1249 | 2-methyl propanoyl | Cl | H | CF₃ | 1,1-dioxothietan-3-yl |
| 1250 | 2-methyl propanoyl | Cl | H | CHF₂ | 1,1-dioxothietan-3-yl |
| 1251 | 2-methyl propanoyl | Cl | H | iPr | 1,1-dioxothietan-3-yl |
| 1252 | 2-methyl propanoyl | Cl | H | cyclopropyl | 1,1-dioxothietan-3-yl |
| 1253 | 2-methyl propanoyl | Cl | methyl | CF₃ | 1,1-dioxothietan-3-yl |
| 1254 | 2-methyl propanoyl | Cl | methyl | CHF₂ | 1,1-dioxothietan-3-yl |
| 1255 | 2-methyl propanoyl | Cl | methyl | iPr | 1,1-dioxothietan-3-yl |
| 1256 | 2-methyl propanoyl | Cl | methyl | cyclopropyl | 1,1-dioxothietan-3-yl |
| 1257 | 2-methyl propanoyl | Cl | methoxymethyl | CF₃ | 1,1-dioxothietan-3-yl |
| 1258 | 2-methyl propanoyl | Cl | methoxymethyl | CHF₂ | 1,1-dioxothietan-3-yl |
| 1259 | 2-methyl propanoyl | Cl | methoxymethyl | iPr | 1,1-dioxothietan-3-yl |
| 1260 | 2-methyl propanoyl | Cl | methoxymethyl | cyclopropyl | 1,1-dioxothietan-3-yl |
| 1261 | 2-methyl propanoyl | Cl | acetyl | CF₃ | 1,1-dioxothietan-3-yl |
| 1262 | 2-methyl propanoyl | Cl | acetyl | CHF₂ | 1,1-dioxothietan-3-yl |
| 1263 | 2-methyl propanoyl | Cl | acetyl | iPr | 1,1-dioxothietan-3-yl |
| 1264 | 2-methyl propanoyl | Cl | acetyl | cyclopropyl | 1,1-dioxothietan-3-yl |
| 1265 | 2-methyl propanoyl | Me | H | CF₃ | 1,1-dioxothietan-3-yl |
| 1266 | 2-methyl propanoyl | Me | H | CHF₂ | 1,1-dioxothietan-3-yl |
| 1267 | 2-methyl propanoyl | Me | H | iPr | 1,1-dioxothietan-3-yl |
| 1268 | 2-methyl propanoyl | Me | H | cyclopropyl | 1,1-dioxothietan-3-yl |
| 1269 | 2-methyl propanoyl | Me | methyl | CF₃ | 1,1-dioxothietan-3-yl |
| 1270 | 2-methyl propanoyl | Me | methyl | CHF₂ | 1,1-dioxothietan-3-yl |
| 1271 | 2-methyl propanoyl | Me | methyl | iPr | 1,1-dioxothietan-3-yl |
| 1272 | 2-methyl propanoyl | Me | methyl | cyclopropyl | 1,1-dioxothietan-3-yl |
| 1273 | 2-methyl propanoyl | Me | methoxymethyl | CF₃ | 1,1-dioxothietan-3-yl |
| 1274 | 2-methyl propanoyl | Me | methoxymethyl | CHF₂ | 1,1-dioxothietan-3-yl |
| 1275 | 2-methyl propanoyl | Me | methoxymethyl | iPr | 1,1-dioxothietan-3-yl |
| 1276 | 2-methyl propanoyl | Me | methoxymethyl | cyclopropyl | 1,1-dioxothietan-3-yl |
| 1277 | 2-methyl propanoyl | Me | acetyl | CF₃ | 1,1-dioxothietan-3-yl |
| 1278 | 2-methyl propanoyl | Me | acetyl | CHF₂ | 1,1-dioxothietan-3-yl |
| 1279 | 2-methyl propanoyl | Me | acetyl | iPr | 1,1-dioxothietan-3-yl |
| 1280 | 2-methyl propanoyl | Me | acetyl | cyclopropyl | 1,1-dioxothietan-3-yl |
| 1281 | H | Cl | H | CF₃ | cyclopropylmethyl |
| 1282 | H | Cl | H | CHF₂ | cyclopropylmethyl |
| 1283 | H | Cl | H | iPr | cyclopropylmethyl |
| 1284 | H | Cl | H | cyclopropyl | cyclopropylmethyl |
| 1285 | H | Cl | methyl | CF₃ | cyclopropylmethyl |
| 1286 | H | Cl | methyl | CHF₂ | cyclopropylmethyl |
| 1287 | H | Cl | methyl | iPr | cyclopropylmethyl |
| 1288 | H | Cl | methyl | cyclopropyl | cyclopropylmethyl |
| 1289 | H | Cl | methoxymethyl | CF₃ | cyclopropylmethyl |
| 1290 | H | Cl | methoxymethyl | CHF₂ | cyclopropylmethyl |
| 1291 | H | Cl | methoxymethyl | iPr | cyclopropylmethyl |
| 1292 | H | Cl | methoxymethyl | cyclopropyl | cyclopropylmethyl |
| 1293 | H | Cl | acetyl | CF₃ | cyclopropylmethyl |
| 1294 | H | Cl | acetyl | CHF₂ | cyclopropylmethyl |
| 1295 | H | Cl | acetyl | iPr | cyclopropylmethyl |
| 1296 | H | Cl | acetyl | cyclopropyl | cyclopropylmethyl |
| 1297 | H | Me | H | CF₃ | cyclopropylmethyl |
| 1298 | H | Me | H | CHF₂ | cyclopropylmethyl |
| 1299 | H | Me | H | iPr | cyclopropylmethyl |
| 1300 | H | Me | H | cyclopropyl | cyclopropylmethyl |
| 1301 | H | Me | methyl | CF₃ | cyclopropylmethyl |
| 1302 | H | Me | methyl | CHF₂ | cyclopropylmethyl |
| 1303 | H | Me | methyl | iPr | cyclopropylmethyl |
| 1304 | H | Me | methyl | cyclopropyl | cyclopropylmethyl |
| 1305 | H | Me | methoxymethyl | CF₃ | cyclopropylmethyl |
| 1306 | H | Me | methoxymethyl | CHF₂ | cyclopropylmethyl |
| 1307 | H | Me | methoxymethyl | iPr | cyclopropylmethyl |
| 1308 | H | Me | methoxymethyl | cyclopropyl | cyclopropylmethyl |
| 1309 | H | Me | acetyl | CF₃ | cyclopropylmethyl |
| 1310 | H | Me | acetyl | CHF₂ | cyclopropylmethyl |
| 1311 | H | Me | acetyl | iPr | cyclopropylmethyl |
| 1312 | H | Me | acetyl | cyclopropyl | cyclopropylmethyl |
| 1313 | methyl | Cl | H | CF₃ | cyclopropylmethyl |
| 1314 | methyl | Cl | H | CHF₂ | cyclopropylmethyl |
| 1315 | methyl | Cl | H | iPr | cyclopropylmethyl |
| 1316 | methyl | Cl | H | cyclopropyl | cyclopropylmethyl |
| 1317 | methyl | Cl | methyl | CF₃ | cyclopropylmethyl |
| 1318 | methyl | Cl | methyl | CHF₂ | cyclopropylmethyl |
| 1319 | methyl | Cl | methyl | iPr | cyclopropylmethyl |
| 1320 | methyl | Cl | methyl | cyclopropyl | cyclopropylmethyl |
| 1321 | methyl | Cl | methoxymethyl | CF₃ | cyclopropylmethyl |
| 1322 | methyl | Cl | methoxymethyl | CHF₂ | cyclopropylmethyl |
| 1323 | methyl | Cl | methoxymethyl | iPr | cyclopropylmethyl |
| 1324 | methyl | Cl | methoxymethyl | cyclopropyl | cyclopropylmethyl |
| 1325 | methyl | Cl | acetyl | CF₃ | cyclopropylmethyl |
| 1326 | methyl | Cl | acetyl | CHF₂ | cyclopropylmethyl |
| 1327 | methyl | Cl | acetyl | iPr | cyclopropylmethyl |
| 1328 | methyl | Cl | acetyl | cyclopropyl | cyclopropylmethyl |
| 1329 | methyl | Me | H | CF₃ | cyclopropylmethyl |
| 1330 | methyl | Me | H | CHF₂ | cyclopropylmethyl |
| 1331 | methyl | Me | H | iPr | cyclopropylmethyl |
| 1332 | methyl | Me | H | cyclopropyl | cyclopropylmethyl |
| 1333 | methyl | Me | methyl | CF₃ | cyclopropylmethyl |
| 1334 | methyl | Me | methyl | CHF₂ | cyclopropylmethyl |
| 1335 | methyl | Me | methyl | iPr | cyclopropylmethyl |
| 1336 | methyl | Me | methyl | cyclopropyl | cyclopropylmethyl |
| 1337 | methyl | Me | methoxymethyl | CF₃ | cyclopropylmethyl |
| 1338 | methyl | Me | methoxymethyl | CHF₂ | cyclopropylmethyl |
| 1339 | methyl | Me | methoxymethyl | iPr | cyclopropylmethyl |
| 1340 | methyl | Me | methoxymethyl | cyclopropyl | cyclopropylmethyl |
| 1341 | methyl | Me | acetyl | CF₃ | cyclopropylmethyl |
| 1342 | methyl | Me | acetyl | CHF₂ | cyclopropylmethyl |
| 1343 | methyl | Me | acetyl | iPr | cyclopropylmethyl |
| 1344 | methyl | Me | acetyl | cyclopropyl | cyclopropylmethyl |
| 1345 | acetyl | Cl | H | CF₃ | cyclopropylmethyl |
| 1346 | acetyl | Cl | H | CHF₂ | cyclopropylmethyl |
| 1347 | acetyl | Cl | H | iPr | cyclopropylmethyl |
| 1348 | acetyl | Cl | H | cyclopropyl | cyclopropylmethyl |
| 1349 | acetyl | Cl | methyl | CF₃ | cyclopropylmethyl |
| 1350 | acetyl | Cl | methyl | CHF₂ | cyclopropylmethyl |
| 1351 | acetyl | Cl | methyl | iPr | cyclopropylmethyl |
| 1352 | acetyl | Cl | methyl | cyclopropyl | cyclopropylmethyl |
| 1353 | acetyl | Cl | methoxymethyl | CF₃ | cyclopropylmethyl |
| 1354 | acetyl | Cl | methoxymethyl | CHF₂ | cyclopropylmethyl |
| 1355 | acetyl | Cl | methoxymethyl | iPr | cyclopropylmethyl |
| 1356 | acetyl | Cl | methoxymethyl | cyclopropyl | cyclopropylmethyl |
| 1357 | acetyl | Cl | acetyl | CF₃ | cyclopropylmethyl |
| 1358 | acetyl | Cl | acetyl | CHF₂ | cyclopropylmethyl |
| 1359 | acetyl | Cl | acetyl | iPr | cyclopropylmethyl |
| 1360 | acetyl | Cl | acetyl | cyclopropyl | cyclopropylmethyl |
| 1361 | acetyl | Me | H | CF₃ | cyclopropylmethyl |
| 1362 | acetyl | Me | H | CHF₂ | cyclopropylmethyl |
| 1363 | acetyl | Me | H | iPr | cyclopropylmethyl |
| 1364 | acetyl | Me | H | cyclopropyl | cyclopropylmethyl |
| 1365 | acetyl | Me | methyl | CF₃ | cyclopropylmethyl |
| 1366 | acetyl | Me | methyl | CHF₂ | cyclopropylmethyl |
| 1367 | acetyl | Me | methyl | iPr | cyclopropylmethyl |
| 1368 | acetyl | Me | methyl | cyclopropyl | cyclopropylmethyl |
| 1369 | acetyl | Me | methoxymethyl | CF₃ | cyclopropylmethyl |
| 1370 | acetyl | Me | methoxymethyl | CHF₂ | cyclopropylmethyl |
| 1371 | acetyl | Me | methoxymethyl | iPr | cyclopropylmethyl |
| 1372 | acetyl | Me | methoxymethyl | cyclopropyl | cyclopropylmethyl |
| 1373 | acetyl | Me | acetyl | CF₃ | cyclopropylmethyl |
| 1374 | acetyl | Me | acetyl | CHF₂ | cyclopropylmethyl |
| 1375 | acetyl | Me | acetyl | iPr | cyclopropylmethyl |
| 1376 | acetyl | Me | acetyl | cyclopropyl | cyclopropylmethyl |
| 1377 | 2-methyl propanoyl | Cl | H | CF₃ | cyclopropylmethyl |
| 1378 | 2-methyl propanoyl | Cl | H | CHF₂ | cyclopropylmethyl |
| 1379 | 2-methyl propanoyl | Cl | H | iPr | cyclopropylmethyl |
| 1380 | 2-methyl propanoyl | Cl | H | cyclopropyl | cyclopropylmethyl |
| 1381 | 2-methyl propanoyl | Cl | methyl | CF₃ | cyclopropylmethyl |
| 1382 | 2-methyl propanoyl | Cl | methyl | CHF₂ | cyclopropylmethyl |
| 1383 | 2-methyl propanoyl | Cl | methyl | iPr | cyclopropylmethyl |
| 1384 | 2-methyl propanoyl | Cl | methyl | cyclopropyl | cyclopropylmethyl |
| 1385 | 2-methyl propanoyl | Cl | methoxymethyl | CF₃ | cyclopropylmethyl |
| 1386 | 2-methyl propanoyl | Cl | methoxymethyl | CHF₂ | cyclopropylmethyl |
| 1387 | 2-methyl propanoyl | Cl | methoxymethyl | iPr | cyclopropylmethyl |
| 1388 | 2-methyl propanoyl | Cl | methoxymethyl | cyclopropyl | cyclopropylmethyl |
| 1389 | 2-methyl propanoyl | Cl | acetyl | CF₃ | cyclopropylmethyl |
| 1390 | 2-methyl propanoyl | Cl | acetyl | CHF₂ | cyclopropylmethyl |
| 1391 | 2-methyl propanoyl | Cl | acetyl | iPr | cyclopropylmethyl |
| 1392 | 2-methyl propanoyl | Cl | acetyl | cyclopropyl | cyclopropylmethyl |
| 1393 | 2-methyl propanoyl | Me | H | CF₃ | cyclopropylmethyl |
| 1394 | 2-methyl propanoyl | Me | H | CHF₂ | cyclopropylmethyl |
| 1395 | 2-methyl propanoyl | Me | H | iPr | cyclopropylmethyl |
| 1396 | 2-methyl propanoyl | Me | H | cyclopropyl | cyclopropylmethyl |
| 1397 | 2-methyl propanoyl | Me | methyl | CF₃ | cyclopropylmethyl |
| 1398 | 2-methyl propanoyl | Me | methyl | CHF₂ | cyclopropylmethyl |
| 1399 | 2-methyl propanoyl | Me | methyl | iPr | cyclopropylmethyl |
| 1400 | 2-methyl propanoyl | Me | methyl | cyclopropyl | cyclopropylmethyl |
| 1401 | 2-methyl propanoyl | Me | methoxymethyl | CF₃ | cyclopropylmethyl |
| 1402 | 2-methyl propanoyl | Me | methoxymethyl | CHF₂ | cyclopropylmethyl |
| 1403 | 2-methyl propanoyl | Me | methoxymethyl | iPr | cyclopropylmethyl |
| 1404 | 2-methyl propanoyl | Me | methoxymethyl | cyclopropyl | cyclopropylmethyl |
| 1405 | 2-methyl propanoyl | Me | acetyl | CF₃ | cyclopropylmethyl |
| 1406 | 2-methyl propanoyl | Me | acetyl | CHF₂ | cyclopropylmethyl |
| 1407 | 2-methyl propanoyl | Me | acetyl | iPr | cyclopropylmethyl |
| 1408 | 2-methyl propanoyl | Me | acetyl | cyclopropyl | cyclopropylmethyl |
| 1409 | H | Cl | H | CHCl₂ | cyclopropyl |
| 1410 | H | Cl | H | CF₃ | bicyclo[1.1.1]pentanyl |
| 1411 | H | Cl | tert-butoxy carbonyl | CF₃ | 1-cyanocyclopropyl |
| 1412 | H | Cl | cyanomethyl | CF₃ | 1-cyanocyclopropyl |
| 1413 | H | Cl | 2- | CF₃ | 1-cyanocyclopropyl |
| | | | methylpropanoyloxy | | |
| | | | methyl | | |
| 1414 | H | Cl | H | CF₃ | tert-butyl |
| 1415 | H | Cl | 2- | CF₃ | cyclopropyl |
| | | | methylpropanoyloxy | | |
| | | | methyl | | |
| 1416 | H | Cl | methoxy carbonyl | CF₃ | cyclopropyl |
| 1417 | H | Cl | cyanomethyl | CF₃ | cyclopropyl |
| 1418 | H | Cl | H | CF₃ | 5-fluoro 3-cyano pyridin-6-yl |
| 1419 | H | Cl | H | CF₃ | 5-fluoro 3-chloro pyridin-6-yl |
| 1420 | H | Cl | H | CF₃ | 5-methyl 3-chloro pyridin-6-yl |
| 1421 | H | Cl | H | CF₃ | 2,4,6-trifluoro phenyl |
| 1422 | H | Cl | H | CF₃ | 2,4-difluoro phenyl |
| 1423 | H | Cl | H | CF₃ | 2-fluoro 4-cyano phenyl |
| 1424 | H | Cl | H | CF₃ | 2-fluoro 4-(methylsulfonyl) phenyl |
| 1425 | H | Cl | H | CF₃ | 2-fluoro 4-chloro phenyl |
| 1426 | H | Me | H | CF₃ | bicyclo[1.1.1]pentanyl |
| 1427 | H | Me | H | CF₃ | 1-[2-(5-cyano-2-pyridyl)-1,2,4-triazol-3- |
| | | | | | yl]ethyl |
| 1428 | H | Me | H | CF₃ | (5-chloro-2-pyridyl)methyl |
| 1429 | H | Me | H | CF₃ | (1E)-1-methoxyimino-2-methyl-propan- |
| | | | | | 2-yl |
| 1430 | H | Me | H | CF₃ | 3-oxo-2-(2,2,2- |
| | | | | | trifluoroethyl)isoxazolidine-4-yl |
| 1431 | H | Me | H | CF₃ | 1-cyclopropyl ethyl |
| 1432 | H | Me | H | CF₃ | (3-chloro-2-pyridyl)methyl |
| 1433 | H | Me | H | CF₃ | 2,4-difluoro phenyl |
| 1434 | H | Me | H | CF₃ | 1-(4-fluorophenyl)-2-oxo-pyrrolidine-3-yl |
| 1435 | H | Me | H | CF₃ | 1-(5-methyl-1,2,4-oxadiazol-3-yl)ethyl |
| 1436 | H | Me | H | CF₃ | cyclopropylmethoxy |
| 1437 | H | Cl | tert-butoxy carbonyl | CF₃ | cyclopropyl |
| 1438 | H | Cl | H | CF₃ | benzyl |
| 1439 | H | Cl | H | CF₃ | 1-cyclopropyl ethyl |
| 1440 | H | Cl | H | CF₃ | 1-cyanocyclopropyl methyl |
| 1441 | H | Cl | H | CF₃ | 3-oxo-2-ethyl isoxazolidine-4-yl |
| 1442 | H | Cl | H | CF₃ | 3-oxo-2-(2,2,2- |
| | | | | | trifluoroethyl)isoxazolidine-4-yl |
| 1443 | H | Cl | H | CF₃ | 3-methyltetrahydrofuran-3-yl |
| 1444 | H | Cl | H | CF₃ | 2-cyclopropylmethylamino-2-oxo ethyl |
| 1445 | H | Cl | H | CF₃ | phenyl |
| 1446 | H | Cl | H | CF₃ | 1-methyl 1-cyano ethyl |
| 1447 | H | Cl | H | CF₃ | 2-methyl 4-fluoro phenyl |

TABLE 2
Makes available 396 compounds of the formula
(I-2a), where the substituents R¹, R², R³, R⁶ and
R⁷ are designated in each row below:

| Index | R² | R³ | R⁶ | R⁷ | R¹ |
|---|---|---|---|---|---|
| 1 | H | Cl | H | CF₃ | ethyl |
| 2 | H | Cl | H | CHF₂ | ethyl |
| 3 | H | Cl | H | iPr | ethyl |
| 4 | H | Cl | methoxymethyl | CF₃ | ethyl |
| 5 | H | Cl | methoxymethyl | CHF₂ | ethyl |
| 6 | H | Cl | methoxymethyl | iPr | ethyl |
| 7 | H | Me | H | CF₃ | ethyl |
| 8 | H | Me | H | CHF₂ | ethyl |
| 9 | H | Me | H | iPr | ethyl |
| 10 | H | Me | methoxymethyl | CF₃ | ethyl |
| 11 | H | Me | methoxymethyl | CHF₂ | ethyl |
| 12 | H | Me | methoxymethyl | iPr | ethyl |
| 13 | H | CN | H | CF₃ | ethyl |
| 14 | H | CN | H | CHF₂ | ethyl |
| 15 | H | CN | H | iPr | ethyl |
| 16 | H | CN | methoxymethyl | CF₃ | ethyl |
| 17 | H | CN | methoxymethyl | CHF₂ | ethyl |
| 18 | H | CN | methoxymethyl | iPr | ethyl |
| 19 | methyl | Cl | H | CF₃ | ethyl |
| 20 | methyl | Cl | H | CHF₂ | ethyl |
| 21 | methyl | Cl | H | iPr | ethyl |
| 22 | methyl | Cl | methoxymethyl | CF₃ | ethyl |
| 23 | methyl | Cl | methoxymethyl | CHF₂ | ethyl |
| 24 | methyl | Cl | methoxymethyl | iPr | ethyl |
| 25 | methyl | Me | H | CF₃ | ethyl |
| 26 | methyl | Me | H | CHF₂ | ethyl |
| 27 | methyl | Me | H | iPr | ethyl |
| 28 | methyl | Me | methoxymethyl | CF₃ | ethyl |
| 29 | methyl | Me | methoxymethyl | CHF₂ | ethyl |
| 30 | methyl | Me | methoxymethyl | iPr | ethyl |
| 31 | methyl | CN | H | CF₃ | ethyl |
| 32 | methyl | CN | H | CHF₂ | ethyl |
| 33 | methyl | CN | H | iPr | ethyl |
| 34 | methyl | CN | methoxymethyl | CF₃ | ethyl |
| 35 | methyl | CN | methoxymethyl | CHF₂ | ethyl |
| 36 | methyl | CN | methoxymethyl | iPr | ethyl |
| 37 | H | Cl | H | CF₃ | isopropyl |
| 38 | H | Cl | H | CHF₂ | isopropyl |
| 39 | H | Cl | H | iPr | isopropyl |
| 40 | H | Cl | methoxymethyl | CF₃ | isopropyl |
| 41 | H | Cl | methoxymethyl | CHF₂ | isopropyl |
| 42 | H | Cl | methoxymethyl | iPr | isopropyl |
| 43 | H | Me | H | CF₃ | isopropyl |
| 44 | H | Me | H | CHF₂ | isopropyl |
| 45 | H | Me | H | iPr | isopropyl |
| 46 | H | Me | methoxymethyl | CF₃ | isopropyl |
| 47 | H | Me | methoxymethyl | CHF₂ | isopropyl |
| 48 | H | Me | methoxymethyl | iPr | isopropyl |
| 49 | H | CN | H | CF₃ | isopropyl |
| 50 | H | CN | H | CHF₂ | isopropyl |
| 51 | H | CN | H | iPr | isopropyl |
| 52 | H | CN | methoxymethyl | CF₃ | isopropyl |
| 53 | H | CN | methoxymethyl | CHF₂ | isopropyl |
| 54 | H | CN | methoxymethyl | iPr | isopropyl |
| 55 | methyl | Cl | H | CF₃ | isopropyl |
| 56 | methyl | Cl | H | CHF₂ | isopropyl |
| 57 | methyl | Cl | H | iPr | isopropyl |
| 58 | methyl | Cl | methoxymethyl | CF₃ | isopropyl |
| 59 | methyl | Cl | methoxymethyl | CHF₂ | isopropyl |
| 60 | methyl | Cl | methoxymethyl | iPr | isopropyl |
| 61 | methyl | Me | H | CF₃ | isopropyl |
| 62 | methyl | Me | H | CHF₂ | isopropyl |
| 63 | methyl | Me | H | iPr | isopropyl |
| 64 | methyl | Me | methoxymethyl | CF₃ | isopropyl |
| 65 | methyl | Me | methoxymethyl | CHF₂ | isopropyl |
| 66 | methyl | Me | methoxymethyl | iPr | isopropyl |
| 67 | methyl | CN | H | CF₃ | isopropyl |
| 68 | methyl | CN | H | CHF₂ | isopropyl |
| 69 | methyl | CN | H | iPr | isopropyl |
| 70 | methyl | CN | methoxymethyl | CF₃ | isopropyl |
| 71 | methyl | CN | methoxymethyl | CHF₂ | isopropyl |
| 72 | methyl | CN | methoxymethyl | iPr | isopropyl |
| 73 | H | Cl | H | CF₃ | propargyl |
| 74 | H | Cl | H | CHF₂ | propargyl |
| 75 | H | Cl | H | iPr | propargyl |
| 76 | H | Cl | methoxymethyl | CF₃ | propargyl |
| 77 | H | Cl | methoxymethyl | CHF₂ | propargyl |
| 78 | H | Cl | methoxymethyl | iPr | propargyl |
| 79 | H | Me | H | CF₃ | propargyl |
| 80 | H | Me | H | CHF₂ | propargyl |
| 81 | H | Me | H | iPr | propargyl |
| 82 | H | Me | methoxymethyl | CF₃ | propargyl |
| 83 | H | Me | methoxymethyl | CHF₂ | propargyl |
| 84 | H | Me | methoxymethyl | iPr | propargyl |
| 85 | H | CN | H | CF₃ | propargyl |
| 86 | H | CN | H | CHF₂ | propargyl |
| 87 | H | CN | H | iPr | propargyl |
| 88 | H | CN | methoxymethyl | CF₃ | propargyl |
| 89 | H | CN | methoxymethyl | CHF₂ | propargyl |
| 90 | H | CN | methoxymethyl | iPr | propargyl |
| 91 | methyl | Cl | H | CF₃ | propargyl |
| 92 | methyl | Cl | H | CHF₂ | propargyl |
| 93 | methyl | Cl | H | iPr | propargyl |
| 94 | methyl | Cl | methoxymethyl | CF₃ | propargyl |
| 95 | methyl | Cl | methoxymethyl | CHF₂ | propargyl |
| 96 | methyl | Cl | methoxymethyl | iPr | propargyl |
| 97 | methyl | Me | H | CF₃ | propargyl |
| 98 | methyl | Me | H | CHF₂ | propargyl |
| 99 | methyl | Me | H | iPr | propargyl |
| 100 | methyl | Me | methoxymethyl | CF₃ | propargyl |
| 101 | methyl | Me | methoxymethyl | CHF₂ | propargyl |
| 102 | methyl | Me | methoxymethyl | iPr | propargyl |
| 103 | methyl | CN | H | CF₃ | propargyl |
| 104 | methyl | CN | H | CHF₂ | propargyl |
| 105 | methyl | CN | H | iPr | propargyl |
| 106 | methyl | CN | methoxymethyl | CF₃ | propargyl |
| 107 | methyl | CN | methoxymethyl | CHF₂ | propargyl |
| 108 | methyl | CN | methoxymethyl | iPr | propargyl |
| 109 | H | Cl | H | CF₃ | 2,2,2-trifluoroethyl |
| 110 | H | Cl | H | CHF₂ | 2,2,2-trifluoroethyl |
| 111 | H | Cl | H | iPr | 2,2,2-trifluoroethyl |
| 112 | H | Cl | methoxymethyl | CF₃ | 2,2,2-trifluoroethyl |
| 113 | H | Cl | methoxymethyl | CHF₂ | 2,2,2-trifluoroethyl |
| 114 | H | Cl | methoxymethyl | iPr | 2,2,2-trifluoroethyl |
| 115 | H | Me | H | CF₃ | 2,2,2-trifluoroethyl |
| 116 | H | Me | H | CHF₂ | 2,2,2-trifluoroethyl |
| 117 | H | Me | H | iPr | 2,2,2-trifluoroethyl |
| 118 | H | Me | methoxymethyl | CF₃ | 2,2,2-trifluoroethyl |
| 119 | H | Me | methoxymethyl | CHF₂ | 2,2,2-trifluoroethyl |
| 120 | H | Me | methoxymethyl | iPr | 2,2,2-trifluoroethyl |
| 121 | H | CN | H | CF₃ | 2,2,2-trifluoroethyl |
| 122 | H | CN | H | CHF₂ | 2,2,2-trifluoroethyl |
| 123 | H | CN | H | iPr | 2,2,2-trifluoroethyl |
| 124 | H | CN | methoxymethyl | CF₃ | 2,2,2-trifluoroethyl |
| 125 | H | CN | methoxymethyl | CHF₂ | 2,2,2-trifluoroethyl |
| 126 | H | CN | methoxymethyl | iPr | 2,2,2-trifluoroethyl |
| 127 | methyl | Cl | H | CF₃ | 2,2,2-trifluoroethyl |
| 128 | methyl | Cl | H | CHF₂ | 2,2,2-trifluoroethyl |
| 129 | methyl | Cl | H | iPr | 2,2,2-trifluoroethyl |
| 130 | methyl | Cl | methoxymethyl | CF₃ | 2,2,2-trifluoroethyl |
| 131 | methyl | Cl | methoxymethyl | CHF₂ | 2,2,2-trifluoroethyl |
| 132 | methyl | Cl | methoxymethyl | iPr | 2,2,2-trifluoroethyl |
| 133 | methyl | Me | H | CF₃ | 2,2,2-trifluoroethyl |
| 134 | methyl | Me | H | CHF₂ | 2,2,2-trifluoroethyl |
| 135 | methyl | Me | H | iPr | 2,2,2-trifluoroethyl |
| 136 | methyl | Me | methoxymethyl | CF₃ | 2,2,2-trifluoroethyl |
| 137 | methyl | Me | methoxymethyl | CHF₂ | 2,2,2-trifluoroethyl |
| 138 | methyl | Me | methoxymethyl | iPr | 2,2,2-trifluoroethyl |
| 139 | methyl | CN | H | CF₃ | 2,2,2-trifluoroethyl |
| 140 | methyl | CN | H | CHF₂ | 2,2,2-trifluoroethyl |
| 141 | methyl | CN | H | iPr | 2,2,2-trifluoroethyl |
| 142 | methyl | CN | methoxymethyl | CF₃ | 2,2,2-trifluoroethyl |
| 143 | methyl | CN | methoxymethyl | CHF₂ | 2,2,2-trifluoroethyl |
| 144 | methyl | CN | methoxymethyl | iPr | 2,2,2-trifluoroethyl |
| 145 | H | Cl | H | CF₃ | 2,2-difluoroethyl |
| 146 | H | Cl | H | CHF₂ | 2,2-difluoroethyl |
| 147 | H | Cl | H | iPr | 2,2-difluoroethyl |
| 148 | H | Cl | methoxymethyl | CF₃ | 2,2-difluoroethyl |
| 149 | H | Cl | methoxymethyl | CHF₂ | 2,2-difluoroethyl |
| 150 | H | Cl | methoxymethyl | iPr | 2,2-difluoroethyl |
| 151 | H | Me | H | CF₃ | 2,2-difluoroethyl |
| 152 | H | Me | H | CHF₂ | 2,2-difluoroethyl |
| 153 | H | Me | H | iPr | 2,2-difluoroethyl |
| 154 | H | Me | methoxymethyl | CF₃ | 2,2-difluoroethyl |
| 155 | H | Me | methoxymethyl | CHF₂ | 2,2-difluoroethyl |
| 156 | H | Me | methoxymethyl | iPr | 2,2-difluoroethyl |
| 157 | H | CN | H | CF₃ | 2,2-difluoroethyl |
| 158 | H | CN | H | CHF₂ | 2,2-difluoroethyl |
| 159 | H | CN | H | iPr | 2,2-difluoroethyl |
| 160 | H | CN | methoxymethyl | CF₃ | 2,2-difluoroethyl |
| 161 | H | CN | methoxymethyl | CHF₂ | 2,2-difluoroethyl |
| 162 | H | CN | methoxymethyl | iPr | 2,2-difluoroethyl |
| 163 | methyl | Cl | H | CF₃ | 2,2-difluoroethyl |
| 164 | methyl | Cl | H | CHF₂ | 2,2-difluoroethyl |
| 165 | methyl | Cl | H | iPr | 2,2-difluoroethyl |
| 166 | methyl | Cl | methoxymethyl | CF₃ | 2,2-difluoroethyl |
| 167 | methyl | Cl | methoxymethyl | CHF₂ | 2,2-difluoroethyl |
| 168 | methyl | Cl | methoxymethyl | iPr | 2,2-difluoroethyl |
| 169 | methyl | Me | H | CF₃ | 2,2-difluoroethyl |
| 170 | methyl | Me | H | CHF₂ | 2,2-difluoroethyl |
| 171 | methyl | Me | H | iPr | 2,2-difluoroethyl |
| 172 | methyl | Me | methoxymethyl | CF₃ | 2,2-difluoroethyl |
| 173 | methyl | Me | methoxymethyl | CHF₂ | 2,2-difluoroethyl |
| 174 | methyl | Me | methoxymethyl | iPr | 2,2-difluoroethyl |
| 175 | methyl | CN | H | CF₃ | 2,2-difluoroethyl |
| 176 | methyl | CN | H | CHF₂ | 2,2-difluoroethyl |
| 177 | methyl | CN | H | iPr | 2,2-difluoroethyl |
| 178 | methyl | CN | methoxymethyl | CF₃ | 2,2-difluoroethyl |
| 179 | methyl | CN | methoxymethyl | CHF₂ | 2,2-difluoroethyl |
| 180 | methyl | CN | methoxymethyl | iPr | 2,2-difluoroethyl |
| 181 | H | Cl | H | CF₃ | cyclopropyl |
| 182 | H | Cl | H | CHF₂ | cyclopropyl |
| 183 | H | Cl | H | iPr | cyclopropyl |
| 184 | H | Cl | methoxymethyl | CF₃ | cyclopropyl |
| 185 | H | Cl | methoxymethyl | CHF₂ | cyclopropyl |
| 186 | H | Cl | methoxymethyl | iPr | cyclopropyl |
| 187 | H | Me | H | CF₃ | cyclopropyl |
| 188 | H | Me | H | CHF₂ | cyclopropyl |
| 189 | H | Me | H | iPr | cyclopropyl |
| 190 | H | Me | methoxymethyl | CF₃ | cyclopropyl |
| 191 | H | Me | methoxymethyl | CHF₂ | cyclopropyl |
| 192 | H | Me | methoxymethyl | iPr | cyclopropyl |
| 193 | H | CN | H | CF₃ | cyclopropyl |
| 194 | H | CN | H | CHF₂ | cyclopropyl |
| 195 | H | CN | H | iPr | cyclopropyl |
| 196 | H | CN | methoxymethyl | CF₃ | cyclopropyl |
| 197 | H | CN | methoxymethyl | CHF₂ | cyclopropyl |
| 198 | H | CN | methoxymethyl | iPr | cyclopropyl |
| 199 | methyl | Cl | H | CF₃ | cyclopropyl |
| 200 | methyl | Cl | H | CHF₂ | cyclopropyl |
| 201 | methyl | Cl | H | iPr | cyclopropyl |
| 202 | methyl | Cl | methoxymethyl | CF₃ | cyclopropyl |
| 203 | methyl | Cl | methoxymethyl | CHF₂ | cyclopropyl |
| 204 | methyl | Cl | methoxymethyl | iPr | cyclopropyl |
| 205 | methyl | Me | H | CF₃ | cyclopropyl |
| 206 | methyl | Me | H | CHF₂ | cyclopropyl |
| 207 | methyl | Me | H | iPr | cyclopropyl |
| 208 | methyl | Me | methoxymethyl | CF₃ | cyclopropyl |
| 209 | methyl | Me | methoxymethyl | CHF₂ | cyclopropyl |
| 210 | methyl | Me | methoxymethyl | iPr | cyclopropyl |
| 211 | methyl | CN | H | CF₃ | cyclopropyl |
| 212 | methyl | CN | H | CHF₂ | cyclopropyl |
| 213 | methyl | CN | H | iPr | cyclopropyl |
| 214 | methyl | CN | methoxymethyl | CF₃ | cyclopropyl |
| 215 | methyl | CN | methoxymethyl | CHF₂ | cyclopropyl |
| 216 | methyl | CN | methoxymethyl | iPr | cyclopropyl |
| 217 | H | Cl | H | CF₃ | 1-cyanocyclopropyl |
| 218 | H | Cl | H | CHF₂ | 1-cyanocyclopropyl |
| 219 | H | Cl | H | iPr | 1-cyanocyclopropyl |
| 220 | H | Cl | methoxymethyl | CF₃ | 1-cyanocyclopropyl |
| 221 | H | Cl | methoxymethyl | CHF₂ | 1-cyanocyclopropyl |
| 222 | H | Cl | methoxymethyl | iPr | 1-cyanocyclopropyl |
| 223 | H | Me | H | CF₃ | 1-cyanocyclopropyl |
| 224 | H | Me | H | CHF₂ | 1-cyanocyclopropyl |
| 225 | H | Me | H | iPr | 1-cyanocyclopropyl |
| 226 | H | Me | methoxymethyl | CF₃ | 1-cyanocyclopropyl |
| 227 | H | Me | methoxymethyl | CHF₂ | 1-cyanocyclopropyl |
| 228 | H | Me | methoxymethyl | iPr | 1-cyanocyclopropyl |
| 229 | H | CN | H | CF₃ | 1-cyanocyclopropyl |
| 230 | H | CN | H | CHF₂ | 1-cyanocyclopropyl |
| 231 | H | CN | H | iPr | 1-cyanocyclopropyl |
| 232 | H | CN | methoxymethyl | CF₃ | 1-cyanocyclopropyl |
| 233 | H | CN | methoxymethyl | CHF₂ | 1-cyanocyclopropyl |
| 234 | H | CN | methoxymethyl | iPr | 1-cyanocyclopropyl |
| 235 | methyl | Cl | H | CF₃ | 1-cyanocyclopropyl |
| 236 | methyl | Cl | H | CHF₂ | 1-cyanocyclopropyl |
| 237 | methyl | Cl | H | iPr | 1-cyanocyclopropyl |
| 238 | methyl | Cl | methoxymethyl | CF₃ | 1-cyanocyclopropyl |
| 239 | methyl | Cl | methoxymethyl | CHF₂ | 1-cyanocyclopropyl |
| 240 | methyl | Cl | methoxymethyl | iPr | 1-cyanocyclopropyl |
| 241 | methyl | Me | H | CF₃ | 1-cyanocyclopropyl |
| 242 | methyl | Me | H | CHF₂ | 1-cyanocyclopropyl |
| 243 | methyl | Me | H | iPr | 1-cyanocyclopropyl |
| 244 | methyl | Me | methoxymethyl | CF₃ | 1-cyanocyclopropyl |
| 245 | methyl | Me | methoxymethyl | CHF₂ | 1-cyanocyclopropyl |
| 246 | methyl | Me | methoxymethyl | iPr | 1-cyanocyclopropyl |
| 247 | methyl | CN | H | CF₃ | 1-cyanocyclopropyl |
| 248 | methyl | CN | H | CHF₂ | 1-cyanocyclopropyl |
| 249 | methyl | CN | H | iPr | 1-cyanocyclopropyl |
| 250 | methyl | CN | methoxymethyl | CF₃ | 1-cyanocyclopropyl |
| 251 | methyl | CN | methoxymethyl | CHF₂ | 1-cyanocyclopropyl |
| 252 | methyl | CN | methoxymethyl | iPr | 1-cyanocyclopropyl |
| 253 | H | Cl | H | CF₃ | 1-cyanocyclobutyl |
| 254 | H | Cl | H | CHF₂ | 1-cyanocyclobutyl |
| 255 | H | Cl | H | iPr | 1-cyanocyclobutyl |
| 256 | H | Cl | methoxymethyl | CF₃ | 1-cyanocyclobutyl |
| 257 | H | Cl | methoxymethyl | CHF₂ | 1-cyanocyclobutyl |
| 258 | H | Cl | methoxymethyl | iPr | 1-cyanocyclobutyl |
| 259 | H | Me | H | CF₃ | 1-cyanocyclobutyl |
| 260 | H | Me | H | CHF₂ | 1-cyanocyclobutyl |
| 261 | H | Me | H | iPr | 1-cyanocyclobutyl |
| 262 | H | Me | methoxymethyl | CF₃ | 1-cyanocyclobutyl |
| 263 | H | Me | methoxymethyl | CHF₂ | 1-cyanocyclobutyl |
| 264 | H | Me | methoxymethyl | iPr | 1-cyanocyclobutyl |
| 265 | H | CN | H | CF₃ | 1-cyanocyclobutyl |
| 266 | H | CN | H | CHF₂ | 1-cyanocyclobutyl |
| 267 | H | CN | H | iPr | 1-cyanocyclobutyl |
| 268 | H | CN | methoxymethyl | CF₃ | 1-cyanocyclobutyl |
| 269 | H | CN | methoxymethyl | CHF₂ | 1-cyanocyclobutyl |
| 270 | H | CN | methoxymethyl | iPr | 1-cyanocyclobutyl |
| 271 | methyl | Cl | H | CF₃ | 1-cyanocyclobutyl |
| 272 | methyl | Cl | H | CHF₂ | 1-cyanocyclobutyl |
| 273 | methyl | Cl | H | iPr | 1-cyanocyclobutyl |
| 274 | methyl | Cl | methoxymethyl | CF₃ | 1-cyanocyclobutyl |
| 275 | methyl | Cl | methoxymethyl | CHF₂ | 1-cyanocyclobutyl |
| 276 | methyl | Cl | methoxymethyl | iPr | 1-cyanocyclobutyl |
| 277 | methyl | Me | H | CF₃ | 1-cyanocyclobutyl |
| 278 | methyl | Me | H | CHF₂ | 1-cyanocyclobutyl |
| 279 | methyl | Me | H | iPr | 1-cyanocyclobutyl |
| 280 | methyl | Me | methoxymethyl | CF₃ | 1-cyanocyclobutyl |
| 281 | methyl | Me | methoxymethyl | CHF₂ | 1-cyanocyclobutyl |
| 282 | methyl | Me | methoxymethyl | iPr | 1-cyanocyclobutyl |
| 283 | methyl | CN | H | CF₃ | 1-cyanocyclobutyl |
| 284 | methyl | CN | H | CHF₂ | 1-cyanocyclobutyl |
| 285 | methyl | CN | H | iPr | 1-cyanocyclobutyl |
| 286 | methyl | CN | methoxymethyl | CF₃ | 1-cyanocyclobutyl |
| 287 | methyl | CN | methoxymethyl | CHF₂ | 1-cyanocyclobutyl |
| 288 | methyl | CN | methoxymethyl | iPr | 1-cyanocyclobutyl |
| 289 | H | Cl | H | CF₃ | 3,3-difluorocyclobutyl |
| 290 | H | Cl | H | CHF₂ | 3,3-difluorocyclobutyl |
| 291 | H | Cl | H | iPr | 3,3-difluorocyclobutyl |
| 292 | H | Cl | methoxymethyl | CF₃ | 3,3-difluorocyclobutyl |
| 293 | H | Cl | methoxymethyl | CHF₂ | 3,3-difluorocyclobutyl |
| 294 | H | Cl | methoxymethyl | iPr | 3,3-difluorocyclobutyl |
| 295 | H | Me | H | CF₃ | 3,3-difluorocyclobutyl |
| 296 | H | Me | H | CHF₂ | 3,3-difluorocyclobutyl |
| 297 | H | Me | H | iPr | 3,3-difluorocyclobutyl |
| 298 | H | Me | methoxymethyl | CF₃ | 3,3-difluorocyclobutyl |
| 299 | H | Me | methoxymethyl | CHF₂ | 3,3-difluorocyclobutyl |
| 300 | H | Me | methoxymethyl | iPr | 3,3-difluorocyclobutyl |
| 301 | H | CN | H | CF₃ | 3,3-difluorocyclobutyl |
| 302 | H | CN | H | CHF₂ | 3,3-difluorocyclobutyl |
| 303 | H | CN | H | iPr | 3,3-difluorocyclobutyl |
| 304 | H | CN | methoxymethyl | CF₃ | 3,3-difluorocyclobutyl |
| 305 | H | CN | methoxymethyl | CHF₂ | 3,3-difluorocyclobutyl |
| 306 | H | CN | methoxymethyl | iPr | 3,3-difluorocyclobutyl |
| 307 | methyl | Cl | H | CF₃ | 3,3-difluorocyclobutyl |
| 308 | methyl | Cl | H | CHF₂ | 3,3-difluorocyclobutyl |
| 309 | methyl | Cl | H | iPr | 3,3-difluorocyclobutyl |
| 310 | methyl | Cl | methoxymethyl | CF₃ | 3,3-difluorocyclobutyl |
| 311 | methyl | Cl | methoxymethyl | CHF₂ | 3,3-difluorocyclobutyl |
| 312 | methyl | Cl | methoxymethyl | iPr | 3,3-difluorocyclobutyl |
| 313 | methyl | Me | H | CF₃ | 3,3-difluorocyclobutyl |
| 314 | methyl | Me | H | CHF₂ | 3,3-difluorocyclobutyl |
| 315 | methyl | Me | H | iPr | 3,3-difluorocyclobutyl |
| 316 | methyl | Me | methoxymethyl | CF₃ | 3,3-difluorocyclobutyl |
| 317 | methyl | Me | methoxymethyl | CHF₂ | 3,3-difluorocyclobutyl |
| 318 | methyl | Me | methoxymethyl | iPr | 3,3-difluorocyclobutyl |
| 319 | methyl | CN | H | CF₃ | 3,3-difluorocyclobutyl |
| 320 | methyl | CN | H | CHF₂ | 3,3-difluorocyclobutyl |
| 321 | methyl | CN | H | iPr | 3,3-difluorocyclobutyl |
| 322 | methyl | CN | methoxymethyl | CF₃ | 3,3-difluorocyclobutyl |
| 323 | methyl | CN | methoxymethyl | CHF₂ | 3,3-difluorocyclobutyl |
| 324 | methyl | CN | methoxymethyl | iPr | 3,3-difluorocyclobutyl |
| 325 | H | Cl | H | CF₃ | 1,1-dioxothietan-3-yl |
| 326 | H | Cl | H | CHF₂ | 1,1-dioxothietan-3-yl |
| 327 | H | Cl | H | iPr | 1,1-dioxothietan-3-yl |
| 328 | H | Cl | methoxymethyl | CF₃ | 1,1-dioxothietan-3-yl |
| 329 | H | Cl | methoxymethyl | CHF₂ | 1,1-dioxothietan-3-yl |
| 330 | H | Cl | methoxymethyl | iPr | 1,1-dioxothietan-3-yl |
| 331 | H | Me | H | CF₃ | 1,1-dioxothietan-3-yl |
| 332 | H | Me | H | CHF₂ | 1,1-dioxothietan-3-yl |
| 333 | H | Me | H | iPr | 1,1-dioxothietan-3-yl |
| 334 | H | Me | methoxymethyl | CF₃ | 1,1-dioxothietan-3-yl |
| 335 | H | Me | methoxymethyl | CHF₂ | 1,1-dioxothietan-3-yl |
| 336 | H | Me | methoxymethyl | iPr | 1,1-dioxothietan-3-yl |
| 337 | H | CN | H | CF₃ | 1,1-dioxothietan-3-yl |
| 338 | H | CN | H | CHF₂ | 1,1-dioxothietan-3-yl |
| 339 | H | CN | H | iPr | 1,1-dioxothietan-3-yl |
| 340 | H | CN | methoxymethyl | CF₃ | 1,1-dioxothietan-3-yl |
| 341 | H | CN | methoxymethyl | CHF₂ | 1,1-dioxothietan-3-yl |
| 342 | H | CN | methoxymethyl | iPr | 1,1-dioxothietan-3-yl |
| 343 | methyl | Cl | H | CF₃ | 1,1-dioxothietan-3-yl |
| 344 | methyl | Cl | H | CHF₂ | 1,1-dioxothietan-3-yl |
| 345 | methyl | Cl | H | iPr | 1,1-dioxothietan-3-yl |
| 346 | methyl | Cl | methoxymethyl | CF₃ | 1,1-dioxothietan-3-yl |
| 347 | methyl | Cl | methoxymethyl | CHF₂ | 1,1-dioxothietan-3-yl |
| 348 | methyl | Cl | methoxymethyl | iPr | 1,1-dioxothietan-3-yl |
| 349 | methyl | Me | H | CF₃ | 1,1-dioxothietan-3-yl |
| 350 | methyl | Me | H | CHF₂ | 1,1-dioxothietan-3-yl |
| 351 | methyl | Me | H | iPr | 1,1-dioxothietan-3-yl |
| 352 | methyl | Me | methoxymethyl | CF₃ | 1,1-dioxothietan-3-yl |
| 353 | methyl | Me | methoxymethyl | CHF₂ | 1,1-dioxothietan-3-yl |
| 354 | methyl | Me | methoxymethyl | iPr | 1,1-dioxothietan-3-yl |
| 355 | methyl | CN | H | CF₃ | 1,1-dioxothietan-3-yl |
| 356 | methyl | CN | H | CHF₂ | 1,1-dioxothietan-3-yl |
| 357 | methyl | CN | H | iPr | 1,1-dioxothietan-3-yl |
| 358 | methyl | CN | methoxymethyl | CF₃ | 1,1-dioxothietan-3-yl |
| 359 | methyl | CN | methoxymethyl | CHF₂ | 1,1-dioxothietan-3-yl |
| 360 | methyl | CN | methoxymethyl | iPr | 1,1-dioxothietan-3-yl |
| 361 | H | Cl | H | CF₃ | cyclopropylmethyl |
| 362 | H | Cl | H | CHF₂ | cyclopropylmethyl |
| 363 | H | Cl | H | iPr | cyclopropylmethyl |
| 364 | H | Cl | methoxymethyl | CF₃ | cyclopropylmethyl |
| 365 | H | Cl | methoxymethyl | CHF₂ | cyclopropylmethyl |
| 366 | H | Cl | methoxymethyl | iPr | cyclopropylmethyl |
| 367 | H | Me | H | CF₃ | cyclopropylmethyl |
| 368 | H | Me | H | CHF₂ | cyclopropylmethyl |
| 369 | H | Me | H | iPr | cyclopropylmethyl |
| 370 | H | Me | methoxymethyl | CF₃ | cyclopropylmethyl |
| 371 | H | Me | methoxymethyl | CHF₂ | cyclopropylmethyl |
| 372 | H | Me | methoxymethyl | iPr | cyclopropylmethyl |
| 373 | H | CN | H | CF₃ | cyclopropylmethyl |
| 374 | H | CN | H | CHF₂ | cyclopropylmethyl |
| 375 | H | CN | H | iPr | cyclopropylmethyl |
| 376 | H | CN | methoxymethyl | CF₃ | cyclopropylmethyl |
| 377 | H | CN | methoxymethyl | CHF₂ | cyclopropylmethyl |
| 378 | H | CN | methoxymethyl | iPr | cyclopropylmethyl |
| 379 | methyl | Cl | H | CF₃ | cyclopropylmethyl |
| 380 | methyl | Cl | H | CHF₂ | cyclopropylmethyl |
| 381 | methyl | Cl | H | iPr | cyclopropylmethyl |
| 382 | methyl | Cl | methoxymethyl | CF₃ | cyclopropylmethyl |
| 383 | methyl | Cl | methoxymethyl | CHF₂ | cyclopropylmethyl |
| 384 | methyl | Cl | methoxymethyl | iPr | cyclopropylmethyl |
| 385 | methyl | Me | H | CF₃ | cyclopropylmethyl |
| 386 | methyl | Me | H | CHF₂ | cyclopropylmethyl |
| 387 | methyl | Me | H | iPr | cyclopropylmethyl |
| 388 | methyl | Me | methoxymethyl | CF₃ | cyclopropylmethyl |
| 389 | methyl | Me | methoxymethyl | CHF₂ | cyclopropylmethyl |
| 390 | methyl | Me | methoxymethyl | iPr | cyclopropylmethyl |
| 391 | methyl | CN | H | CF₃ | cyclopropylmethyl |
| 392 | methyl | CN | H | CHF₂ | cyclopropylmethyl |
| 393 | methyl | CN | H | iPr | cyclopropylmethyl |
| 394 | methyl | CN | methoxymethyl | CF₃ | cyclopropylmethyl |
| 395 | methyl | CN | methoxymethyl | CHF₂ | cyclopropylmethyl |
| 396 | methyl | CN | methoxymethyl | iPr | cyclopropylmethyl |

TABLE 3
Makes available 133 compounds of the formula
(I-3a), where the substituents R¹, R², R³, R⁶ and
R⁷ are designated in each row below.

| Index | R² | R³ | R⁶ | R⁷ | R¹ |
|---|---|---|---|---|---|
| 1 | H | Me | H | CF₃ | ethyl |
| 2 | H | Me | H | CHF₂ | ethyl |
| 3 | H | Me | H | iPr | ethyl |
| 4 | H | Me | methoxymethyl | CF₃ | ethyl |
| 5 | H | Me | methoxymethyl | CHF₂ | ethyl |
| 6 | H | Me | methoxymethyl | iPr | ethyl |
| 7 | methyl | Me | H | CF₃ | ethyl |
| 8 | methyl | Me | H | CHF₂ | ethyl |
| 9 | methyl | Me | H | iPr | ethyl |
| 10 | methyl | Me | methoxymethyl | CF₃ | ethyl |
| 11 | methyl | Me | methoxymethyl | CHF₂ | ethyl |
| 12 | methyl | Me | methoxymethyl | iPr | ethyl |
| 13 | H | Me | H | CF₃ | isopropyl |
| 14 | H | Me | H | CHF₂ | isopropyl |
| 15 | H | Me | H | iPr | isopropyl |
| 16 | H | Me | methoxymethyl | CF₃ | isopropyl |
| 17 | H | Me | methoxymethyl | CHF₂ | isopropyl |
| 18 | H | Me | methoxymethyl | iPr | isopropyl |
| 19 | methyl | Me | H | CF₃ | isopropyl |
| 20 | methyl | Me | H | CHF₂ | isopropyl |
| 21 | methyl | Me | H | iPr | isopropyl |
| 22 | methyl | Me | methoxymethyl | CF₃ | isopropyl |
| 23 | methyl | Me | methoxymethyl | CHF₂ | isopropyl |
| 24 | methyl | Me | methoxymethyl | iPr | isopropyl |
| 25 | H | Me | H | CF₃ | propargyl |
| 26 | H | Me | H | CHF₂ | propargyl |
| 27 | H | Me | H | iPr | propargyl |
| 28 | H | Me | methoxymethyl | CF₃ | propargyl |
| 29 | H | Me | methoxymethyl | CHF₂ | propargyl |
| 30 | H | Me | methoxymethyl | iPr | propargyl |
| 31 | methyl | Me | H | CF₃ | propargyl |
| 32 | methyl | Me | H | CHF₂ | propargyl |
| 33 | methyl | Me | H | iPr | propargyl |
| 34 | methyl | Me | methoxymethyl | CF₃ | propargyl |
| 35 | methyl | Me | methoxymethyl | CHF₂ | propargyl |
| 36 | methyl | Me | methoxymethyl | iPr | propargyl |
| 37 | H | Me | H | CF₃ | 2,2,2-trifluoroethyl |
| 38 | H | Me | H | CHF₂ | 2,2,2-trifluoroethyl |
| 39 | H | Me | H | iPr | 2,2,2-trifluoroethyl |
| 40 | H | Me | methoxymethyl | CF₃ | 2,2,2-trifluoroethyl |
| 41 | H | Me | methoxymethyl | CHF₂ | 2,2,2-trifluoroethyl |
| 42 | H | Me | methoxymethyl | iPr | 2,2,2-trifluoroethyl |
| 43 | methyl | Me | H | CF₃ | 2,2,2-trifluoroethyl |
| 44 | methyl | Me | H | CHF₂ | 2,2,2-trifluoroethyl |
| 45 | methyl | Me | H | iPr | 2,2,2-trifluoroethyl |
| 46 | methyl | Me | methoxymethyl | CF₃ | 2,2,2-trifluoroethyl |
| 47 | methyl | Me | methoxymethyl | CHF₂ | 2,2,2-trifluoroethyl |
| 48 | methyl | Me | methoxymethyl | iPr | 2,2,2-trifluoroethyl |
| 49 | H | Me | H | CF₃ | 2,2-difluoroethyl |
| 50 | H | Me | H | CHF₂ | 2,2-difluoroethyl |
| 51 | H | Me | H | iPr | 2,2-difluoroethyl |
| 52 | H | Me | methoxymethyl | CF₃ | 2,2-difluoroethyl |
| 53 | H | Me | methoxymethyl | CHF₂ | 2,2-difluoroethyl |
| 54 | H | Me | methoxymethyl | iPr | 2,2-difluoroethyl |
| 55 | methyl | Me | H | CF₃ | 2,2-difluoroethyl |
| 56 | methyl | Me | H | CHF₂ | 2,2-difluoroethyl |
| 57 | methyl | Me | H | iPr | 2,2-difluoroethyl |
| 58 | methyl | Me | methoxymethyl | CF₃ | 2,2-difluoroethyl |
| 59 | methyl | Me | methoxymethyl | CHF₂ | 2,2-difluoroethyl |
| 60 | methyl | Me | methoxymethyl | iPr | 2,2-difluoroethyl |
| 61 | H | Me | H | CF₃ | cyclopropyl |
| 62 | H | Me | H | CHF₂ | cyclopropyl |
| 63 | H | Me | H | iPr | cyclopropyl |
| 64 | H | Me | methoxymethyl | CF₃ | cyclopropyl |
| 65 | H | Me | methoxymethyl | CHF₂ | cyclopropyl |
| 66 | H | Me | methoxymethyl | iPr | cyclopropyl |
| 67 | methyl | Me | H | CF₃ | cyclopropyl |
| 68 | methyl | Me | H | CHF₂ | cyclopropyl |
| 69 | methyl | Me | H | iPr | cyclopropyl |
| 70 | methyl | Me | methoxymethyl | CF₃ | cyclopropyl |
| 71 | methyl | Me | methoxymethyl | CHF₂ | cyclopropyl |
| 72 | methyl | Me | methoxymethyl | iPr | cyclopropyl |
| 73 | H | Me | H | CF₃ | 1-cyanocyclopropyl |
| 74 | H | Me | H | CHF₂ | 1-cyanocyclopropyl |
| 75 | H | Me | H | iPr | 1-cyanocyclopropyl |
| 76 | H | Me | methoxymethyl | CF₃ | 1-cyanocyclopropyl |
| 77 | H | Me | methoxymethyl | CHF₂ | 1-cyanocyclopropyl |
| 78 | H | Me | methoxymethyl | iPr | 1-cyanocyclopropyl |
| 79 | methyl | Me | H | CF₃ | 1-cyanocyclopropyl |
| 80 | methyl | Me | H | CHF₂ | 1-cyanocyclopropyl |
| 81 | methyl | Me | H | iPr | 1-cyanocyclopropyl |
| 82 | methyl | Me | methoxymethyl | CF₃ | 1-cyanocyclopropyl |
| 83 | methyl | Me | methoxymethyl | CHF₂ | 1-cyanocyclopropyl |
| 84 | methyl | Me | methoxymethyl | iPr | 1-cyanocyclopropyl |
| 85 | H | Me | H | CF₃ | 1-cyanocyclobutyl |
| 86 | H | Me | H | CHF₂ | 1-cyanocyclobutyl |
| 87 | H | Me | H | iPr | 1-cyanocyclobutyl |
| 88 | H | Me | methoxymethyl | CF₃ | 1-cyanocyclobutyl |
| 89 | H | Me | methoxymethyl | CHF₂ | 1-cyanocyclobutyl |
| 90 | H | Me | methoxymethyl | iPr | 1-cyanocyclobutyl |
| 91 | methyl | Me | H | CF₃ | 1-cyanocyclobutyl |
| 92 | methyl | Me | H | CHF₂ | 1-cyanocyclobutyl |
| 93 | methyl | Me | H | iPr | 1-cyanocyclobutyl |
| 94 | methyl | Me | methoxymethyl | CF₃ | 1-cyanocyclobutyl |
| 95 | methyl | Me | methoxymethyl | CHF₂ | 1-cyanocyclobutyl |
| 96 | methyl | Me | methoxymethyl | iPr | 1-cyanocyclobutyl |
| 97 | H | Me | H | CF₃ | 3,3-difluorocyclobutyl |
| 98 | H | Me | H | CHF₂ | 3,3-difluorocyclobutyl |
| 99 | H | Me | H | iPr | 3,3-difluorocyclobutyl |
| 100 | H | Me | methoxymethyl | CF₃ | 3,3-difluorocyclobutyl |
| 101 | H | Me | methoxymethyl | CHF₂ | 3,3-difluorocyclobutyl |
| 102 | H | Me | methoxymethyl | iPr | 3,3-difluorocyclobutyl |
| 103 | methyl | Me | H | CF₃ | 3,3-difluorocyclobutyl |
| 104 | methyl | Me | H | CHF₂ | 3,3-difluorocyclobutyl |
| 105 | methyl | Me | H | iPr | 3,3-difluorocyclobutyl |
| 106 | methyl | Me | methoxymethyl | CF₃ | 3,3-difluorocyclobutyl |
| 107 | methyl | Me | methoxymethyl | CHF₂ | 3,3-difluorocyclobutyl |
| 108 | methyl | Me | methoxymethyl | iPr | 3,3-difluorocyclobutyl |
| 109 | H | Me | H | CF₃ | 1,1-dioxothietan-3-yl |
| 110 | H | Me | H | CHF₂ | 1,1-dioxothietan-3-yl |
| 111 | H | Me | H | iPr | 1,1-dioxothietan-3-yl |
| 112 | H | Me | methoxymethyl | CF₃ | 1,1-dioxothietan-3-yl |
| 113 | H | Me | methoxymethyl | CHF₂ | 1,1-dioxothietan-3-yl |
| 114 | H | Me | methoxymethyl | iPr | 1,1-dioxothietan-3-yl |
| 115 | methyl | Me | H | CF₃ | 1,1-dioxothietan-3-yl |
| 116 | methyl | Me | H | CHF₂ | 1,1-dioxothietan-3-yl |
| 117 | methyl | Me | H | iPr | 1,1-dioxothietan-3-yl |
| 118 | methyl | Me | methoxymethyl | CF₃ | 1,1-dioxothietan-3-yl |
| 119 | methyl | Me | methoxymethyl | CHF₂ | 1,1-dioxothietan-3-yl |
| 120 | methyl | Me | methoxymethyl | iPr | 1,1-dioxothietan-3-yl |
| 121 | H | Me | H | CF₃ | cyclopropylmethyl |
| 122 | H | Me | H | CHF₂ | cyclopropylmethyl |
| 123 | H | Me | H | iPr | cyclopropylmethyl |
| 124 | H | Me | methoxymethyl | CF₃ | cyclopropylmethyl |
| 125 | H | Me | methoxymethyl | CHF₂ | cyclopropylmethyl |
| 126 | H | Me | methoxymethyl | iPr | cyclopropylmethyl |
| 127 | methyl | Me | H | CF₃ | cyclopropylmethyl |
| 128 | methyl | Me | H | CHF₂ | cyclopropylmethyl |
| 129 | methyl | Me | H | iPr | cyclopropylmethyl |
| 130 | methyl | Me | methoxymethyl | CF₃ | cyclopropylmethyl |
| 131 | methyl | Me | methoxymethyl | CHF₂ | cyclopropylmethyl |
| 132 | methyl | Me | methoxymethyl | iPr | cyclopropylmethyl |
| 133 | H | Cl | H | CF₃ | 2-bicyclo[1.1.1]pentanyl |

TABLE 4
Makes available 282 compounds of the formula
(I-4a), where the substituents R¹, R², R³, R⁶ and
R⁷ are designated in each row below.

| Index | R² | R³ | R⁶ | R⁷ | R¹ |
|---|---|---|---|---|---|
| 1 | H | Cl | H | CF₃ | ethyl |
| 2 | H | Cl | H | CHF₂ | ethyl |
| 3 | H | Cl | H | iPr | ethyl |
| 4 | H | Cl | methoxymethyl | CF₃ | ethyl |
| 5 | H | Cl | methoxymethyl | CHF₂ | ethyl |
| 6 | H | Cl | methoxymethyl | iPr | ethyl |
| 7 | H | Me | H | CF₃ | ethyl |
| 8 | H | Me | H | CHF₂ | ethyl |
| 9 | H | Me | H | iPr | ethyl |
| 10 | H | Me | methoxymethyl | CF₃ | ethyl |
| 11 | H | Me | methoxymethyl | CHF₂ | ethyl |
| 12 | H | Me | methoxymethyl | iPr | ethyl |
| 13 | methyl | Cl | H | CF₃ | ethyl |
| 14 | methyl | Cl | H | CHF₂ | ethyl |
| 15 | methyl | Cl | H | iPr | ethyl |
| 16 | methyl | Cl | methoxymethyl | CF₃ | ethyl |
| 17 | methyl | Cl | methoxymethyl | CHF₂ | ethyl |
| 18 | methyl | Cl | methoxymethyl | iPr | ethyl |
| 19 | methyl | Me | H | CF₃ | ethyl |
| 20 | methyl | Me | H | CHF₂ | ethyl |
| 21 | methyl | Me | H | iPr | ethyl |
| 22 | methyl | Me | methoxymethyl | CF₃ | ethyl |
| 23 | methyl | Me | methoxymethyl | CHF₂ | ethyl |
| 24 | methyl | Me | methoxymethyl | iPr | ethyl |
| 25 | H | Cl | H | CF₃ | isopropyl |
| 26 | H | Cl | H | CHF₂ | isopropyl |
| 27 | H | Cl | H | iPr | isopropyl |
| 28 | H | Cl | methoxymethyl | CF₃ | isopropyl |
| 29 | H | Cl | methoxymethyl | CHF₂ | isopropyl |
| 30 | H | Cl | methoxymethyl | iPr | isopropyl |
| 31 | H | Me | H | CF₃ | isopropyl |
| 32 | H | Me | H | CHF₂ | isopropyl |
| 33 | H | Me | H | iPr | isopropyl |
| 34 | H | Me | methoxymethyl | CF₃ | isopropyl |
| 35 | H | Me | methoxymethyl | CHF₂ | isopropyl |
| 36 | H | Me | methoxymethyl | iPr | isopropyl |
| 37 | methyl | Cl | H | CF₃ | isopropyl |
| 38 | methyl | Cl | H | CHF₂ | isopropyl |
| 39 | methyl | Cl | H | iPr | isopropyl |
| 40 | methyl | Cl | methoxymethyl | CF₃ | isopropyl |
| 41 | methyl | Cl | methoxymethyl | CHF₂ | isopropyl |
| 42 | methyl | Cl | methoxymethyl | iPr | isopropyl |
| 43 | methyl | Me | H | CF₃ | isopropyl |
| 44 | methyl | Me | H | CHF₂ | isopropyl |
| 45 | methyl | Me | H | iPr | isopropyl |
| 46 | methyl | Me | methoxymethyl | CF₃ | isopropyl |
| 47 | methyl | Me | methoxymethyl | CHF₂ | isopropyl |
| 48 | methyl | Me | methoxymethyl | iPr | isopropyl |
| 49 | H | Cl | H | CF₃ | propargyl |
| 50 | H | Cl | H | CHF₂ | propargyl |
| 51 | H | Cl | H | iPr | propargyl |
| 52 | H | Cl | methoxymethyl | CF₃ | propargyl |
| 53 | H | Cl | methoxymethyl | CHF₂ | propargyl |
| 54 | H | Cl | methoxymethyl | iPr | propargyl |
| 55 | H | Me | H | CF₃ | propargyl |
| 56 | H | Me | H | CHF₂ | propargyl |
| 57 | H | Me | H | iPr | propargyl |
| 58 | H | Me | methoxymethyl | CF₃ | propargyl |
| 59 | H | Me | methoxymethyl | CHF₂ | propargyl |
| 60 | H | Me | methoxymethyl | iPr | propargyl |
| 61 | methyl | Cl | H | CF₃ | propargyl |
| 62 | methyl | Cl | H | CHF₂ | propargyl |
| 63 | methyl | Cl | H | iPr | propargyl |
| 64 | methyl | Cl | methoxymethyl | CF₃ | propargyl |
| 65 | methyl | Cl | methoxymethyl | CHF₂ | propargyl |
| 66 | methyl | Cl | methoxymethyl | iPr | propargyl |
| 67 | methyl | Me | H | CF₃ | propargyl |
| 68 | methyl | Me | H | CHF₂ | propargyl |
| 69 | methyl | Me | H | iPr | propargyl |
| 70 | methyl | Me | methoxymethyl | CF₃ | propargyl |
| 71 | methyl | Me | methoxymethyl | CHF₂ | propargyl |
| 72 | methyl | Me | methoxymethyl | iPr | propargyl |
| 73 | H | Cl | H | CF₃ | 2,2,2-trifluoroethyl |
| 74 | H | Cl | H | CHF₂ | 2,2,2-trifluoroethyl |
| 75 | H | Cl | H | iPr | 2,2,2-trifluoroethyl |
| 76 | H | Cl | methoxymethyl | CF₃ | 2,2,2-trifluoroethyl |
| 77 | H | Cl | methoxymethyl | CHF₂ | 2,2,2-trifluoroethyl |
| 78 | H | Cl | methoxymethyl | iPr | 2,2,2-trifluoroethyl |
| 79 | H | Me | H | CF₃ | 2,2,2-trifluoroethyl |
| 80 | H | Me | H | CHF₂ | 2,2,2-trifluoroethyl |
| 81 | H | Me | H | iPr | 2,2,2-trifluoroethyl |
| 82 | H | Me | methoxymethyl | CF₃ | 2,2,2-trifluoroethyl |
| 83 | H | Me | methoxymethyl | CHF₂ | 2,2,2-trifluoroethyl |
| 84 | H | Me | methoxymethyl | iPr | 2,2,2-trifluoroethyl |
| 85 | methyl | Cl | H | CF₃ | 2,2,2-trifluoroethyl |
| 86 | methyl | Cl | H | CHF₂ | 2,2,2-trifluoroethyl |
| 87 | methyl | Cl | H | iPr | 2,2,2-trifluoroethyl |
| 88 | methyl | Cl | methoxymethyl | CF₃ | 2,2,2-trifluoroethyl |
| 89 | methyl | Cl | methoxymethyl | CHF₂ | 2,2,2-trifluoroethyl |
| 90 | methyl | Cl | methoxymethyl | iPr | 2,2,2-trifluoroethyl |
| 91 | methyl | Me | H | CF₃ | 2,2,2-trifluoroethyl |
| 92 | methyl | Me | H | CHF₂ | 2,2,2-trifluoroethyl |
| 93 | methyl | Me | H | iPr | 2,2,2-trifluoroethyl |
| 94 | methyl | Me | methoxymethyl | CF₃ | 2,2,2-trifluoroethyl |
| 95 | methyl | Me | methoxymethyl | CHF₂ | 2,2,2-trifluoroethyl |
| 96 | methyl | Me | methoxymethyl | iPr | 2,2,2-trifluoroethyl |
| 97 | H | Cl | H | CF₃ | 2,2-difluoroethyl |
| 98 | H | Cl | H | CHF₂ | 2,2-difluoroethyl |
| 99 | H | Cl | H | iPr | 2,2-difluoroethyl |
| 100 | H | Cl | methoxymethyl | CF₃ | 2,2-difluoroethyl |
| 101 | H | Cl | methoxymethyl | CHF₂ | 2,2-difluoroethyl |
| 102 | H | Cl | methoxymethyl | iPr | 2,2-difluoroethyl |
| 103 | H | Me | H | CF₃ | 2,2-difluoroethyl |
| 104 | H | Me | H | CHF₂ | 2,2-difluoroethyl |
| 105 | H | Me | H | iPr | 2,2-difluoroethyl |
| 106 | H | Me | methoxymethyl | CF₃ | 2,2-difluoroethyl |
| 107 | H | Me | methoxymethyl | CHF₂ | 2,2-difluoroethyl |
| 108 | H | Me | methoxymethyl | iPr | 2,2-difluoroethyl |
| 109 | methyl | Cl | H | CF₃ | 2,2-difluoroethyl |
| 110 | methyl | Cl | H | CHF₂ | 2,2-difluoroethyl |
| 111 | methyl | Cl | H | iPr | 2,2-difluoroethyl |
| 112 | methyl | Cl | methoxymethyl | CF₃ | 2,2-difluoroethyl |
| 113 | methyl | Cl | methoxymethyl | CHF₂ | 2,2-difluoroethyl |
| 114 | methyl | Cl | methoxymethyl | iPr | 2,2-difluoroethyl |
| 115 | methyl | Me | H | CF₃ | 2,2-difluoroethyl |
| 116 | methyl | Me | H | CHF₂ | 2,2-difluoroethyl |
| 117 | methyl | Me | H | iPr | 2,2-difluoroethyl |
| 118 | methyl | Me | methoxymethyl | CF₃ | 2,2-difluoroethyl |
| 119 | methyl | Me | methoxymethyl | CHF₂ | 2,2-difluoroethyl |
| 120 | methyl | Me | methoxymethyl | iPr | 2,2-difluoroethyl |
| 121 | H | Cl | H | CF₃ | cyclopropyl |
| 122 | H | Cl | H | CHF₂ | cyclopropyl |
| 123 | H | Cl | H | iPr | cyclopropyl |
| 124 | H | Cl | methoxymethyl | CF₃ | cyclopropyl |
| 125 | H | Cl | methoxymethyl | CHF₂ | cyclopropyl |
| 126 | H | Cl | methoxymethyl | iPr | cyclopropyl |
| 127 | H | Me | H | CF₃ | cyclopropyl |
| 128 | H | Me | H | CHF₂ | cyclopropyl |
| 129 | H | Me | H | iPr | cyclopropyl |
| 130 | H | Me | methoxymethyl | CF₃ | cyclopropyl |
| 131 | H | Me | methoxymethyl | CHF₂ | cyclopropyl |
| 132 | H | Me | methoxymethyl | iPr | cyclopropyl |
| 133 | methyl | Cl | H | CF₃ | cyclopropyl |
| 134 | methyl | Cl | H | CHF₂ | cyclopropyl |
| 135 | methyl | Cl | H | iPr | cyclopropyl |
| 136 | methyl | Cl | methoxymethyl | CF₃ | cyclopropyl |
| 137 | methyl | Cl | methoxymethyl | CHF₂ | cyclopropyl |
| 138 | methyl | Cl | methoxymethyl | iPr | cyclopropyl |
| 139 | methyl | Me | H | CF₃ | cyclopropyl |
| 140 | methyl | Me | H | CHF₂ | cyclopropyl |
| 141 | methyl | Me | H | iPr | cyclopropyl |
| 142 | methyl | Me | methoxymethyl | CF₃ | cyclopropyl |
| 143 | methyl | Me | methoxymethyl | CHF₂ | cyclopropyl |
| 144 | methyl | Me | methoxymethyl | iPr | cyclopropyl |
| 145 | H | Cl | H | CF₃ | 1-cyanocyclopropyl |
| 146 | H | Cl | H | CHF₂ | 1-cyanocyclopropyl |
| 147 | H | Cl | H | iPr | 1-cyanocyclopropyl |
| 148 | H | Cl | methoxymethyl | CF₃ | 1-cyanocyclopropyl |
| 149 | H | Cl | methoxymethyl | CHF₂ | 1-cyanocyclopropyl |
| 150 | H | Cl | methoxymethyl | iPr | 1-cyanocyclopropyl |
| 151 | H | Me | H | CF₃ | 1-cyanocyclopropyl |
| 152 | H | Me | H | CHF₂ | 1-cyanocyclopropyl |
| 153 | H | Me | H | iPr | 1-cyanocyclopropyl |
| 154 | H | Me | methoxymethyl | CF₃ | 1-cyanocyclopropyl |
| 155 | H | Me | methoxymethyl | CHF₂ | 1-cyanocyclopropyl |
| 156 | H | Me | methoxymethyl | iPr | 1-cyanocyclopropyl |
| 157 | methyl | Cl | H | CF₃ | 1-cyanocyclopropyl |
| 158 | methyl | Cl | H | CHF₂ | 1-cyanocyclopropyl |
| 159 | methyl | Cl | H | iPr | 1-cyanocyclopropyl |
| 160 | methyl | Cl | methoxymethyl | CF₃ | 1-cyanocyclopropyl |
| 161 | methyl | Cl | methoxymethyl | CHF₂ | 1-cyanocyclopropyl |
| 162 | methyl | Cl | methoxymethyl | iPr | 1-cyanocyclopropyl |
| 163 | methyl | Me | H | CF₃ | 1-cyanocyclopropyl |
| 164 | methyl | Me | H | CHF₂ | 1-cyanocyclopropyl |
| 165 | methyl | Me | H | iPr | 1-cyanocyclopropyl |
| 166 | methyl | Me | methoxymethyl | CF₃ | 1-cyanocyclopropyl |
| 167 | methyl | Me | methoxymethyl | CHF₂ | 1-cyanocyclopropyl |
| 168 | methyl | Me | methoxymethyl | iPr | 1-cyanocyclopropyl |
| 169 | H | Cl | H | CF₃ | 1-cyanocyclobutyl |
| 170 | H | Cl | H | CHF₂ | 1-cyanocyclobutyl |
| 171 | H | Cl | H | iPr | 1-cyanocyclobutyl |
| 172 | H | Cl | methoxymethyl | CF₃ | 1-cyanocyclobutyl |
| 173 | H | Cl | methoxymethyl | CHF₂ | 1-cyanocyclobutyl |
| 174 | H | Cl | methoxymethyl | iPr | 1-cyanocyclobutyl |
| 175 | H | Me | H | CF₃ | 1-cyanocyclobutyl |
| 176 | H | Me | H | CHF₂ | 1-cyanocyclobutyl |
| 177 | H | Me | H | iPr | 1-cyanocyclobutyl |
| 178 | H | Me | methoxymethyl | CF₃ | 1-cyanocyclobutyl |
| 179 | H | Me | methoxymethyl | CHF₂ | 1-cyanocyclobutyl |
| 180 | H | Me | methoxymethyl | iPr | 1-cyanocyclobutyl |
| 181 | methyl | Cl | H | CF₃ | 1-cyanocyclobutyl |
| 182 | methyl | Cl | H | CHF₂ | 1-cyanocyclobutyl |
| 183 | methyl | Cl | H | iPr | 1-cyanocyclobutyl |
| 184 | methyl | Cl | methoxymethyl | CF₃ | 1-cyanocyclobutyl |
| 185 | methyl | Cl | methoxymethyl | CHF₂ | 1-cyanocyclobutyl |
| 186 | methyl | Cl | methoxymethyl | iPr | 1-cyanocyclobutyl |
| 187 | methyl | Me | H | CF₃ | 1-cyanocyclobutyl |
| 188 | methyl | Me | H | CHF₂ | 1-cyanocyclobutyl |
| 189 | methyl | Me | H | iPr | 1-cyanocyclobutyl |
| 190 | methyl | Me | methoxymethyl | CF₃ | 1-cyanocyclobutyl |
| 191 | methyl | Me | methoxymethyl | CHF₂ | 1-cyanocyclobutyl |
| 192 | methyl | Me | methoxymethyl | iPr | 1-cyanocyclobutyl |
| 193 | H | Cl | H | CF₃ | 3,3-difluorocyclobutyl |
| 194 | H | Cl | H | CHF₂ | 3,3-difluorocyclobutyl |
| 195 | H | Cl | H | iPr | 3,3-difluorocyclobutyl |
| 196 | H | Cl | methoxymethyl | CF₃ | 3,3-difluorocyclobutyl |
| 197 | H | Cl | methoxymethyl | CHF₂ | 3,3-difluorocyclobutyl |
| 198 | H | Cl | methoxymethyl | iPr | 3,3-difluorocyclobutyl |
| 199 | H | Me | H | CF₃ | 3,3-difluorocyclobutyl |
| 200 | H | Me | H | CHF₂ | 3,3-difluorocyclobutyl |
| 201 | H | Me | H | iPr | 3,3-difluorocyclobutyl |
| 202 | H | Me | methoxymethyl | CF₃ | 3,3-difluorocyclobutyl |
| 203 | H | Me | methoxymethyl | CHF₂ | 3,3-difluorocyclobutyl |
| 204 | H | Me | methoxymethyl | iPr | 3,3-difluorocyclobutyl |
| 205 | methyl | Cl | H | CF₃ | 3,3-difluorocyclobutyl |
| 206 | methyl | Cl | H | CHF₂ | 3,3-difluorocyclobutyl |
| 207 | methyl | Cl | H | iPr | 3,3-difluorocyclobutyl |
| 208 | methyl | Cl | methoxymethyl | CF₃ | 3,3-difluorocyclobutyl |
| 209 | methyl | Cl | methoxymethyl | CHF₂ | 3,3-difluorocyclobutyl |
| 210 | methyl | Cl | methoxymethyl | iPr | 3,3-difluorocyclobutyl |
| 211 | methyl | Me | H | CF₃ | 3,3-difluorocyclobutyl |
| 212 | methyl | Me | H | CHF₂ | 3,3-difluorocyclobutyl |
| 213 | methyl | Me | H | iPr | 3,3-difluorocyclobutyl |
| 214 | methyl | Me | methoxymethyl | CF₃ | 3,3-difluorocyclobutyl |
| 215 | methyl | Me | methoxymethyl | CHF₂ | 3,3-difluorocyclobutyl |
| 216 | methyl | Me | methoxymethyl | iPr | 3,3-difluorocyclobutyl |
| 217 | H | Cl | H | CF₃ | 1,1-dioxothietan-3-yl |
| 218 | H | Cl | H | CHF₂ | 1,1-dioxothietan-3-yl |
| 219 | H | Cl | H | iPr | 1,1-dioxothietan-3-yl |
| 220 | H | Cl | methoxymethyl | CF₃ | 1,1-dioxothietan-3-yl |
| 221 | H | Cl | methoxymethyl | CHF₂ | 1,1-dioxothietan-3-yl |
| 222 | H | Cl | methoxymethyl | iPr | 1,1-dioxothietan-3-yl |
| 223 | H | Me | H | CF₃ | 1,1-dioxothietan-3-yl |
| 224 | H | Me | H | CHF₂ | 1,1-dioxothietan-3-yl |
| 225 | H | Me | H | iPr | 1,1-dioxothietan-3-yl |
| 226 | H | Me | methoxymethyl | CF₃ | 1,1-dioxothietan-3-yl |
| 227 | H | Me | methoxymethyl | CHF₂ | 1,1-dioxothietan-3-yl |
| 228 | H | Me | methoxymethyl | iPr | 1,1-dioxothietan-3-yl |
| 229 | methyl | Cl | H | CF₃ | 1,1-dioxothietan-3-yl |
| 230 | methyl | Cl | H | CHF₂ | 1,1-dioxothietan-3-yl |
| 231 | methyl | Cl | H | iPr | 1,1-dioxothietan-3-yl |
| 232 | methyl | Cl | methoxymethyl | CF₃ | 1,1-dioxothietan-3-yl |
| 233 | methyl | Cl | methoxymethyl | CHF₂ | 1,1-dioxothietan-3-yl |
| 234 | methyl | Cl | methoxymethyl | iPr | 1,1-dioxothietan-3-yl |
| 235 | methyl | Me | H | CF₃ | 1,1-dioxothietan-3-yl |
| 236 | methyl | Me | H | CHF₂ | 1,1-dioxothietan-3-yl |
| 237 | methyl | Me | H | iPr | 1,1-dioxothietan-3-yl |
| 238 | methyl | Me | methoxymethyl | CF₃ | 1,1-dioxothietan-3-yl |
| 239 | methyl | Me | methoxymethyl | CHF₂ | 1,1-dioxothietan-3-yl |
| 240 | methyl | Me | methoxymethyl | iPr | 1,1-dioxothietan-3-yl |
| 241 | H | Cl | H | CF₃ | cyclopropylmethyl |
| 242 | H | Cl | H | CHF₂ | cyclopropylmethyl |
| 243 | H | Cl | H | iPr | cyclopropylmethyl |
| 244 | H | Cl | methoxymethyl | CF₃ | cyclopropylmethyl |
| 245 | H | Cl | methoxymethyl | CHF₂ | cyclopropylmethyl |
| 246 | H | Cl | methoxymethyl | iPr | cyclopropylmethyl |
| 247 | H | Me | H | CF₃ | cyclopropylmethyl |
| 248 | H | Me | H | CHF₂ | cyclopropylmethyl |
| 249 | H | Me | H | iPr | cyclopropylmethyl |
| 250 | H | Me | methoxymethyl | CF₃ | cyclopropylmethyl |
| 251 | H | Me | methoxymethyl | CHF₂ | cyclopropylmethyl |
| 252 | H | Me | methoxymethyl | iPr | cyclopropylmethyl |
| 253 | methyl | Cl | H | CF₃ | cyclopropylmethyl |
| 254 | methyl | Cl | H | CHF₂ | cyclopropylmethyl |
| 255 | methyl | Cl | H | iPr | cyclopropylmethyl |
| 256 | methyl | Cl | methoxymethyl | CF₃ | cyclopropylmethyl |
| 257 | methyl | Cl | methoxymethyl | CHF₂ | cyclopropylmethyl |
| 258 | methyl | Cl | methoxymethyl | iPr | cyclopropylmethyl |
| 259 | methyl | Me | H | CF₃ | cyclopropylmethyl |
| 260 | methyl | Me | H | CHF₂ | cyclopropylmethyl |
| 261 | methyl | Me | H | iPr | cyclopropylmethyl |
| 262 | methyl | Me | methoxymethyl | CF₃ | cyclopropylmethyl |
| 263 | methyl | Me | methoxymethyl | CHF₂ | cyclopropylmethyl |
| 264 | methyl | Me | methoxymethyl | iPr | cyclopropylmethyl |
| 265 | H | Me | H | CF₃ | 2-bicyclo[1.1.1]pentanyl |
| 266 | H | Me | H | CF₃ | 4-ethylthiazole-5-yl |
| 267 | H | Me | H | CF₃ | (1,3-dihydroisobenzofuran- |
| | | | | | 5-yl) methyl |
| 268 | H | Me | H | CF₃ | 3-oxo-2-(2,2,2-trifluoro- |
| | | | | | ethyl)isoxazolidine-4-yl |
| 269 | H | Me | H | CF₃ | 2-(2-fluorophenoxy)ethyl |
| 270 | H | Me | H | CF₃ | 6-methoxy-2-methyl-hex-3- |
| | | | | | yn-2-yl |
| 271 | H | Me | H | CF₃ | 1-cyclopropyl ethyl |
| 272 | H | Me | H | CF₃ | 1-(2-ethylthiazol-4-yl)ethyl |
| 273 | H | Me | H | CF₃ | 2-fluoro 3-cyano phenyl |
| 274 | H | Me | H | CF₃ | benzyl |
| 275 | H | Me | H | CF₃ | 1-(3-pyridyl)ethyl |
| 276 | H | Me | methoxymethyl | CF₃ | 3-oxo-2-ethyl |
| | | | | | isoxazolidine-4-yl |
| 277 | H | Br | methoxymethyl | CF₃ | cyclopropyl |
| 278 | H | Br | methoxymethyl | CF₃ | 1-cyano cyclopropyl |
| 279 | H | Br | H | CF₃ | cyclopropyl |
| 280 | H | Br | H | CF₃ | 1-cyano cyclopropyl |
| 281 | H | Br | H | CF₃ | 3-oxo-2-ethyl |
| | | | | | isoxazolidine-4-yl |
| 282 | H | Br | H | CF₃ | 2,2,2-trifluoroethyl |

TABLE 5
Makes available 54 compounds of the formula
(I-5a), where the substituents R¹, R³, X,
Y and Z are designated in each row below:

| Index | Z | X | Y | R¹ | R³ |
|---|---|---|---|---|---|
| 1 | CH | CH | N | 2,2,2-trifluoroethyl | Cl |
| 2 | CH | CH | N | cyclopropyl | Cl |
| 3 | CH | CH | N | 1-cyanocyclopropyl | Cl |
| 4 | CH | N | CH | 2,2,2-trifluoroethyl | Cl |
| 5 | CH | N | CH | cyclopropyl | Cl |
| 6 | CH | N | CH | 1-cyanocyclopropyl | Cl |
| 7 | CH | N | N | 2,2,2-trifluoroethyl | Cl |
| 8 | CH | N | N | cyclopropyl | Cl |
| 9 | CH | N | N | 1-cyanocyclopropyl | Cl |
| 10 | N | CH | CH | 2,2,2-trifluoroethyl | Cl |
| 11 | N | CH | CH | cyclopropyl | Cl |
| 12 | N | CH | CH | 1-cyanocyclopropyl | Cl |
| 13 | N | CH | N | 2,2,2-trifluoroethyl | Cl |
| 14 | N | CH | N | cyclopropyl | Cl |
| 15 | N | CH | N | 1-cyanocyclopropyl | Cl |
| 16 | N | N | CH | 2,2,2-trifluoroethyl | Cl |
| 17 | N | N | CH | cyclopropyl | Cl |
| 18 | N | N | CH | 1-cyanocyclopropyl | Cl |
| 19 | CH | CH | N | 2,2,2-trifluoroethyl | Br |
| 20 | CH | CH | N | cyclopropyl | Br |
| 21 | CH | CH | N | 1-cyanocyclopropyl | Br |
| 22 | CH | N | CH | 2,2,2-trifluoroethyl | Br |
| 23 | CH | N | CH | cyclopropyl | Br |
| 24 | CH | N | CH | 1-cyanocyclopropyl | Br |
| 25 | CH | N | N | 2,2,2-trifluoroethyl | Br |
| 26 | CH | N | N | cyclopropyl | Br |
| 27 | CH | N | N | 1-cyanocyclopropyl | Br |
| 28 | N | CH | CH | 2,2,2-trifluoroethyl | Br |
| 29 | N | CH | CH | cyclopropyl | Br |
| 30 | N | CH | CH | 1-cyanocyclopropyl | Br |
| 31 | N | CH | N | 2,2,2-trifluoroethyl | Br |
| 32 | N | CH | N | cyclopropyl | Br |
| 33 | N | CH | N | 1-cyanocyclopropyl | Br |
| 34 | N | N | CH | 2,2,2-trifluoroethyl | Br |
| 35 | N | N | CH | cyclopropyl | Br |
| 36 | N | N | CH | 1-cyanocyclopropyl | Br |
| 37 | CH | CH | N | 2,2,2-trifluoroethyl | Me |
| 38 | CH | CH | N | cyclopropyl | Me |
| 39 | CH | CH | N | 1-cyanocyclopropyl | Me |
| 40 | CH | N | CH | 2,2,2-trifluoroethyl | Me |
| 41 | CH | N | CH | cyclopropyl | Me |
| 42 | CH | N | CH | 1-cyanocyclopropyl | Me |
| 43 | CH | N | N | 2,2,2-trifluoroethyl | Me |
| 44 | CH | N | N | cyclopropyl | Me |
| 45 | CH | N | N | 1-cyanocyclopropyl | Me |
| 46 | N | CH | CH | 2,2,2-trifluoroethyl | Me |
| 47 | N | CH | CH | cyclopropyl | Me |
| 48 | N | CH | CH | 1-cyanocyclopropyl | Me |
| 49 | N | CH | N | 2,2,2-trifluoroethyl | Me |
| 50 | N | CH | N | cyclopropyl | Me |
| 51 | N | CH | N | 1-cyanocyclopropyl | Me |
| 52 | N | N | CH | 2,2,2-trifluoroethyl | Me |
| 53 | N | N | CH | cyclopropyl | Me |
| 54 | N | N | CH | 1-cyanocyclopropyl | Me |

Also made available are certain intermediate compounds, some of which are novel.

Accordingly, made available herein are:
A. compounds of formulae (II-1), (II-2), (II-3) and (II-4), wherein R³, R⁶ and R⁷ are as defined in Tables 1 to 5 respectively.

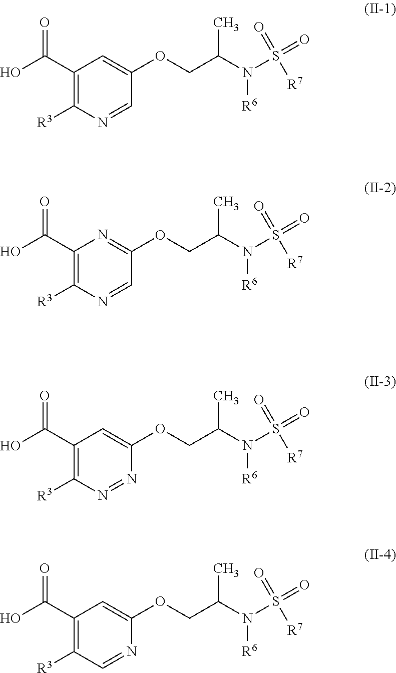

B. compounds of formulae (IV-1), (IV-2), (IV-3), and (IV-4), wherein R³, R⁶ and R⁷ are as defined in Tables 1 to 5 respectively, and wherein G is halogen or G₁ to G₄.

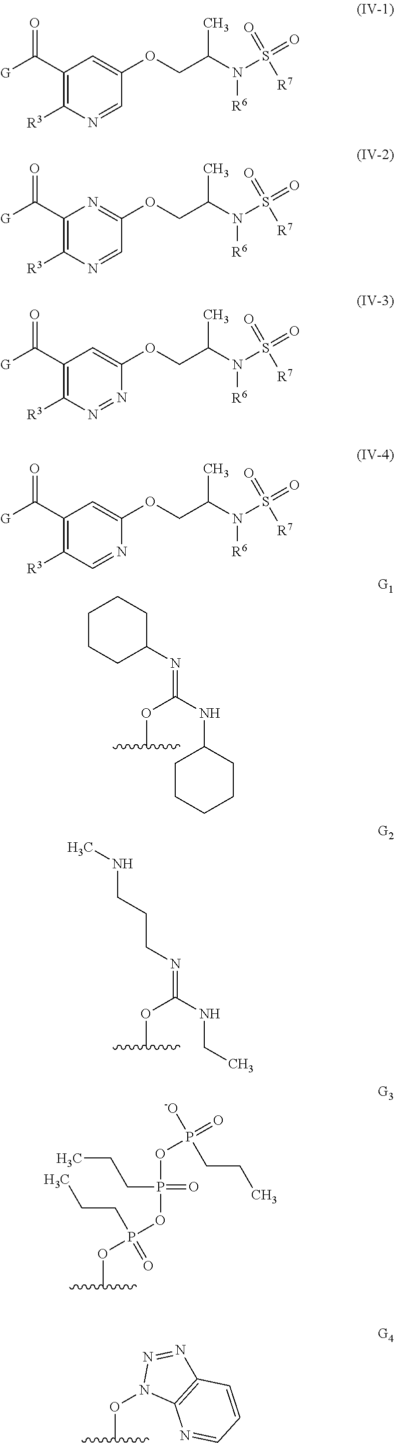

C. compounds of formulae (V-1), (V-2), (V-3), and (V-4), wherein R³, R⁶ and R⁷ are as defined in Tables 1 to 5, and wherein R²⁰ is methyl or ethyl.

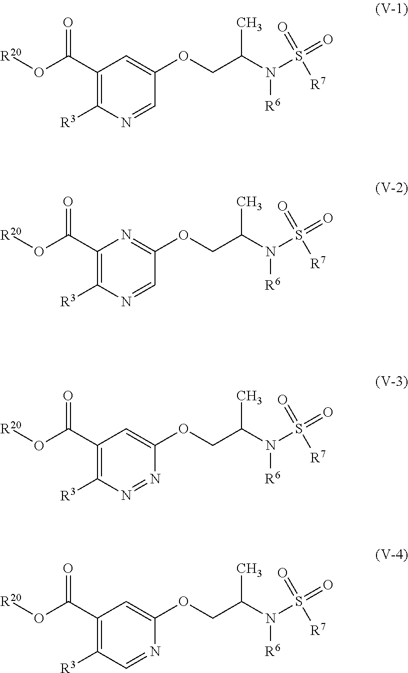

D. compounds of formulae (VI-1), (VI-2), (VI-3), and (VI-4), wherein R¹, R² and R³ are as defined in Tables 1 to 5 respectively; and wherein R²⁰ is methyl or ethyl.

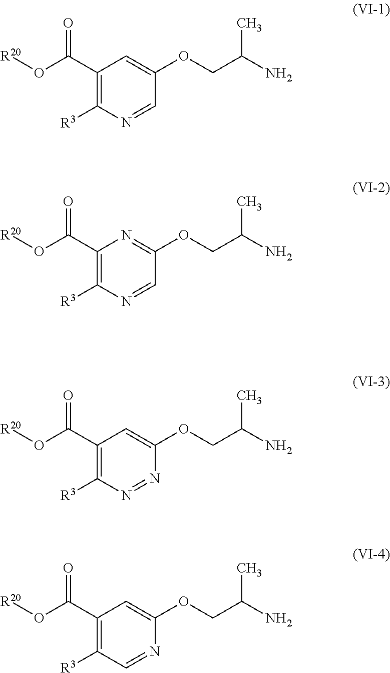

E. compounds of formulae (VIII-1), (VIII-2), (VIII-3), and (VIII-4), wherein R³ is as defined in Tables 1 to 5 respectively; and wherein R²⁰ is methyl and R²¹ is methyl or ethyl; or R²⁰ is ethyl and R²¹ is methyl or ethyl.

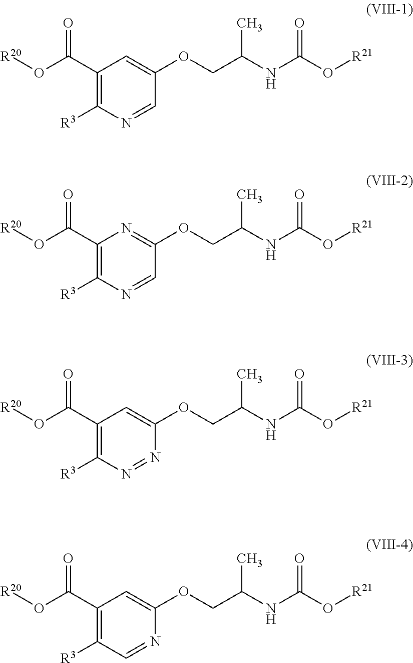

F. compounds of formulae (XIII-1), (XIII-2), (XIII-3), and (XIII-4), wherein R¹, R² and R³ are as defined in Tables 1 to 5 respectively.

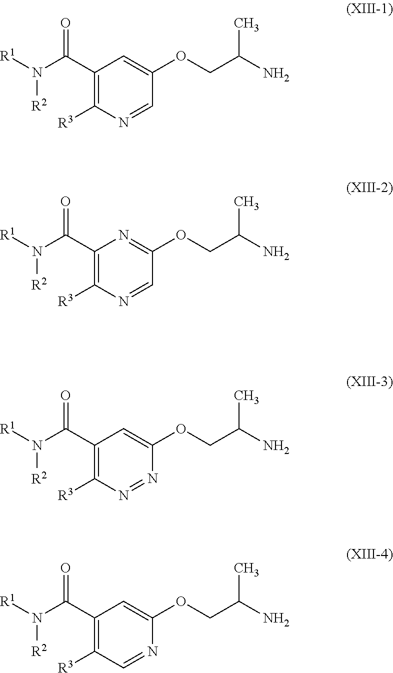

G. compounds of formulae (XIV-1), (XIV-2), (XIV-3), and (XIV-4), wherein R¹, R² and R³ are as defined in Tables 1 to 5 respectively, and wherein R²¹ is methyl or ethyl.

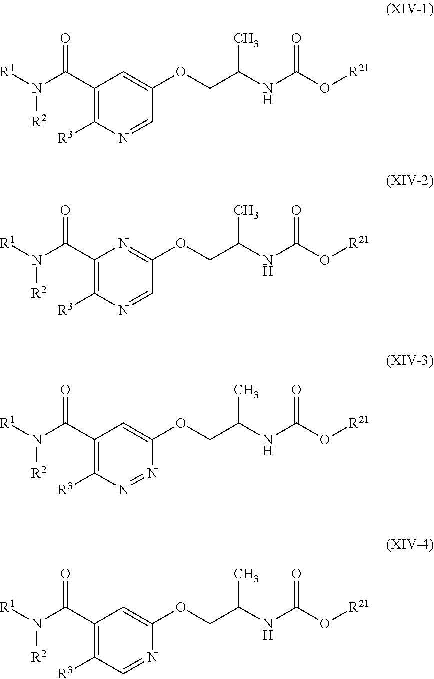

H. compounds of formulae (XV-1), (XV-2), (XV-3) and (XV-4), wherein R¹, R² and R³ are as defined in Tables 1 to 5.

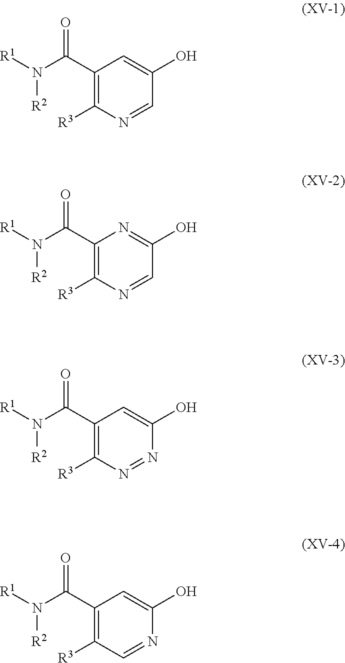

I. compounds of formula (II-5), wherein X, Y, Z, R³, R⁶, and R⁷ are as defined as in Table 5.

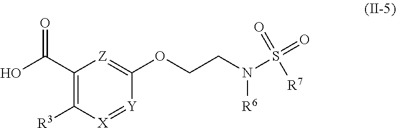

J. compounds of formula (IV-5), wherein X, Y, Z, R³, R⁶, and R⁷ are as defined as in Table 5, and wherein G is halogen or G₁ to G₄

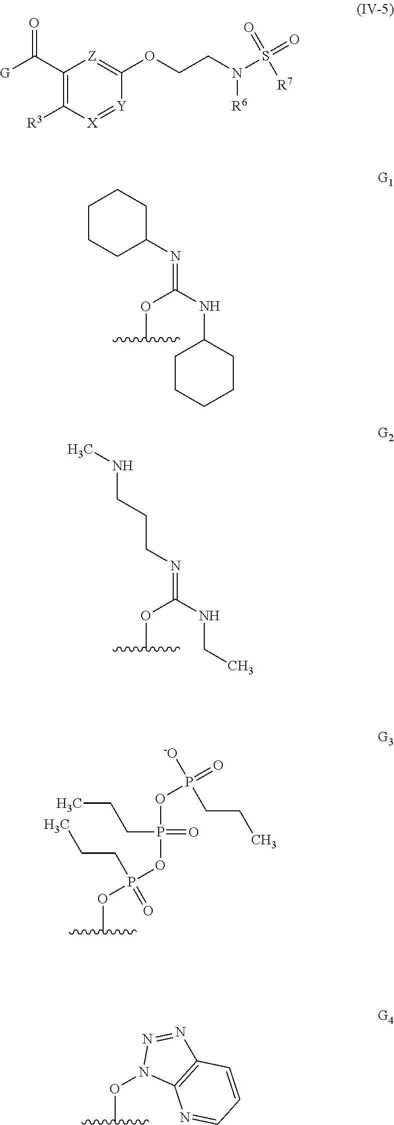

K. compounds of formula (V-5), wherein X, Y, Z, R³, R⁶, and R⁷ are as defined as in Table 5, and wherein R²⁰ is methyl or ethyl

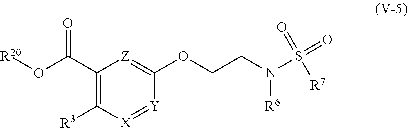

L. compounds of formula (VI-5), wherein X, Y, Z, and R³ are as defined as in Table 5, and wherein R²⁰ is methyl or ethyl

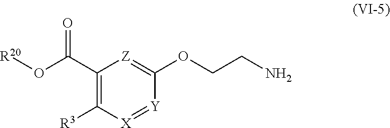

M. compounds of formula (VIII-5), wherein X, Y, Z, and R³ are as defined as in Table 5, and wherein R²⁰ is methyl and R²¹ is methyl or ethyl; or R²⁰ is ethyl and R²¹ is methyl or ethyl.

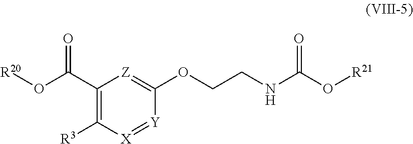

N. compounds of formula (XIII-5), wherein X, Y, Z, R¹ and R³ are as defined as in Table 5.

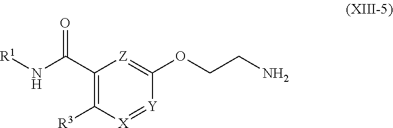

O. compounds of formula (XIV-5), wherein X, Y, Z, R¹ and R³ are as defined as in Table 5, and wherein R²¹ is methyl or ethyl.

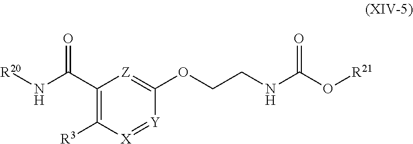

The compounds of formula (I) according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the above mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Meligethes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens;
Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris,
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltiajaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

In a particular embodiment, a compound of the formula (I) can control mites, rust mites and spider mites in crops, tress, and plants selected from vegetables (especially tomatoes and cucurbits), citrus, pome fruits, stone fruit, tree nuts, cotton, tropical crops, avocados, ornamentals, beans, soybean, strawberry, and grapes.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubéreux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The compounds of formula (I) may be particularly suitable for control of mites, spider mites and rust mites, for example, Acarapis spp; Acarapis woodi; Acarus siro; Acarus spp; Aceria sheldoni; Aculops pelekassi; Aculops spp; Aculus schlechtendali; Aculus spp; Amblyseius fallacis; Brevipalpus spp; Brevipalpus phoenicis; Bryobia praetiosa; Bryobia rubrioculus; Caloglyphus spp; Cheyletiella blakei; Cheyletiella spp; Cheyletiella yasguri; Chorioptes bovis; Chorioptes spp; Cytodites spp; Demodex bovis; Demodex caballi; Demodex canis; Demodex caprae; Demodex equi; Demodex ovis; Demodex spp; Demodex suis; Dermanyssus gallinae; Dermanyssus spp; Eotetranychus spp; Eotetranychus willamettei; Epitrimerus pyri; Eriophyes ribis; Eriophyes spp; Eriophyes vitis; Eutetranychus spp; Halotydeus destructor; Hemitarsonemus spp; Knemidocoptes spp; Laminosioptes spp; Listrophorus spp; Myobia spp; Neoschongastia xerothermobia; Neotrombicula autumnalis; Neotrombicula desaleri; Notoedres cati; Notoedres spp; Oligonychus coffeae; Oligonychus ilicis; Oligonychus spp; Ornithocheyletia spp; Ornithonyssus bursa; Ornithonyssus spp; Ornithonyssus sylviarum; Otodectes cynotis; Otodectes spp; Panonychus citri; Panonychus spp; Panonychus ulmi; Phyllocoptruta oleivora; Phyllocoptruta spp.; Phytoseiulus spp.; Pneumonyssoides caninum; Polyphagotarsonemus latus; Polyphagotarsonemus spp; Psorergates ovis; Psorergates spp; Psoroptes cuniculi; Psoroptes equi; Psoroptes ovis; Psoroptes spp; Pterolichus spp; Raillietia spp; Rhizoglyphus spp; Sarcoptes bovis; Sarcoptes canis; Sarcoptes caprae; Sarcoptes equi; Sarcoptes ovis; Sarcoptes rupicaprae; Sarcoptes spp; Sarcoptes suis; Steneotarsonemus spinki; Steneotarsonemus spp; Sternostoma spp; Tarsonemus spp; Tetranychus cinnabarinus; Tetranychus kanzawai; Tetranychus spp; Tetranychus urticae; Trombicula akamushi; Trombicula spp; Typhlodromus occidentalis; Tyrophagus spp; Varroa jacobsoni; Varroa spp; Vasates lycopersici; and Zetzellia mali.

In an embodiment, a compound of formula (I) may control one or more of: Aceria sheldoni; Aculus lycopersici; Aculus pelekassi; Aculus schlechtendali; Brevipalpus phoenicis; Brevipalpus spp.; Bryobia rubrioculus; Eotetranychus carpini; Eotetranychus spp.; Epitrimerus pyri; Eriophyes piri; Eriophyes spp.; Eriophyes vitis; Eutetranychus africanus; Eutetranychus orientalis; Oligonychus pratensis; Panonychus citri; Panonychus ulmi; Phyllocoptes vitis; Phyllocoptruta oleivora; Polyphagotarsonemus latus; Tetranychus cinnabarinus; Tetranychus kanzawai; Tetranychus spp.; and Tetranychus urticae.

In a further embodiment, a compound of formula (I) may especially suitable for controlling one or more of: Aceria sheldoni; Aculus pelekassi; Brevipalpus phoenicis; Brevipalpus spp.; Eriophyes piri; Eriophyes vitis; Eutetranychus africanus; Eutetranychus orientalis; Oligonychus pratensis; Panonychus ulmi; Phyllocoptes vitis; Phyllocoptruta oleivora; Polyphagotarsonemus latus; Tetranychus cinnabarinus; Tetranychus kanzawai; Tetranychus spp.; and Tetranychus urticae.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO95/33818) or protein or polypeptide factors involved in plant pathogen defence (so-called “plant disease resistance genes”, as described in WO03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention provides a compound of the first aspect for use in therapy. The present invention provides a compound of the first aspect, for use in controlling parasites in or on an animal. The present invention further provides a compound of the first aspect, for use in controlling ectoparasites on an animal. The present invention further provides a compound of the first aspect, for use in preventing and/or treating diseases transmitted by ectoparasites.

The present invention provides the use of a compound of the first aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites. The present invention provides the use of a compound of the first aspect, in controlling parasites in or on an animal. The present invention further provides the use of a compound of the first aspect, in controlling ectoparasites on an animal.

The term “controlling” when used in context of parasites in or on an animal refers to reducing the number of pests or parasites, eliminating pests or parasites and/or preventing further pest or parasite infestation.

The term “treating” when used in context of parasites in or on an animal refers to restraining, slowing, stopping or reversing the progression or severity of an existing symptom or disease.

The term “preventing” when used in context of parasites in or on an animal refers to the avoidance of a symptom or disease developing in the animal.

The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camelids, pigs, sheep, goats and horses. Companion animals include, but are not limited to, dogs, cats and rabbits.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola Ovis and Bovicola Bovis.

The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of or involvement or the severity of the disease or disorder; the response of the individual; the particular compound administered; the mode of administration; the bioavailability characteristics of the preparation administered; the dose regimen selected; the use of concomitant medication; and other relevant circumstances.

The compounds of the invention may be administered to the animal by any route which has the desired effect including, but not limited to topically, orally, parenterally and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.

Salt forms of the compounds of the invention include both pharmaceutically acceptable salts and veterinary acceptable salts, which can be different to agrochemically acceptable salts. Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.

The present invention may also provide a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO2008/151984, WO2003/034823, U.S. Pat. No. 5,631,072, WO2005/64072, WO2006/128870, EP 1724392, WO2005113886 or WO2007/090739.

Further areas of use of the compositions according to the invention may be the field of tree injection/trunk treatment for all ornamental trees as well all sort of fruit and nut trees.

In the field of tree injection/trunk treatment, the compounds according to the present invention may be especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, Serviceberry, Hawthorn |
| | Chrysobothris | Apple, Apricot, Beech, Boxelder, Cherry, Chestnut, |
| | femorata | Currant, Elm, Hawthorn, Hackberry, Hickory, |
| | | Horsechestnut, Linden, Maple, Mountain-ash, Oak, |
| | | Pecan, Pear, Peach, Persimmon, Plum, Poplar, |
| | | Quince, Redbud, Serviceberry, Sycamore, Walnut, |
| | | Willow |
| | Texania campestris | Basswood, Beech, Maple, Oak, Sycamore, Willow, |
| | | Yellow-poplar |
| Cerambycidae | Goes pulverulentus | Beech, Elm, Nuttall, Willow, Black oak, Cherrybark |
| | | oak, Water oak, Sycamore |
| | Goes tigrinus | Oak |
| | Neoclytus | Ash, Hickory, Oak, Walnut, Birch, Beech, Maple, |
| | acuminatus | Eastern hophornbeam, Dogwood, Persimmon, |
| | | Redbud, Holly, Hackberry, Black locust, Honeylocust, |
| | | Yellow-poplar, Chestnut, Osage-orange, Sassafras, |
| | | Lilac, Mountain-mahogany, Pear, Cherry, Plum, |
| | | Peach, Apple, Elm, Basswood, Sweetgum |
| | Neoptychodes | Fig, Alder, Mulberry, Willow, Netleaf hackberry |
| | trilineatus | |
| | Oberea ocellata | Sumac, Apple, Peach, Plum, Pear, Currant, Blackberry |
| | Oberea tripunctata | Dogwood, Viburnum, Elm, Sourwood, Blueberry, |
| | | Rhododendron, Azalea, Laurel, Poplar, Willow, |
| | | Mulberry |
| | Oncideres cingulata | Hickory, Pecan, Persimmon, Elm, Sourwood, Basswood, |
| | | Honeylocust, Dogwood, Eucalyptus, Oak, Hackberry, |
| | | Maple, Fruit trees |
| | Saperda calcarata | Poplar |
| | Strophiona nitens | Chestnut, Oak, Hickory, Walnut, Beech, Maple |
| Scolytidae | Corthylus columbianus | Maple, Oak, Yellow-poplar, Beech, Boxelder, |
| | | Sycamore, Birch, Basswood, Chestnut, Elm |
| | Dendroctonus frontalis | Pine |
| | Dryocoetes betulae | Birch, Sweetgum, Wild cherry, Beech, Pear |
| | Monarthrum | Oak, Maple, Birch, Chestnut, Sweetgum, Blackgum, |
| | fasciatum | Poplar, Hickory, Mimosa, Apple, Peach, Pine |
| | Phloeotribus | Peach, Cherry, Plum, Black cherry, Elm, Mulberry, |
| | liminaris | Mountain-ash |
| | Pseudopityophthorus | Oak, American beech, Black cherry, Chickasaw plum, |
| | pruinosus | Chestnut, Maple, Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, Apricot, Almond, |
| | | Black cherry |
| | Synanthedon pictipes | Peach, Plum, Cherry, Beach, Black Cherry |
| | Synanthedon rubrofascia | Tupelo |
| | Synanthedon scitula | Dogwood, Pecan, Hickory, Oak, Chestnut, Beech, |
| | | Birch, Black cherry, Elm, Mountain-ash, Viburnum, |
| | | Willow, Apple, Loquat, Ninebark, Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged, Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention may be active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention may also be suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds of formulae I, I-1, I-2, I-3, I-4, I-1a, I-2a, I-3a, and I-4a, or salts thereof, are especially suitable for controlling one or more pests selected from the genus: Spodoptera spp, Helicoverpa spp, Heliothis spp, Leucinodes spp, Tuta spp, Plutella spp, Cydia spp, Lobesia spp, Tortrix spp, Amyelois spp, Maruca spp, Chrysodeixis spp, Agrotis spp, Elasmopalpus spp, Dalbulus spp, Sternechus spp, Phyllotreta spp, Popillia spp, Scirpophaga spp, Chilo spp, Cnaphalocrosis spp, Tetranychus spp, Panonychus spp, Polyphagotarsonemus spp, Phyllocoptruta spp, Aculus spp, Brevipalpus spp, Oligonychus spp, Aculops spp, Nilaparvata spp, Sogatella spp, Laodelphax spp, Nephotettix spp, Diabrotica spp, Agriotes spp, Hypnoidus spp, Limonius spp, Melanotus spp, Conoderus spp, Delia spp, Amphimallon spp, Popillia spp, Euschistus spp, Piezodorus spp, Nezara spp, Dichelops spp, Lygus spp, Leptocorisa spp, Eurygaster spp, Halymorpha spp, Thrips spp, Scirtothrips spp, Frankliniella spp, Anthonomus spp, Melingethes spp, Phyllotreta spp, Leptinotarsa spp, Bemisia spp, Trialeurodes spp, Aphis spp, and Myzus spp.

In a preferred embodiment of each aspect, a compound TX (where the abbreviation “TX” means “one compound selected from the compounds defined in Tables 1 to 5 and Table P”) controls one or more of pests selected from the genus: Spodoptera spp, Helicoverpa spp, Heliothis spp, Leucinodes spp, Tuta spp, Plutella spp, Cydia spp, Lobesia spp, Tortrix spp, Amyelois spp, Maruca spp, Chrysodeixis spp, Agrotis spp, Elasmopalpus spp, Dalbulus spp, Sternechus spp, Phyllotreta spp, Popillia spp, Scirpophaga spp, Chilo spp, Cnaphalocrosis spp, Tetranychus spp, Panonychus spp, Polyphagotarsonemus spp, Phyllocoptruta spp, Aculus spp, Brevipalpus spp, Oligonychus spp, Aculops spp, Nilaparvata spp, Sogatella spp, Laodelphax spp, Nephotettix spp, Diabrotica spp, Agriotes spp, Hypnoidus spp, Limonius spp, Melanotus spp, Conoderus spp, Delia spp, Amphimallon spp, Popillia spp, Euschistus spp, Piezodorus spp, Nezara spp, Dichelops spp, Lygus spp, Leptocorisa spp, Eurygaster spp, Halymorpha spp, Thrips spp, Scirtothrips spp, Frankliniella spp, Anthonomus spp, Melingethes spp, Phyllotreta spp, Leptinotarsa spp, Bemisia spp, Trialeurodes spp, Aphis spp, and Myzus spp.

The compounds of formulae I, I-1, I-2, I-3, I-4, I-1a, I-2a, I-3a, and I-4a, or salts thereof, are especially suitable for controlling one or more pests selected from: Spodoptera spp (for example, Spodoptera frugiperda, Spodoptera littoralis), Helicoverpa armigera, Heliothis virescens, Leucinodes orbonalis, Tuta absoluta, Plutella xylostella, Cydia pomonella, Lobesia spp, Tortrix spp, Maruca vitrata, Chrysodeixis includens, Agrotis ipsilon, Elasmopalpus lignosellus, Dalbulus maidis, Phyllotreta spp, Popillia japonica, Scirpophaga incertulas, Chilo suppressalis, Cnaphalocrosis medinalis, Tetranychus urticae, Panonychus ulmi, Polyphagotarsonemus latus, Phyllocoptruta oleivora, Brevipalpus spp, Aculops lycopersici, Nilaparvata lugens, Sogatella frucifera, Laodelphax striatellus, Nephotettix spp, Diabrotica vigifera, Agriotes spp, Hypnoidus bicolor, Limonius canus, Delia radicum, Popillia japonica, Euschistus heros, Piezodorus lituratus, Nezara viridula, Dichelops furcatus, Lygus sp., Leptocorisa acuta, Halyomorpha halys, Thrips tabaci, Scirtothrips dorsalis, Frankliniella occidentalis, Anthonomus grandis, Meligethes aeneus, Phyllotreta spp, Leptinotarsa decemlineata, Bemisia tabaci, Trialeurodes vaporariorum, Aphis gossypii, and Myzus persicae.

In a preferred embodiment of each aspect, a compound TX (where the abbreviation “TX” means “one compound selected from the compounds defined in Tables 1 to 5 and Table P”) controls one or more of pests selected from the genus: Spodoptera spp (for example, Spodoptera frugiperda, Spodoptera littoralis)+TX, Helicoverpa armigera+TX, Heliothis virescens+TX, Leucinodes orbonalis+TX, Tuta absoluta+TX, Plutella xylostella+TX, Cydia pomonella+TX, Lobesia spp+TX, Tortrix spp+TX, Maruca vitrata+TX, Chrysodeixis includens+TX, Agrotis ipsilon+TX, Elasmopalpus lignosellus+TX, Dalbulus maidis+TX, Phyllotreta spp+TX, Popillia japonica+TX, Scirpophaga incertulas+TX, Chilo suppressalis+TX, Cnaphalocrosis medinalis+TX, Tetranychus urticae+TX, Panonychus ulmi+TX, Polyphagotarsonemus latus+TX, Phyllocoptruta oleivora+TX Brevipalpus spp+TX, Aculops lycopersici+TX, Nilaparvata lugens+TX, Sogatella frucifera+TX, Laodelphax striatellus+TX, Nephotettix spp+TX, Diabrotica vigifera+TX, Agriotes spp+TX, Hypnoidus bicolor+TX, Limonius canus+TX, Delia radicum+TX, Popillia japonica+TX, Euschistus heros+TX, Piezodorus lituratus+TX, Nezara viridula+TX, Dichelops furcatus+TX, Lygus spp+TX, Leptocorisa acuta+TX, Halyomorpha haly+TX s, Thrips tabaci+TX, Scirtothrips dorsalis+TX, Frankliniella occidentalis+TX, Anthonomus grandis+TX, Meligethes aeneus+TX, Phyllotreta spp+TX, Leptinotarsa decemlineata+TX, Bemisia tabaci+TX, Trialeurodes vaporariorum+TX, Aphis gossypii+TX, and Myzus persicae+TX.

The compounds of formulae I, I-1, I-2, I-3, I-4, I-1a, I-2a, I-3a, and I-4a, or salts thereof, are especially suitable for controlling in the crops listed in the Table below the pests listed.

| Crop | Pests |
|---|---|
| leafy | Spodoptera spp, Helicoverpa armigera, Heliothis virescens, Leucinodes orbonalis, |
| vegetables | Tuta absoluta, Plutella xylostella, Cydia pomonella, Lobesia spp, Tortrix spp, Maruca |
| | vitrata, Tetranychus urticae, Panonychus ulmi, Polyphagotarsonemus latus, |
| | Phyllocoptruta oleivora, Brevipalpus spp, Aculops lycopersici |
| solanacea | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Tetranychus urticae, Panonychus ulmi, Polyphagotarsonemus latus, |
| | Phyllocoptruta oleivora, Brevipalpus spp, Aculops lycopersici |
| brassica | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata |
| okra | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata |
| pome fruits | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Tetranychus urticae, Panonychus ulmi, Polyphagotarsonemus latus, |
| | Phyllocoptruta oleivora, Brevipalpus spp, Aculops lycopersici |
| vegetables | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Bemisia tabaci, Trialeurodes vaporariorum, Aphis gossypii, Myzus persicae |
| grapes | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Thrips tabaci, Scirtothrips dorsalis, Frankliniella occidentalis |
| nuts | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata |
| plantation | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Tetranychus urticae, Panonychus ulmi, Polyphagotarsonemus latus, |
| | Phyllocoptruta oleivora, Brevipalpus spp, Aculops lycopersici |
| eggplant | Spodoptera spp, Helicoverpa armigera, Heliothis virescens, Leucinodes orbonalis, |
| | Tuta absoluta, Plutella xylostella, Cydia pomonella, Lobesia spp, Tortrix spp, Maruca |
| | vitrata, Tetranychus urticae, Panonychus ulmi, Polyphagotarsonemus latus, |
| | Phyllocoptruta oleivora, Brevipalpus spp, Aculops lycopersici |
| corn | Spodoptera spp (for example, Spodoptera frugiperda, Spodoptera littoralis), |
| | Helicoverpa armigera, Heliothis virescens, Chrysodeixis includens, Agrotis ipsilon, |
| | Elasmopalpus lignosellus, Agrotis ipsilon, Dalbulus maidis, Phyllotreta sp., Popillia |
| | japonica, Tetranychus urticae, Panonychus ulmi, Polyphagotarsonemus latus, |
| | Phyllocoptruta oleivora, Brevipalpus spp, Aculops lycopersici, Diabrotica virgifera |
| | vigifera, Agriotes spp, Hypnoidus bicolor, Limonius canus, Delia radicum, Euschistus |
| | heros, Piezodorus lituratus, Nezara viridula, Dichelops furcatus, Lygus spp, |
| | Leptocorisa acuta, Halyomorpha halys, Anthonomus grandis, Meligethes aeneus, |
| | Phyllotreta spp, Leptinotarsa decemlineata |
| soybean | Spodoptera spp (for example, Spodoptera frugiperda, Spodoptera littoralis), |
| | Helicoverpa armigera, Heliothis virescens, Chrysodeixis includens, Agrotis ipsilon, |
| | Elasmopalpus lignosellus, Agrotis ipsilon, Dalbulus maidis, Phyllotreta sp., Popillia |
| | japonica, Tetranychus urticae, Panonychus ulmi, Polyphagotarsonemus latus, |
| | Phyllocoptruta oleivora, Brevipalpus spp, Aculops lycopersici, Euschistus heros, |
| | Piezodorus lituratus, Nezara viridula, Dichelops furcatus, Lygus spp, Leptocorisa |
| | acuta, Halyomorpha halys, Thrips tabaci, Scirtothrips dorsalis, Frankliniella |
| | occidentalis, Anthonomus grandis, Meligethes aeneus, Phyllotreta spp, Leptinotarsa |
| | decemlineata |
| cotton | Spodoptera spp (for example, Spodoptera frugiperda, Spodoptera littoralis), |
| | Helicoverpa armigera, Heliothis virescens, Chrysodeixis includens, Agrotis ipsilon, |
| | Elasmopalpus lignosellus, Agrotis ipsilon, Dalbulus maidis, Phyllotreta sp., Popillia |
| | japonica, Tetranychus urticae, Panonychus ulmi, Polyphagotarsonemus latus, |
| | Phyllocoptruta oleivora, Brevipalpus spp, Aculops lycopersici, Diabrotica virgifera |
| | vigifera, Agriotes spp, Hypnoidus bicolor, Limonius canus, Delia radicum, Euschistus |
| | heros, Piezodorus lituratus, Nezara viridula, Dichelops furcatus, Lygus spp, |
| | Leptocorisa acuta, Halyomorpha halys, Thrips tabaci, Scirtothrips dorsalis, |
| | Frankliniella occidentalis, Anthonomus grandis, Meligethes aeneus, Phyllotreta spp, |
| | Leptinotarsa decemlineata, Bemisia tabaci, Trialeurodes vaporariorum, Aphis |
| | gossypii, Myzus persicae |
| canola | Spodoptera frugiperda, Elasmopalpus lignosellus, Agrotis ipsilon, Dalbulus maidis, |
| | Phyllotreta spp, Popillia japonica |
| rice | Scirpophaga incertulas, Chilo suppressalis, Cnaphalocrosis medinalis, Nilaparvata |
| | lugens, Sogatella frucifera, Laodelphax striatellus, Nephotettix spp, Euschistus heros, |
| | Piezodorus lituratus, Nezara viridula, Dichelops furcatus, Lygus spp, Leptocorisa |
| | acuta, Halyomorpha halys, Anthonomus grandis, Meligethes aeneus, Phyllotreta spp, |
| | Leptinotarsa decemlineata |
| citrus | Tetranychus urticae, Panonychus ulmi, Polyphagotarsonemus latus, Phyllocoptruta |
| | oleivora, Brevipalpus spp, Aculops lycopersici, Thrips tabaci, Scirtothrips dorsalis, |
| | Frankliniella occidentalis |
| potato | Tetranychus urticae, Panonychus ulmi, Polyphagotarsonemus latus, Phyllocoptruta |
| | oleivora, Brevipalpus spp, Aculops lycopersici, Diabrotica virgifera vigifera, Agriotes |
| | spp, Hypnoidus bicolor, Limonius canus, Delia radicum, Popillia japonica, Anthonomus |
| | grandis, Meligethes aeneus, Phyllotreta spp, Leptinotarsa decemlineata |
| Cereal (wheat, | Diabrotica virgifera vigifera, Agriotes spp, Hypnoidus bicolor, Limonius canus, Delia |
| sorghum, | radicum, Popillia japonica, Anthonomus grandis, Meligethes aeneus, Phyllotreta spp, |
| barley) | Leptinotarsa decemlineata |
| diverse field | Diabrotica virgifera vigifera, Agriotes spp, Hypnoidus bicolor, Limonius canus, Delia |
| crops | radicum, Popillia japonica |
| fruiting | Thrips tabaci, Scirtothrips dorsalis, Frankliniella occidentalis, |
| vegetables | |
| peanut | Thrips tabaci, Scirtothrips dorsalis, Frankliniella occidentalis |
| onion | Thrips tabaci, Scirtothrips dorsalis, Frankliniella occidentalis |
| oil seed rape | Anthonomus grandis, Meligethes aeneus, Phyllotreta spp, Leptinotarsa decemlineata |
| ornamentals | Thrips tabaci, Scirtothrips dorsalis, Frankliniella occidentalis |
| speciality crops (as defined in | Bemisia tabaci, Trialeurodes vaporariorum, Aphis gossypii, Myzus persicae |
| https://www.ams.usda.gov/ | |
| services/grants/scbgp/ | |
| specialty-crop) | |

In a preferred embodiment of each aspect, a compound TX (where the abbreviation “TX” means “one compound selected from the compounds defined in Tables 1 to 5 and Table P”) controls one or more of Spodoptera spp (for example, Spodoptera frugiperda, Spodoptera littoralis), Helicoverpa armigera, Heliothis virescens, Leucinodes orbonalis, Tuta absoluta, Plutella xylostella, Cydia pomonella, Lobesia spp, Tortrix sp., Maruca vitrata, Chrysodeixis includens, Agrotis ipsilon, Elasmopalpus lignosellus, Dalbulus maidis, Phyllotreta spp, Popillia japonica, Scirpophaga incertulas, Chilo suppressalis, Cnaphalocrosis medinalis, Nilaparvata lugens, Sogatella frucifera, Laodelphax striatellus, Nephotettix spp, Bemisia tabaci, Trialeurodes vaporariorum, Aphis gossypii, and Myzus persicae; such as Spodoptera spp (for example, Spodoptera frugiperda, Spodoptera littoralis)+TX, Helicoverpa armigera+TX, Heliothis virescens+TX, Leucinodes orbonalis+TX, Tuta absoluta+TX, Plutella xylostella+TX, Cydia pomonella+TX, Lobesia spp+TX, Tortrix spp+TX, Maruca vitrata+TX, Chrysodeixis includens+TX, Agrotis ipsilon+TX, Elasmopalpus lignosellus+TX, Dalbulus maidis+TX, Phyllotreta spp+TX, Popillia japonica+TX, Scirpophaga incertulas+TX, Chilo suppressalis+TX, Cnaphalocrosis medinalis+TX, Nilaparvata lugens+TX, Sogatella frucifera+TX, Laodelphax striatellus+TX, Nephotettix spp+TX, Bemisia tabaci+TX, Trialeurodes vaporariorum+TX, Aphis gossypii+TX, and Myzus persicae+TX.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile (against non-target organisms above and below ground (such as fish, birds and bees), improved physico-chemical properties, or increased biodegradability). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The activity of the compositions according to the invention can be broadened considerably, and adapted to prevailing circumstances, by adding other insecticidally, acaricidally and/or fungicidally active ingredients. The mixtures of the compounds of formula (I) with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridinylmethyleneamino derivatives, macrolides, neonicotinoids and Bacillus thuringiensis preparations.

The following mixtures of a compound of formula (I) with an active substances are preferred (the abbreviation “TX” means “one compound selected from the compounds defined in Tables 1 to 5 and Table P”):
an adjuvant selected from the group of substances consisting of petroleum oils (alternative name) (628)+TX;
abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX. CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, CAS number: 2719848-60-7+TX, CAS number: 1956329-03-5+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyroxystrobin+TX, flupyrimin+TX, fluralaner+TX, fluvalinate+TX, fluxametamide+TX, fosthiazate+TX, gamma-cyhalothrin+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-Cyhalothrin+TX, ledprona+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirobudifen+TX; spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tiorantraniliprole+TX; tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, trifluenfuronate+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX;
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, Gossyplure® (alternative name; 11 mixture of the (Z,E) and (Z,Z) isomers of hexadeca-7,11-dien-1-yl-acetate) (420)+TX, grandlure (421)+TX, grandlure I (alternative name) (421)+TX, grandlure II (alternative name) (421)+TX, grandlure Ill (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure Ill+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone Ill+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene Ill+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl+TX, R-metalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, ethyl 1-[[4-[[2-(trifluoromethyl)-1,3-dioxolan-2-yl]methoxy]phenyl]methyl]pyrazole-3-carboxylate+TX (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate+TX (may be prepared from the methods described in WO 2020/056090), methyl N-[[4-[1-(4-cyclopropyl-2,6-difluoro-phenyl)pyrazol-4-yl]-2-methyl-phenyl]methyl]carbamate+TX (may be prepared from the methods described in WO 2020/097012), methyl N-[[4-[1-(2,6-difluoro-4-isopropyl-phenyl)pyrazol-4-yl]-2-methyl-phenyl]methyl]carbamate+TX (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide+TX (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide+TX (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide+TX (may be prepared from the methods described in WO 2020/109391), N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1, 1-dimethyethyl)-α-[4′-(trifluoromethoxy) [1, 1′-biphenyl]-4-yl]-5-pyrimidinemethanol+TX, fluoxapiprolin+TX, enoxastrobin+TX, methyl (Z)-3-methoxy-2-[2-methyl-5-[4-(trifluoromethyl)triazol-2-yl]phenoxy]prop-2-enoate+TX, methyl (Z)-3-methoxy-2-[2-methyl-5-(4-propyltriazol-2-yl)phenoxy]prop-2-enoate+TX, methyl (Z)-2-[5-(3-isopropylpyrazol-1-yl)-2-methyl-phenoxy]-3-methoxy-prop-2-enoate+TX, methyl (Z)-3-methoxy-2-[2-methyl-5-(3-propylpyrazol-1-yl)phenoxy]prop-2-enoate+TX, methyl (Z)-3-methoxy-2-[2-methyl-5-[3-(trifluoromethyl)pyrazol-1-yl]phenoxy]prop-2-enoate+TX (these compounds may be prepared from the methods described in WO2020/079111), methyl (Z)-2-(5-cyclohexyl-2-methyl-phenoxy)-3-methoxy-prop-2-enoate+TX, methyl (Z)-2-(5-cyclopentyl-2-methyl-phenoxy)-3-methoxy-prop-2-enoate+TX (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum (MicroAZ®, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (e.g. Biostart™, formerly Rhizoboost®)+TX, Bacillus licheniformis strain 3086 (EcoGuard®, Green Releaf®)+TX, Bacillus circulans+TX, Bacillus firmus (BioSafe®, BioNem-WP®, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®, Serenade®, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®, Aquabac®, VectoBac®)+TX, Bacillus thuringiensis kurstaki (Javelin®, Deliver®, CryMax®, Bonide®, Scutella WP®, Turilav WP®, Astuto®, Dipel WP®, Biobit®, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®, DiPel®)+TX, bacteria spp. (GROWMEND®, GROWSWEET®, Shootup®)+TX, bacteriophage of Clavipacter michiganensis (AgriPhage®)+TX, Bakflor®+TX, Beauveria bassiana (Beaugenic®, Brocaril WP®)+TX, Beauveria bassiana GHA (Mycotrol ES®, Mycotrol O®, BotaniGuard®)+TX, Beauveria brongniartii (Engerlingspilz®, Schweizer Beauveria®, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®, Intercept®, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®, Madex® Plus®, Madex® Max, Carpovirusine Evo2®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®, Biofox C®)+TX, Fusarium proliferatum+TX, Fusarium spp.+TX, Galactomyces geotrichum+TX, Gliocladium catenulatum (Primastop®, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97 ®, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®, Polyversum®)+TX, Pythium periplocum+TX, Rhanella aquatilis+TX, Rhanella spp.+TX, Rhizobia (Dormal®, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®, PlantShield HC®, RootShield®, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural Ill)+TX, various fungi (Millennium Microbes®)+TX, Verticillium chlamydosporium+TX, Verticillium lecanii (Mycotal®, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus+TX;
Plant extracts including: pine oil (Retenol®)+TX, azadirachtin (Plasma Neem Oil®, AzaGuard®, MeemAzal®, Molt-X®, Botanical IGR (Neemazad®, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®, Sakalia®)+TX, rotenone (Eco Roten®)+TX, Rutaceae plant extract (Soleo®)+TX, soybean oil (Ortho Ecosense®)+TX, Melaleuca alternifolia extract (also called tea tree oil) (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®)+TX; pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (3E,8Z,11Z)-3,8,11-Tetradecatrienyl acetate+TX, (7Z,11Z,13E)-7,11,13-Hexadecatrienal+TX, (E,Z)-7,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate+TX;
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®, Andersoni-System®)+TX, Amblyseius californicus (Amblyline®, Spical®)+TX, Amblyseius cucumeris (Thripex®, Bugline cucumeris®)+TX, Amblyseius fallacis (Fallacis®)+TX, Amblyseius swirskii (Bugline Swirskii®, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®, Digline®)+TX, Dacnusa sibirica (DacDigline®, Minex®)+TX, Diversinervus spp.+TX, Encarsia citrina+TX, Encarsia formosa (Encarsia Max®, Encarline®, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®, Eretline E®)+TX, Eretmocerus eremicus (Bemimix®)+TX, Eretmocerus hayati+TX, Eretmocerus mundus (Bemipar®, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®, Nemaseek®, Terranem-Nam®, Terranem®, Larvanem®, B-Green®, NemAttack®, Nematop®)+TX, Heterorhabditis megidis (Nemasys H®, BioNem H®, Exhibitline Hm®, Larvanem-M®)+TX, Hippodamia convergens+TX, Hypoaspis aculeifer (Aculeifer-System®, Entomite-A®)+TX, Hypoaspis miles (Hypoline m®, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®, Macroline c®, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®, Nesibug®)+TX, Ophyra aenescens (Biofly®)+TX, Orius insidiosus (Thripor-I®, Oriline I®)+TX, Orius laevigatus (Thripor-L®, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®, Millenium®, BioNem C®, NemAttack®, Nemastar®, Capsanem®)+TX, Steinernema feltiae (NemaShield®, Nemasys F®, BioNem F®, Steinernema-System®, NemAttack®, Nemaplus®, Exhibitline Sf®, Scia-rid®, Entonem®)+TX, Steinernema kraussei (Nemasys L®, BioNem L®, Exhibitline Srb®)+TX, Steinernema riobrave (BioVector®, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator+TX;
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®, Ni-HIBIT Gold CST®)+TX, fatty acids derived from a natural by-product of extra virgin olive oil (FLIPPER®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®, Rebell Amarillo®)+TX and Traps (Takitrapline y+B®)+TX;
(1) antibacterial agents selected from the group of:
(1.1) bacteria, examples of which are Bacillus mojavensis strain R3B (Accession No. NCAIM (P) B001389) (WO 2013/034938) from Certis USA LLC+TX; Bacillus pumilus, in particular strain BU F-33, having NRRL Accession No. 50185 (CARTISSA® from BASF, EPA Reg. No. 71840-19)+TX; Bacillus subtilis, in particular strain QST713/AQ713 (SERENADE OPTI or SERENADE ASO from Bayer CropScience LP, US, having NRRL Accession No. B21661, U.S. Pat. No. 6,060,051)+TX; Bacillus subtilis strain BU1814 (VELONDIS® PLUS, VELONDIS® FLEX and VELONDIS® EXTRA from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus subtilis CX-9060 from Certis USA LLC+TX; Bacillus sp., in particular strain D747 (available as DOUBLE NICKEL® from Kumiai Chemical Industry Co., Ltd.), having Accession No. FERM BP-8234, U.S. Pat. No. 7,094,592+TX; Paenibacillus sp. strain having Accession No. NRRL B-50972 or Accession No. NRRL B-67129, WO 2016/154297+TX; Paenibacillus polymyxa, in particular strain AC-1 (e.g. TOPSEED® from Green Biotech Company Ltd.)+TX; Pantoea agglomerans, in particular strain E325 (Accession No. NRRL B-21856) (available as BLOOMTIME BIOLOGICAL™ FD BIOPESTICIDE from Northwest Agri Products)+TX; Pseudomonas proradix (e.g. PRORADIX® from Sourcon Padena)+TX; and
(1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH)+TX; Pseudozyma aphidis (as disclosed in WO2011/151819 by Yissum Research Development Company of the Hebrew University of Jerusalem)+TX; Saccharomyces cerevisiae, in particular strains CNCM No. 1-3936, CNCM No. 1-3937, CNCM No. 1-3938 or CNCM No. 1-3939 (as disclosed in WO 2010/086790 from Lesaffre et Compagnie, FR)+TX;
(2) biological fungicides selected from the group of:
(2.1) bacteria, examples of which are Agrobacterium radiobacter strain K84 (e.g. GALLTROL-A® from AgBioChem, CA)+TX; Agrobacterium radiobacter strain K1026 (e.g. NOGALL™ from BASF SE)+TX; Bacillus subtilis var. amyloliquefaciens strain FZB24 having Accession No. DSM 10271 (available from Novozymes as TAEGRO® or TAEGRO® ECO (EPA Registration No. 70127-5))+TX; Bacillus amyloliquefaciens, in particular strain D747 (available as Double Nickel™ from Kumiai Chemical Industry Co., Ltd., having accession number FERM BP-8234, U.S. Pat. No. 7,094,592)+TX; Bacillus amyloliquefaciens strain F727 (also known as strain MBI110) (NRRL Accession No. B-50768, WO 2014/028521) (STARGUS® from Marrone Bio Innovations)+TX; Bacillus amyloliquefaciens strain FZB42, Accession No. DSM 23117 (available as RHIZOVITAL® from ABiTEP, DE)+TX; Bacillus amyloliquefaciens isolate B246 (e.g. AVOGREEN™ from University of Pretoria)+TX; Bacillus licheniformis, in particular strain SB3086, having Accession No. ATCC 55406, WO 2003/000051 (available as ECOGUARD® Biofungicide and GREEN RELEAF™ from Novozymes)+TX; Bacillus licheniformis FMCH001 and Bacillus subtilis FMCHOO2 (QUARTZO® (WG) and PRESENCE® (WP) from FMC Corp.)+TX; Bacillus methylotrophicus strain BAC-9912 (from Chinese Academy of Sciences' Institute of Applied Ecology)+TX; Bacillus mojavensis strain R3B (Accession No. NCAIM (P) B001389) (WO 2013/034938) from Certis USA LLC+TX; Bacillus mycoides, isolate, having Accession No. B-30890 (available as BMJ TGAI® or WG and LifeGard™ from Certis USA LLC.)+TX; Bacillus pumilus, in particular strain QST2808 (available as SONATA® from Bayer CropScience LP, US, having Accession No. NRRL B-30087 and described in U.S. Pat. No. 6,245,551)+TX; Bacillus pumilus, in particular strain GB34 (available as Yield Shield® from Bayer AG, DE)+TX; Bacillus pumilus, in particular strain BU F-33, having NRRL Accession No. 50185 (available as part of the CARTISSA product from BASF, EPA Reg. No. 71840-19)+TX; Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051)+TX; Bacillus subtilis Y1336 (available as BIOBAC® WP from Bion-Tech, Taiwan, registered as a biological fungicide in Taiwan under Registration Nos. 4764, 5454, 5096 and 5277)+TX; Bacillus subtilis strain MBI 600 (available as SUBTILEX from BASF SE), having Accession Number NRRL B-50595, U.S. Pat. No. 5,061,495+TX; Bacillus subtilis strain GB03 (available as Kodiak® from Bayer AG, DE)+TX; Bacillus subtilis strain BU1814, (available as VELONDIS® PLUS, VELONDIS® FLEX and VELONDIS® EXTRA from BASF SE)+TX; Bacillus subtilis CX-9060 from Certis USA LLC, a subsidiary of Mitsui & Co.+TX; Bacillus subtilis KTSB strain (FOLIACTIVE® from Donaghys)+TX; Bacillus subtilis IAB/BSO3 (AVIV™ from STK Bio-Ag Technologies, PORTENTO® from Idai Nature)+TX; Bacillus subtilis strain Y1336 (available as BIOBAC® WP from Bion-Tech, Taiwan, registered as a biological fungicide in Taiwan under Registration Nos. 4764, 5454, 5096 and 5277)+TX; Paenibacillus epiphyticus (WO 2016/020371) from BASF SE+TX; Paenibacillus polymyxa ssp. plantarum (WO 2016/020371) from BASF SE+TX; Paenibacillus sp. strain having Accession No. NRRL B-50972 or Accession No. NRRL B-67129, WO 2016/154297+TX; Pseudomonas chlororaphis strain AFS009, having Accession No. NRRL B-50897, WO 2017/019448 (e.g., HOWLER™ and ZIO® from AgBiome Innovations, US)+TX; Pseudomonas chlororaphis, in particular strain MA342 (e.g. CEDOMON®, CERALL®, and CEDRESS® by Bioagri and Koppert)+TX; Pseudomonas fluorescens strain A506 (e.g. BLIGHTBAN® A506 by NuFarm)+TX; Pseudomonas proradix (e.g. PRORADIX® from Sourcon Padena)+TX; Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475)+TX; Streptomyces lydicus strain WYEC108 (also known as Streptomyces lydicus strain WYCD108US) (ACTINO-IRON® and ACTINOVATE® from Novozymes)+TX; and
(2.2) fungi, examples of which are Ampelomyces quisqualis, in particular strain AQ 10 (e.g. AQ 10® by IntrachemBio Italia)+TX; Ampelomyces quisqualis strain AQ10, having Accession No. CNCM 1-807 (e.g., AQ 10® by IntrachemBio Italia)+TX; Aspergillus flavus strain NRRL 21882 (products known as AFLA-GUARD® from Syngenta/ChemChina)+TX; Aureobasidium pullulans, in particular blastospores of strain DSM14940+TX; Aureobasidium pullulans, in particular blastospores of strain DSM 14941+TX; Aureobasidium pullulans, in particular mixtures of blastospores of strains DSM14940 and DSM 14941 (e.g. Botector® by bio-ferm, CH)+TX; Chaetomium cupreum (Accession No. CABI 353812) (e.g. BIOKUPRUM™ by AgriLife)+TX; Chaetomium globosum (available as RIVADIOM® by Rivale)+TX; Cladosporium cladosporioides, strain H39, having Accession No. CBS122244, US 2010/0291039 (by Stichting Dienst Landbouwkundig Onderzoek)+TX; Coniothyrium minitans, in particular strain CON/M/91-8 (Accession No. DSM9660, e.g. Contans® from Bayer CropScience Biologics GmbH)+TX; Cryptococcus flavescens, strain 3C (NRRL Y-50378), (B2.2.99)+TX; Dactylaria candida+TX; Dilophosphora alopecuri (available as TWIST FUNGUS®)+TX; Fusarium oxysporum, strain Fo47 (available as FUSACLEAN® by Natural Plant Protection)+TX; Gliocladium catenulatum (Synonym: Clonostachys rosea f. catenulate) strain J1446 (e.g. Prestop® by Lallemand)+TX; Gliocladium roseum (also known as Clonostachys rosea f rosea), in particular strain 321 U from Adjuvants Plus, strain ACM941 as disclosed in Xue A. G. (Efficacy of Clonostachys rosea strain ACM941 and fungicide seed treatments for controlling the root tot complex of field pea, Can. J. Plant Sci. 2003, 83(3): 519-524), or strain IK726 (Jensen D F, et al. Development of a biocontrol agent for plant disease control with special emphasis on the near commercial fungal antagonist Clonostachys rosea strain ‘IK726’, Australasian Plant Pathol. 2007, 36 (2), 95-101)+TX; Lecanicillium lecanii (formerly known as Verticillium lecanii) conidia of strain KV01 (e.g. Vertalec® by Koppert/Arysta)+TX; Metschnikowia fructicola, in particular strain NRRL Y-30752, (B2.2.3)+TX; Microsphaeropsis ochracea+TX; Muscodor roseus, in particular strain A3-5 (Accession No. NRRL 30548)+TX; Penicillium steckii (DSM 27859, WO 2015/067800) from BASF SE+TX; Penicillium vermiculatum+TX; Phlebiopsis gigantea strain VRA 1992 (ROTSTOP® C from Danstar Ferment)+TX; Pichia anomala, strain WRL-076 (NRRL Y-30842), U.S. Pat. No. 7,579,183+TX; Pseudozyma flocculosa, strain PF-A22 UL (available as SPORODEX® L by Plant Products Co., CA)+TX; Saccharomyces cerevisiae, in particular strain LASO2 (from Agro-Levures et Derives), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR+TX; Simplicillium lanosoniveum+TX; Talaromyces flavus, strain V117b+TX; Trichoderma asperelloides JM41R (Accession No. NRRL B-50759) (TRICHO PLUS® from BASF SE)+TX; Trichoderma asperellum, in particular, strain kd (e.g. T-Gro from Andermatt Biocontrol)+TX; Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S.L., ES) or strain ICC 012 from Isagro+TX; Trichoderma atroviride, in particular strain SC1 (Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Ltd.)+TX; Trichoderma atroviride, strain CNCM 1-1237 (e.g. Esquive® WP from Agrauxine, FR)+TX; Trichoderma atroviride, strain no. V08/002387+TX; Trichoderma atroviride, strain NMI no. V08/002388+TX; Trichoderma atroviride, strain NMI no. V08/002389+TX; Trichoderma atroviride, strain NMI no. V08/002390+TX; Trichoderma atroviride, strain LC52 (e.g. Tenet by Agrimm Technologies Ltd.)+TX; Trichoderma atroviride, strain ATCC 20476 (IMI 206040)+TX; Trichoderma atroviride, strain T11 (IM1352941/CECT20498)+TX; Trichoderma atroviride, strain SKT-1 (FERM P-16510), JP Patent Publication (Kokai) 11-253151 A+TX; Trichoderma atroviride, strain SKT-2 (FERM P-16511), JP Patent Publication (Kokai) 11-253151 A+TX; Trichoderma atroviride, strain SKT-3 (FERM P-17021), JP Patent Publication (Kokai) 11-253151 A+TX; Trichoderma fertile (e.g. product TrichoPlus from BASF)+TX; Trichoderma gamsii (formerly T. viride), strain ICCO80 (IMI CC 392151 CABI, e.g. BioDerma by AGROBIOSOL DE MEXICO, S.A. DE C.V.)+TX; Trichoderma gamsii (formerly T. viride), strain ICC 080 (IMI CC 392151 CABI) (available as BIODERMA® by AGROBIOSOL DE MEXICO, S.A. DE C.V.)+TX; Trichoderma harmatum+TX; Trichoderma harmatum, having Accession No. ATCC 28012+TX; Trichoderma harzianum strain T-22 (e.g. Trianum-P from Andermatt Biocontrol or Koppert) or strain Cepa SimbT5 (from Simbiose Agro)+TX; Trichoderma harzianum+TX; Trichoderma harzianum rifai T39 (e.g. Trichodex® from Makhteshim, US)+TX; Trichoderma harzianum, strain ITEM 908 (e.g. Trianum-P from Koppert)+TX; Trichoderma harzianum, strain TH35 (e.g. Root-Pro by Mycontrol)+TX; Trichoderma harzianum, strain DB 103 (available as T-GRO®7456 by Dagutat Biolab)+TX; Trichoderma polysporum, strain IMI 206039 (e.g. Binab TF WP by BINAB Bio-Innovation AB, Sweden)+TX; Trichoderma stromaticum, having Accession No. Ts3550 (e.g. Tricovab by CEPLAC, Brazil)+TX; Trichoderma virens (also known as Gliocladium virens), in particular strain GL-21 (e.g. SoilGard by Certis, US)+TX; Trichoderma virens strain G-41, formerly known as Gliocladium virens (Accession No. ATCC 20906) (e.g., ROOTSHIELD® PLUS WP and TURFSHIELD® PLUS WP from BioWorks, US)+TX; Trichoderma viride, strain TV1 (e.g. Trianum-P by Koppert)+TX; Trichoderma viride, in particular strain B35 (Pietr et al., 1993, Zesz. Nauk. A R w Szczecinie 161: 125-137)+TX; mixtures of Trichoderma asperellum strain ICC 012 (also known as Trichoderma harzianum I00012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.)+TX; Ulocladium oudemansii strain U3, having Accession No. NM 99/06216 (e.g., BOTRY-ZEN® by Botry-Zen Ltd, New Zealand and BOTRYSTOP® from BioWorks, Inc.)+TX; Verticillium albo-atrum (formerly V. dahliae), strain WCS850 having Accession No. WCS850, deposited at the Central Bureau for Fungi Cultures (e.g., DUTCH TRIG® by Tree Care Innovations)+TX; Verticillium chlamydosporium+TX;
(3) biological control agents having an effect for improving plant growth and/or plant health selected from the group of:
(3.1) bacteria, examples of which are Azospirillum brasilense (e.g., VIGOR® from KALO, Inc.)+TX; Azospirillum lipoferum (e.g., VERTEX-IF™ from TerraMax, Inc.)+TX; Azorhizobium caulinodans, in particular strain ZB-SK-5+TX; Azotobacter chroococcum, in particular strain H23+TX; Azotobacter vinelandii, in particular strain ATCC 12837+TX; a mixture of Azotobacter vinelandii and Clostridium pasteurianum (available as INVIGORATE® from Agrinos)+TX; Bacillus amyloliquefaciens pm414 (LOLI-PEPTA® from Biofilm Crop Protection)+TX; Bacillus amyloliquefaciens SB3281 (ATCC #PTA-7542, WO 2017/205258)+TX; Bacillus amyloliquefaciens TJ1000 (available as QUIKROOTS® from Novozymes)+TX; Bacillus amyloliquefaciens, in particular strain IN937a+TX; Bacillus amyloliquefaciens, in particular strain FZB42 (e.g. RHIZOVITAL® from ABiTEP, DE)+TX; Bacillus amyloliquefaciens BS27 (Accession No. NRRL B-5015)+TX; Bacillus cereus family member EE128 (NRRL No. B-50917)+TX; Bacillus cereus family member EE349 (NRRL No. B-50928)+TX; Bacillus cereus, in particular strain BPO1 (ATCC 55675, e.g. MEPICHLOR® from Arysta Lifescience, US)+TX; Bacillus firmus, in particular strain CNMC 1-1582 (e.g. VOTIVO® from BASF SE)+TX; Bacillus mycoides BT155 (NRRL No. B-50921)+TX; Bacillus mycoides EE118 (NRRL No. B-50918)+TX; Bacillus mycoides EE141 (NRRL No. B-50916)+TX; Bacillus mycoides BT46-3 (NRRL No. B-50922)+TX; Bacillus pumilus, in particular strain QST2808 (Accession No. NRRL No. B-30087)+TX; Bacillus pumilus, in particular strain GB34 (e.g. YIELD SHIELD® from Bayer Crop Science, DE)+TX; Bacillus siamensis, in particular strain KCTC 13613T+TX; Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US)+TX; Bacillus subtilis, in particular strain AQ30002 (Accession No. NRRL B-50421 and described in U.S. patent application Ser. No. 13/330,576)+TX; Bacillus subtilis, in particular strain AQ30004 (NRRL No. B-50455 and described in U.S. patent application Ser. No. 13/330,576)+TX; Bacillus subtilis strain BU1814, (available as TEQUALIS® from BASF SE), Bacillus subtilis rm303 (RHIZOMAX® from Biofilm Crop Protection)+TX; Bacillus thuringiensis BT013A (NRRL No. B-50924) also known as Bacillus thuringiensis 4Q7+TX; a mixture of Bacillus licheniformis FMCH001 and Bacillus subtilis FMCHOO2 (available as QUARTZO® (WG), PRESENCE® (WP) from FMC Corporation)+TX; Bacillus subtilis, in particular strain MBI 600 (e.g. SUBTILEX® from BASF SE)+TX; Bacillus tequilensis, in particular strain NIl-0943+TX; Bradyrhizobium japonicum (e.g. OPTIMIZE® from Novozymes)+TX; Delftia acidovorans, in particular strain RAY209 (e.g. BIOBOOST® from Brett Young Seeds)+TX; Mesorhizobium cicer (e.g., NODULATOR from BASF SE)+TX; Lactobacillus sp. (e.g. LACTOPLANT® from LactoPAFI)+TX; Rhizobium leguminosarium biovar viciae (e.g., NODULATOR from BASF SE)+TX; Pseudomonas proradix (e.g. PRORADIX® from Sourcon Padena)+TX; Pseudomonas aeruginosa, in particular strain PN1+TX; Rhizobium leguminosarum, in particular bv. viceae strain Z25 (Accession No. CECT 4585)+TX; Paenibacillus polymyxa, in particular strain AC-1 (e.g. TOPSEED® from Green Biotech Company Ltd.)+TX; Serratia marcescens, in particular strain SRM (Accession No. MTCC 8708)+TX; Sinorhizobium meliloti strain NRG-185-1 (NITRAGIN® GOLD from Bayer CropScience)+TX; Thiobacillus sp. (e.g. CROPAID® from Cropaid Ltd UK)+TX; and
(3.2) fungi, examples of which are Purpureocillium lilacinum (previously known as Paecilomyces lilacinus) strain 251 (AGAL 89/030550, e.g. BioAct from Bayer CropScience Biologics GmbH)+TX; Penicillium bilaii, strain ATCC 22348 (e.g. JumpStart® from Acceleron BioAg), Talaromyces flavus, strain V117b+TX; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161: 125-137)+TX; Trichoderma atroviride strain LC52 (also known as Trichoderma atroviride strain LU132, e.g. Sentinel® from Agrimm Technologies Ltd.)+TX; Trichoderma atroviride strain SC1 (described in WO2009/116106)+TX; Trichoderma asperellum strain kd (e.g. T-Gro from Andermatt Biocontrol)+TX; Trichoderma asperellum strain (Eco-T from Plant Health Products, ZA)+TX, Trichoderma harzianum strain T-22 (e.g. Trianum-P from Andermatt Biocontrol or Koppert)+TX; Myrothecium verrucaria strain AARC-0255 (e.g. DiTera™ from Valent Biosciences)+TX; Penicillium bilaii strain ATCC ATCC20851+TX; Pythium oligandrum strain M1 (ATCC 38472, e.g. Polyversum from Bioprepraty, CZ)+TX; Trichoderma virens strain GL-21 (e.g. SoilGard® from Certis, USA)+TX; Verticillium albo-atrum (formerly V. dahliae) strain WCS850 (CBS 276.92, e.g. Dutch Trig from Tree Care Innovations)+TX; Trichoderma atroviride, in particular strain no. V08/002387, strain no. NMI No. V08/002388, strain no. NMI No. V08/002389, strain no. NMI No. V08/002390+TX; Trichoderma harzianum strain ITEM 908, Trichoderma harzianum, strain TSTh20+TX; Trichoderma harzianum strain 1295-22+TX; Pythium oligandrum strain DV74+TX; Rhizopogon amylopogon (e.g. Myco-Sol from Agri-Enterprise, LLC, formerly Helena Chem. Company)+TX; Rhizopogon fulvigleba (e.g. Myco-Sol from Agri-Enterprise, LLC, formerly Helena Chem. Company)+TX; Trichoderma virens strain GI-3+TX;
(4) insecticidally active biological control agents selected from
(4.1) bacteria, examples of which are Agrobacterium radiobacter strain K84 (Galltrol from AgBiochem Inc.)+TX; Bacillus amyloliquefaciens, in particular strain PTS-4838 (e.g. AVEO from Valent Biosciences, US)+TX; Bacillus firmus, in particular strain CNMC 1-1582 (e.g. VOTIVO® from BASF SE)+TX; Bacillus mycoides, isolate J. (e.g. BmJ from Certis USA LLC)+TX; Bacillus sphaericus, in particular Serotype H5a5b strain 2362 (strain ABTS-1743) (e.g. VECTOLEX® from Valent BioSciences, US)+TX; Bacillus thuringiensis subsp. aizawai, in particular strain ABTS-1857 (SD-1372, e.g. XENTARI® from Valent BioSciences)+TX; Bacillus thuringiensis subsp. aizawai, in particular serotype H-7 (e.g. FLORBAC® WG from Valent BioSciences, US)+TX; Bacillus thuringiensis israelensis strain BMP 144 (e.g. AQUABAC® by Becker Microbial Products IL)+TX; Bacillus thuringiensis subsp. israelensis (serotype H-14) strain AM65-52 (Accession No. ATCC 1276) (e.g. VECTOBAC® by Valent BioSciences, US)+TX; Bacillus thuringiensis subsp. aizawai strain GC-91+TX; Bacillus thuringiensis var. Colmeri (e.g. TIANBAOBTC by Changzhou Jianghai Chemical Factory)+TX; Bacillus thuringiensis var. japonensis strain Buibui+TX; Bacillus thuringiensis subsp. kurstaki strain BMP 123 (from Becker Microbial Products, IL)+TX; Bacillus thuringiensis subsp. kurstaki strain BMP 123 by Becker Microbial Products, IL, e.g. BARITONE from Bayer CropScience+TX; Bacillus thuringiensis subsp. kurstaki strain HD-1 (e.g. DIPEL® ES from Valent BioSciences, US)+TX; Bacillus thuringiensis var. kurstaki strain EVB-113-19 (e.g., BIOPROTEC® from AEF Global)+TX; Bacillus thuringiensis subsp. kurstaki strain ABTS 351+TX; Bacillus thuringiensis subsp. kurstaki strain PB 54+TX; Bacillus thuringiensis subsp. kurstaki strain SA 11 (JAVELIN® from Certis, US)+TX; Bacillus thuringiensis subsp. kurstaki strain SA 12 (THURICIDE from Certis, US)+TX; Bacillus thuringiensis subsp. kurstaki strain EG 2348 (LEPINOX® from Certis, US)+TX; Bacillus thuringiensis subsp. kurstaki strain EG 7841 (CryMAX® from Certis, US)+TX; Bacillus thuringiensis subsp. tenebrionis strain NB 176 (SD-5428, e.g. NOVODOR® FC from BioFa DE)+TX; Brevibacillus laterosporus (LATERAL® from Ecolibrium Biologicals Ltd.)+TX; Burkholderia spp., in particular Burkholderia rinojensis strain A396 (also known as Burkholderia rinojensis strain MBI 305) (Accession No. NRRL B-50319; WO 2011/106491 and WO 2013/032693; e.g. MB1206 TGAI and ZELTO® from Marrone Bio Innovations)+TX; Chromobacterium subtsugae, in particular strain PRAA4-1T (e.g. MBI-203; e.g. GRANDEVO® from Marrone Bio Innovations)+TX; Lecanicillium muscarium Ve6 (MYCOTAL from Koppert)+TX; Paenibacillus popilliae (formerly Bacillus popilliae; e.g. MILKY SPORE POWDER™ or MILKY SPORE GRANULAR™ from St. Gabriel Laboratories)+TX; Pasteuria nishizawae strain Pn1 (CLARIVA from Syngenta/ChemChina)+TX; Serratia entomophila (e.g. INVADE® by Wrightson Seeds)+TX; Serratia marcescens, in particular strain SRM (Accession No. MTCC 8708)+TX; Trichoderma asperellum (TRICHODERMAX from Novozymes)+TX; Wolbachia pipientis ZAP strain (e.g., ZAP MALES® from MosquitoMate)+TX; and
(4.2) fungi, examples of which are Beauveria bassiana strain ATCC 74040 (e.g. NATURALIS® from Intrachem Bio Italia)+TX; Beauveria bassiana strain GHA (Accession No. ATCC74250, e.g. BOTANIGUARD® ES and MYCONTROL-O® from Laverlam Int. Corp.)+TX; Beauveria bassiana strain ATP02 (Accession No. DSM 24665)+TX; Isaria fumosorosea (previously known as Paecilomyces fumosoroseus, strain Apopka 97, e.g. PREFERAL® from SePRO)+TX; Metarhizium anisopliae 3213-1 (deposited under NRRL accession number 67074, disclosed in WO 2017/066094; Pioneer Hi-Bred International)+TX; Metarhizium robertsii 15013-1 (deposited under NRRL accession number 67073)+TX; Metarhizium robertsii 23013-3 (deposited under NRRL accession number 67075)+TX; Paecilomyces lilacinus strain 251 (MELOCON® from Certis, US)+TX; Zoophtora radicans+TX;
(5) Viruses selected from the group consisting of Adoxophyes orana (summer fruit tortrix) granulosis virus (GV)+TX; Cydia pomonella (codling moth) granulosis virus (GV)+TX; Helicoverpa armigera (cotton bollworm) nuclear polyhedrosis virus (NPV)+TX; Spodoptera exigua (beet armyworm) mNPV+TX; Spodoptera frugiperda (fall armyworm) mNPV+TX; Spodoptera littoralis (African cotton leafworm) NPV+TX;
(6) Bacteria and fungi which can be added as ‘inoculant’ to plants or plant parts or plant organs and which, by virtue of their particular properties, promote plant growth and plant health selected from Agrobacterium spp.+TX; Azorhizobium caulinodans+TX; Azospirillum spp.+TX; Azotobacter spp.+TX; Bradyrhizobium spp.+TX; Burkholderia spp., in particular Burkholderia cepacia (formerly known as Pseudomonas cepacia)+TX; Gigaspora spp., or Gigaspora monosporum+TX; Glomus spp.+TX; Laccaria spp.+TX; LactoBacillus buchneri+TX; Paraglomus spp.+TX; Pisolithus tinctorus+TX; Pseudomonas spp.+TX; Rhizobium spp., in particular Rhizobium trifolii+TX; Rhizopogon spp.+TX; Scleroderma spp.+TX; Suillus spp.+TX; Streptomyces spp.+TX;
(7) Plant extracts and products formed by microorganisms including proteins and secondary metabolites which can be used as biological control agents, selected from Allium sativum (NEMGUARD from Eco-Spray; BRALIC from ADAMA)+TX; Armour-Zen+TX; Artemisia absinthium+TX; Azadirachtin (e.g. AZATIN XL from Certis, US)+TX; Biokeeper WP+TX; Brassicaceae extract, in particular oilseed rape powder or mustard powder+TX; Cassia nigricans+TX; Celastrus angulatus+TX; Chenopodium anthelminticum+TX; Chitin+TX; Dryopteris filix-mas+TX; Equisetum arvense+TX; Fortune Aza+TX; Fungastop+TX; Chenopodium quinoa saponin extract from quinoa seeds (e.g. Heads Up® (Saponins of Quinoa) from Heads Up plant Protectants, CA)+TX; naturally occurring Blad polypeptide extracted from Lupin seeds (PROBLAD® from Certis EU)+TX; naturally occurring Blad polypeptide extracted from Lupin seeds (FRACTURE® from FMC)+TX; Pyrethrum/Pyrethrins+TX; Quassia amara+TX; Quercus+TX; Quillaja extract (QL AGRI 35 from BASF)+TX; Reynoutria sachalinensis extract (REGALLIA®, REGALIA® MAXX from Marrone Bio)+TX; “Requiem™ Insecticide”+TX; Rotenone+TX; ryania/ryanodine+TX; Symphytum officinale+TX; Tanacetum vulgare+TX; Thymol+TX; Thymol mixed with Geraniol (CEDROZ from Eden Research)+TX; Thymol mixed with Geraniol and Eugenol (MEVALONE® from Eden Research)+TX; Triact 70+TX; TriCon+TX; Tropaeulum majus+TX; Melaleuca alternifolia extract (TIMOREX GOLD® from STK)+TX; Urtica dioica+TX; Veratrin+TX; and Viscum album+TX; and
a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The tradenames in brackets behind the active ingredient refer to the commercially available product or products comprising this active ingredient.

The active ingredient mixture of the compounds of formula (I) selected from the compounds defined in the Tables 1 to 5 and Table P with active ingredients described above comprises a compound selected from one compound defined in the Tables 1 to 5 and Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from the compounds defined in the Tables 1 to 5 and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of formula (I) of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I. Further, it is hereby made available, a composition comprising a plant propagation material treated with a compound of formula I.

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections. The disclosure in the present application makes available each and every combination of embodiments disclosed herein.

It should be noted that the disclosure herein in respect of a compound of formula I applies equally in respect of a compound of each of formulae I-1, I-2, I-3, I-4, I-1a, I-2a, I-3a, I-4a, I-5a, II-1, II-2, II-3, II-4, II-5, V-1, V-2, V-3, V-4, V-5, VI-1, VI-2, VI-3, VI-4, VI-5, VIII-1, VIII-2, VIII-3, VIII-4, VIII-5, XIII-1, XIII-2, XIII-3, XIII-4, XIII-5, XIV-1, XIV-2, XIV-3, XIV-4, XIV-5, XV-1, XV-2, XV-3, or XV-4, and Tables 1 to 5 and P.

### EXAMPLES

The following Examples further illustrate, but do not limit, the invention.

The compounds of the invention can be distinguished from known compounds by virtue of greater efficacy at low application rates, which can be verified by a person skilled in the art using the experimental procedures outlined in the Examples, using lower application rates, if necessary, for example 60 ppm, 20 ppm or 2 ppm.

Throughout this description, temperatures are given in degrees Celsius (° C.) and “mp.” means melting point. LC/MS means Liquid Chromatography Mass Spectrometry and the description of the apparatus, and the methods are as follows ¹H NMR and ¹⁹F NMR measurements were recorded on a Bruker 400 MHz spectrometer, chemical shifts are given in ppm relevant to a TMS (¹H) or CFCl₃ (¹⁹F) standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

### LC-MS and GC-MS Methods:

### Method 1:

Spectra were recorded on a Mass Spectrometer from Waters (SQD2 or QDA Single quadrupole mass spectrometer) equipped with an electrospray source (Polarity: Positive and Negative Polarity Switch), Capillary: 0.8-3.00 kV, Cone range: 25 Source Temperature: 120-150° C., Desolvation Temperature: 500-600° C., Cone Gas Flow: 50 L/h, Desolvation Gas Flow: 1000 L/h, Mass range: 110 to 850 Da) and an Acquity UPLC from Waters: Quaternary solvent manager, heated column compartment, diode-array detector. Column: Acquity UPLC HSS T3 C18, 1.8 μm, 30×2.1 mm, Temp: 40° C., DAD Wavelength range (nm): 200 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.6 min 50-100% B; 0.6-1.3 min 100% B; 1.3-1.4 min 100-10% B; 1.4-1.6 min 10% B; Flow (mL/min) 0.6.

### Method 2:

Spectra were recorded on a Mass Spectrometer from Agilent Technologies (6410 Triple Quadrupole mass spectrometer) equipped with an electrospray source (Polarity: positive or negative ions, MS2 Scan, Capillary: 4.00 kV, Fragmentor: 100 V, Desolvation Temperature: 350° C., Gas Flow: 11 L/min, Nebulizer Gas: 45 psi, Mass range: 110 to 1000 Da) and a 1200 Series HPLC from Agilent: quaternary pump, heated column compartment and VWD detector. Column: KINETEX EVO C18, 2.6 μm, 50×4.6 mm, Temp: 40° C., Detector VWD Wavelength: 254 nm, Solvent Gradient: A=water+5% Acetonitrile+0.1% HCOOH, B=Acetonitrile+0.1% HCOOH: gradient: 0 min 10% B, 90% A; 0.9-1.8 min 100% B; 1.8-2.2 min 100-10% B; 2.2-2.5 min 10% B; Flow (mL/min) 1.8.

### Method 3:

Spectra were recorded on a Mass Spectrometer from Agilent Technologies (MSD-IQ mass spectrometer) equipped with an electrospray source (Polarity: positive or negative ions, MS2 Scan, Capillary: 3.5 kV, Fragmentor: 110 V, Desolvation Temperature: 325° C., Gas Flow: 13 L/min, Nebulizer Gas: 55 psi, Mass range: 110 to 850 Da) and a 1290 Series HPLC from Agilent: quaternary pump, heated column compartment and diode-array detector. Column: AGILENT POROSHELL 120 EC-C18, 1.9 μm, 50×2.1 mm, Temp: 40° C., DAD Wavelength range (nm): 190 to 400, Solvent Gradient: A=water+5% Acetonitrile+0.1% HCOOH, B=Acetonitrile+0.1% HCOOH: gradient: 0-0.5 min 10% B, 90% A; 1.2-1.5 min 95% B, 05% A; 1.8-2.5 min 10% B, 90% A; Flow (mL/min) 0.8.

### Method 4:

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 I/h, Desolvation Gas Flow: 650-1000 I/h, Mass range: 110 to 950 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2 min, 100-10% B in 0.05 min, 10% B isocratic for 0.05 min.

### Method 5:

Spectra were recorded on a ACQUITY Mass Spectrometer from Waters Corporations (QDa or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 3.0 min; Flow (ml/min) 0.75.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible

Abbreviations

| | |
|---|---|
| CDCl₃ | Deuterated chloroform |
| DMF | Dimethylformamide |
| DMSO | Dimethyl sulfoxide |
| DMSO-d6 | Deuterated Dimethyl sulfoxide |
| EtOAc | ethyl acetate |
| HCl | hydrochloric acid |
| hr | hour/hours |
| iPr | iso-propyl |
| LCMS | Liquid Chromatography Mass Spectrometry |
| rh | relative humidity |
| rt | room temperature |
| Rt | Retention time |
| TBME | methyl tert-butyl ether or tert-butyl methyl ether |
| THF | tetrahydrofuran |

### Preparatory Examples

“Mp” means melting point in ° C. Free radicals represent methyl groups.

¹H NMR and ¹⁹F NMR measurements were recorded on a Bruker 400 MHz spectrometer, chemical shifts are given in ppm relevant to a TMS (¹H) or CFCl₃ (¹⁹F) standard. Spectra measured in deuterated solvents as indicated.

Either one of the LCMS methods below was used to characterize the compounds. The characteristic LCMS values obtained for each compound were the retention time (“Rt”, recorded in minutes) and the measured molecular ion (M+H)⁺ or (M−H).

### Example P1: Preparation of 2-chloro-N-cyclopropyl-5-[(2S)-2-(trifluoromethylsulfonylamino) propoxyl]pyridine-3-carboxamide (Compound P-1, Table P)

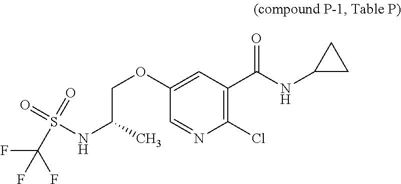 [SMILES: C[C@@H](COc1cnc(Cl)c(C(=O)NC2CC2)c1)NS(=O)(=O)C(F)(F)F]

### Step A: Preparation of methyl 2-chloro-5-(4,4,5,5-tetramethyl-1,3,2-dioxaborolan-2-yl)pyridine-3-carboxylate

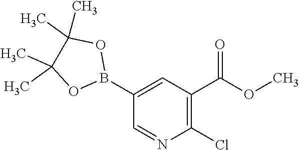

A solution of methyl 5-bromo-2-chloro-pyridine-3-carboxylate (15.0 g, 59.9 mmol), bis(pinacolato)diboron (23.0 g, 89.8 mmol, 1.50 equiv.) and potassium carbonate (20.8 g, 209 mmol, 3.50 equiv.) in anhydrous 2-methyltetrahydrofuran (900 mL) was degassed with argon for 20 minutes. Then 1,1′-bis(diphenylphosphino)ferrocene dichloropalladium (II) (2.21 g, 2.99 mmol, 0.050 equiv.) was added and the reaction mixture was stirred overnight at 60° C. After cooling down to room temperature, the reaction mixture was diluted with water and extracted with ethyl acetate. The combined organic layers were washed with brine, dried over sodium sulfate, filtered and concentrated under reduced pressure to afford crude methyl 2-chloro-5-(4,4,5,5-tetramethyl-1,3,2-dioxaborolan-2-yl)pyridine-3-carboxylate, which was used as such in next step.

### Step B: Preparation of methyl 2-chloro-5-hydroxy-pyridine-3-carboxylate

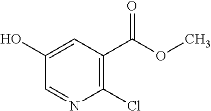

To a solution of crude methyl 2-chloro-5-(4,4,5,5-tetramethyl-1,3,2-dioxaborolan-2-yl)pyridine-3-carboxylate (39.0 g, 131 mmol) in ethanol (655 mL) was added at room temperature hydrogen peroxide (30% in water, 44.6 mL, 393 mmol, 3.00 equiv.). The reaction mixture was stirred overnight at room temperature. It was then diluted with ethyl acetate and quenched with sodium metabisulfite sat. aq. until no peroxide present using Quantofix strips. After dilution with water, the two layers were separated and the aqueous layer was extracted with more ethyl acetate. The combined organic layers were dried over sodium sulfate, filtered and concentrated under reduced pressure. First purification of the crude product by column chromatography and a second purification by reverse phase column chromatography gave methyl 2-chloro-5-hydroxy-pyridine-3-carboxylate. LC-MS (method 1): retention time 0.36 min, m/z 187.9 [M+H⁺]; ¹H NMR (400 MHz, DMSO-d6) δ ppm: 9.22-12.17 (m, 1H) 8.10 (d, 1H) 7.58 (d, 1H) 3.86 (s, 3H)

### Step C: Preparation of [(2S)-2-(tert-butoxycarbonylamino)propyl] methanesulfonate

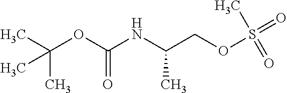 [SMILES: C[C@@H](COS(C)(=O)=O)NC(=O)OC(C)(C)C]

To a solution of tert-butyl N-[(1S)-2-hydroxy-1-methyl-ethyl]carbamate (5.00 g, 28.5 mmol) in tetrahydrofuran (100 mL) was added at 0° C. triethylamine (5.97 mL, 42.8 mmol, 1.50 equiv.), followed by methanesulfonyl chloride (2.49 mL, 31.4 mmol, 1.10 equiv.). The reaction mixture was stirred for 2.5 hours at room temperature. It was then diluted with ethyl acetate and quenched with saturated sodium bicarbonate solution. The aqueous layer was extracted with more ethyl acetate. The combined organic layers were dried over sodium sulfate, filtered and concentrated under reduced pressure to afford crude [(2S)-2-(tert-butoxycarbonylamino)propyl]methanesulfonate, which was used as such in next step. ¹H NMR (400 MHz, CDCl₃) δ ppm: 4.58-4.74 (m, 1H) 4.22 (br s, 1H) 4.12-4.19 (m, 1H) 3.98 (br s, 1H) 3.04 (s, 3H) 1.45 (s, 9H) 1.24 (d, 3H)

### Step D: Preparation of methyl 5-[(2S)-2-(tert-butoxycarbonylamino)propoxyl]-2-chloro-pyridine-3-carboxylate

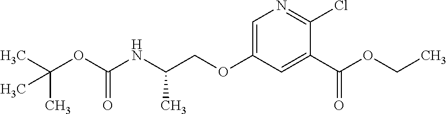 [SMILES: CCOC(=O)c1cc(OC[C@H](C)NC(=O)OC(C)(C)C)cnc1Cl]

To a solution of methyl 2-chloro-5-hydroxy-pyridine-3-carboxylate (1.70 g, 9.10 mmol) and [(2S)-2-(tert-butoxycarbonylamino)propyl]methanesulfonate (2.50 g, 10.0 mmol, 1.10 equiv.) in N,N-dimethylformamide (34 mL) was added at room temperature cesium carbonate (3.50 g, 18.0 mmol, 2.00 equiv.). The reaction mixture was stirred overnight at 60° C. After cooling down to room temperature, the reaction mixture was diluted with water and extracted with ethyl acetate. The combined organic layers were washed with brine, dried over sodium sulfate, filtered and concentrated under reduced pressure. Purification of the crude product by column chromatography gave methyl 5-[(2S)-2-(tert-butoxycarbonylamino)propoxy]-2-chloro-pyridine-3-carboxylate. LC-MS (method 1): retention time 1.45 min, m/z 288.9 [M+H⁺-^{t}Bu]; ¹H NMR (400 MHz, CDCl₃) δ ppm: 8.21 (d, 1H) 7.70 (d, 1H) 4.69 (br s, 1H) 4.05-4.14 (m, 1H) 3.99-4.03 (m, 2H) 3.95 (s, 3H) 1.45 (s, 9H) 1.30 (d, 3H)

### Step E: Preparation of methyl 5-[(2S)-2-aminopropoxy]-2-chloro-pyridine-3-carboxylate dihydrochloride

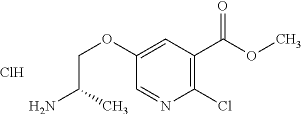 [SMILES: Cl ; COC(=O)c1cc(OC[C@H](C)N)cnc1Cl]

To a solution of methyl 5-[(2S)-2-(tert-butoxycarbonylamino)propoxy]-2-chloro-pyridine-3-carboxylate (2.00 g, 5.80 mmol) in 1,4-dioxane (8 mL) was added dropwise at 0° C. hydrogen chloride (4 M in 1,4-dioxane, 22 mL). The reaction mixture was stirred overnight at room temperature. It was then concentrated under reduced pressure to afford crude methyl 5-[(2S)-2-aminopropoxy]-2-chloro-pyridine-3-carboxylate dihydrochloride, which was used as such in next step. LC-MS (method 1): retention time 0.32 min, m/z 244.8 [M+H⁺]; ¹H NMR (400 MHz, DMSO-d6) δ ppm: 8.37 (d, 1H) 8.27-8.34 (m, 2H) 7.89 (d, 1H) 4.30 (m, 1H) 4.15 (m, 1H) 3.89 (s, 3H) 3.75 (br s, 1H) 1.29 (d, 3H)

### Step F: Preparation of methyl 2-chloro-5-[(2S)-2-(trifluoromethylsulfonylamino)propoxyl]pyridine-3-carboxylate

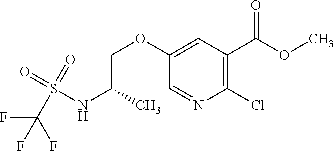 [SMILES: COC(=O)c1cc(OC[C@H](C)NS(=O)(=O)C(F)(F)F)cnc1Cl]

To a solution of crude methyl 5-[(2S)-2-aminopropoxy]-2-chloro-pyridine-3-carboxylate dihydrochloride (1.50 g, 4.70 mmol) in dichloromethane (30 mL) was added at 0° C. triethylamine (4.00 mL, 28.0 mmol, 6.00 equiv.), followed by trifluoromethanesulfonic anhydride (1.00 mL, 5.00 mmol, 1.05 equiv.). The reaction mixture was stirred overnight at room temperature. It was then concentrated under reduced pressure. Purification of the crude product by column chromatography gave methyl 2-chloro-5-[(2S)-2-(trifluoromethylsulfonylamino)propoxy]pyridine-3-carboxylate. LC-MS (method 1): retention time 1.13 min, m/z 377.3 [M+H⁺]; ¹H NMR (400 MHz, CDCl₃) δ ppm: 8.21 (d, 1H) 7.69 (d, 1H) 5.59 (br s, 1H) 4.08-4.26 (m, 2H) 4.01-4.07 (m, 1H) 3.96-3.99 (s, 3H) 1.49 (d, 3H); ¹⁹F NMR (377 MHz, CDCl₃) δ ppm: −77.72 (s, CF₃)

### Step G: Preparation of 2-chloro-5-[(2S)-2-(trifluoromethylsulfonylamino)propoxyl]pyridine-3-carboxylic acid

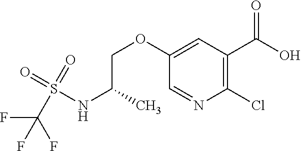 [SMILES: C[C@@H](COc1cnc(Cl)c(C(=O)O)c1)NS(=O)(=O)C(F)(F)F]

To a solution of methyl 2-chloro-5-[(2S)-2-(trifluoromethylsulfonylamino)propoxy]pyridine-3-carboxylate (0.900 g, 2.39 mmol) in tetrahydrofuran (18 mL) and methanol (3 mL) was added at room temperature a solution of lithium hydroxide (0.171 g, 7.17 mmol, 3.00 equiv.) in water (6 mL). The reaction mixture was stirred for 2 hours at room temperature. It was then diluted with water, then acidified with citric acid solution and extracted with ethyl acetate. The combined organic layers were dried over sodium sulfate, filtered and concentrated under reduced pressure. Washing the resulting gum with n-pentane gave 2-chloro-5-[(2S)-2-(trifluoromethylsulfonylamino)propoxy]pyridine-3-carboxylic acid. LC-MS (method 1): retention time 1.31 min, m/z 362.8 [M+H⁺]; ¹H NMR (400 MHz, DMSO-d6) δ ppm: 13.96-14.62 (m, 1H) 9.97-10.27 (m, 1H) 8.68 (d, 1H) 8.20 (d, 1H) 4.58 (m, 1H) 4.39-4.48 (m, 1H) 4.25-4.36 (m, 1H) 1.67 (d, 3H); ¹⁹F NMR (377 MHz, DMSO-d6) δ ppm: −77.68 (s, CF₃)

### Step H: Preparation of 2-chloro-N-cyclopropyl-5-[(2S)-2-(trifluoromethylsulfonylamino)propoxyl]pyridine-3-carboxamide (Compound P-1, Table P)

A solution of cyclopropanamine (0.0463 mL, 0.662 mmol, 1.20 equiv.), triethylamine (0.386 mL, 2.76 mmol, 5.00 equiv.) and 1-propanephosphonic anhydride (50 mass % in ethyl acetate, 0.975 mL, 1.65 mmol, 3.00 equiv.) in ethyl acetate (3 mL) was added at room temperature to a solution of 2-chloro-5-[(2S)-2-(trifluoromethylsulfonylamino)propoxy]pyridine-3-carboxylic acid (0.200 g, 0.551 mmol). The reaction mixture was stirred overnight at room temperature. It was then diluted with water and extracted with ethyl acetate. The combined organic layers were dried over sodium sulfate, filtered and concentrated under reduced pressure. Purification of the crude product by column chromatography gave 2-chloro-N-cyclopropyl-5-[(2S)-2-(trifluoromethylsulfonylamino)propoxy]pyridine-3-carboxamide.

LC-MS (method 1): retention time 1.03 min, m/z 402.3 [M+H⁺]; ¹H NMR (400 MHz, CDCl₃) δ ppm: 8.09 (d, 1H) 7.57 (d, 1H) 6.82 (br s, 1H) 6.20 (br d, 1H) 4.04-4.15 (m, 2H) 3.90-4.05 (m, 1H) 2.94 (m, 1H) 1.46 (d, 3H) 0.87-1.02 (m, 2H) 0.64-0.77 (m, 2H); ¹⁹F NMR (377 MHz, CDCl₃) δ ppm: −77.74 (s, CF₃)

### Example P2: Preparation of N-(1-bicyclo[1.1.1]pentanyl)-2-methyl-5-[(2S)-2 (trifluoromethylsulfonylamino)propoxyl]pyridine-3-carboxamide (Compound P-5, Table P)

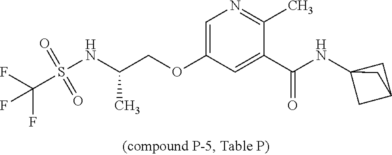 [SMILES: Cc1ncc(OC[C@H](C)NS(=O)(=O)C(F)(F)F)cc1C(=O)NC12CC(C1)C2]

### Step A: Preparation of ethyl 5-[(2S)-2-(tert-butoxycarbonylamino)propoxy]-2-methyl-pyridine-3-carboxylate

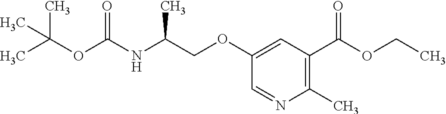 [SMILES: CCOC(=O)c1cc(OC[C@H](C)NC(=O)OC(C)(C)C)cnc1C]

To a solution of ethyl 5-hydroxy-2-methyl-pyridine-3-carboxylate (11.5 g, 63.5 mmol, 1.00 equiv.) and [(2S)-2-(tert-butoxycarbonylamino)propyl]methanesulfonate (3.35 g, 140 mmol, 2.2 equiv.) in acetonitrile (173 mL, 15 mL/g) was added cesium carbonate (57.57 g, 298.4 mmol, 4.70 equiv. The reaction mixture was stirred at 60° C. for 16 hr.

The reaction mixture was quenched with water and extracted with EtOAc. The combined organic layers were washed with brine, dried over anhydrous sodium sulfate, filtered, and concentrated under reduced pressure. The crude residue was purified by silica gel column chromatography (cyclohexane/EtOAc) to afford ethyl 5-[(2S)-2-(tert-butoxycarbonylamino)propoxy]-2-methyl-pyridine-3-carboxylate.

¹H NMR (400 MHz, CDCl₃) δ ppm: 1.26-1.37 (m, 3H) 1.41-1.49 (m, 12H) 2.77 (s, 3H) 4.01 (d, J=4.13 Hz, 2H) 4.06-4.16 (m, 1H) 4.40 (d, J=7.13 Hz, 2H) 4.61-4.88 (m, 1H) 7.73 (d, J=2.88 Hz, 1H) 8.34 (d, J=3.00 Hz, 1H)

### Step B: Preparation of [(1S)-2-(5-ethoxycarbonyl-6-methyl-pyridin-1-ium-3-yl)oxy-1-methyl-ethyl]ammonium dichloride

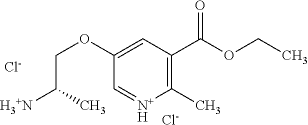

To an ice-cooled solution of ethyl 5-[(2S)-2-(tert-butoxycarbonylamino)propoxy]-2-methyl-pyridine-3-carboxylate (10.5 g, 31.0 mmol, 1.00 equiv.) in EtOAc (5 mL/g) was added dropwise hydrochloric acid (4 mol/L) in EtOAc (52.5 mL, 210 mmol, 6.77 equiv.). The reaction mixture was stirred at rt for 16 hr. The reaction mixture was then concentrated under reduced pressure to afford [(1S)-2-(5-ethoxycarbonyl-6-methyl-pyridin-1-ium-3-yl)oxy-1-methyl-ethyl]ammonium; dichloride as an off white solid.

¹H NMR (400 MHz, DMSO-d6) δ ppm: 1.29-1.38 (m, 6H) 2.76 (s, 3H) 4.24 (br d, J=7.13 Hz, 2H) 4.32-4.42 (m, 3H) 8.07 (d, J=2.88 Hz, 1H) 8.46 (br s, 3H) 8.58 (d, J=2.88 Hz, 1H)

### Step C: Preparation of ethyl 2-methyl-5-[(2S)-2-(trifluoromethylsulfonylamino)propoxyl]pyridine-3-carboxylate

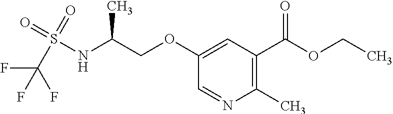 [SMILES: CCOC(=O)c1cc(OC[C@H](C)NS(=O)(=O)C(F)(F)F)cnc1C]

To an ice-cooled solution of [(1S)-2-(5-ethoxycarbonyl-6-methyl-pyridin-1-ium-3-yl)oxy-1-methyl-ethyl]ammonium dichloride (8.5 g, 27 mmol, 1.00 equiv.) and triethylamine (23 mL, 160 mmol, 6.00 equiv.) in acetonitrile (170 mL, 20 mL/g) was added trifluoromethylsulfonyl trifluoromethanesulfonate (5.5 mL, 33 mmol, 1.20 equiv.). The reaction mixture was allowed to come back to rt and stirred 16 hr. The reaction mixture was concentrated under reduced pressure and the crude residue was purified by silica gel column chromatography (cyclohexane/EtOAc) to afford ethyl 2-methyl-5-[(2S)-2-(trifluoromethylsulfonylamino)propoxy]pyridine-3-carboxylate.

¹H NMR (400 MHz, CDCl₃) δ ppm: 1.41-1.52 (m, 6H) 2.78 (s, 3H) 3.99-4.08 (m, 1H) 4.09-4.20 (m, 2H) 4.41 (q, J=7.13 Hz, 2H) 6.54 (br d, J=7.00 Hz, 1H) 7.71 (d, J=3.00 Hz, 1H) 8.35 (d, J=3.00 Hz, 1H)

### Step D: Preparation of 2-methyl-5-[(2S)-2-(trifluoromethylsulfonylamino)propoxyl]pyridine-3-carboxylic acid

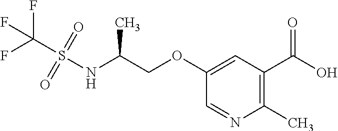 [SMILES: Cc1ncc(OC[C@H](C)NS(=O)(=O)C(F)(F)F)cc1C(=O)O]

To a solution of ethyl 2-methyl-5-[(2S)-2-(trifluoromethylsulfonylamino)propoxy]pyridine-3-carboxylate (3.8 g, 10 mmol, 1.00 equiv.) in ethanol (51 mL, 5 mL/mmol) was added sodium hydroxide (1 mol/L) in H₂O (51 mL, 100 mmol, 10 equiv.). The reaction mixture was stirred at rt for 2 hr. Water and citric acid were added, and the reaction mixture was extracted with EtOAc. The combined organic layers were washed with brine, dried over Na₂SO₄, filtered, and concentrated under reduced pressure. The obtained solid was washed with pentane and filtered off to afford 2-methyl-5-[(2S)-2-(trifluoromethylsulfonylamino)propoxy]pyridine-3-carboxylic acid.

¹H NMR (400 MHz, DMSO-d6) δ ppm: 1.27 (d, J=6.75 Hz, 3H) 2.64 (s, 3H) 3.81-3.93 (m, 1H) 3.98 (dd, J=9.94, 7.69 Hz, 1H) 4.12 (dd, J=10.07, 4.19 Hz, 1H) 7.70 (d, J=2.88 Hz, 1H) 8.33 (d, J=2.88 Hz, 1H) 9.63 (br d, J=8.13 Hz, 1H) 12.60-14.19 (m, 1H)

### Step E: Preparation of N-(1-bicyclo[1.1.1]Pentanyl)-2-methyl-5-[(2S)-2 (trifluoromethylsulfonyl amino)propoxyl]pyridine-3-carboxamide (Compound P-5, Table P)

2-methyl-5-[(2S)-2-(trifluoromethylsulfonylamino)propoxy]pyridine-3-carboxylic acid (0,014 g, 0.040 mmol, 1.00 equiv.), bicyclo[1.1.1]pentan-1-amine (0.007 g, 0.060 mmol, 1.50 equiv.), N-ethyl-N-isopropyl-propan-2-amine (0.042 mL, 0.240 mmol, 6.00 equiv.) were dissolved in N,N-Dimethylacetamide (0.90 mL) and the mixture was purged with a stream of argon for 5 minutes. The reaction mixture was heated at 60° C. for 3 hr. The mixture was cooled down and concentrated under reduced pressure The crude residue was dissolved in dimethylacetamide/methanol (3:1) and purified over reverse phase preparative HPLC to afford N-(1-bicyclo[1.1.1]pentanyl)-2-methyl-5-[(2S)-2 (trifluoromethylsulfonylamino)propoxy]pyridine-3-carboxamide.

LC-MS (method 5): retention time 1.22 min, m/z 408.4 [M+H]

### Example P3: Preparation of N-[(5-chloro-2-pyridyl)methyl]-2-methyl-5-[(2S)-2-(trifluoromethylsulfonylamino)propoxyl]pyridine-3-carboxamide (Compound P-28, Table)

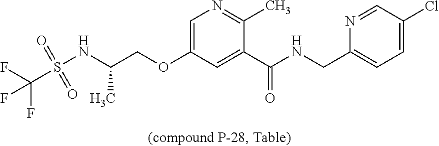 [SMILES: Cc1ncc(OC[C@H](C)NS(=O)(=O)C(F)(F)F)cc1C(=O)NCc1ccc(Cl)cn1]

### Step A: Preparation of N-[(5-chloro-2-pyridyl)methyl]-2-methyl-5-[(2S)-2-(trifluoromethylsulfonyl amino)propoxyl]pyridine-3-carboxamide (Compound P-28, Table P)

2-methyl-5-[(2S)-2-(trifluoromethylsulfonylamino)propoxy]pyridine-3-carboxylic acid (0.014 g, 0.040 mmol, 1.00 equiv.), (5-chloro-2-pyridyl)methanamine (0.0086 g, 0.060 mmol, 1.50 equiv.), N-ethyl-N-isopropyl-propan-2-amine (0.0415 mL, 0.240 mmol, 6.00 equiv.) were dissolved in N,N-Dimethylacetamide (0.90 mL) and the mixture was purged with a stream of argon for 5 minutes. The reaction mixture was heated at 60° C. for 3 hr. The mixture was cooled down and concentrated under reduced pressure The crude residue was dissolved in dimethylacetamide/methanol (3:1) and purified over reverse phase preparative HPLC to afford N-[(5-chloro-2-pyridyl)methyl]-2-methyl-5-[(2S)-2-(trifluoromethylsulfonylamino)propoxy]pyridine-3-carboxamide.

LC-MS (method 5): retention time 1.21 min, m/z 467.4 [M+H]

### Example P4: Preparation of Preparation of 5-bromo-N-cyclopropyl-2-[(2S)-2-(trifluoromethylsulfonylamino)propoxyl]pyridine-4-carboxamide (Compound P-83, Table P)

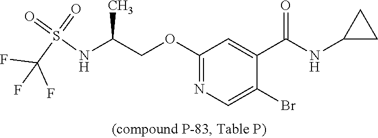 [SMILES: C[C@@H](COc1cc(C(=O)NC2CC2)c(Br)cn1)NS(=O)(=O)C(F)(F)F]

### Step A: Preparation of methyl 5-bromo-2-[(2S)-2-(tert-butoxycarbonylamino)propoxyl]pyridine-4-carboxylate

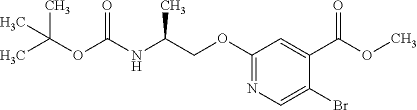 [SMILES: COC(=O)c1cc(OC[C@H](C)NC(=O)OC(C)(C)C)ncc1Br]

To a solution of methyl 5-bromo-2-hydroxy-pyridine-4-carboxylate (10.8 g, 46.5 mmol, 1.00 equiv.) and [(2S)-2-(tert-butoxycarbonylamino)propyl]methanesulfonate (17.7 g, 69.8 mmol, 1.50 equiv.) in dimethylformamide (582 mL, 12.5 mL/mmol) was added potassium carbonate (19.5 g, 140 mmol, 3.00 equiv.). The reaction mixture was stirred at 60° C. for 16 hr. The reaction mixture was then cooled to rt, diluted with water, and extracted with EtOAc. The combined organic layers were washed with water, dried over Na₂SO₄, filtered, and concentrated under reduced pressure. The reaction mixture was concentrated underreduced pressure and the crude residue was purified by silica gel column chromatography (cyclohexane/EtOAc) to afford methyl 5-bromo-2-[(2S)-2-(tert-butoxycarbonylamino)propoxy]pyridine-4-carboxylate as a light yellowish thick liquid.

¹H NMR (400 MHz, CDCl₃) δ ppm 1.19-1.28 (m, 3H) 1.43 (s, 9H) 3.94 (s, 3H) 3.99-4.17 (m, 1H) 4.25 (d, 2H) 4.73 (br s, 1H) 7.11 (s, 1H) 8.32 (s, 1H).

### Step B: Preparation of [(1S)-2-(5-bromo-4-methoxycarbonyl-pyridin-1-ium-2-yl)oxy-1-methyl-ethyl]ammonium; dichloride

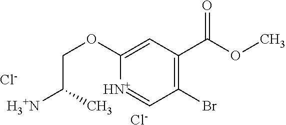

To an ice-cooled solution of methyl 5-bromo-2-[(2S)-2-(tert-butoxycarbonylamino)propoxy]pyridine-4-carboxylate (9.9 g, 25 mmol, 1.00 equiv.) in EtOAc (41 mL 4.1 mL/g) was added hydrochloric acid (4 mol/L) in EtOAc (110 mL, 440 mmol, 17 equiv.). The reaction mixture was stirred at rt for 16 hr. The reaction mixture was then concentrated under reduced pressure, the residue was washed with tert-butyl methyl ether, filtered and dried under reduced pressure to afford [(1S)-2-(5-bromo-4-methoxycarbonyl-pyridin-1-ium-2-yl)oxy-1-methyl-ethyl]ammonium; dichloride as white solid.

¹H NMR (400 MHz, DMSO-d6) δ ppm 1.28 (d, 3H) 3.59 (m, 1H) 3.90 (s, 3H) 4.31 (m, 1H) 4.39-4.47 (m, 1H) 7.21 (s, 1H) 8.05-8.43 (m, 3H) 8.49 (s, 1H).

### Step C: Preparation of methyl 5-bromo-2-[(2S)-2-(trifluoromethylsulfonylamino)propoxyl]pyridine-4-carboxylate

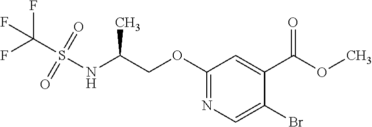 [SMILES: COC(=O)c1cc(OC[C@H](C)NS(=O)(=O)C(F)(F)F)ncc1Br]

To an ice-cooled solution of [(1S)-2-(5-bromo-4-methoxycarbonyl-pyridin-1-ium-2-yl)oxy-1-methyl-ethyl]ammonium; dichloride (4 g, 11.05 mmol, 1.00 equiv.) in acetonitrile (80 mL, 20 mL/g) was added Triethylamine (9.29 mL, 66.29 mmol, 6.00 equiv.) followed by trifluoromethylsulfonyl trifluoromethanesulfonate (2.230 mL, 13.27 mmol, 1.20 equiv.). The reaction mixture was allowed to reach rt slowly and stirred for 16 hr. Water and citric acid were added, and the reaction mixture was extracted with EtOAc. The combined organic layers were washed with brine, dried over Na₂SO₄, filtered, and concentrated under reduced pressure. The reaction mixture was concentrated under reduced pressure and the crude residue was purified by silica gel column chromatography (cyclohexane/EtOAc) to afford methyl 5-bromo-2-[(2S)-2-(trifluoromethylsulfonylamino)propoxy]pyridine-4-carboxylate as slightly yellowish thick liquid.

¹H NMR (400 MHz, CDCl₃) δ ppm 1.39 (d, 3H) 3.96 (s, 3H) 4.01-4.15 (m, 1H) 4.28-4.43 (m, 2H) 5.96 (br d, 1H) 7.13 (s, 1H) 8.32 (s, 1H).

### Step D: Preparation of 5-bromo-2-[(2S)-2-(trifluoromethylsulfonylamino)propoxy]pyridine-4-carboxylic acid

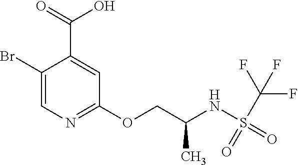 [SMILES: C[C@@H](COc1cc(C(=O)O)c(Br)cn1)NS(=O)(=O)C(F)(F)F]

To a solution of methyl 5-bromo-2-[(2S)-2-(trifluoromethylsulfonylamino)propoxy]pyridine-4-carboxylate (2.5 g, 5.9 mmol, 1.00 equiv.) in tetrahydrofuran (50 mL) was added lithium hydroxide hydrate (0.750 g, 18 mmol, 3.00 equiv.). The reaction mixture was stirred at rt for 2 hr. The reaction mixture was concentrated under reduced pressure, water and citric acid were added, and the reaction mixture was extracted with EtOAc. The combined organic layers were washed with brine, dried over Na₂SO₄, filtered, and concentrated under reduced pressure. The obtained solid was washed with pentane and filtered off to afford 5-bromo-2-[(2S)-2-(trifluoromethylsulfonylamino)propoxy]pyridine-4-carboxylic acid

¹H NMR (400 MHz, DMSO-d6) δ ppm: 1.24 (d, 3H) 3.81-3.95 (m, 1H) 4.14 (m, 1H) 4.32 (m, 1H) 7.06 (s, 1H) 8.42 (s, 1H) 9.61 (m, 1H) 13.71-14.63 (m, 1H).

### Step E: Preparation of 5-bromo-N-cyclopropyl-2-[(2S)-2-(trifluoromethylsulfonylamino)propoxyl]pyridine-4-carboxamide (Compound P-83, Table P)

To a solution of 5-bromo-2-[(2S)-2-(trifluoromethylsulfonylamino)propoxy]pyridine-4-carboxylic acid (0.200 g, 0.49 mmol, 1.00 equiv.), cyclopropanamine (0.034 g, 0.59 mmol, 1.20 equiv.) and N,N-diethylethanamine (0.250 g, 2.4560 mmol, 5.00 equiv.) in EtOAc (2.45 mL, 5 mL/mmol) was added 1-propanephosphonic anhydride (50 mass %) in EtOAc (0.868 mL, 1.47 mmol, 3.00 equiv.). The reaction mixture was stirred at rt for 16 hr. The reaction mixture was diluted with water, acidified with citric acid solution, and extracted with EtOAc. The combined organic layers were washed with brine, dried over Na₂SO₄, filtered, and concentrated under reduced pressure. The crude residue was purified by silica gel column chromatography (cyclohexane/EtOAc). The obtained solid was washed with pentane and filtered off to afford 5-bromo-N-cyclopropyl-2-[(2S)-2-(trifluoromethylsulfonylamino)propoxy]pyridine-4-carboxamide as white solid.
1H NMR (400 MHz, DMSO-d6) δ ppm: 0.47-0.60 (m, 2H) 0.65-0.78 (m, 2H) 1.24 (d, 3H) 2.71-2.90 (m, 1H) 3.88 (br s, 1H) 4.13 (m, 1H) 4.28 (m, 1H) 6.81 (s, 1H) 8.34 (s, 1H) 8.64 (d, 1H) 9.61 (br s, 1H)

### Example P5: Preparation of 5-bromo-N-cyclopropyl-2-[(2S)-2-[methoxymethyl (trifluoromethylsulfonyl)amino]propoxy]pyridine-4-carboxamide (Compound P-81, Table P)

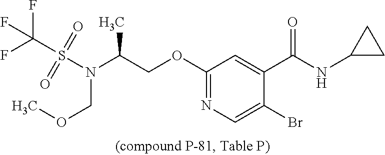 [SMILES: COCN([C@@H](C)COc1cc(C(=O)NC2CC2)c(Br)cn1)S(=O)(=O)C(F)(F)F]

### Step A: Preparation of 5-bromo-N-cyclopropyl-2-[(2S)-2-[methoxymethyl (trifluoromethylsulfonyl)amino]propoxy]pyridine-4-carboxamide

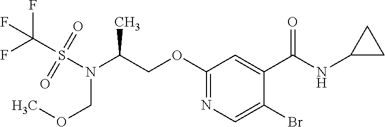 [SMILES: COCN([C@@H](C)COc1cc(C(=O)NC2CC2)c(Br)cn1)S(=O)(=O)C(F)(F)F]

To an ice-salt freezing mixture solution of 5-bromo-N-cyclopropyl-2-[(2S)-2-(trifluoromethylsulfonylamino)propoxy]pyridine-4-carboxamide (100 mg, 0.2241 mmol, 1.00 equiv.) and Triethylamine (0.094 mL, 0.67 mmol, 3.00 equiv.) in acetonitrile (2 mL, 20 mL/g) was added chloro(methoxy)methane (0.020 mL, 0.268 mmol, 1.20 equiv.). The reaction mixture was stirred in the same ice-salt freezing mixture for 3 hr. The reaction mixture was diluted with water, acidified with citric acid solution, and extracted with EtOAc. The combined organic layers were washed with brine, dried over Na₂SO₄, filtered, and concentrated under reduced pressure. The crude residue was purified by silica gel column chromatography (cyclohexane/EtOAc) to afford 5-bromo-N-cyclopropyl-2-[(2S)-2-[methoxymethyl(trifluoromethylsulfonyl)amino]propoxy]pyridine-4-carboxamide as a gummy mass.
1H NMR (400 MHz, CDCl₃) δ ppm 0.60-0.78 (m, 2H) 0.83-0.97 (m, 2H) 1.42 (br d, 3H) 2.90 (m, 1H) 3.40 (s, 3H) 4.28-4.57 (m, 3H) 4.85 (br s, 2H) 6.10 (br s, 1H) 6.89 (s, 1H) 8.25 (s, 1H).

### Example P6: Preparation of 5-bromo-N-[(4R)-2-ethyl-3-oxo-isoxazolidin-4-yl]-2-[(2S)-2-(trifluoromethylsulfonylamino)propoxyl]pyridine-4-carboxamide (Compound P-84, Table P)

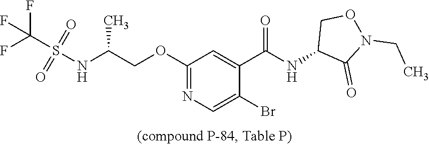 [SMILES: CCN1OC[C@@H](NC(=O)c2cc(OC[C@@H](C)NS(=O)(=O)C(F)(F)F)ncc2Br)C1=O]

### Step A: Preparation of 5-bromo-N-[(4R)-2-ethyl-3-oxo-isoxazolidin-4-yl]-2-[(2S)-2-(trifluoromethylsulfonylamino)propoxy]pyridine-4-carboxamide

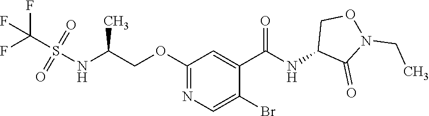 [SMILES: CCN1OC[C@@H](NC(=O)c2cc(OC[C@H](C)NS(=O)(=O)C(F)(F)F)ncc2Br)C1=O]

To a solution of 5-bromo-2-[(2S)-2-(trifluoromethylsulfonylamino)propoxy]pyridine-4-carboxylic acid (0.200 g, 0.49 mmol, 1.00 equiv.), [(4R)-2-ethyl-3-oxo-isoxazolidin-4-yl]ammonium chloride (0.098 g, 0.589 mmol, 1.20 equiv.) and N,N-diethylethanamine (0.250 g, 2.456 mmol, 5.00 equiv.) in EtOAc (2.45 mL, 5 mL/mmol) was added 1-propanephosphonic anhydride (50 mass %) in EtOAc (0.868 mL, 1.47 mmol, 3.00 equiv.). The reaction mixture was stirred at rt for 16 hr. The reaction mixture was diluted with water, acidified with citric acid solution, and extracted with EtOAc. The combined organic layers were washed with brine, dried over Na₂SO₄, filtered, and concentrated under reduced pressure. The crude residue was purified by silica gel column chromatography (cyclohexane/EtOAc). The obtained solid was washed with pentane and filtered off to afford 5-bromo-N-[(4R)-2-ethyl-3-oxo-isoxazolidin-4-yl]-2-[(2S)-2-(trifluoromethylsulfonylamino)propoxy]pyridine-4-carboxamide as a white solid.
1H NMR (400 MHz, DMSO-d6) δ ppm: 1.15 (t, 3H) 1.25 (d, 3H) 3.54 (m, 2H) 3.83-3.98 (m, 1H) 4.08 (t, 1H) 4.15 (m 1H) 4.31 (m, 1H) 4.63 (t, 1H) 4.98 (m, 1H) 6.86 (s, 1H) 8.39 (s, 1H) 9.30 (d, 1H) 9.63 (br d, 1H).

### Example P7: Preparation of 5-chloro-N-cyclopropyl-2-[(2S)-2-(trifluoromethylsulfonyl amino)propoxyl]pyridine-4-carboxamide (Compound P-87, Table P)

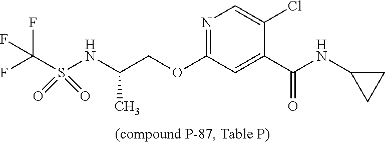 [SMILES: C[C@@H](COc1cc(C(=O)NC2CC2)c(Cl)cn1)NS(=O)(=O)C(F)(F)F]

### Step A: Preparation of ethyl 2,5-dichloropyridine-4-carboxylate

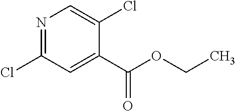

To a solution of 2,5-dichloropyridine-4-carboxylic acid (3.70 g, 18.3 mmol, 1.00 equiv.) in EtOAc (91.5 mL) was added oxalyl dichloride (1.93 mL, 22.0 mmol, 1.20 equiv.) and then one drop of N,N-dimethylformamide. The reaction mixture was stirred at rt for 2 hr. Ethanol (17.8 g, 21.8 mL, 366 mmol) was added and the reaction mixture was stirred at rt for 30 minutes. The crude mixture was concentrated in reduced pressure and diluted with EtOAc and treated with a saturated solution of NaHCO₃. The desired material was extracted with EtOAc, the combined organic layers were washed with water, brine, dried over sodium sulfate, filtered, and concentrated under reduced pressure. The crude residue was purified over silica gel cartridge (cyclohexane/EtOAc) to afford ethyl 2,5-dichloropyridine-4-carboxylate as a colorless oil.

¹H NMR (400 MHz, CDCl₃) δ ppm: 8.37-8.54 (m, 1H) 7.69 (s, 1H) 4.45 (q, J=7.02 Hz, 2H) 1.43 (t, J=7.27 Hz, 3H)

### Step B: Preparation of ethyl 5-chloro-2-hydroxy-pyridine-4-carboxylate

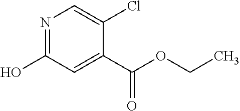

To a solution of ethyl 2,5-dichloropyridine-4-carboxylate (1.0 g, 4.5 mmol) in acetic acid (13 mL, 220 mmol, 48 equiv.) was added sodium acetate (1.9 g, 23 mmol, 5.0 equiv.). The reaction mixture was irradiated in the microwave at 170° C. for 8 hr. The reaction mixture was poured into ice and extracted with EtOAc. The combined organic layers were washed with brine, dried over Na₂SO₄, filtered, and concentrated in reduced pressure to afford ethyl 5-chloro-2-hydroxy-pyridine-4-carboxylate as a beige solid.

¹H NMR (400 MHz, CDCl₃) δ ppm: 7.54 (s, 1H) 6.95 (s, 1H) 4.37-4.49 (m, 2H) 1.41 (t, J=7.27 Hz, 3H)

### Step C: Preparation of ethyl 2-[(2S)-2-(tert-butoxycarbonylamino)propoxy]-5-chloro-pyridine-4-carboxylate

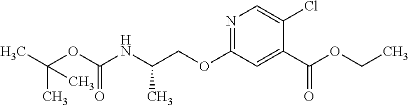 [SMILES: CCOC(=O)c1cc(OC[C@H](C)NC(=O)OC(C)(C)C)ncc1Cl]

To a solution of ethyl 5-chloro-2-hydroxy-pyridine-4-carboxylate (0.770 g, 3.82 mmol, 1.00 equiv.) and cesium carbonate (2.62 g, 7.64 mmol, 2.00 equiv.) in DMF (15.3 mL) was added [(2S)-2-(tert-butoxycarbonylamino)propyl]methanesulfonate (1.35 g, 5.35 mmol, 1.40 equiv.). The reaction mixture was stirred at 60° C. 6.5 hr and then at rt for 16 hr. The crude mixture was diluted with EtOAc and treated with a saturated solution of NaHCO₃. The desired material was extracted with EtOAc, the combined organic layers were washed with water, brine, dried over sodium sulfate, filtered, and concentrated under reduced pressure. The crude residue was purified over silica gel cartridge (cyclohexane/EtOAc) to afford ethyl 2-[(2S)-2-(tert-butoxycarbonylamino)propoxy]-5-chloro-pyridine-4-carboxylate as a colorless oil.

¹H NMR (400 MHz, CDCl₃) δ ppm 8.20 (s, 1H) 7.12 (s, 1H) 4.71 (br s, 1H) 4.42 (q, J=7.27 Hz, 2H) 4.27 (d, J=4.72 Hz, 2H) 4.08 (br s, 1H) 1.45 (s, 9H) 1.41 (t, J=7.27 Hz, 3H) 1.25 (d, J=6.90 Hz, 3H)

### Step D: Preparation of [(1S)-2-[(5-chloro-4-ethoxycarbonyl-2-pyridyl)oxy]-1-methyl-ethyl]ammonium chloride

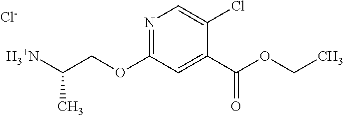

To an ice-cooled solution of ethyl 2-[(2S)-2-(tert-butoxycarbonylamino)propoxy]-5-chloro-pyridine-4-carboxylate (0.40 g, 1.10 mmol, 1.0 equiv.) in dioxane (0.45 mL) was added dropwise Hydrogen chloride solution 4.0M in dioxane (2.8 mL, 11.0 mmol, 10.0 equiv.). The reaction mixture was stirred at rt for 16 hr. The reaction mixture was concentrated under reduced pressure to afford [(1S)-2-[(5-chloro-4-ethoxycarbonyl-2-pyridyl)oxy]-1-methyl-ethyl]ammonium chloride as a white solid.

¹H NMR (400 MHz, DMSO-d6) δ ppm: 8.40 (s, 1H) 8.23 (br s, 3H) 7.22 (s, 1H) 4.43 (dd, J=11.26, 4.36 Hz, 1H) 4.37 (q, J=6.90 Hz, 2H) 4.31 (dd, J=11.26, 6.90 Hz, 1H) 3.61 (td, J=6.81, 4.18 Hz, 1H) 1.33 (t, J=7.08 Hz, 3H) 1.28 (d, J=6.54 Hz, 3H)

### Step E: Preparation of ethyl 5-chloro-2-[(2S)-2-(trifluoromethylsulfonylamino)propoxyl]pyridine-4-carboxylate

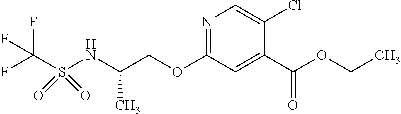 [SMILES: CCOC(=O)c1cc(OC[C@H](C)NS(=O)(=O)C(F)(F)F)ncc1Cl]

To an ice-cooled solution of [(1S)-2-[(5-chloro-4-ethoxycarbonyl-2-pyridyl)oxy]-1-methyl-ethyl]ammonium chloride (0.31 g, 1.10 mmol, 1.0 equiv.) in tetrahydrofuran (5.3 mL) were added triethylamine (0.37 g, 0.51 mL, 3.70 mmol, 3.50 equiv.) and then slowly trifluoromethanesulfonic anhydride (0.33 g, 0.19 mL, 1.20 mmol, 1.10 equiv.). The reaction mixture was stirred at 0° C. for 1 hour. The reaction mixture was poured into ice/water and extracted with EtOAc. The combined organic layers were washed with a saturated solution of NaHCO₃, brine, dried over Na₂SO₄, filtered, and concentrated under reduced pressure. The crude residue was purified over silica gel cartridge (cyclohexane/EtOAc) to afford ethyl 5-chloro-2-[(2S)-2-(trifluoromethylsulfonylamino)propoxy]pyridine-4-carboxylate as a yellow resin.

¹H NMR (400 MHz, CDCl₃) δ ppm: 8.20 (s, 1H) 7.15 (s, 1H) 5.75 (br d, J=8.36 Hz, 1H) 4.43 (q, J=7.15 Hz, 2H) 4.37 (t, J=4.72 Hz, 2H) 4.04-4.12 (m, 1H) 1.40-1.44 (m, 6H)

### Step F: Preparation of 5-chloro-2-[(2S)-2-(trifluoromethylsulfonylamino)propoxyl]pyridine-4-carboxylic acid

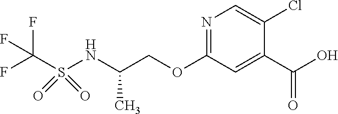 [SMILES: C[C@@H](COc1cc(C(=O)O)c(Cl)cn1)NS(=O)(=O)C(F)(F)F]

To a solution of ethyl 5-chloro-2-[(2S)-2-(trifluoromethylsulfonylamino)propoxy]pyridine-4-carboxylate (0.040 g, 0.10 mmol, 1.00 equiv.) in tetrahydrofuran (0.31 mL) and Water (0.31 mL) was added lithium hydroxide monohydrate (0.007 g, 0.15 mmol, 1.50 equiv.). The reaction mixture was stirred at rt for 6.5 hr. The reaction mixture was diluted with water/NaOH 0.5M and extracted with tert-butyl-methylether. The aqueous layer was acidified with HCl 2M until pH 3 and extracted with EtOAc. The organic layer was washed with a saturated solution of NH₄Cl, dried over Na₂SO₄, filtered and concentrated under reduced pressure to afford 5-chloro-2-[(2S)-2-(trifluoromethylsulfonylamino)propoxy]pyridine-4-carboxylic acid as a white solid.

¹H NMR (400 MHz, CDCl₃) δ ppm 8.26 (s, 1H) 7.32 (s, 1H) 5.75 (br d, J=8.36 Hz, 1H) 4.34-4.45 (m, 2H) 4.03-4.11 (m, 1H) 1.43 (d, J=6.90 Hz, 3H)

### Step G: Preparation of 5-chloro-N-cyclopropyl-2-[(2S)-2-(trifluoromethylsulfonylamino)propoxyl]pyridine-4-carboxamide (Compound P-87, Table P)

To a solution of 5-chloro-2-[(2S)-2-(trifluoromethylsulfonylamino)propoxy]pyridine-4-carboxylic acid (0.035 g, 0.096 mmol, 1.00 equiv.), cyclopropanamine (0.007 g, 0.0088 mL, 0.13 mmol, 1.30 equiv.) and N, N-Diisopropylethylamine (0.031 g, 0.042 mL, 0.24 mmol, 2.50 equiv.) in N,N-Dimethylformamide (0.096 mL) was added HATU (0.042 g, 0.11 mmol, 1.10 equiv.). The reaction mixture was stirred at rt for 3 hr. The reaction mixture was diluted with water and extracted with EtOAc. The combined organic layers were washed with brine, dried over Na₂SO₄, filtered, and concentrated under reduced pressure. The crude residue was purified by silica gel column chromatography (cyclohexane/EtOAc). The obtained solid was washed with pentane and filtered off to afford 5-chloro-N-cyclopropyl-2-[(2S)-2-(trifluoromethylsulfonylamino)propoxy]pyridine-4-carboxamide as a white solid.
1H NMR (400 MHz, CDCl₃) δ ppm 8.14 (s, 1H) 7.06 (s, 1H) 6.34 (br s, 1H) 5.92 (br d, J=8.36 Hz, 1H) 4.36-4.41 (m, 1H) 4.31-4.36 (m, 1H) 4.03-4.12 (m, 1H) 2.93 (tq, J=7.08, 3.63 Hz, 1H) 1.40 (d, J=6.90 Hz, 3H) 0.89-0.95 (m, 2H) 0.65-0.70 (m, 2H)

### Example P8: Preparation of 3-chloro-N-cyclopropyl-6-[(2S)-2-(trifluoromethylsulfonyl amino)propoxy]pyridazine-4-carboxamide (Compound P-88, Table P)

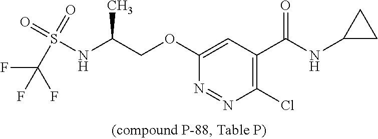 [SMILES: C[C@@H](COc1cc(C(=O)NC2CC2)c(Cl)nn1)NS(=O)(=O)C(F)(F)F]

### Step A: Preparation of 6-[(2S)-2-(tert-butoxycarbonylamino)propoxy]-3-chloro-pyridazine-4-carboxylic acid

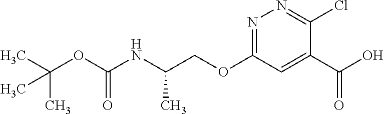 [SMILES: C[C@@H](COc1cc(C(=O)O)c(Cl)nn1)NC(=O)OC(C)(C)C]

To a solution of tert-butyl N-[(1S)-2-hydroxy-1-methyl-ethyl]carbamate (2.200 g, 12.56 mmol, 1 equiv.) and 3,6-dichloropyridazine-4-carboxylic acid (2.66 g, 13.81 mmol, 1.1 equiv.) in DMSO (35 mL) at 0° C. was added dropwise an aqueous solution of benzytrimethylammonium hydroxide (14.87 mL, 37.7 mmol, 40 mass %, 3 equiv.). The resulting mixture was then warmed to rt and stirred for 2 hr. Next the mixture was diluted with water, acidified to neutral pH with HCl_{aq} (2M) and extracted with EtOAc. Next the aqueous phase was further acidified to pH 5 with HCl_{aq} (2M) and extracted several times with EtOAc. The combined organic phases were washed with water, dried over sodium sulfate, and concentrated in vacuo. The desired product was obtained and used in the next step without further purification.

¹H NMR (400 MHz, DMSO-d6) δ ppm 1.12 (d, J=6.90 Hz, 3H) 1.36 (s, 9H) 3.11-3.34 (m, 1H) 3.86-3.97 (m, 1H) 4.23-4.33 (m, 1H) 4.34-4.45 (m, 1H) 7.52-7.59 (m, 1H) 13.39-15.25 (m, 1H)

### Step B: tert-butyl N-[(1S)-2-[6-chloro-5-(cyclopropylcarbamoyl)pyridazin-3-yl]oxy-1-methyl-ethyl]carbamate

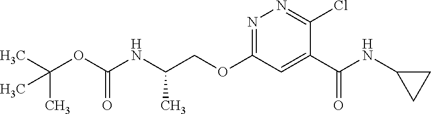 [SMILES: C[C@@H](COc1cc(C(=O)NC2CC2)c(Cl)nn1)NC(=O)OC(C)(C)C]

To a solution of 6-[(2S)-2-(tert-butoxycarbonylamino)propoxy]-3-chloro-pyridazine-4-carboxylic acid (0.20 g, 0.48 mmol, 1.00 equiv.), cyclopropanamine (0.033 g, 0.040 mL, 0.58 mmol, 1.20 equiv.) and triethylamine (0.147 g, 0.203 mL, 1.45 mmol, 3.0 equiv.) in acetonitrile (4.82 mL) was added a 1.7 M solution of 1-propanephosphonic anhydride in EtOAc (0.965 mmol, 2.0 equiv.) and the reaction was stirred at rt for 15 min. Next the mixture was diluted with EtOAc and treated with a saturated NaHCO₃ aqueous solution. The aqueous layer was extracted several times with EtOAc, the combined organic layers were dried over sodium sulfate and concentrated in vacuo. The desired compound was obtained and used in the next step without further purification.
1H NMR DMSO-d6) δ ppm 0.53 (br dd, J=3.81, 2.36 Hz, 2H) 0.73 (dd, J=7.08, 2.00 Hz, 2H) 1.12 (d, J=6.90 Hz, 3H) 1.38 (s, 9H) 2.81 (td, J=7.36, 3.81 Hz, 1H) 3.91 (dt, J=13.90, 6.68 Hz, 1H) 4.19-4.40 (m, 3H) 6.92 (br d, J=7.99 Hz, 1H) 7.39 (s, 1H) 8.79 (d, J=4.00 Hz, 1H)

### Step C: Preparation of [(1S)-2-[6-chloro-5-(cyclopropylcarbamoyl)pyridazin-3-yl]oxy-1-methyl-ethyl]ammonium chloride

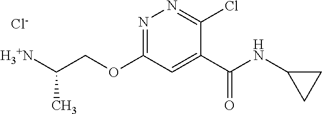

Tert-butyl N-[(1S)-2-[6-chloro-5-(cyclopropylcarbamoyl)pyridazin-3-yl]oxy-1-methyl-ethyl]carbamate (0.21 g, 0.57 mmol, 1.0 equiv.) was dissolved in a 1.25 M solution of HCl in MeOH (4 ml, 5.00 mmol). The mixture was stirred at rt for 30 min and then at 65° C. for 1 hour. The resulting mixture was concentrated using a rotatory evaporator to yield the title compound, which was used in the next step without further purification.

¹H NMR (400 MHz, DMSO-d6) δ ppm 0.48-0.58 (m, 2H) 0.67-0.82 (m, 2H) 1.26-1.34 (m, 3H) 2.82 (td, J=7.36, 3.81 Hz, 1H) 2.98-3.10 (m, 1H) 4.45-4.64 (m, 2H) 7.37 (s, 1H) 8.36 (br s, 3H) 8.91 (br d, J=2.54 Hz, 1H).

### Step D: Preparation of cyclopropyl 3-chloro-6-[(2S)-2-(trifluoromethylsulfonylamino)propoxy]pyridazine-4-carboxylate (Compound P-88, Table P)

To a solution of [(1S)-2-[6-chloro-5-(cyclopropylcarbamoyl)pyridazin-3-yl]oxy-1-methyl-ethyl]ammonium chloride (0.20 g, 0.65 mmol, 1 equiv.) in acetonitrile (6.5 mL) were added triethylamine (0.367 ml, 2.60 mmol, 4 equiv.) at −40° C. was added dropwise trifluoromethanesulfonic anhydride (0.11 ml, 0.65 mmol, 1.0 equiv.). The resulting mixture was stirred and the warmed to rt in 30 min. Next the mixture was diluted with EtOAc and treated with a saturated NaHCO₃ aqueous solution, and the aqueous layer was extracted several times with EtOAc. The combined organic phases were dried over sodium sulfate and concentrated in vacuo. Purification by column chromatography on silica gel (eluent: mixtures EtOAc/cyclohexane) afforded the title compound as colorless oil.

¹H NMR (400 MHz, DMSO-d6) δ ppm 0.52-0.58 (m, 2H) 0.70-0.76 (m, 2H) 1.28 (d, J=6.90 Hz, 3H) 2.81 (td, J=7.36, 3.81 Hz, 1H) 3.93-4.00 (m, 1H) 4.32 (dd, J=11.26, 7.63 Hz, 1H) 4.50 (dd, J=11.26, 4.36 Hz, 1H) 7.38 (s, 1H) 8.82 (d, J=4.36 Hz, 1H) 9.65 (br s, 1H).

¹⁹F NMR (377 MHz, DMSO-d6) δ ppm −77.79 (s, 3F).

Further examples of synthesized compounds of formula (I) are shown in Table P.

TABLE P
Physical data of compounds of formula I

| | | RT | [M + H] | | MP |
|---|---|---|---|---|---|
| Entry | IUPAC name | (min) | (measured) | Method | ° C. |
| P-1 | 2-chloro-N-cyclopropyl-5-[(2S)-2- | 1.03 | 402.3 | 1 | 110-112 |
| | (trifluoromethylsulfonylamino)propoxy]pyridine-3-carboxamide | | | | |
| P-2 | 2-chloro-N-(1-cyanocyclopropyl)-5-[(2S)-2- | 1.05 | 427.3 | 1 | 175-177 |
| | (trifluoromethylsulfonylamino)propoxy]pyridine-3-carboxamide | | | | |
| P-3 | 2-chloro-N-(2,2,2-trifluoroethyl)-5-[(2S)-2- | 1.10 | 444.2 | 1 | 118-120 |
| | (trifluoromethylsulfonylamino)propoxy]pyridine-3-carboxamide | | | | |
| P-4 | 2-chloro-N-cyclopropyl-5-[(2S)-2- | 0.81 | 418.03 | 4 | |
| | (dichloromethylsulfonylamino)propoxy]pyridine-3-carboxamide | | | | |
| P-5 | N-(1-bicyclo[1.1.1]pentanyl)-2-chloro-5-[(2S)-2- | 1.12 | 428.1 | 1 | |
| | (trifluoromethylsulfonylamino)propoxy]pyridine-3-carboxamide | | | | |
| P-6 | tert-butyl N-[(1S)-2-[6-chloro-5-[(1- | 1.68 | 527.0 | 3 | |
| | cyanocyclopropyl)carbamoyl]-3-pyridyl]oxy]-1-methyl-ethyl]- | | | | |
| | N-(trifluoromethylsulfonyl)carbamate | | | | |
| P-7 | 2-chloro-N-(1-cyanocyclopropyl)-5-[(2S)-2- | 1.56 | 441.0 | 3 | |
| | [methyl(trifluoromethylsulfonyl)amino]propoxy]pyridine-3- | | | | |
| | carboxamide | | | | |
| P-8 | 2-chloro-N-(1-cyanocyclopropyl)-5-[(2S)-2- | 1.40 | 466.1 | 2 | 168-170 |
| | [cyanomethyl(trifluoromethylsulfonyl)amino] | | | | |
| | propoxy]pyridine-3-carboxamide | | | | |
| P-9 | [[(1S)-2-[[6-chloro-5-[(1-cyanocyclopropyl)carbamoyl]-3- | 1.64 | 527.1 | 3 | |
| | pyridyl]oxy]-1-methyl-ethyl]-(trifluoromethylsulfonyl)amino] | | | | |
| | methyl 2-methylpropanoate | | | | |
| P-10 | 2-chloro-N-cyclopropyl-5-[(25)-2- | 0.98 | 384.1 | 1 | |
| | (difluoromethylsulfonylamino)propoxy]pyridine-3-carboxamide | | | | |
| P-11 | N-tert-butyl-2-chloro-5-[(2S)-2- | 1.59 | 418.0 | 3 | 152-154 |
| | (trifluoromethylsulfonylamino)propoxy]pyridine-3- | | | | |
| | carboxamide | | | | |
| P-12 | N-acetyl-2-chloro-N-cyclopropyl-5-[(2S)-2- | 1.12 | 488.1 | 1 | |
| | [methoxymethyl(trifluoromethylsulfonyl)amino] | | | | |
| | propoxy]pyridine-3-carboxamide | | | | |
| P-13 | [[(1S)-2-[[6-chloro-5-(cyclopropylcarbamoyl)-3-pyridyl]oxy]- | 1.27 | 502.1 | 1 | |
| | 1-methyl-ethyl]-(trifluoromethylsulfonyl)amino]methyl 2- | | | | |
| | methylpropanoate | | | | |
| P-14 | methyl N-[(1S)-2-[[6-chloro-5-(cyclopropylcarbamoyl)-3- | 1.07 | 416.2 [(M + | 1 | |
| | pyridyl]oxy]-1-methyl-ethyl]-N- | | H) + −43] | | |
| | (trifluoromethylsulfonyl)carbamate | | | | |
| P-15 | 2-chloro-5-[(2S)-2-[cyanomethyl | 1.54 | 441.0 | 3 | |
| | (trifluoromethylsulfonyl)amino]propoxy]-N-cyclopropyl- | | | | |
| | pyridine-3-carboxamide | | | | |
| P-16 | 2-chloro-N-cyclopropyl-5-[(2S)-2- | 1.56 | 416 | 3 | |
| | [methyl(trifluoromethylsulfonyl)amino]propoxy]pyridine-3- | | | | |
| | carboxamide | | | | |
| P-17 | 2-chloro-N-cyclopropyl-5-[2-(trifluoromethylsulfonylamino) | 0.99 | 388.2 | 1 | 151-153 |
| | ethoxy]pyridine-3-carboxamide | | | | |
| P-18 | 2-chloro-N-(5-cyano-3-fluoro-2-pyridyl)-5-[(2S)-2- | 1.29 | 482.37 | 5 | |
| | (trifluoromethylsulfonylamino)propoxy]pyridine-3-carboxamide | | | | |
| P-19 | 2-chloro-N-(5-chloro-3-fluoro-2-pyridyl)-5-[(2S)-2- | 1.41 | 491.34 | 5 | |
| | (trifluoromethylsulfonylamino)propoxy]pyridine-3-carboxamide | | | | |
| P-20 | 2-chloro-N-(5-chloro-3-methyl-2-pyridyl)-5-[(2S)-2- | 1.45 | 487.37 | 5 | |
| | (trifluoromethylsulfonylamino)propoxy]pyridine-3-carboxamide | | | | |
| P-21 | 2-chloro-5-[(2S)-2-(trifluoromethylsulfonylamino)propoxy]- | 1.46 | 492.36 | 5 | |
| | N-(2,4,6-trifluorophenyl)pyridine-3-carboxamide | | | | |
| P-22 | 2-chloro-N-(4-cyano-2-fluoro-phenyl)-5-[(2S)-2- | 1.48 | 481.37 | 5 | |
| | (trifluoromethylsulfonylamino)propoxy]pyridine-3-carboxamide | | | | |
| P-23 | 2-chloro-N-(2,4-difluorophenyl)-5-[(2S)-2- | 1.5 | 474.37 | 5 | |
| | (trifluoromethylsulfonylamino)propoxy]pyridine-3-carboxamide | | | | |
| P-24 | 2-chloro-N-(2-fluoro-4-methylsulfonyl-phenyl)-5-[(2S)-2- | 1.33 | 534.38 | 5 | |
| | (trifluoromethylsulfonylamino)propoxy]pyridine-3- | | | | |
| | carboxamide | | | | |
| P-25 | 2-chloro-N-(4-chloro-2-fluoro-phenyl)-5-[(25)-2- | 1.64 | 490.34 | 5 | |
| | (trifluoromethylsulfonylamino)propoxy]pyridine-3-carboxamide | | | | |
| P-26 | N-(1-bicyclo[1.1.1]pentanyl)-2-methyl-5-[(2S)-2- | 1.22 | 408.42 | 5 | |
| | (trifluoromethylsulfonylamino)propoxy]pyridine-3-carboxamide | | | | |
| P-27 | N-[1-[2-(5-cyano-2-pyridyl)-1,2,4-triazol-3-yl]ethyl]-2- | 1.2 | 539.48 | 5 | |
| | methyl-5-[(2S)-2-(trifluoromethylsulfonylamino)propoxy] | | | | |
| | pyridine-3-carboxamide | | | | |
| P-28 | N-[(5-chloro-2-pyridyl)methyl]-2-methyl-5-[(2S)-2- | 1.21 | 467.39 | 5 | |
| | (trifluoromethylsulfonylamino)propoxy]pyridine-3-carboxamide | | | | |
| P-29 | N-[(2E)-2-methoxyimino-1,1-dimethyl-ethyl]-2-methyl-5- | 1.22 | 441.47 | 5 | |
| | [(2S)-2-(trifluoromethylsulfonylamino)propoxy]pyridine-3- | | | | |
| | carboxamide | | | | |
| P-30 | 2-methyl-N-[3-oxo-2-(2,2,2-trifluoroethyl)isoxazolidin-4-yl]- | 1.19 | 509.41 | 5 | |
| | 5-[(2S)-2-(trifluoromethylsulfonylamino)propoxy]pyridine-3- | | | | |
| | carboxamide | | | | |
| P-31 | N-(1-cyclopropylethyl)-2-methyl-5-[(2S)-2- | 1.21 | 410.47 | 5 | |
| | (trifluoromethylsulfonylamino)propoxy]pyridine-3- | | | | |
| | carboxamide | | | | |
| P-32 | N-[(3-chloro-2-pyridyl)methyl]-2-methyl-5-[(2S)-2- | 1.19 | 467.42 | 5 | |
| | (trifluoromethylsulfonylamino)propoxy]pyridine-3-carboxamide | | | | |
| P-33 | N-(2,4-difluorophenyl)-2-methyl-5-[(2S)-2- | 1.34 | 454.39 | 5 | |
| | (trifluoromethylsulfonylamino)propoxy]pyridine-3-carboxamide | | | | |
| P-34 | N-isopropyl-2-methyl-5-[(2S)-2- | 1.06 | 384.43 | 5 | |
| | (trifluoromethylsulfonylamino)propoxy]pyridine-3-carboxamide | | | | |
| P-35 | N-[1-(4-fluorophenyl)-2-oxo-pyrrolidin-3-yl]-2-methyl-5- | 1.24 | 519.5 | 5 | |
| | [(2S)-2-(trifluoromethylsulfonylamino)propoxy]pyridine-3- | | | | |
| | carboxamide | | | | |
| P-36 | N-ethyl-2-methyl-5-[(2S)-2-(trifluoromethylsulfonylamino) | 0.95 | 370.43 | 5 | |
| | propoxy]pyridine-3-carboxamide | | | | |
| P-37 | N-(3,3-difluorocyclobutyl)-2-methyl-5-[(2S)-2- | 1.17 | 432.41 | 5 | |
| | (trifluoromethylsulfonylamino)propoxy]pyridine-3-carboxamide | | | | |
| P-38 | 2-methyl-N-[1-(5-methyl-1,2,4-oxadiazol-3-yl)ethyl]-5-[(2S)- | 1.05 | 452.43 | 5 | |
| | 2-(trifluoromethylsulfonylamino)propoxy]pyridine-3-carboxamide | | | | |
| P-39 | N-(2,2-difluoroethyl)-2-methyl-5-[(2S)-2- | 1.03 | 406.41 | 5 | |
| | (trifluoromethylsulfonylamino)propoxy]pyridine-3-carboxamide | | | | |
| P-40 | N-cyclopropyl-N,2-dimethyl-5-[(2S)-2- | 1.11 | 396.42 | 5 | |
| | (trifluoromethylsulfonylamino)propoxy]pyridine-3-carboxamide | | | | |
| P-41 | N-(cyclopropylmethoxy)-2-methyl-5-[(25)-2- | 1.12 | 412.43 | 5 | |
| | (trifluoromethylsulfonylamino)propoxy]pyridine-3-carboxamide | | | | |
| P-42 | tert-butyl N-[(1S)-2-[[6-chloro-5-(cyclopropylcarbamoyl)-3- | 1.68 | 502.0 | 3 | |
| | pyridyl]oxy]-1-methyl-ethyl]-N-(trifluoromethylsulfonyl)carbamate | | | | |
| P-43 | 5-[(2S)-2-[acetyl(trifluoromethylsulfonyl)amino]propoxy]-2- | 1.04 | 444.2 | 1 | |
| | chloro-N-cyclopropyl-pyridine-3-carboxamide | | | | |
| P-44 | N-benzyl-2-chloro-5-[(2S)-2-(trifluoromethylsulfonylamino) | 1.42 | 452.43 | 5 | |
| | propoxy]pyridine-3-carboxamide | | | | |
| P-45 | 2-chloro-N-(1-cyclopropylethyl)-5-[(2S)-2- | 1.4 | 430.4 | 5 | |
| | (trifluoromethylsulfonylamino)propoxy]pyridine-3-carboxamide | | | | |
| P-46 | 2-chloro-N-[(1-cyanocyclopropyl)methyl]-5-[(25)-2- | 1.18 | 441.36 | 5 | |
| | (trifluoromethylsulfonylamino)propoxy]pyridine-3-carboxamide | | | | |
| P-47 | 2-chloro-N-(1-cyanocyclopropyl)-N-methyl-5-[(2S)-2- | 1.29 | 441.39 | 5 | |
| | (trifluoromethylsulfonylamino)propoxy]pyridine-3-carboxamide | | | | |
| P-48 | 2-chloro-N-(1-cyanocyclobutyl)-5-[(2S)-2- | 1.27 | 441.4 | 5 | |
| | (trifluoromethylsulfonylamino)propoxy]pyridine-3-carboxamide | | | | |
| P-49 | 2-chloro-N-[(4S)-2-ethyl-3-oxo-isoxazolidin-4-yl]-5-[(2S)-2- | 1.17 | 475.41 | 5 | 178-180 |
| | (trifluoromethylsulfonylamino)propoxy]pyridine-3-carboxamide | | | | |
| P-50 | 2-chloro-N-ethyl-5-[(2S)-2- | 1.15 | 390.36 | 5 | |
| | (trifluoromethylsulfonylamino)propoxy]pyridine-3-carboxamide | | | | |
| P-51 | 2-chloro-N-(3-methyltetrahydrofuran-3-yl)-5-[(2S)-2- | 1.2 | 446.43 | 5 | |
| | (trifluoromethylsulfonylamino)propoxy]pyridine-3-carboxamide | | | | |
| P-52 | 2-chloro-N-prop-2-ynyl-5-[(2S)-2- | 1.15 | 400.37 | 5 | |
| | (trifluoromethylsulfonylamino)propoxy]pyridine-3-carboxamide | | | | |
| P-53 | 2-chloro-N-[2-(cyclopropylmethylamino)-2-oxo-ethyl]-5- | 1.18 | 473.42 | 5 | |
| | [(2S)-2-(trifluoromethylsulfonylamino)propoxy]pyridine-3- | | | | |
| | carboxamide | | | | |
| P-54 | 2-chloro-N-[3-oxo-2-(2,2,2-trifluoroethyl)isoxazolidin-4-yl]-5- | 1.32 | 529.33 | 5 | 72-76 |
| | [(2S)-2-(trifluoromethylsulfonylamino)propoxy]pyridine-3- | | | | |
| | carboxamide | | | | |
| P-55 | 2-chloro-N-(2,2-difluoroethyl)-5-[(2S)-2- | 1.2 | 426.35 | 5 | |
| | (trifluoromethylsulfonylamino)propoxy]pyridine-3-carboxamide | | | | |
| P-56 | 2-chloro-N-phenyl-5-[(2S)-2- | 1.45 | 438.4 | 5 | |
| | (trifluoromethylsulfonylamino)propoxy]pyridine-3-carboxamide | | | | |
| P-57 | 2-chloro-N-(1-cyano-1-methyl-ethyl)-5-[(2S)-2- | 1.23 | 429.4 | 5 | 175-178 |
| | (trifluoromethylsulfonylamino)propoxy]pyridine-3-carboxamide | | | | |
| P-58 | 2-chloro-N-cyclopropyl-N-methyl-5-[(2S)-2- | 1.32 | 416.37 | 5 | |
| | (trifluoromethylsulfonylamino)propoxy]pyridine-3-carboxamide | | | | |
| P-59 | 2-chloro-N-(2,2-difluoroethyl)-N-methyl-5-[(2S)-2- | 1.32 | 440.38 | 5 | |
| | (trifluoromethylsulfonylamino)propoxy]pyridine-3-carboxamide | | | | |
| P-60 | 2-chloro-N-(4-fluoro-2-methyl-phenyl)-5-[(2S)-2- | 1.49 | 470.43 | 5 | 146-150 |
| | (trifluoromethylsulfonylamino)propoxy]pyridine-3-carboxamide | | | | |
| P-61 | N-cyclopropyl-5-[(2S)-2-[methoxymethyl | 0.98 | 426.3 | 1 | |
| | (trifluoromethylsulfonyl)amino]propoxy]-2-methyl-pyridine- | | | | |
| | 3-carboxamide | | | | |
| P-62 | N-cyclopropyl-2-methyl-5-[(2S)-2- | 0.98 | 382.3 | 1 | |
| | (trifluoromethylsulfonylamino)propoxy]pyridine-3-carboxamide | | | | |
| P-63 | 2-chloro-5-[(2S)-2- | 1.06 | 488.0 | 1 | |
| | [methoxymethyl(trifluoromethylsulfonyl)amino]propoxy]-N- | | | | |
| | (2,2,2-trifluoroethyl)pyridine-3-carboxamide | | | | |
| P-64 | 2-chloro-N-cyclopropyl-5-[(2S)-2- | 1.10 | 446.1 | 1 | |
| | [methoxymethyl(trifluoromethylsulfonyl)amino]propoxy] | | | | |
| | pyridine-3-carboxamide | | | | |
| P-65 | 2-chloro-N-[(4R)-2-ethyl-3-oxo-isoxazolidin-4-yl]-5-[(2S)-2- | 1.05 | 475.3 | 1 | 115-118 |
| | (trifluoromethylsulfonylamino)propoxy]pyridine-3-carboxamide | | | | |
| P-66 | N-(1-bicyclo[1.1.1]pentanyl)-5-methyl-2-[(2S)-2- | 1.45 | 408.16 | 5 | |
| | (trifluoromethylsulfonylamino)propoxy]pyridine-4-carboxamide | | | | |
| P-67 | N-(1-cyanocyclopropyl)-N,5-dimethyl-2-[(2S)-2- | 1.31 | 421.16 | 5 | |
| | (trifluoromethylsulfonylamino)propoxy]pyridine-4-carboxamide | | | | |
| P-68 | N-(4-ethylthiazol-5-yl)-5-methyl-2-[(2S)-2- | 1.37 | 453.14 | 5 | |
| | (trifluoromethylsulfonylamino)propoxy]pyridine-4-carboxamide | | | | |
| P-69 | N-(1,3-dihydroisobenzofuran-5-yl methyl)-5-methyl-2-[(2S)-2- | 1.36 | 474.19 | 5 | |
| | (trifluoromethylsulfonylamino)propoxy]pyridine-4-carboxamide | | | | |
| P-70 | 5-methyl-N-[2-oxo-1-(2,2,2-trifluoroethyl)pyrrolidin-3-yl]-2- | 1.3 | 507.15 | 5 | |
| | [(2S)-2-(trifluoromethylsulfonylamino)propoxy]pyridine-4- | | | | |
| | carboxamide | | | | |
| P-71 | N-[2-(2-fluorophenoxy)ethyl]-5-methyl-2-[(25)-2- | 1.51 | 480.17 | 5 | |
| | (trifluoromethylsulfonylamino)propoxy]pyridine-4-carboxamide | | | | |
| P-72 | N-(5-methoxy-1,1-dimethyl-pent-2-ynyl)-5-methyl-2-[(2S)-2- | 1.47 | 466.22 | 5 | |
| | (trifluoromethylsulfonylamino)propoxy]pyridine-4-carboxamide | | | | |
| P-73 | N-(1-cyclopropylethyl)-5-methyl-2-[(25)-2- | 1.44 | 410.17 | 5 | |
| | (trifluoromethylsulfonylamino)propoxy]pyridine-4-carboxamide | | | | |
| P-74 | N-[1-(2-ethylthiazol-4-yl)ethyl]-5-methyl-2-[(2S)-2- | 1.49 | 481.17 | 5 | |
| | (trifluoromethylsulfonylamino)propoxy]pyridine-4-carboxamide | | | | |
| P-75 | N-(3-cyano-2-fluoro-phenyl)-5-methyl-2-[(2S)-2- | 1.49 | 461.13 | 5 | |
| | (trifluoromethylsulfonylamino)propoxy]pyridine-4-carboxamide | | | | |
| P-76 | 5-methyl-N-[1-(3-pyridyl)ethyl]-2-[(25)-2- | 0.93 | 447.17 | 5 | |
| | (trifluoromethylsulfonylamino)propoxy]pyridine-4-carboxamide | | | | |
| P-77 | N-benzyl-5-methyl-2-[(2S)-2- | 1.47 | 432.16 | 5 | |
| | (trifluoromethylsulfonylamino)propoxy]pyridine-4-carboxamide | | | | |
| P-78 | N-[(4R)-2-ethyl-3-oxo-isoxazolidin-4-yl]-2-[(2S)-2- | 1.59 | 499 | 3 | |
| | [methoxymethyl(trifluoromethylsulfonyl)amino]propoxy]-5- | | | | |
| | methyl-pyridine-4-carboxamide | | | | |
| P-79 | N-(1-cyanocyclopropyl)-2-[(2S)-2- | 1.03 | 451.6 | 1 | |
| | [methoxymethyl(trifluoromethylsulfonyl)amino]propoxy]-5- | | | | |
| | methyl-pyridine-4-carboxamide | | | | |
| P-80 | 5-methyl-N-(2,2,2-trifluoroethyl)-2-[(2S)-2- | 1.59 | 424.1 | 3 | 120-124 |
| | (trifluoromethylsulfonylamino)propoxy]pyridine-4-carboxamide | | | | |
| P-81 | 5-bromo-N-cyclopropyl-2-[(2S)-2- | 1.15 | 490.0 | 1 | |
| | [methoxymethyl(trifluoromethylsulfonyl)amino]propoxy] | | | | |
| | pyridine-4-carboxamide | | | | |
| P-82 | 5-bromo-N-(1-cyanocyclopropyl)-2-[(2S)-2- | 1.09 | 515.0 | 1 | |
| | [methoxymethyl(trifluoromethylsulfonyl)amino]propoxy] | | | | |
| | pyridine-4-carboxamide | | | | |
| P-83 | 5-bromo-N-cyclopropyl-2-[(2S)-2- | 1.11 | 446.2 | 1 | 138-141 |
| | (trifluoromethylsulfonylamino)propoxy]pyridine-4-carboxamide | | | | |
| P-84 | 5-bromo-N-[(4R)-2-ethyl-3-oxo-isoxazolidin-4-yl]-2-[(2S)-2- | 1.09 | 519.0 | 1 | |
| | (trifluoromethylsulfonylamino)propoxy]pyridine-4-carboxamide | | | | |
| P-85 | 5-bromo-N-(2,2,2-trifluoroethyl)-2-[(2S)-2- | 1.13 | 488.1 | 1 | 125-128 |
| | (trifluoromethylsulfonylamino)propoxy]pyridine-4-carboxamide | | | | |
| P-86 | 5-bromo-N-(1-cyanocyclopropyl)-2-[(25)-2- | 1.10 | 471.2 | 1 | 137-140 |
| | (trifluoromethylsulfonylamino)propoxy]pyridine-4-carboxamide | | | | |
| P-87 | 5-chloro-N-cyclopropyl-2-[(25)-2- | 0.91 | 402 | 4 | 122-126 |
| | (trifluoromethylsulfonylamino)propoxy]pyridine-4-carboxamide | | | | |
| P-88 | 3-chloro-N-cyclopropyl-6-[(2S)-2- | 0.81 | 402.99 | 4 | |
| | (trifluoromethylsulfonylamino)propoxy]pyridazine-4-carboxamide | | | | |
| P-89 | N-(1-bicyclo[1.1.1]pentanyl)-3-chloro-6-[(2S)-2- | 0.92 | 429 | 4 | |
| | (trifluoromethylsulfonylamino)propoxy]pyridazine-4-carboxamide | | | | |

### Biological Examples

Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 24 ppm, 12.5 ppm, 6 ppm, 3 ppm, 1.5 ppm, 0.8 ppm or 0.2 ppm.

### Example B1: Bemisia tabaci (Cotton White Fly): Feeding/Contact Activity

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm:
P-1, P-2, P-3, P-6, P-7, P-8, P-9, P-12, P-13, P-14, P-15, P-17, P-20, P-23, P-28, P-31, P-43, P-46, P-47, P-52, P-54, P-56, P-57, P-58, P-63, P-64, P-70, P-82, P-86

### Example B2: Chilo suppressalis (Striped Rice Stemborer)

24-well microtiter plates with artificial diet were treated with aqueous test solutions prepared from 10′000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.

The following compounds resulted in at least 80% control at an application rate of 200 ppm:
P-2, P-63

### Example B3: Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm:
P-1, P-2, P-7, P-21, P-22, P-64, P-84

### Example B4: Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm:
P-1, P-2, P-3, P-6, P-8, P-12, P-14, P-15, P-16, P-42, P-43, P-50, P-58, P-61, P-63, P-64, P-66, P-71

### Example B5: Frankliniella occidentalis (Western Flower Thrips): Feeding/Contact Activity

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm:
P-1, P-2, P-3, P-6, P-7, P-9, P-30, P-35, P-42, P-43, P-47, P-48, P-62, P-64

### Example B6: Myzus persicae (Green Peach Aphid): Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm:
P-1, P-2, P-3, P-4, P-5, P-6, P-8, P-9, P-11, P-12, P-13, P-14, P-15, P-16, P-17, P-26, P-32, P-34, P-36, P-40, P-42, P-43, P-45, P-46, P-47, P-48, P-49, P-50, P-52, P-53, P-54, P-55, P-57, P-58, P-59, P-64, P-67, P-68, P-69, P-70, P-72, P-73, P-75, P-76, P-78, P-83, P-87

### Example B7: Myzus persicae (Green Peach Aphid). Systemic Activity

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm:
P-1, P-2, P-5, P-6, P-7, P-8, P-10, P-13, P-14, P-15, P-16, P-26, P-28, P-43, P-48, P-54, P-57, P-58, P-64, P-83

### Example B8: Myzus persicae (Green Peach Aphid). Intrinsic Activity

Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds resulted in at least 80% mortality at a test rate of 12 ppm:
P-1, P-2, P-3, P-4, P-5, P-6, P-7, P-8, P-9, P-10, P-11, P-12, P-13, P-14, P-15, P-16, P-17, P-19, P-20, P-23, P-26, P-42, P-43, P-45, P-47, P-48, P-50, P-54, P-55, P-57, P-58, P-60, P-63, P-64, P-81, P-83, P-87

### Example B9: Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm:
P-1, P-5, P-23, P-25, P-27, P-38, P-41, P-60

### Example B10: Spodoptera littoralis (Eaytian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm:
P-2, P-60, P-65

### Example B11: Tetranychus urticae (Two-Spotted Spider Mite): Feeding/Contact Activity

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm:
P-1, P-3, P-4, P-5, P-6, P-8, P-10, P-11, P-12, P-13, P-14, P-15, P-16, P-18, P-20, P-21, P-22, P-23, P-24, P-25, P-26, P-31, P-32, P-33, P-37, P-39, P-42, P-43, P-44, P-45, P-48, P-49, P-51, P-52, P-56, P-57, P-60, P-63, P-64, P-65, P-77, P-78, P-79, P-80, P-82, P-87

### Example B12: Myzus persicae (Green Peach Aphid) Feeding/Contact Activity

Eggplant leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% growth inhibition at an application rate of 200 ppm:
P89

1. A compound of the formula (I) 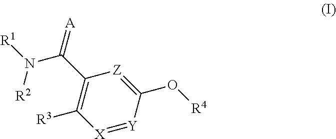
wherein:
A is O, or S;
X is N, or CR⁸;
Y is N or CR⁸;
Z is N or CR⁸;
with the provision that at least one of X, Y, Z is N;
R¹ is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkoxy-C₁-C₆-alkyl, C₃-C₆-cycloalkyl-C₁-C₆-alkyl, C₃-C₆-halocycloalkyl-C₁-C₆-alkyl, cyano-C₁-C₆-alkyl, cyano-C₃-C₆-cycloalkyl, cyano-C₃-C₆-cycloalkyl-C₁-C₆-alkyl, cyano-heterocycloalkyl, hydroxy-C₃-C₆-cycloalkyl, C₁-C₆-alkyl-carbonyloxy-C₃-C₆-cycloalkyl, C₁-C₆-alkylsulfonyloxy-C₃-C₆-cycloalkyl, C₁-C₆-alkoxy-C₃-C₆-cycloalkyl, C₁-C₆-haloalkoxy-C₃-C₆-cycloalkyl, C₁-C₆-alkylthio-C₃-C₆-cycloalkyl, C₁-C₆-haloalkylthio-C₃-C₆-cycloalkyl, C₁-C₆-alkylsulfinyl-C₃-C₆-cycloalkyl, C₁-C₆-haloalkylsulfinyl-C₃-C₆-cycloalkyl, C₁-C₆-alkylsulfonyl-C₃-C₆-cycloalkyl, C₁-C₆-haloalkylsulfonyl-C₃-C₆-cycloalkyl, C₂-C₆-alkynyl-C₃-C₆-cycloalkyl, C₁-C₆-alkoxycarbonyl-C₃-C₆-cycloalkyl, carboxy-C₃-C₆-cycloalkyl, carbamoyl-C₃-C₆-cycloalkyl, C₁-C₆-alkylaminocarbonyl-C₃-C₆-cycloalkyl (which may be mono- or poly-substituted by R⁸), C₄-C₈-bicycloalkyl, C₁-C₆-alkoxyimino, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono- or poly-substituted by R¹⁰), heteroaryl-C₃-C₆-cycloalkyl (the heteroaryl may be mono- or poly-substituted by R¹⁰), heterocycloalkyl (which may be substituted by R⁸), heterocycloalkyl-C₁-C₆-alkyl (which may be substituted by R⁸), or heterocycloalkyl-C₃-C₆-cycloalkyl (the heterocycloalkyl may be substituted by R⁸);
R² is hydrogen, C₁-C₆-alkyl, C₁-C₆-haloalkyl, C₃-C₆-cycloalkyl, C₃-C₆-halocycloalkyl, C₂-C₆-alkenyl, C₂-C₆-alkenyl, C₂-C₆-haloalkenyl, C₂-C₆-alkynyl, C₂-C₆-haloalkynyl, C₁-C₆-alkoxy, C₁-C₆-alkoxy-C₁-C₆-alkyl, C₁-C₆-haloalkoxy, C₃-C₆-cycloalkyl-C₁-C₆-alkyl, C₃-C₆-halocycloalkyl-C₁-C₆-alkyl, phenyl (which may be mono- or poly-substituted by R¹⁰), phenyl-C₁-C₆-alkyl (the phenyl may be mono- or poly-substituted by R¹⁰), heteroaryl (the heteroaryl may be mono- or poly-substituted by R¹⁰), heteroaryl-C₁-C₃-alkyl (the heteroaryl may be mono-substituted or polysubstituted by R¹⁰), cyano, cyano-C₁-C₆-alkyl, cyano-C₃-C₆-cycloalkyl, hydroxy, formyl, C₁-C₆-alkylcarbonyl, C₁-C₆-alkoxycarbonyl, C₃-C₆-cycloalkylcarbonyl, benzoyl that is mono or polysubstituted by R¹⁰, C₁-C₆-haloalkoxycarbonyl, C₂-C₆-alkenyloxycarbonyl, carbamoyl, C₁-C₆-alkylaminocarbonyl (the amino group may be substituted by R⁸), C₁-C₆-alkoxycarbonyl C₁-C₆-alkylamino (the amino group may be substituted by R⁸), C₁-C₆-alkoxycarbonyl amino (the amino group may be substituted by R⁸), (C₁-C₆-alkylthio)carbonyl, (C₁-C₆-alkyl)thiocarbonyl, (C₁-C₆-alkoxy)thiocarbonyl, thiocarbamoyl, C₁-C₆-alkylamino-thiocarbonyl (the amino group may be substituted by R⁸), C₁-C₆-alkylsulfonyl, C₁-C₆-haloalkylsulphonyl, sulfamoyl, C₁-C₆-alkylaminosulfonyl (the amino group may be substituted by R⁸), C₁-C₆-alkoxycarbonyl-C₁-C₆-alkyl, aminocarbonyl-C₁-C₆-alkyl, C₁-C₆-alkylaminocarbonyl-C₁-C₆-alkyl (the amino group may be substituted by R⁸), C₃-C₆-cycloalkylaminocarbonyl-C₁-C₆-alkyl (the amino group may be substituted by R⁸), C₁-C₆-haloalkylaminocarbonyl-C₁-C₆-alkyl (the amino group may be substituted by R⁸), C₁-C₆-alkylcarbonyl-C₁-C₆-alkyl, C₁-C₆-haloalkylcarbonyl-C₁-C₆-alkyl, C₁-C₆-alkylcarbonyloxy-C₁-C₆-alkyl, C₁-C₆-alkoxycarbonyloxy-C₁-C₆-alkyl, C₁-C₆-alkoxycarbonylamino-C₁-C₆-alkyl (the amino group may be substituted by R⁸), hydroxyimino, hydroxyamino-C₁-C₆-alkyl, C₁-C₆-alkoxyimino, C₁-C₆-alkoxyimino-C₁-C₆-alkyl, or C₁-C₆-haloalkoxyimino-C₁-C₆-alkyl;
R³ is halogen, cyano, C₁-C₆-alkyl, C₁-C₆-alkoxy, C₃-C₆-cycloalkyl, C₁-C₆-haloalkyl, or C₁-C₆-haloalkoxy;
R⁴ is 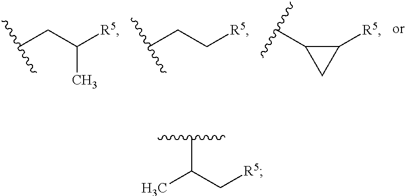
or
R⁵ is C₁-C₃-alkylsulfonylamino, C₁-C₃-haloalkylsulfonylamino, or C₃-C₆-cycloalkylsulfonylamino, wherein any of said amino-groups is unsubstituted or substituted by R⁶; and wherein any of said cycloalkyl-groups may be mono- or polysubstituted by R⁹;
R⁶ is C₁-C₆-alkyl, C₁-C₆-alkoxy, C₁-C₆-alkoxy-C₁-C₆-alkyl, cyano-C₁-C₆-alkyl, C₁-C₆-haloalkyl, C₃-C₆-cycloalkyl, C₃-C₆-cycloalkyl-C₁-C₆-alkyl, C₁-C₆-alkylcarbonyl, or C₁-C₆-alkoxycarbonyl;
R⁸ is hydrogen, halogen, C₁-C₃-alkyl, C₁-C₃-alkoxy, C₁-C₃-haloalkyl, or cyano;
R⁹ is halogen, C₁-C₃-alkyl, C₁-C₃-alkoxy, C₁-C₃-haloalkyl, or cyano; and
R¹⁰ is halogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulphonyl, C₁-C₆-alkylthio-C₁-C₆-alkyl, C₁-C₆-haloalkylthio-C₁-C₆-alkyl, C₁-C₆-alkylsulfonyloxy, C₁-C₆-haloalkylsulfonyloxy, phenyl (which may be mono- or poly-substituted independently selected from halogen, C₁-C₆-alkyl and C₁-C₆-haloalkyl), phenyl-C₁-C₆-alkyl, phenyl-C₁-C₆-alkoxy, cyano, and nitro;
or an agrochemically acceptable salt, stereoisomer, enantiomer, tautomer and N-oxide of the compound of formula (I).

2. The compound according to claim 1, wherein the compound of formula (I) is represented by the formula (I-1), (I-2), (I-3), or (I-4), where A, R¹, R², R³ and R⁴ are as defined in claim 1.

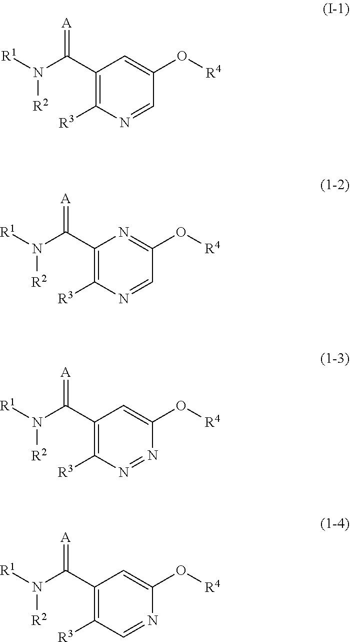

3. The compound according to claim 1, wherein the compound of formula (I) is represented by the formula (I-1a), (I-2a), (I-3a), or (I-4a), where R¹, R², R³ and R⁶ are as defined in claim 1; and wherein R⁷ is, independent of (I-1a), (I-2a), (I-3a), and (I-4a), selected from C₁-C₃-alkyl, C₁-C₃-haloalkyl, or C₃-C₆-cycloalkyl, wherein said cycloalkyl-group may be mono or polysubstituted by R⁹, wherein R⁹ is as defined in claim 1.

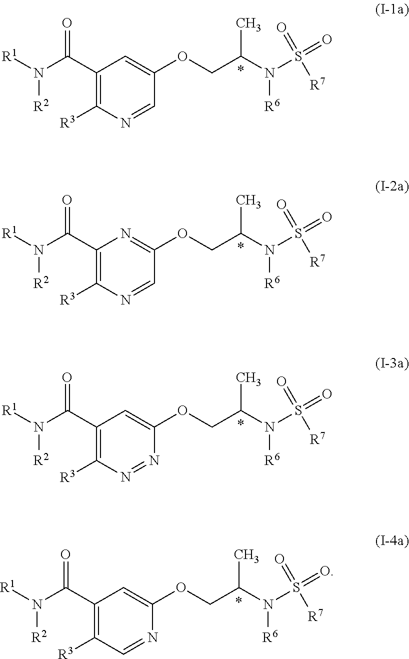

4. The compounds according to claim 3 wherein the compound of formula (I) represented by the formula (I-1a), (I-2a), (I-3a), or (I-4a), is an (S) enantiomer.

5. The compound according to claim 1, wherein R¹ is hydrogen, C₁-C₆-alkyl, C₁-C₆-haloalkyl, C₂-C₆-alkynyl, C₃-C₆-cycloalkyl, cyano-C₃-C₆-cycloalkyl, C₃-C₆-halocycloalkyl, C₃-C₆-cycloalkyl-C₁-C₆-alkyl, heterocyclyl, or C₁-C₆-alkoxy-C₃-C₆-cycloalkyl; and R² is hydrogen, C₁-C₆-alkyl, C₁-C₆-haloalkyl, C₃-C₆-cycloalkyl, C₃-C₆-cycloalkyl-C₁-C₆-alkyl, C₁-C₆-alkoxy-C₃-C₆-cycloalkyl, C₁-C₆-alkoxy-C₁-C₆-alkyl, or C₁-C₆-alkylcarbonyl.

6. The compound according to claim 1, wherein R¹ is C₁-C₃-alkyl, C₁-C₃-haloalkyl, C₂-C₄-alkynyl, C₃-C₄-cycloalkyl, C₃-C₄-halocycloalkyl, C₃-C₄-cycloalkyl-C₁-C₃-alkyl, 4-membered heterocyclyl having a SO₂, or cyano-C₃-C₄-cycloalkyl; and R² is hydrogen, C₁-C₃-alkyl, C₁-C₃-alkylcarbonyl, or C₁-C₃-alkoxycarbonyl.

7. The compound according to claim 1, wherein R³ is halogen, C₁-C₆-alkyl, or cyano.

8. The compound according to claim 7, wherein R³ is chlorine, bromine or methyl.

9. The compound according to claim 1, wherein R⁵ is isopropylsulfonylamino, difluoromethylsulfonylamino, trifluoromethylsulfonylamino, difluoromethylsulfonylamino, or cyclopropylsulfonylamino.

10. The compound according to claim 1, wherein R⁶ is hydrogen, C₁-C₃-alkyl, C₁-C₃-alkoxy-C₁-C₃-alkyl, C₃-C₄-cycloalkyl-C₁-C₃-alkyl, or C₁-C₃-alkylcarbonyl.

11. A process for preparing a compound of formula (I) (wherein A is O) comprising transformation of a compound of formula (II), wherein R³, R⁴, X, Y, Z are as defined in claim 1, with a compound of formula (III), wherein Rr and R² are as defined in claim 1, via an intermediate acid chloride or activated acylating agent of formula (IV):

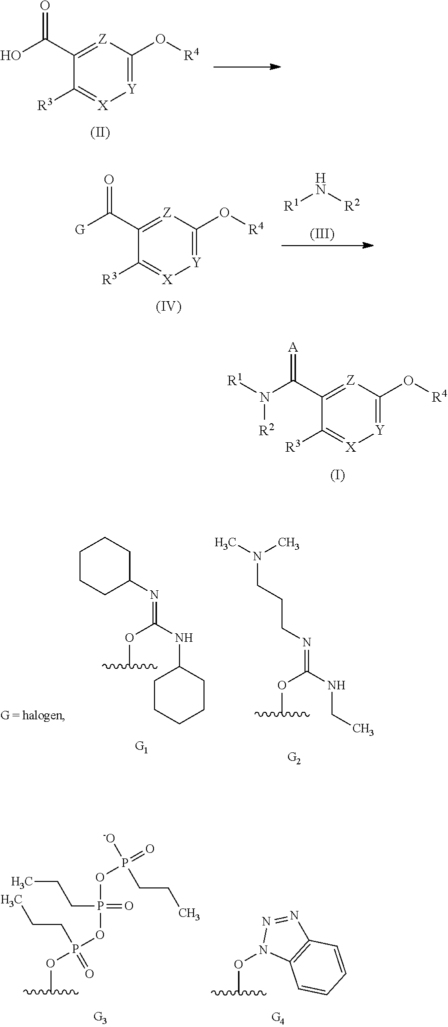

12. A composition comprising a compound as defined in claim 1, one or more auxiliaries and diluent, and optionally one or more other active ingredient.

13. A method of combating and controlling insects, acarines or molluscs which comprises applying to a pest, to a locus of a pest, or to a plant susceptible to attack by a pest an insecticidally, acaricidally, or molluscicidally effective amount of a compound of formula I as defined in claim 1 or a composition thereof.

14. A method for the protection of plant propagation material from the attack by insects, acarines, or molluscs, which comprises treating the propagation material or the site, where the propagation material is planted, with an effective amount of a compound of formula I as defined in claim 1 or a composition thereof.

15. A plant propagation material, such as a seed, comprising, or treated with or adhered thereto, a compound of formula I as defined in claim 1.
